(19) United States
(12) Patent Application Publication
(10) Pub. No.: US 2008/0262228 A1
(43) Pub. Date: Oct. 23, 2008

(54) Metabolites of 5-fluoro-8- quinoline and methods of preparation and uses thereof

(75) Inventors: Jianyao Wang, Monmouth Junction, NJ (US); Alana Upthagrove, Bedminster, NJ (US); Lin Deng, Mont Clare, PA (US); Wayne Everett Childers, New Hope, PA (US); Zhongqi Shen, Plainsboro, NJ (US); William Demaio, Collegeville, PA (US); Robin D. Moore, Yardley, PA (US); Zeen Tong, Collegeville, PA (US); Li Shen, Audubon, PA (US); Pixu Li, Nanuet, NY (US); Michael K. May, Nyack, NY (US)

Correspondence Address: WilmerHale/Wyeth, 60 STATE STREET, BOSTON, MA, 02109, US

(73) Assignee: Wyeth, Madison, NJ (US)

(21) Appl. No.: 11/986,895

(22) Filed: Nov. 27, 2007

Related U.S. Application Data
(60) Provisional application No. 60/861,408, filed on Nov. 28, 2006.

(51) Int. Cl. A61K 31/496; C07D 401/14; A61P 25/00

(52) U.S. Cl. 544/363; 514/253.06

(57) ABSTRACT

The present invention relates to novel metabolites of 5-Fluoro-8-{4-[4-(6-methoxyquinolin-8-yl)piperazin-1-yl]piperidin-1-yl}quinoline, which can be useful in treating CNS disorders. The present invention further relates to processes for their preparation, to pharmaceutical compositions comprising them, and to methods of using them.

### CROSS REFERENCE TO RELATED APPLICATIONS

This application claims the benefit of priority under 35 U.S.C. §119(e) to U.S. Patent Application Ser. No. 60/861,408 filed on Nov. 28, 2006 and is hereby incorporated by reference in its entirety.

### FIELD OF THE INVENTION

The present invention relates to novel metabolites of 5-Fluoro-8-{4-[4-(6-methoxyquinolin-8-yl)piperazin-1-yl]piperidin-1-yl}quinoline, which can be useful in treating CNS disorders; to processes for their preparation; to pharmaceutical compositions comprising them; and to methods of using them.

### BACKGROUND OF THE INVENTION

Certain N-aryl-piperazine derivatives possess pharmaceutical activity. In particular, certain N-aryl piperazine derivatives act on the CNS(CNS) by binding to 5-HT receptors. In pharmacological testing, it has been shown that the certain N-aryl-piperazine derivatives bind to receptors of the 5-HT_{1A} type. Many of the N-aryl piperazine derivatives exhibit activity as 5-HT_{1A} antagonists. See, for example, W. C. Childers, et al., J. Med. Chem., 48: 3467-3470 (2005), U.S. Pat. Nos. 6,465,482, 6,127,357, 6,469,007, and 6,586,436, PCT Publication No. WO 97/03982, and co-pending U.S. patent application Ser. No. 11/450,942, filed on Jun. 9, 2006, published as US2007/0027160A1, the disclosures of which are incorporated herein by reference in their entireties.

The compound 5-Fluoro-8-{4-[4-(6-methoxyquinolin-8-yl)piperazin-1-yl]piperidin-1-yl}quinoline (hereinafter “COMPOUND I”) has the following structure:

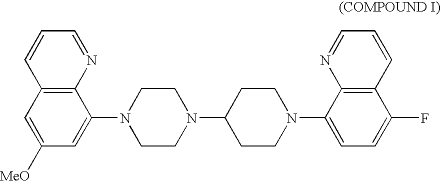

and is a potent 5-HT_{1A} receptor antagonist that displays cognitive enhancing effects in animal models of learning and memory. Thus, COMPOUND I can be useful to treat a wide variety of CNS diseases, disorders and conditions, such as cognition disorders, anxiety disorders, and depression.

COMPOUND I is converted, in several in vitro models, into various metabolites. It can be seen that these metabolites are of interest in treating those CNS diseases, disorders, or conditions treatable by COMPOUND I itself or as a prodrug, which converts to COMPOUND I. These metabolites could also be useful for further studying the effects of COMPOUND I. This invention is directed to these, as well as other, important ends.

### SUMMARY OF THE INVENTION

In one aspect, the present invention provides a metabolite of 5-Fluoro-8-{4-[4-(6-methoxyquinolin-8-yl)piperazin-1-yl]piperidin-1-yl}quinoline (COMPOUND I), or an enantiomer, diastereomer, tautomer, or pharmaceutically acceptable salt or solvate of the metabolite. In another aspect, the present invention provides a metabolite of COMPOUND I made by treating 5-Fluoro-8-{4-[4-(6-methoxyquinolin-8-yl)piperazin-1-yl]piperidin-1-yl}quinoline or its pharmaceutically acceptable salt with rat, mouse, dog, monkey or human liver microsomes. In yet another aspect, the present invention provides a metabolite of COMPOUND I made by treating 5-Fluoro-8-{4-[4-(6-methoxyquinolin-8-yl)piperazin-1-yl]piperidin-1-yl}quinoline or its pharmaceutically acceptable salt with rat, mouse, dog, monkey or human liver S9 fractions. In yet another aspect, the present invention provides a metabolite of COMPOUND I made by treating 5-Fluoro-8-{4-[4-(6-methoxyquinolin-8-yl)piperazin-1-yl]piperidin-1-yl}quinoline or its pharmaceutically acceptable salt with cryopreserved rat, dog, or human hepatocytes. In yet another aspect, the present invention provides a metabolite of COMPOUND I made by administering 5-Fluoro-8-{4-[4-(6-methoxyquinolin-8-yl)piperazin-1-yl]piperidin-1-yl}quinoline or its pharmaceutically acceptable salt to a mammal, such as a rat, mouse, dog, monkey or human. In yet another aspect, the present invention provides a metabolite of 5-Fluoro-8-{4-[4-(6-methoxyquinolin-8-yl)piperazin-1-yl]piperidin-1-yl}quinoline (COMPOUND I), wherein the metabolite is not isolated.

In another aspect, the present invention provides a purified and isolated metabolite of 5-Fluoro-8-{4-[4-(6-methoxyquinolin-8-yl)piperazin-1-yl]piperidin-1-yl}quinoline (COMPOUND I), or an enantiomer, diastereomer, tautomer, or pharmaceutically acceptable salt or solvate of the metabolite. In another aspect, the present invention provides a purified and isolated metabolite of COMPOUND I made by treating 5-Fluoro-8-{4-[4-(6-methoxyquinolin-8-yl)piperazin-1-yl]piperidin-1-yl}quinoline or its pharmaceutically acceptable salt with rat, mouse, dog, monkey or human liver microsomes. In yet another aspect, the present invention provides a purified and isolated metabolite of COMPOUND I made by treating 5-Fluoro-8-{4-[4-(6-methoxyquinolin-8-yl)piperazin-1-yl]piperidin-1-yl}quinoline or its pharmaceutically acceptable salt with rat, mouse, dog, monkey or human S9 fractions. In yet another aspect, the present invention provides a purified and isolated metabolite of COMPOUND I made by treating 5-Fluoro-8-{4-[4-(6-methoxyquinolin-8-yl)piperazin-1-yl]piperidin-1-yl}quinoline or its pharmaceutically acceptable salt with cryopreserved rat, dog, or human hepatocytes. In yet another aspect, the present invention provides a purified and isolated metabolite of COMPOUND I made by administering 5-Fluoro-8-{4-[4-(6-methoxyquinolin-8-yl)piperazin-1-yl]piperidin-1-yl}quinoline or its pharmaceutically acceptable salt to a mammal, such as a rat, mouse, dog, monkey or human.

In yet another aspect, the present invention provides a metabolite of COMPOUND I, preferably a purified and isolated metabolite of COMPOUND I, which exhibits a mass spectral peak [M+H]⁺ at m/z 244. In yet another aspect, the present invention provides a metabolite of COMPOUND I, preferably a purified and isolated metabolite of COMPOUND I, which exhibits a mass spectral peak [M+H]⁺ at m/z 662. In yet another aspect, the present invention provides a metabolite of COMPOUND I, preferably a purified and isolated metabolite of COMPOUND I, which exhibits a mass spectral peak [M+H]⁺ at m/z 680. In yet another aspect, the present invention provides a metabolite of COMPOUND I, preferably a purified and isolated metabolite of COMPOUND I, which exhibits a mass spectral peak [M+H]⁺ at m/z 646. In yet another aspect, the present invention provides a metabolite of COMPOUND I, preferably a purified and isolated metabolite of COMPOUND I, which exhibits a mass spectral peak [M+H]⁺ at m/z 506. In yet another aspect, the present invention provides a metabolite of COMPOUND I, preferably a purified and isolated metabolite of COMPOUND I, which exhibits a mass spectral peak [M+H]⁺ at m/z 664.

In yet another aspect, the present invention provides a metabolite of COMPOUND I, preferably a purified and isolated metabolite of COMPOUND I, which exhibits a mass spectral peak [M+H]⁺ at m/z 484. In yet another aspect, the present invention provides a metabolite of COMPOUND I, preferably a purified and isolated metabolite of COMPOUND I, which exhibits a mass spectral peak [M+H]⁺ at m/z 504. In yet another aspect, the present invention provides a metabolite of COMPOUND I, preferably a purified and isolated metabolite of COMPOUND I, which exhibits a mass spectral peak [M+H]⁺ at m/z 470. In yet another aspect, the present invention provides a metabolite of COMPOUND I, preferably a purified and isolated metabolite of COMPOUND I, which exhibits a mass spectral peak [M+H]⁺ at m/z 488. In yet another aspect, the present invention provides a metabolite of COMPOUND I, preferably a purified and isolated metabolite of COMPOUND I, which exhibits a mass spectral peak [M+H]⁺ at m/z 458. In yet another aspect, the present invention provides a metabolite of COMPOUND I, preferably a purified and isolated metabolite of COMPOUND I, which exhibits a mass spectral peak [M+H]⁺ at m/z 472. In yet another aspect, the present invention provides a metabolite of COMPOUND I, preferably a purified and isolated metabolite of COMPOUND I, which exhibits a mass spectral peak [M+H]⁺ at m/z 568. In yet another aspect, the present invention provides a metabolite of COMPOUND I, preferably a purified and isolated metabolite of COMPOUND I, which exhibits a mass spectral peak [M+H]⁺ at m/z 634. In yet another aspect, the present invention provides a metabolite of COMPOUND I, preferably a purified and isolated metabolite of COMPOUND I, which exhibits a mass spectral peak [M+H]⁺ at m/z 538.

In a further aspect, the present invention provides a metabolite of COMPOUND I, preferably a purified and isolated metabolite of COMPOUND I, having the following structure:

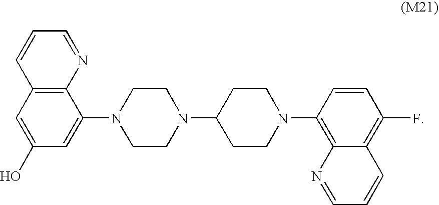

In yet another aspect, the present invention provides a metabolite of COMPOUND I, preferably a purified and isolated metabolite of COMPOUND I, having the following structure:

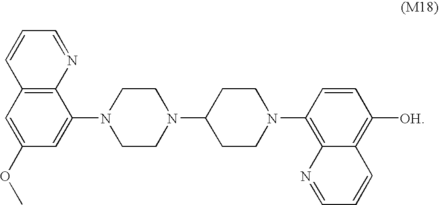

In yet another aspect, the present invention provides a metabolite of COMPOUND I, preferably a purified and isolated metabolite of COMPOUND I, having the following structure, wherein the hydroxyl group may be substituted at any available position of the 6-methoxy-quinoline ring (within the dotted box):

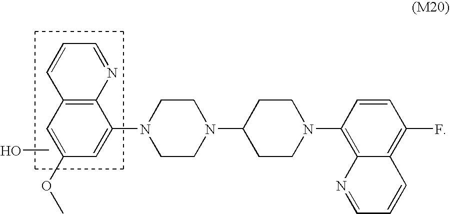

In yet another aspect, the present invention provides a metabolite of COMPOUND I, preferably a purified and isolated metabolite of COMPOUND I, having the following structure, wherein the hydroxyl group may be substituted at any available position of the 5-fluoro-quinoline ring:

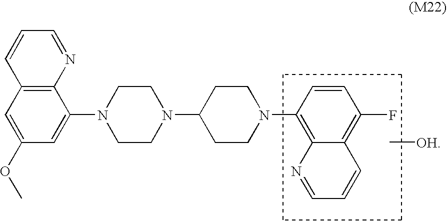

In yet another aspect, the present invention provides a metabolite of COMPOUND I, preferably a purified and isolated metabolite of COMPOUND I, having the following structure, wherein the 5-keto may alternatively be substituted at 2, 3, 4, or 7 position of the quinone ring:

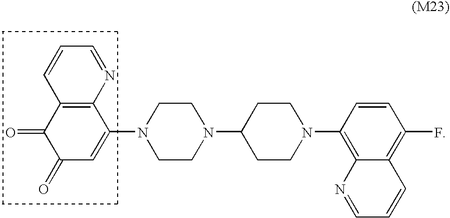

In yet another aspect, the present invention provides a metabolite of COMPOUND I, preferably a purified and isolated metabolite of COMPOUND I, having the following structure:

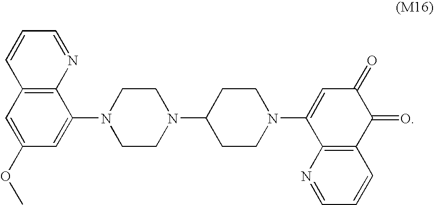

In yet another aspect, the present invention provides a metabolite of COMPOUND I, preferably a purified and isolated metabolite of COMPOUND I, having the following structure, wherein each of the two hydroxyl groups may be substituted at any available position of its respective quinoline ring:

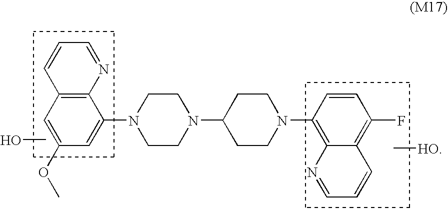

In yet another aspect, the present invention provides a metabolite of COMPOUND I, preferably a purified and isolated metabolite of COMPOUND I, having the following structure, wherein the dihydrodiol may be substituted at any available position of the 5-fluoro-quinoline ring:

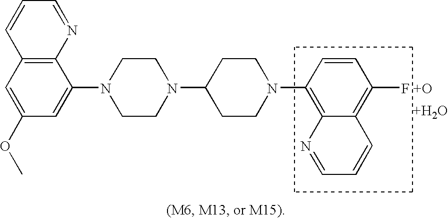

In yet another aspect, the present invention provides a metabolite of COMPOUND I, preferably a purified and isolated metabolite of COMPOUND I, having the following structure,

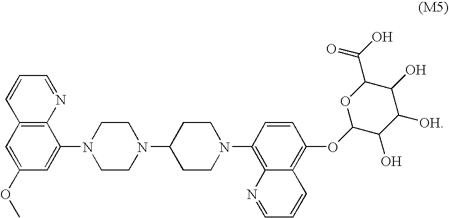

In yet another aspect, the present invention provides a metabolite of COMPOUND I, preferably a purified and isolated metabolite of COMPOUND I, having the following structure, wherein in addition to the 5-OH group in the quinoline ring (within the dotted box), the other hydroxyl group may be substituted at any available position of the quinoline ring, and wherein the glucuronide may be attached to either one of the two hydroxyl groups.

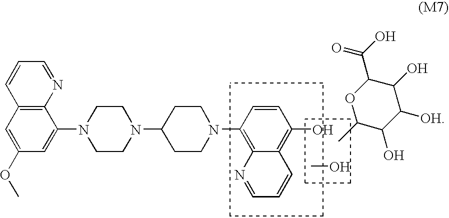

In yet another aspect, the present invention provides a metabolite of COMPOUND I, preferably a purified and isolated metabolite of COMPOUND I, having the following structure, wherein the O-glucuronide may be substituted at any available position of the 5-fluoro-quinoline ring:

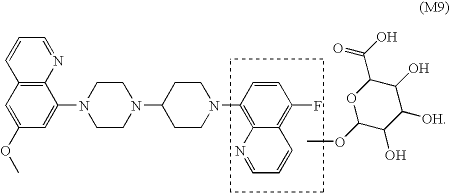

In yet another aspect, the present invention provides a metabolite of COMPOUND I, preferably a purified and isolated metabolite of COMPOUND I, having the following structure, wherein the hydroxyl may be substituted at any available position of the 6-methoxy-quinoline ring and the piperazine ring:

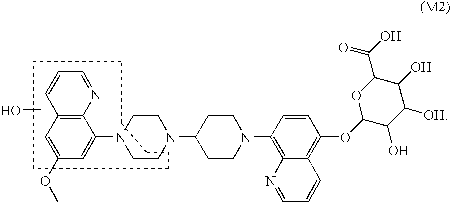

In yet another aspect, the present invention provides a metabolite of COMPOUND I, preferably a purified and isolated metabolite of COMPOUND I, having the following structure, wherein the hydroxyl may be substituted at any available position of the 6-methoxy-quinoline ring, and wherein the O-glucuronide may be substituted at any available position of the 5-fluoro-quinoline ring:

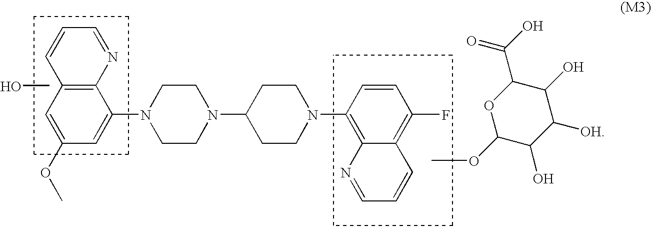

In yet another aspect, the present invention provides a metabolite of COMPOUND I, preferably a purified and isolated metabolite of COMPOUND I, having the following structure, wherein the O-glucuronide may be substituted at any available position of the 6-methoxy-quinoline ring:

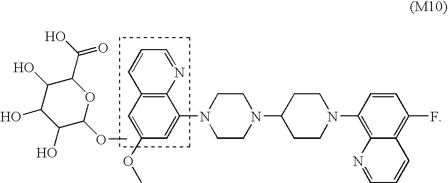

In yet another aspect, the present invention provides a metabolite of COMPOUND I, preferably a purified and isolated metabolite of COMPOUND I, having the following structure, wherein the HOSO₃— may be substituted at any available position of the 6-methoxy-quinoline ring:

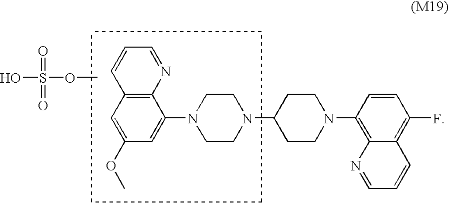

In yet another aspect, the present invention provides a metabolite of COMPOUND I, preferably a purified and isolated metabolite of COMPOUND I, having the following structure:

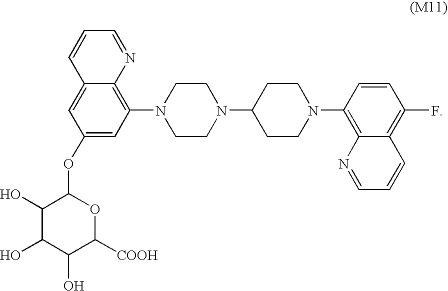

In yet another aspect, the present invention provides a metabolite of COMPOUND I, preferably a purified and isolated metabolite of COMPOUND I, having the following structure:

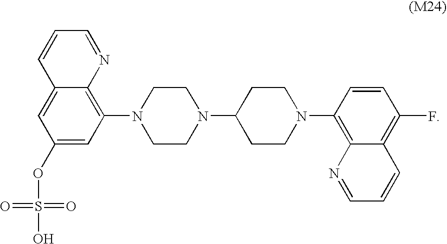

In yet another aspect, the present invention provides a metabolite of COMPOUND I, preferably a purified and isolated metabolite of COMPOUND I, having the following structure:

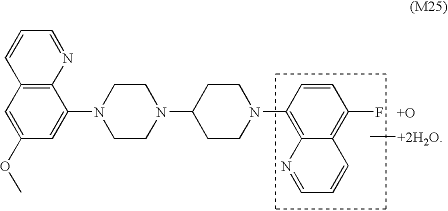

In yet another aspect, the present invention provides a metabolite of COMPOUND I, preferably a purified and isolated metabolite of COMPOUND I, having the following structure:

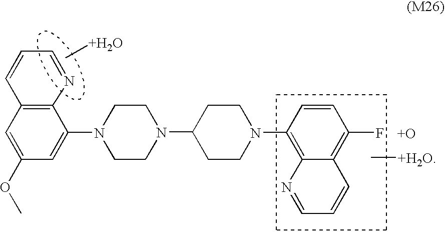

In a further aspect, the present invention provides a purified and isolated metabolite of COMPOUND I in substantially pure form.

In another aspect, the present invention provides a pharmaceutical composition comprising at least one purified and isolated metabolite of COMPOUND I and a pharmaceutically acceptable carrier, diluent, or excipient.

In yet another aspect, the present invention provides a method of preparing a purified and isolated metabolite of 5-Fluoro-8-{4-[4-(6-methoxyquinolin-8-yl)piperazin-1-yl]piperidin-1-yl}quinoline, comprising:

(i) treating 5-Fluoro-8-{4-[4-(6-methoxyquinolin-8-yl)piperazin-1-yl]piperidin-1-yl}quinoline or its pharmaceutically acceptable salt with rat, mouse, dog, monkey or human liver microsomes;

(ii) treating 5-Fluoro-8-{4-[4-(6-methoxyquinolin-8-yl)piperazin-1-yl]piperidin-1-yl}quinoline or its pharmaceutically acceptable salt with rat, mouse, dog, monkey or human liver S9 fractions; or

(iii) cryopreserved rat, dog, or human hepatocytes.

In yet another aspect, the present invention provides a method for preparing a compound of formula (M21),

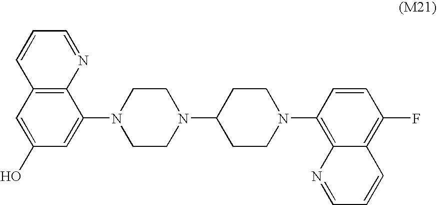

comprising demethylating the methoxy group of COMPOUND I,

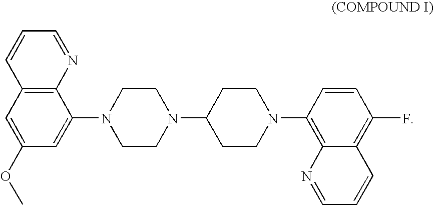

In a further aspect, the present invention provides a method for preparing a compound of formula (M21),

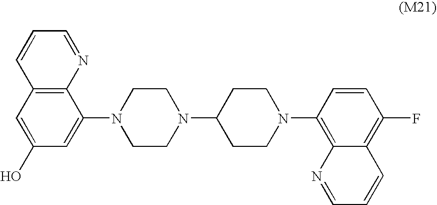

comprising:

(i) contacting a compound of formula (A),

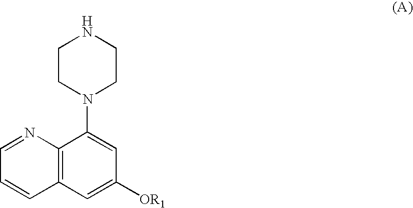

wherein R₁ is a hydroxyl protecting group;
with a compound of formula (B),

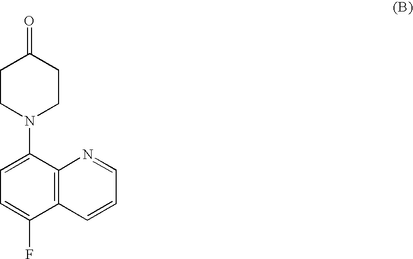

to provide a compound of formula (C); and

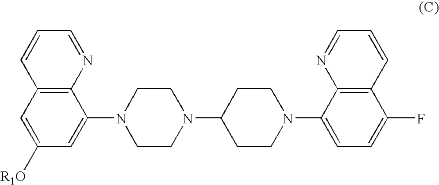

(ii) deprotect the hydroxyl protecting group R₁ of the compound of formula (C) to provide the compound of formula (M21).

In another aspect, the present invention provides a compound of formula (M21) prepared by the methods of as described hereinabove.

In yet another aspect, the present invention provides a method for treating a 5-HT_{1A}-related disorder in a patient in need thereof, the method comprising administering to the patient a therapeutically effective amount of at least one purified and isolated metabolite of COMPOUND I. This invention also provides use of at least one purified and isolated metabolite of COMPOUND I in the preparation of a medicament for treating a 5-HT_{1A}-related disorder in a patient. In certain embodiments, the 5-HT_{1A}-related disorder is a cognition-related disorder or an anxiety-related disorder. In certain other embodiments, the cognition-related disorder is dementia, Parkinson's disease, Huntington's disease, Alzheimer's disease, cognitive deficits associated with Alzheimer's disease, mild cognitive impairment, or schizophrenia. In yet other embodiments, the anxiety-related disorder is attention deficit disorder, obsessive compulsive disorder, substance addiction, withdrawal from substance addiction, premenstrual dysphoric disorder, social anxiety disorder, anorexia nervosa, or bulimia nervosa.

In yet another aspect, the present invention provides a method for treating a 5-HT_{1A}-related disorder in a patient in need thereof, the method comprising administering to the patient a therapeutically effective amount of at least one purified and isolated metabolite of COMPOUND I in combination with a second therapeutic agent. In certain embodiments, the second therapeutic agent is an anti-depressant agent, an anti-anxiety agent, anti-psychotic agent, or a cognitive enhancer. In certain other embodiments, the second therapeutic agent is a selective serotonin reuptake inhibitor, an SNRI, or a cholinesterase inhibitor. This invention also provides a product comprising at least one purified and isolated metabolite of COMPOUND I and a second therapeutic agent as a combined preparation for simultaneous, sequential or separate use in the treatment of a 5-HT_{1A}-related disorder in a patient.

In yet another aspect, the present invention provides a method for treating Alzheimer's disease, mild cognitive impairment, or depression to a patient in need thereof, the method comprising administering to the patient a therapeutically effective amount of at least one purified and isolated metabolite of COMPOUND I. This invention also provides use of at least one purified and isolated metabolite of COMPOUND I in the preparation of a medicament for treating Alzheimer's disease, mild cognitive impairment, or depression in a patient.

In a further aspect, the present invention provides a method for treating sexual dysfunction associated with drug treatment, and/or improving sexual function in a patient in need thereof, the method comprising administering to the patient a therapeutically effective amount of at least one purified and isolated metabolite of COMPOUND I. This invention also provides use of at least one purified and isolated metabolite of COMPOUND I in the preparation of a medicament for treating sexual dysfunction associated with drug treatment, and/or improving sexual function in a patient.

In another aspect, the present invention provides a radiolabeled compound of formula (G), or an enantiomer, diastereomer, tautomer, or pharmaceutically acceptable salt or solvate thereof:

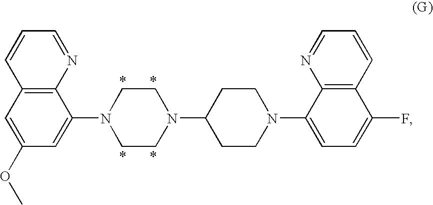

wherein each * represents a carbon-14.

In yet another aspect, the present invention provides a radiolabeled compound of formula (G), or a trisuccinate salt or solvate thereof:

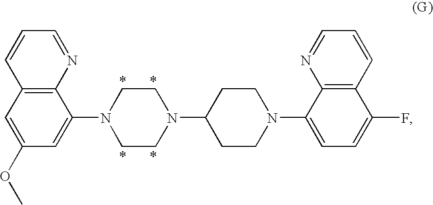

wherein each * represents a carbon-14.

In yet another aspect, the present invention provides a method for preparing a radiolabeled compound of formula (F), wherein each * represents a carbon-14,

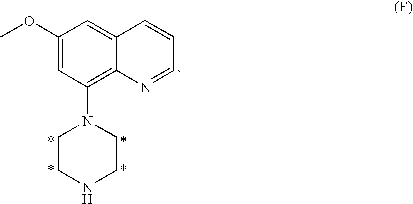

comprising contacting a compound of formula (D),

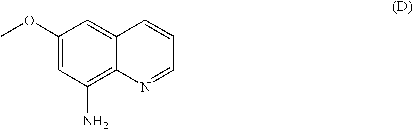

with a radiolabeled compound of formula (E) or a pharmaceutically acceptable salt thereof, wherein each * represents a carbon-14,

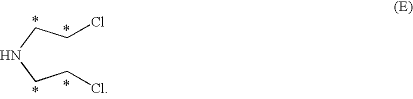

In yet another aspect, the present invention provides a method for preparing a radiolabeled compound of formula (G), wherein each * represents a carbon-14,

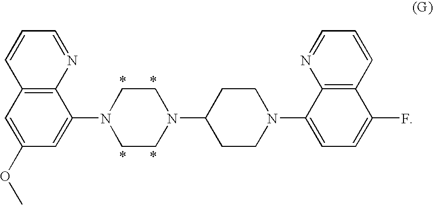

comprising:

(a) contacting a compound of formula (D),

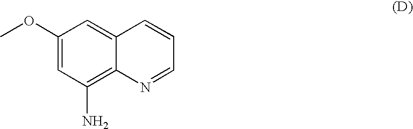

with a radiolabeled compound of formula (E) or a pharmaceutically acceptable salt thereof, wherein each * represents a carbon-14,

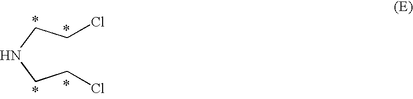

to give a radiolabeled compound of formula (F), wherein each * represents a carbon-14,

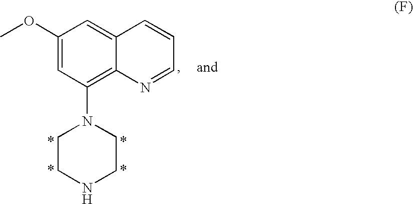

(b) contacting the compound of formula (F) with a compound of formula (B),

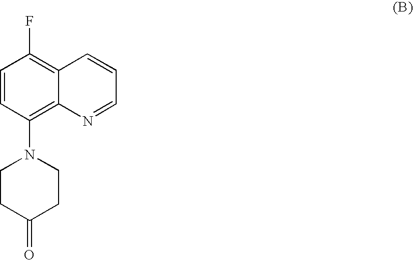

to provide a compound of formula (G).

### BRIEF DESCRIPTION OF THE DRAWINGS

FIG. 1 shows UV chromatograms of metabolite profiles of COMPOUND I in mouse, rat, monkey and human liver microsomes in the presence of NADPH.

FIG. 2 shows UV chromatograms of metabolite profiles of COMPOUND I in mouse, rat, monkey and human liver microsomes in the presence of NADPH and UDPGA.

FIG. 3 shows UV chromatograms of metabolite profiles of COMPOUND I in mouse, rat, monkey and human liver S9 in the presence of NADPH, UDPGA and acetyl CoA.

FIG. 4 is the proposed fragmentation scheme and product ions of m/z 472 mass spectra for COMPOUND I.

FIG. 5 is the proposed fragmentation scheme and product ions of m/z 244 mass spectra for P1.

FIG. 6 is the proposed fragmentation scheme and product ions of m/z 190 mass spectrum for M1.

FIG. 7 is the proposed fragmentation scheme and product ions of m/z 662 mass spectra for M2.

FIG. 8 is the proposed fragmentation scheme and product ions of m/z 680 mass spectra for M3.

FIG. 9 is the proposed fragmentation scheme and product ions of m/z 646 mass spectra for M5.

FIG. 10 is the proposed fragmentation scheme and product ions of m/z 506 mass spectra for M15.

FIG. 11 is the proposed fragmentation scheme and product ions of m/z 662 mass spectra for M7.

FIG. 12 is the proposed fragmentation scheme and product ions of m/z 331 mass spectra for M8.

FIG. 13 is the proposed fragmentation scheme and product ions of m/z 664 mass spectra for M9.

FIG. 14 is the proposed fragmentation scheme and product ions of m/z 664 mass spectra for M10.

FIG. 15 is the proposed fragmentation scheme and product ions of m/z 327 mass spectra for M12.

FIG. 16 is the proposed fragmentation scheme and product ions of m/z 315 mass spectra for M14.

FIG. 17 is the proposed fragmentation scheme and product ions of m/z 484 mass spectra for M16.

FIG. 18 is the proposed fragmentation scheme and product ions of m/z 504 mass spectra for M17.

FIG. 19 is the proposed fragmentation scheme and product ions of m/z 470 mass spectra for M18.

FIG. 20 is the proposed fragmentation scheme and product ions of m/z 488 mass spectra for M20.

FIG. 21 is the proposed fragmentation scheme and product ions of m/z 458 mass spectra for M21.

FIG. 22 is the proposed fragmentation scheme and product ions of m/z 488 mass spectra for M22.

FIG. 23 is the proposed fragmentation scheme and product ions of m/z 472 mass spectra for M23.

FIG. 24 is a scheme showing the proposed metabolic pathways for COMPOUND I in mouse, rat, monkey and human liver microsomes and S9.

FIG. 25 shows radiochromatographic profiles of [¹⁴C]COMPOUND I (20 μM) incubations in cryopreserved rat, dog, and human hepatocytes at 37° for 1 hr.

FIG. 26 is the proposed fragmentation scheme and product ions of m/z 472 mass spectrum for COMPOUND I.

FIG. 27 is the proposed fragmentation scheme and product ions of m/z 244 mass spectrum for P1.

FIG. 28 is the proposed fragmentation scheme and product ions of m/z 327 mass spectrum for M12.

FIG. 29 is the proposed fragmentation scheme and product ions of m/z 315 mass spectrum for M14.

FIG. 30 is the proposed fragmentation scheme and product ions of m/z 470 mass spectrum for M18.

FIG. 31 is the proposed fragmentation scheme and product ions of m/z 568 mass spectrum for M19.

FIG. 32 is the proposed fragmentation scheme and product ions of m/z 488 mass spectrum for M20.

FIG. 33 is the proposed fragmentation scheme and product ions of m/z 458 mass spectrum for M21.

FIG. 34 is the proposed fragmentation scheme and product ions of m/z 488 mass spectrum for M22.

FIG. 35 is a scheme showing the proposed metabolic pathways for COMPOUND I in cryopreserved rat, dog, and human hepatocytes.

FIG. 36 shows radiochromatograms of pooled plasma samples from rats following a single oral administration of 5 mg/kg of [¹⁴C]COMPOUND I.

FIG. 37 shows radiochromatograms of pooled brain samples from rats following a single oral administration of 5 mg/kg of [¹⁴C]COMPOUND I.

FIG. 38 shows radiochromatogram of pooled 0-24 hr fecal samples from male rats following a single oral administration of 5 mg/kg of [¹⁴C]COMPOUND I.

FIG. 39 is the proposed fragmentation scheme and product ions of m/z 472 mass spectrum for COMPOUND I.

FIG. 40 is the proposed fragmentation scheme and product ions of m/z 646 mass spectrum for M5.

FIG. 41 is the proposed fragmentation scheme and product ions of m/z 664 mass spectrum for M9.

FIG. 42 is the proposed fragmentation scheme and product ions of m/z 634 mass spectrum for M11.

FIG. 43 is the proposed fragmentation scheme and product ions of m/z 315 mass spectrum for M14.

FIG. 44 is the proposed fragmentation scheme and product ions of m/z 458 mass spectrum for M21.

FIG. 45 is the proposed fragmentation scheme and product ions of m/z 488 mass spectrum for M22.

FIG. 46 is a scheme showing the proposed metabolic pathways for COMPOUND I in rats.

FIG. 47 shows mean cumulative recovery of radioactivity in male dogs following a single 3 mg/kg oral dose of [¹⁴C]COMPOUND I.

FIG. 48 shows radiochromatograms of pooled plasma samples from male dogs following a single oral dose of 3 mg/kg of [¹⁴C]COMPOUND I.

FIG. 49 shows radiochromatograms of pooled homogenates of 0-24 and 24-48 hour fecal samples from male dogs following a single oral dose of 3 mg/kg of [¹⁴C]COMPOUND I.

FIG. 50 is the proposed fragmentation scheme and product ions of m/z 472 mass spectrum for COMPOUND I.

FIG. 51 is the proposed fragmentation scheme and product ions of m/z 664 mass spectrum for M10.

FIG. 52 is the proposed fragmentation scheme and product Ions of m/z 327 mass spectrum for M12.

FIG. 53 is the proposed fragmentation scheme and product ions of m/z 315 mass spectrum for M14.

FIG. 54 is the proposed fragmentation scheme and product ions of m/z 458 mass spectrum for M21.

FIG. 55 is the proposed fragmentation scheme and product ions of m/z 538 mass spectrum for M24.

FIG. 56 is a scheme showing the proposed metabolic pathways for COMPOUND I in dogs.

FIG. 57 is the proposed fragmentation scheme and product ions of m/z 524 and of m/z 506 mass spectra for M25 and M26.

### DETAILED DESCRIPTION OF THE INVENTION

The term “pharmaceutically acceptable salt” can refer to acid addition salts or base addition salts of the compounds in the present disclosure. A pharmaceutically acceptable salt is any salt which retains the activity of the parent compound and does not impart any deleterious or undesirable effect on the subject to whom it is administered and in the context in which it is administered.

Pharmaceutically acceptable salts include metal complexes and salts of both inorganic and organic acids. Pharmaceutically acceptable salts include metal salts such as aluminum, calcium, iron, magnesium, manganese and complex salts. Pharmaceutically acceptable salts include acid salts such as acetic, aspartic, alkylsulfonic, arylsulfonic, axetil, benzenesulfonic, benzoic, bicarbonic, bisulfuric, bitartaric, butyric, calcium edetate, camsylic, carbonic, chlorobenzoic, -32-cilexetil, citric, edetic, edisylic, estolic, esyl, esylic, formic, fumaric, gluceptic, gluconic, glutamic, glycolic, glycolylarsanilic, hexamic, hexylresorcjnoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanlic, sulfonic, sulfuric, tannic, tartaric, teoclic, toluenesulfonic, and the like. Pharmaceutically acceptable salts may be derived from amino acids, including but not limited to cysteine. Other acceptable salts may be found, for example, in Stahl et al., Pharmaceutical Salts: Properties, Selection, and Use, Wiley-VCH; 1st edition (Jun. 15, 2002).

The term “therapeutically effective amount” refers to that amount of a compound that results in prevention or amelioration of symptoms in a patient or a desired biological outcome, e.g., improved clinical signs, delayed onset of disease, reduced/elevated levels of lymphocytes and/or antibodies, etc. The effective amount can be determined as described herein. The selected dosage level will depend upon the activity of the particular compound, the route of administration, the severity of the condition being treated, and the condition and prior medical history of the patient being treated. However, it is within the skill of the art to start doses of the compound at levels lower than required to achieve the desired therapeutic effect and to gradually increase the dosage until the desired effect is achieved. In one embodiment, the data obtained from the assays can be used in formulating a range of dosage for use in humans.

When a functional group is termed “protected”, this means that the group is in modified form to mitigate, especially preclude, undesired side reactions at the protected site. Suitable protecting groups for the methods and compounds described herein include, without limitation, those described in standard textbooks, such as Greene, T. W. et al., Protective Groups in Organic Synthesis, Wiley, N.Y. (1999). Specifically, suitable hydroxyl protecting groups include, but are not limited to, ethers such as methyl ether, substituted methyl ethers, substituted ethyl ethers, substituted benzyl ethers, silyl ethers; and esters such as formate, acetate, benzoate, carbonates, sulfonates, etc. Suitable amine protecting groups include, but are not limited to, 9-fluorenylmethoxycarbonyl protecting group and organoxycarbonyl group, i.e. where the amine is protected as a carbamate. Carbamates include, without limitation, t-butyl carbamate, methyl carbamate, ethyl carbamate, 2,2,2-trichloroethyl carbamate, 2-(trimethylsilyl)ethyl carbamate, 1,1-dimethyl-2,2,2-trichloroethyl carbamate, benzyl carbamate, p-methoxybenzyl carbamate, p-nitrobenzylcarbamate, p-bromobenzyl carbamate, p-chlorobenzyl carbamate, and 2,4-dichlorobenzyl carbamate. Suitable ketone protecting groups include, but are not limited to, acetals and ketals, such as 1,3-dioxanes and 1,3-dioxolanes.

Prodrugs and solvates of the compounds of the present invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates include, for example, hydrates.

Compounds of the present invention, and salts thereof, may exist in their tautomeric form (for example, as an amide or imino ether). All such tautomeric forms are contemplated herein as part of the present invention.

All stereoisomers of the compounds of the present invention (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the IUPAC 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

Metabolites of the present invention are, subsequent to their preparation, preferably isolated and purified, e.g., to make pure or substantially pure. The term “substantially pure” refers to a metabolite that is at least 80% pure, preferably at least 90% pure, and more preferably at least 95% pure. Such “substantially pure” metabolites are contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

The term “NADPH” refers to nicotinamide adenine dinucleotide phosphate, which is a cofactor used for drug metabolism studies in animals.

The term “UDPGA” refers to uridine 5′-diphosphosphoglucuronic acid, which is a cofactor used for drug metabolism studies in animals.

The term “liver microsomes” refers to closed vesicles of fragmented endoplasmic reticulum created when liver cells or tissue are disrupted by homogenization.

The term “S9 fraction” refers to post-mitochondrial supernatant fraction, which is a mixture of microsomes and cytosol. Accordingly, S9 fraction contains a wide variety of phase I and phase II enzymes including P450 enzymes, flavin-monooxygenases, carboxylesterases, epoxide hydrolase, UDP-glucuronosyltransferases, sulfotransferases, methyltransferases, acetyltransferases, glutathione S-transferases and other drug-metabolizing enzymes. S9 fraction requires exogenous cofactors for activity. The cofactors used consist of an NADPH-regenerating system (phase I oxidation), uridine 5′-diphosphoglucuronic acid (UDPGA; phase II glucuronidation), and/or 3′-phosphoadenosine-5′-phosphosulphate (PAPS; phase II sulfation). Incubations are usually conducted in 50 to 100 mM Tris buffer. Other buffers may be used, depending on the analytical method requirements. Thus, S9 fraction can be useful for studying xenobiotic metabolism.

Throughout the specifications, groups and substituents thereof may be chosen to provide stable moieties and compounds. The terms “the compounds of the present invention,” “the compounds provided herein,” “the compounds disclosed herein,” and “metabolites of the present invention” may be used interchangeably and all refer to novel metabolites of COMPOUND I, and preferably purified and isolated metabolites of COMPOUND I.

### In Vitro Metabolism of COMPOUND I in Rat, Dog, Monkey and Human Liver Microsomes and S9 Fractions

COMPOUND I is a selective 5-HT_{1A} receptor antagonist that is being developed for the treatment of cognitive dysfunctions associated with Alzheimer's disease (AD) and other dementias. The metabolism of COMPOUND I was investigated in both liver microsomes and S9 from male Sprague-Dawley rats, male beagle dogs, male Cynomolgus monkeys and male humans. Intrinsic clearance was determined by LC/MS. Metabolite profiles were determined by HPLC with UV detection and metabolites were identified by LC/MS.

In the presence of NADPH and UDPGA, COMPOUND I was moderately to highly metabolized in liver microsomes from rats, dogs, monkeys and humans with intrinsic clearance values of 0.332, 0.055, 0.312 and 0.100 mL/min/mg, respectively (t_{1/2}=2, 13, 2 and 7 min., respectively).

In the presence of NADPH and UDPGA (microsomes and S9 preparations) and acetyl CoA (S9 preparations only), eleven metabolites of COMPOUND I were characterized by LC/MS in human liver microsomes and S9. The metabolism of COMPOUND I in liver microsomes was more extensive than in the corresponding S9 preparation for each species examined. Based upon UV detection, hydroxyl desfluoro COMPOUND I (M18) and hydroxy COMPOUND I (M20) were the predominant metabolites of COMPOUND I in human liver microsomal incubations. Other COMPOUND I metabolites identified in human liver microsomes were 6-methoxy-quinoline-5,8-dione (M1), hydroxy desfluoro COMPOUND I glucuronide (M5), dihydroxy desfluoro COMPOUND I glucuronide (M7), hydroxy COMPOUND I glucuronide (M10), N-desfluoroquinolinyl COMPOUND I (M12), N-desmethoxyquinolinyl COMPOUND I (M14), desfluoro COMPOUND I quinone (M16), O-desmethyl COMPOUND I (M21) and desmethyl COMPOUND I quinone (M23). Nine of the metabolites observed in human samples (M1, M5, M10, M12, M14, M18, M20, M21 and M23) were also observed in rat, dog and monkey samples. M7 and M16 were also observed in rat and monkey samples. Dihydroxy desfluoro COMPOUND I glucuronide (M2), dihydroxy COMPOUND I glucuronide (M3), COMPOUND I dihydrodiol (M6, M13 and M15), hydroxy N-desmethoxyquinolinyl COMPOUND I (M8), hydroxy COMPOUND I glucuronide (M9) and dihydroxy COMPOUND I (M17) were observed only in rat liver microsomal incubations. COMPOUND I tetrahydro triol (M25 and M26) were also observed in rat liver microsomal incubations. Hydroxy COMPOUND I (M22) was observed in rat and monkey liver microsomes. COMPOUND I metabolism in liver S9 was less extensive than in the corresponding microsomal preparation for each species examined. M10, M12, M14 and M20 were observed in human as well as in monkey liver S9 preparations. M10 was also observed in rat liver S9 incubations. M12 was also observed in rat and dog liver S9 incubations. M14 was also observed in rat liver S9 incubations. M21 was present in dog and monkey liver S9 incubations. M22 was present in rat liver S9 incubations.

In summary, species differences in COMPOUND I metabolism were minor. Extensive metabolism of COMPOUND I was observed in all species examined with more extensive metabolism in rat and monkey. Similar metabolites were observed in all species examined. Rat liver microsomes and S9 generated eight COMPOUND I metabolites not observed in dog, monkey or human incubations. N-Dealkylation, oxidative defluorination, hydroxylation and glucuronidation were the main metabolic pathways observed. All metabolites observed in incubations with human liver microsomes and S9 were observed in liver microsomes or S9 from at least two other species.

COMPOUND I can be prepared according to a method as described in co-pending U.S. patent application Ser. No. 11/450,942, filed on Jun. 9, 2006. A more detailed synthesis of COMPOUND I is described in Example 1 below. The internal standard, furosemide, was purchased from Sigma-Aldrich (Milwaukee, Wis., USA). HPLC grade water, methanol, and acetonitrile were obtained from E. M. Science (Gibbstown, N.J.). Deuterium oxide was obtained from Cambridge Isotope Laboratories (Andover, Mass.). All other chemicals were reagent grade or better.

Liver microsomes from male Sprague-Dawley rats (L31040-113; pool of 3 animals; 13.2 mg/mL protein; total P450 content of 0.64 nmol/mg protein) were made in-house. Liver microsomes from male beagle dogs (pool of 4 animals; 20 mg/mL protein; total P450 content of 0.53 nmol/mg protein), Cynomolgus monkeys (pool of 10 animals; 20 mg/mL protein; total P450 content of 1.2 nmol/mg protein) and humans (pool of 50 male donors; 20 mg/mL protein; total P450 content of 0.42 nmol/mg protein) were purchased from XenoTech LLC (Kansas City, Kans.). Liver S9 fractions from male Sprague-Dawley rats (pool of 198 animals; 20 mg/mL protein; total P450 content of 0.23 nmol/mg protein), male beagle dogs (pool of 4 animals; 20 mg/mL protein; total P450 content of 0.13 nmol/mg protein), Cynomolgus monkeys (pool of 7 animals; 20 mg/mL protein; total P450 content of 0.30 nmol/mg protein) and humans (pool of 50 male donors; 20 mg/mL protein; total P450 content of 0.09 nmol/mg protein) were also purchased from XenoTech LLC (Kansas City, Kans.).

Experiments were conducted to determine the intrinsic clearance by substrate depletion for COMPOUND I metabolism in liver microsomes in the presence of NADPH and UDPGA. Incubations containing midazolam (0.2 μM with 0.1 mg/mL liver microsomes) or diclofenac (1 μM with 1 mg/mL liver microsomes) were also performed as positive controls for oxidative and glucuronidation activity, respectively. Sample preparation and incubation were performed on a MultiProbe IIEX Robotic Liquid Handling System (Perkin-Elmer, Shelton, Conn.) and a Micromix 5 (Packard, Downers Grove, Ill.). Samples were pre-incubated at 37° C. Final incubation concentrations of all reagents are listed in Table 1. Control incubations were conducted under the same conditions without cofactors. At specified time points (0, 10, 20 and 30 min), 150 μL aliquots were transferred to tubes with 500 μL acetonitrile containing 30 ng/mL of the internal standard (furosemide) to precipitate the protein. Samples were centrifuged at 4° C. for 10 min at 3400 rpm (ThermoForma, Marietta, Ohio). The supernatant (400 μL) was transferred to a clean test tube and the acetonitrile evaporated under a stream of nitrogen in a Turbo Vap (Caliper Life Sciences, Hopkinton, Mass.). The samples were then reconstituted in 200 μL of 20% methanol in water and analyzed by LC/MS.

Microsomal incubations for metabolite profiling were similar to those described above for intrinsic clearance determination (see Table 1). Aliquots of COMPOUND I (10 μL), dissolved in DMSO:methanol (1:9), were added to a 96-well plate containing phosphate buffer, and MgCl₂ and the liver microsomes at 37° C. using the same automation apparatus as for the stability study described hereinabove. The reactions were initiated by the addition of the UDPGA and NADPH generating system. The final incubation volume for all samples was 1 mL and the length of incubation was 30 min. Control incubations were conducted under the same conditions without cofactors. Three 250 μL aliquots were transferred to a 96-well plate containing 900 μL acetonitrile to precipitate the protein. Samples were centrifuged at 4° C. for 10 min at 3400 rpm (ThermoForma, Marietta, Ohio). The supernatant (900 μL) was transferred to a clean 96-well plate and the acetonitrile evaporated under a stream of nitrogen in a Turbo Vap (Caliper Life Sciences). The samples were then reconstituted in 150 μL of 20% methanol in water. The three aliquots for each sample were pooled and analyzed by LC/MS.

TABLE 1
Reagents Utilized in Liver Microsomal Incubations

| Reagents | Final Concentration | |
|---|---|---|
| MgCl₂ | 10 | mM |
| COMPOUND I: | | |
| Intrinsic Clearance Determination | 1 | μM |
| Metabolite characterization | 10 | μM |
| NADPH Regenerating System: | | |
| Glucose-6-phosphate | 3.6 | mM |
| NADP⁺ | 1.3 | mM |
| Glucose-6-phosphate dehydrogenase | 0.4 | units/mL |
| UDPGA | 4 | mM |

Incubations in liver S9 for metabolite profiling contained COMPOUND I (10 μM) and other reagents as listed in Table 2 below. Sample preparation and incubations were performed on a MultiProbe IIEX Robotic Liquid Handling System as described for liver microsomes hereinabove. S9 incubation buffer and cofactors were chosen to allow N-acetylation and carbamoyl glucuronidation in addition to oxidative and glucuronidation pathways possible in liver microsomes. Incubations of sulfamethazine and SCA-136 with liver S9 were used as positive controls for N-acetylation and carbamoyl glucuronidation, respectively. Following a 30 min. incubation, 750 μL from each sample were added to 900 μL acetonitrile. Following centrifugation and evaporation of the supernatant, the samples were reconstituted in 450 μL of 20% methanol in water for analysis by HPLC/UV and LC/MS (see below).

TABLE 2
Reagents Utilized in S9 Incubations^{a}

| | |
|---|---|
| Reagents | Final Concentration |

| | | |
|---|---|---|
| MgCl₂ | 10 | mM |
| Carbonate buffer, pH 7.4 | 100 | mM |
| Liver S9 fraction | 1 | mg/mL |
| COMPOUND I | 10 | μM |
| NADPH Regenerating System: | | |
| Glucose-6-phosphate | 3.6 | mM |
| NADP | 1.3 | mM |
| Glucose-6-phosphate dehydrogenase | 0.4 | units/mL |
| UDPGA | 4 | mM |
| Acetyl CoA | 0.1 | mM |
| CoA Regenerating System: | | |
| Acetyl carnitine | 4.5 | mM |
| Carnitine acetyl transferase | 0.2 | units/mL |

^{a}Incubations carried out for 30 min. at 37° C.

The HPLC system used was a Surveyor HPLC (Thermo Electron Corp., San Jose, Calif.). Separations were accomplished on a Supelcosil LC-C18 column (150×4.6 mm, 5 μm) (Supelco, Bellefonte, Pa.). The column temperature was maintained at ambient and the sample chamber was at 10° C. Mobile phase A was 5 mM ammonium acetate in water and mobile phase B was methanol. The linear mobile phase gradient is shown in Table 3.

TABLE 3
HPLC Gradient (Intrinsic Clearance Determination)

| | | | Flow rate |
|---|---|---|---|
| Time (min) | % A | % B | (mL/min) |
| 0.0 | 75 | 25 | 1.5 |
| 1.0 | 75 | 25 | 1.5 |
| 1.5 | 100 | 0 | 1.5 |
| 4.0 | 100 | 0 | 1.5 |
| 4.01 | 75 | 25 | 1.5 |
| 4.5 | 75 | 25 | 1.5 |

The mass spectrometer used for microsomal stability analysis was a TSQ Quantum (Thermo Electron Corp.; San Jose, Calif.) equipped with an electrospray ionization (ESI) source and operated in the positive ionization mode. Selected reaction monitoring (SRM) was used for selective detection of COMPOUND I and furosemide, the internal standard (IS). Settings for the mass spectrometer are listed in Table 4. SRM analysis conditions are summarized in Table 5.

TABLE 4
TSQ Mass Spectrometer Settings
(Intrinsic Clearance Determination)

| | |
|---|---|
| Spray voltage | 4.0 kV |
| Heated capillary temp. | 350° C. |
| Nebulizer gas | 85 |
| Auxiliary gas | 40 |
| Collision energy | 30 eV |

TABLE 5
SRM Analysis Conditions for Intrinsic Clearance Determination

| | Precursor ion | Production |
|---|---|---|
| Compound | (m/z, nominal mass) | (m/z, nominal mass) |
| COMPOUND I | 472 | 227 |
| Furosemide (IS) | 329 | 205 |

The HPLC system used for mass spectrometric analysis was an Agilent 1100 HPLC (Agilent Technologies, Palo Alto, Calif.) equipped with a binary pump and a diode array UV detector. The UV detector was set to monitor 190-400 nm. Separations were accomplished on a Luna C18 column (150×2.1 mm, 5 μm) (Phenomenex Incorporated, Torrance, Calif.). Mobile phase A was 5 mM ammonium acetate in water and mobile phase B was acetonitrile. The linear mobile phase gradient is shown in Table 2.2.5-1. During LC/MS sample analysis, up to 4.0 min of the initial flow was diverted away from the mass spectrometer prior to evaluation of metabolites.

TABLE 6
LC/MS HPLC Gradient for Metabolite Profiling

| | | | Flow Rate |
|---|---|---|---|
| Time (min) | A (%) | B (%) | (mL/min) |
| 0 | 95 | 5 | 0.25 |
| 5 | 95 | 5 | 0.25 |
| 16 | 50 | 50 | 0.25 |
| 26 | 0 | 100 | 0.25 |
| 29 | 0 | 100 | 0.25 |
| 30 | 95 | 5 | 0.25 |
| 35 | 95 | 5 | 0.25 |

The mass spectrometer used for metabolite characterization was a Finnigan LCQ Deca ion trap mass spectrometer (Thermo Electron Corp.). It was equipped with an electrospray ionization (ESI) source and operated in the positive ionization mode. Full scan and data dependent MS₂ and MS₃ mass spectra were recorded. Settings for the mass spectrometer are listed in Table 2.2.5-2. Finnigan Xcalibur software (version 1.3) was used for control of equipment and recording of data from UV and LC/MS analyses.

TABLE 7
Finnigan LCQ Ion Trap Mass Spectrometer Settings

| Spray voltage | 4.0 kV |
|---|---|
| Heated capillary temp. | 350° C. |
| Nebulizer gas setting | 70 |
| Auxiliary gas setting | 35 |
| Relative collision energy | 40% |

Peak area ratios for COMPOUND I against the internal standard were used to express the % remaining at given time points. The percent of remaining COMPOUND I was calculated by dividing the peak area ratio obtained at 0 min. by the corresponding ratio at each time point. In vitro intrinsic clearance (CLᵢₙₜ) of COMPOUND I was calculated by linear regression of log % remaining vs time plots using Microsoft Excel, 7.0 and normalizing to mg protein content. Metabolic half-life (t_{1/2}) was calculated by dividing 0.693 by the intrinsic clearance.

The intrinsic clearance of COMPOUND I (1 μM) in male Sprague-Dawley rat, male beagle dog, male Cynomolgus monkey and pooled male and female human liver microsomes was assessed by substrate depletion in the presence of NADPH and UDPGA and is presented in Table 8. In the presence of NADPH and UDPGA, COMPOUND I was moderately to highly metabolized in liver microsomes from rats, dogs, monkeys and humans with intrinsic clearance values of 0.332, 0.055, 0.312 and 0.100 mL/min/mg, respectively (t_{1/2}=2, 13, 2 and 7 min, respectively). Oxidative and glucuronidation activity were confirmed in the rat, dog, monkey and human liver microsomes by substrate depletion of the positive controls, midazolam and diclofenac.

TABLE 8
Intrinsic clearance of COMPOUND I in Male Sprague-Dawley
Rat, Male Beagle Dog, Male Cynomolgus Monkey and Pooled
Male and Female Human Liver Microsomes

| | Species | | | |
|---|---|---|---|---|
| | Rat | Dog | Monkey | Human |
| Intrinsic Clearance (mL/min/mg) | 0.332 | 0.055 | 0.312 | 0.100 |
| t_{1/2} (minutes) | 2 | 13 | 2 | 7 |

1 μM COMPOUND I in 1 mg microsomal protein per mL in the presence of NADPH and UDPGA

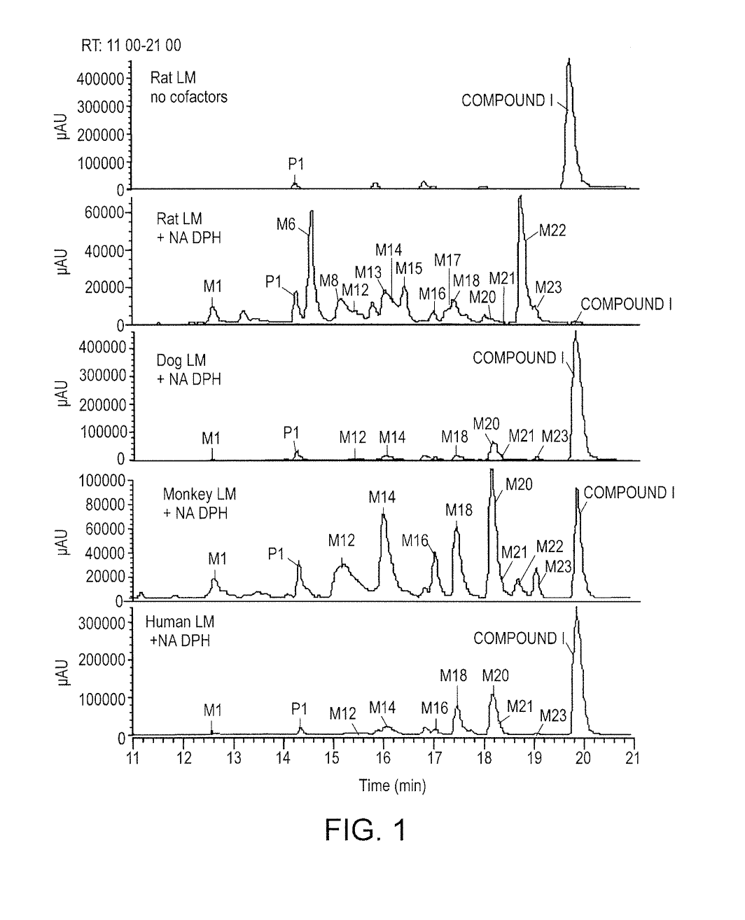

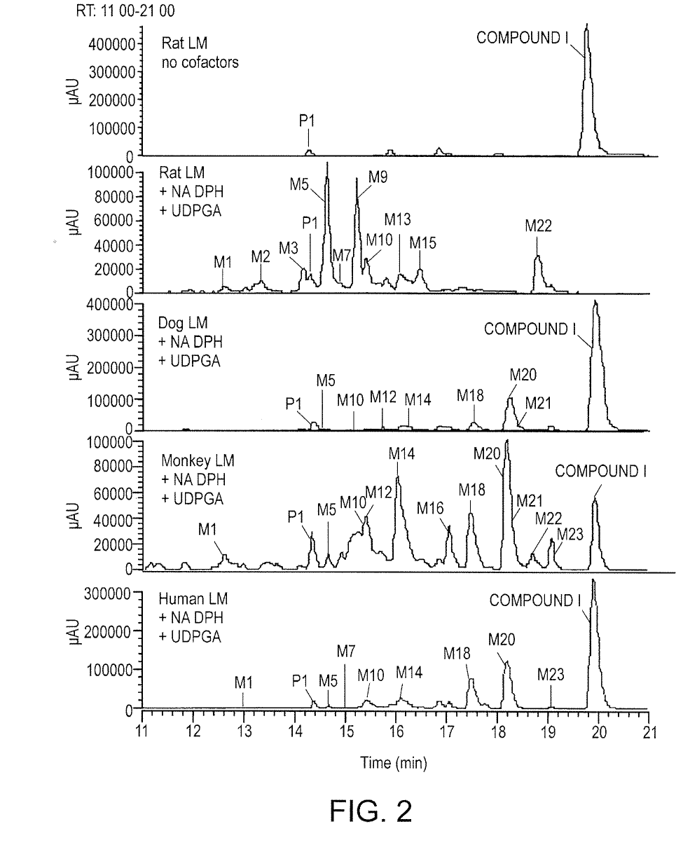

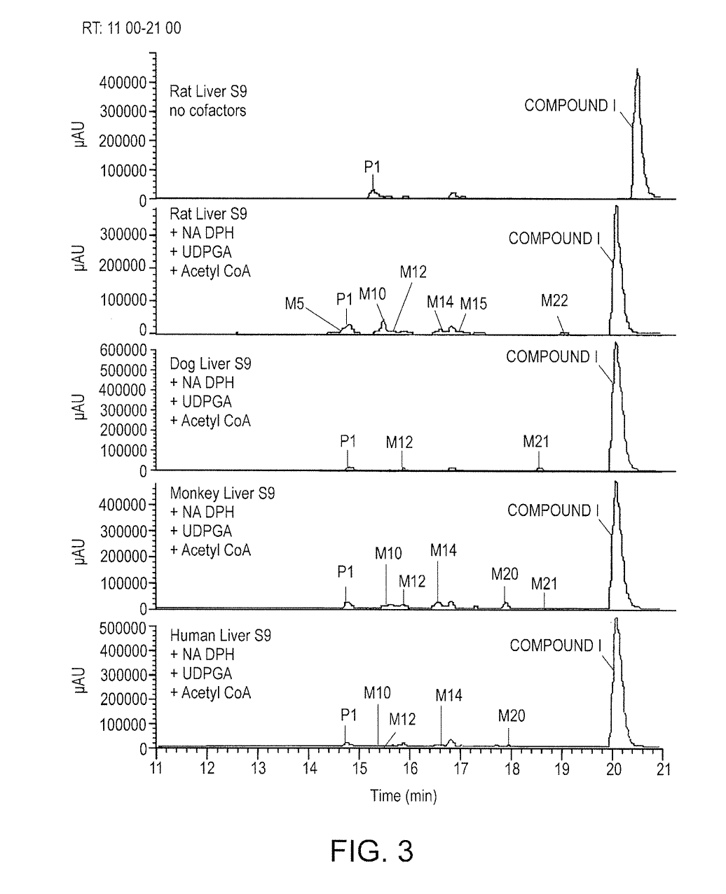

Chromatographic profiles of COMPOUND I (10 μM) metabolites in incubations with liver microsomes in the presence of NADPH are presented in FIG. 1. Chromatographic profiles of COMPOUND I (10 μM) metabolites in incubations with liver microsomes in the presence of NADPH and UDPGA are presented in FIG. 2. Chromatographic profiles of COMPOUND I (10 μM) metabolites in incubations with liver S9 in the presence of NADPH, UDPGA and acetyl coenzyme A (CoA) are presented in FIG. 3. In incubations containing NADPH, UDPGA and acetyl CoA, fourteen Phase I metabolites (M1, M6, M8, M12, M13, M14, M15, M16, M17, M18, M20, M21, M22 and M23) and six Phase II metabolites (M2, M3, M5, M7, M9 and M10) were observed. In incubations without cofactors, no metabolites were observed.

LC/MS analysis was conducted on extracts of mouse, rat, monkey and human liver microsomes and S9. A summary of metabolites of COMPOUND I observed in these samples is presented in Table 9. The mass spectral data for the characterized COMPOUND I metabolites are discussed below.

TABLE 9
Metabolites of COMPOUND I Characterized in Mouse, Rat, Dog, Monkey and Human Liver Microsomes and S9

| | t_{R} | | | | |
|---|---|---|---|---|---|
| Peak | (min)^{a} | [M + H]⁺ | Site of Metabolism | Metabolite Name | Species |
| P1 | 14.3 | 244 | Piperidine ring | Methoxyquinolinyl-piperazine | R, D, M, H |
| M1 | 12.6 | 190 | Methoxyquinoline | 6-Methoxy-quinoline-5,8-dione | R, D, M, H |
| M2 | 13.3 | 662 | Methoxyquinoline and | Dihydroxy desfluoro COMPOUND I glucuronide | R |
| | | | fluoroquinoline | | |
| M3 | 14.1 | 680 | Methoxyquinoline and | Dihydroxy COMPOUND I glucuronide | R |
| | | | fluoroquinoline | | |
| M5 | 14.6 | 646 | Fluoroquinoline | Hydroxy desfluoro COMPOUND I glucuonide | R, D, M, H |
| M6 | 14.6 | 506 | Fluoroquinoline | COMPOUND I dihydrodiol | R |
| M7 | 14.9 | 662 | Fluoroquinoline | Dihydroxy desfluoro COMPOUND I glucuronide | R, M, H |
| M8 | 15.2 | 331 | Methoxyquinoline and | Hydroxy N-desmethoxyquinolinyl COMPOUND I | R |
| | | | fluoroquinoline | | |
| M9 | 15.2 | 664 | Fluoroquinoline | Hydroxy COMPOUND I glucuronide | R |
| M10 | 15.4 | 664 | Methoxyquinoline | Hydroxy COMPOUND I glucuronide | R, D, M, H |
| M12 | 15.5 | 327 | Fluoroquinoline | N-Desfluoroquinolinyl COMPOUND I | R, D, M, H |
| M13 | 16.1 | 506 | Fluoroquinoline | COMPOUND I dihydrodiol | R |
| M14 | 16.15 | 315 | Methoxyquinoline | N-Desmethoxyquinolinyl COMPOUND I | R, D, M, H |
| M15 | 16.5 | 506 | Fluoroquinoline | COMPOUND I dihydrodiol | R |
| M16 | 17.0 | 484 | Fluoroquinoline | Desfluoro COMPOUND I quinone | R, M, H |
| M17 | 17.3 | 504 | Fluoroquinoline and | Dihydroxy COMPOUND I | R |
| | | | methoxyquinoline | | |
| M18 | 17.4 | 470 | Fluoroquinoline | Hydroxy desfluoro COMPOUND I | R, D, M, H |
| M20 | 18.2 | 488 | Methoxyquinoline | Hydroxy COMPOUND I | R, D, M, H |
| M21 | 18.6 | 458 | Methoxy group | O-Desmethyl COMPOUND I | R, D, M, H |
| M22 | 18.8 | 488c | Fluoroquinoline | Hydroxy COMPOUND I | R, M |
| M23 | 19.1 | 472 | Methoxyquinoline | Desmethyl COMPOUND I quinone | R, D, M, H |
| COMPOUND I | 20.0 | 472 | None | COMPOUND I | R, D, M, H |

^{a}Retention times obtained from UV chromatograms and may differ from LC/MS retention times

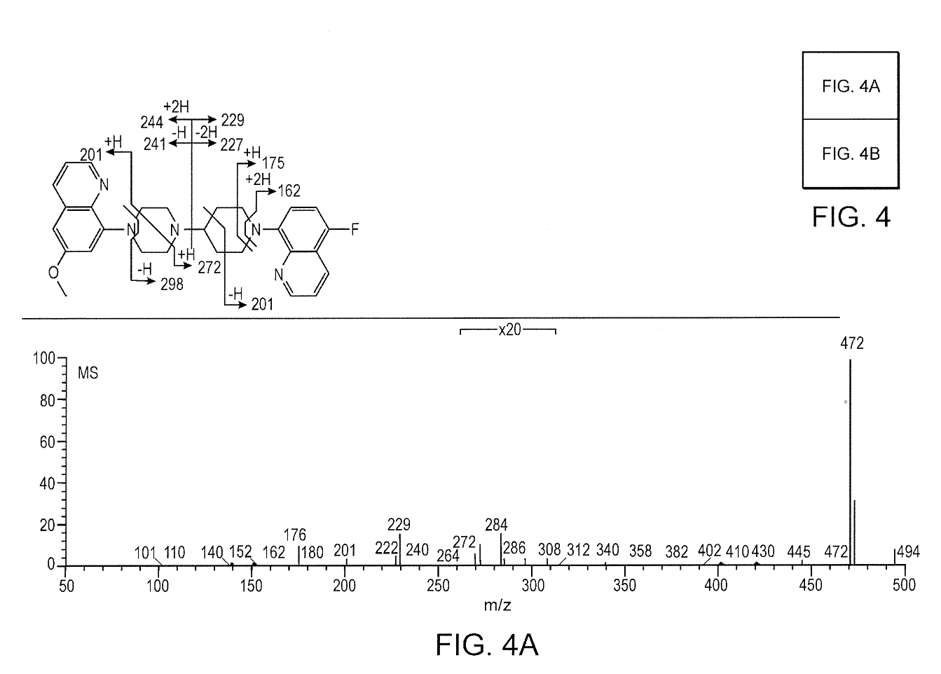

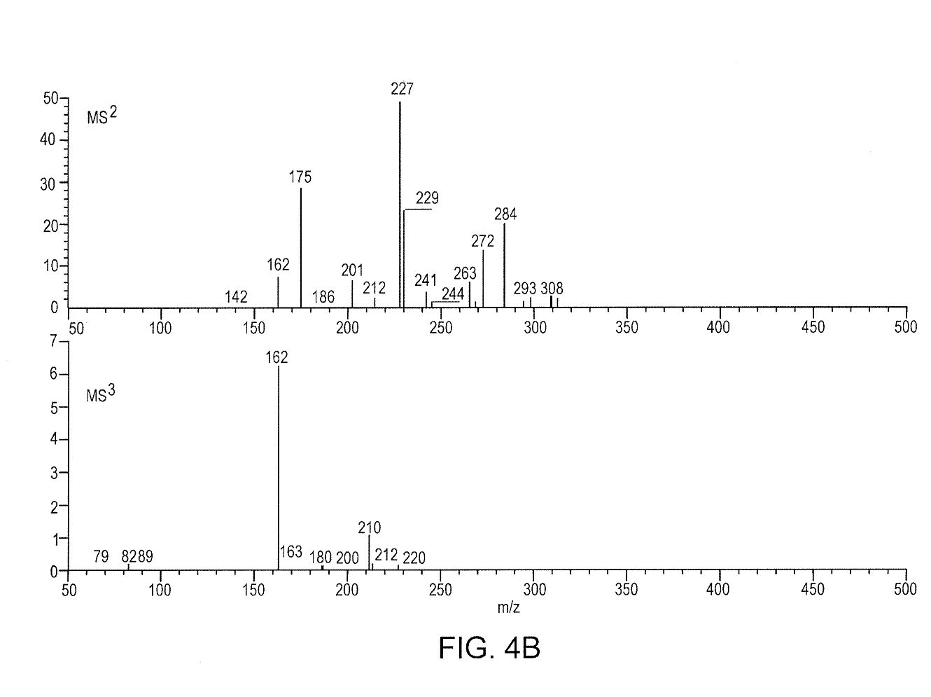

The mass spectral characteristics of synthetic COMPOUND I were examined for comparison with metabolites. In the LC/MS spectrum of COMPOUND I, the protonated molecular ion, [M+H]⁺ was observed at m/z 472. LC/MS with D₂O substituted for H₂O in the mobile phase generated [M+D]⁺ at m/z 473 (data not shown), consistent with COMPOUND I having no exchangeable hydrogens. The MS² and MS³ spectra obtained from collision activated dissociation of m/z 472 from COMPOUND I and the proposed fragmentation scheme are shown in FIG. 4. Fragmentation of the piperazine-piperidine bond with charge retention on the methoxyquinoline half of the molecule yielded m/z 244 and 241. The same fragmentation with charge retention on the fluoroquinoline half of the molecule yielded m/z 229 and 227. Fragmentation of the piperazine ring with charge retention on the product ions with the fluoroquinoline yielded m/z 298 and 272. Fragmentation of the piperidine ring generated fluoroquinoline-containing ions at m/z 175 and 162. Two assignments for the m/z 201 product ion were made based on product ions of ¹⁴C[M+H]⁺ (m/z 474) and of ¹⁴C₂[M+H]⁺ (m/z 476) mass spectral data for radiolabeled COMPOUND I (data not shown) for which all four carbon atoms of the piperazine ring were radiolabeled. One m/z 201 product ion originated from cleavage of the piperidine ring with charge retention on the moiety containing the fluoroquinoline. The other m/z 201 product ion originated from cleavage of the piperazine ring.

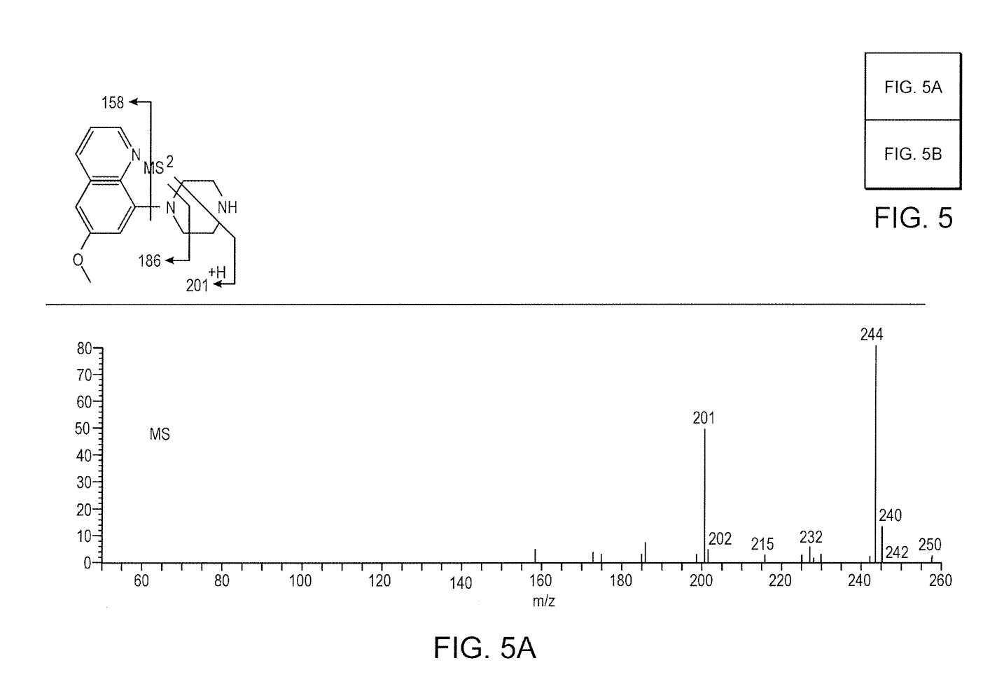

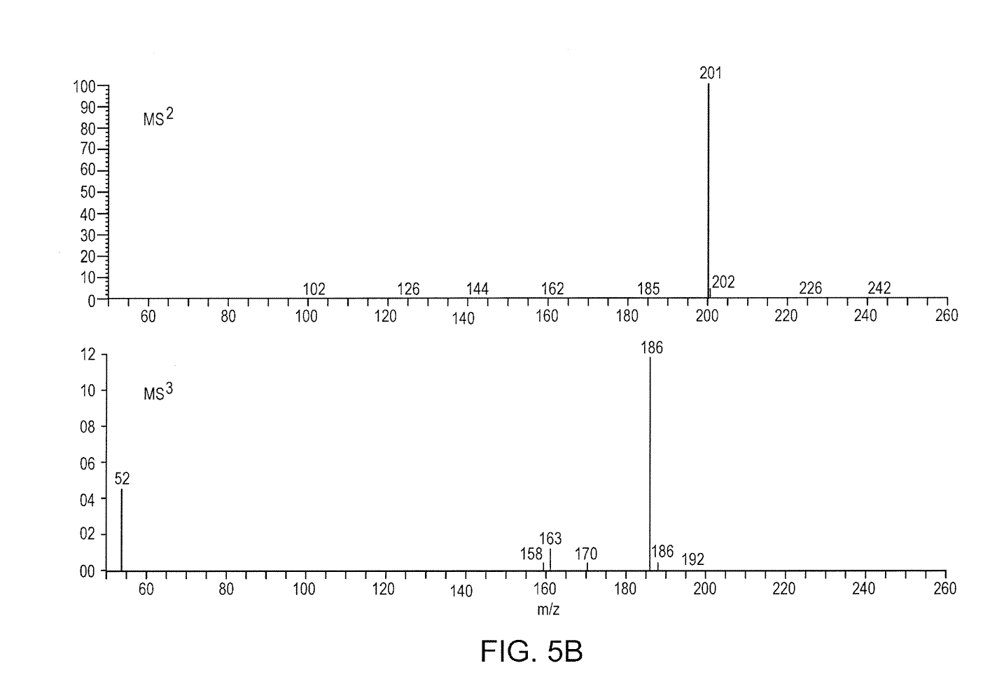

P1 was observed in all liver microsomal and S9 incubations including incubations with no cofactors. The [M+H]⁺ for P1 was observed at m/z 244, which was 228 Da less than COMPOUND I. LC/MS with D₂O substituted for H₂O in the mobile phase generated [M+D]⁺ at m/z 246 (data not shown). These data indicated one exchangeable hydrogen, which was one more than COMPOUND I and consistent with N-dealkylation of the COMPOUND I molecule. The product ions of m/z 244 mass spectra and the proposed fragmentation scheme for P1 are presented in FIG. 5. The product ion at m/z 201 was also observed for COMPOUND I, which suggested an intact methoxyquinolinyl-piperazine moiety. Fragmentation of the piperazine ring and quinoline-piperazine bond generated m/z 186 and 158, respectively, which were consistent with methoxyquinolinyl-piperazine. Therefore, P1 was identified as methoxyquinolinyl-piperazine.

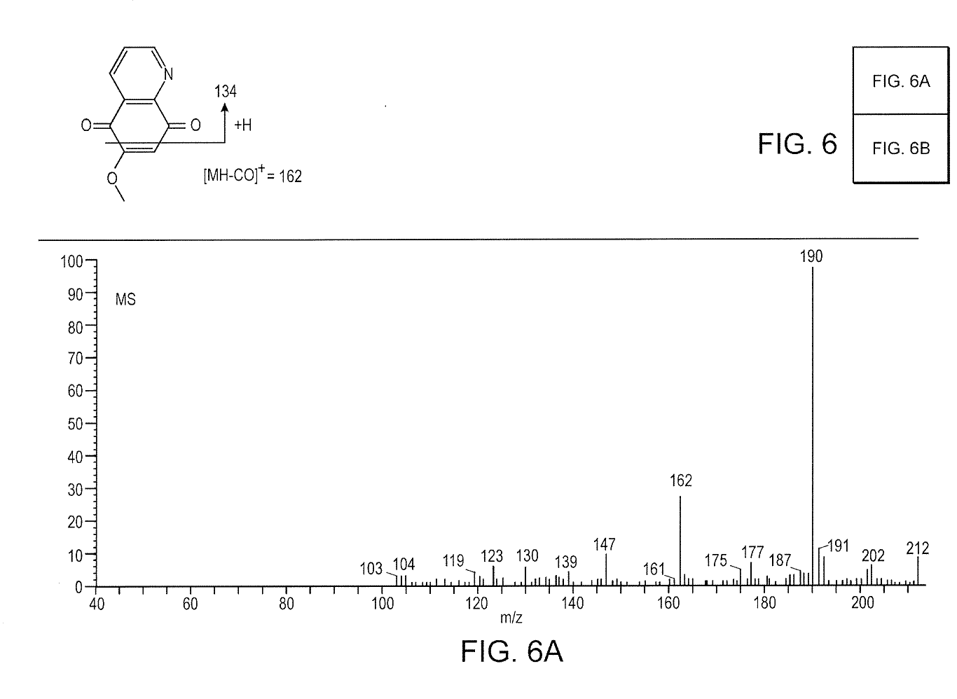

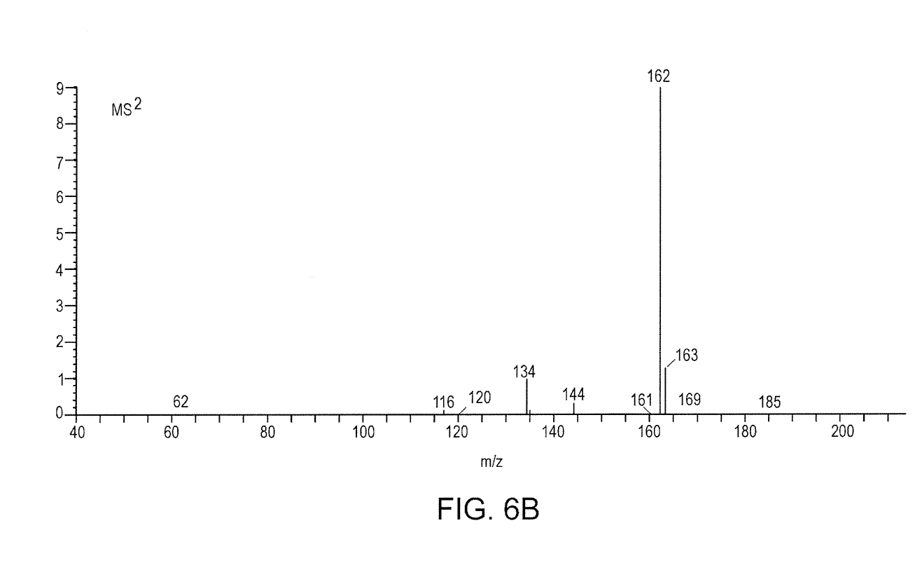

Metabolite M1 produced a [M+H]⁺ at m/z 190, which was 282 Da less than COMPOUND I and was consistent with cleavage of the COMPOUND I molecule. LC/MS with D₂O substituted for H₂O in the mobile phase generated [M+D]⁺ at m/z 191, which indicated no exchangeable hydrogens, the same as for COMPOUND I. The product ions of m/z 190 mass spectrum and the proposed fragmentation scheme for M1 are presented in FIG. 6. The product ion at m/z 134, resulted from loss of 56 Da (C₃H₄O) from [M+H]⁺. Loss of 28 Da (CO) from [M+H]⁺ yielded m/z 162, which indicated the presence of a carbonyl group. These data were consistent with a quinine byproduct of N-dealkylation of the methoxyquinoline moiety of COMPOUND I. Therefore, M1 was proposed to be 6-methoxy-quinoline-5,8-dione.

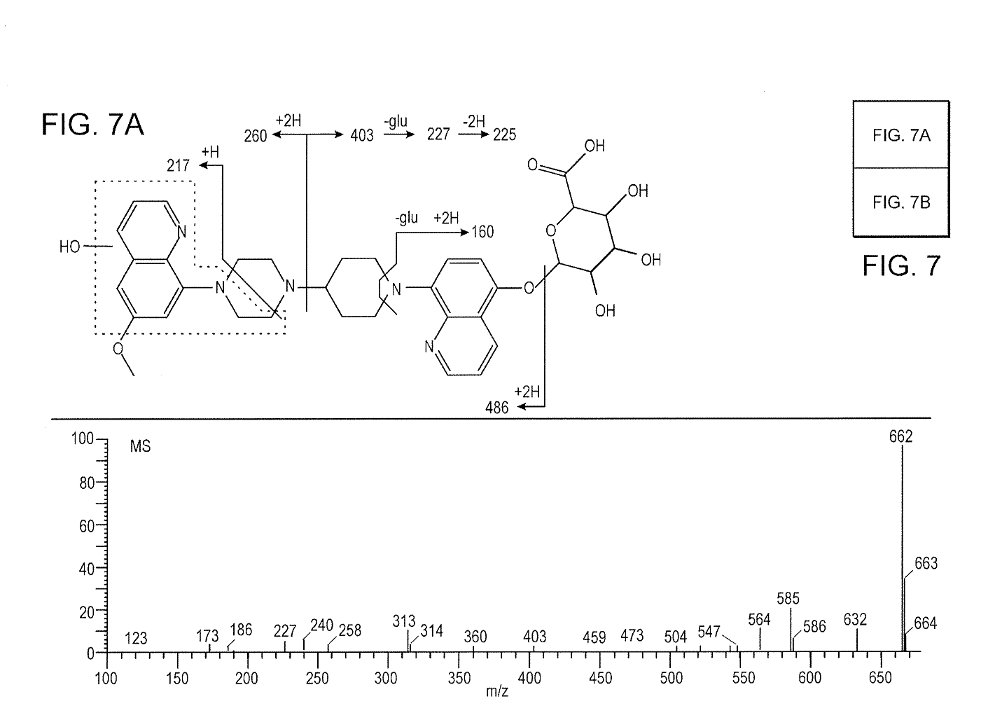

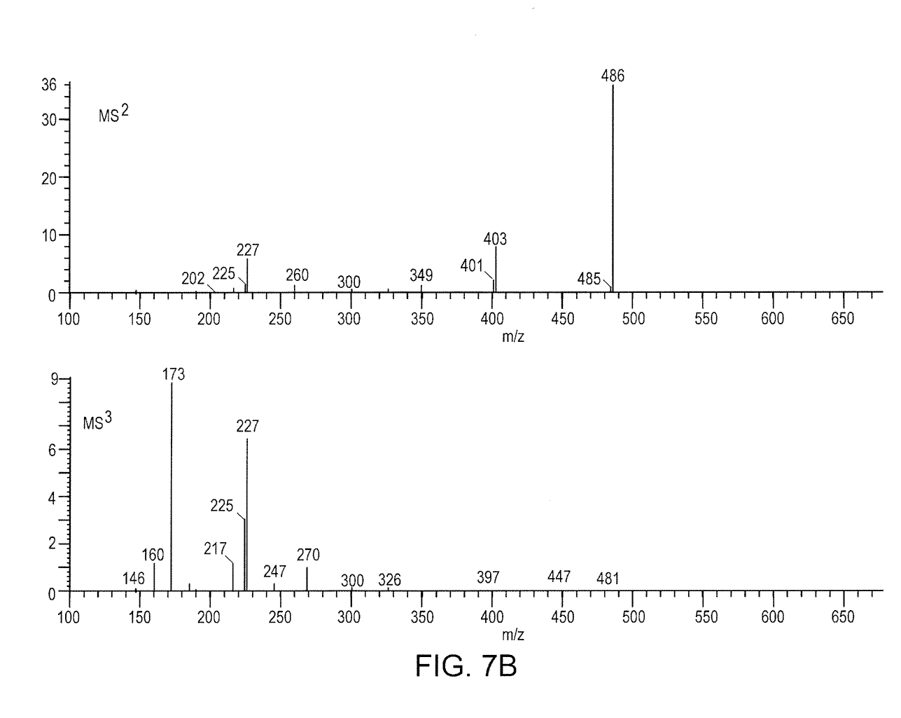

The [M+H]⁺ for M2 was observed at m/z 662, which was 190 Da larger than COMPOUND I. LC/MS with D₂O substituted for H₂O in the mobile phase generated [M+D]⁺ at m/z 668, which indicated five exchangeable hydrogens. These data were consistent with a glucuronide of a hydroxyl COMPOUND I metabolite. The proposed fragmentation scheme and product ions of m/z 662 mass spectra for M2 are shown in FIG. 7. Loss of 176 Da from [M+H]⁺ yielded m/z 486, which was 2 Da less than the [M+H]⁺ for a hydroxy COMPOUND I and indicated that M2 was a glucuronide. Product ions at m/z 227, 225 and 160 were 2 Da less than the corresponding ions at m/z 229, 227 and 162, respectively, for COMPOUND I, which indicated oxidative defluorination of the fluoroquinoline. The product ion at m/z 403 was 176 Da larger than m/z 227, which indicated that the hydroxyl group resulting from the oxidative defluorination was the site of glucuronidation. Product ions at m/z 260 and 270, of which m/z 260 and 217 were 16 Da larger than the corresponding methoxyquinolinyl ions at m/z 244 and 201, respectively, for COMPOUND I indicated hydroxylation of the methoxyquinolinyl-aminoethylene moiety. Therefore, M2 was identified as a dihydroxy desfluoro COMPOUND I glucuronide.

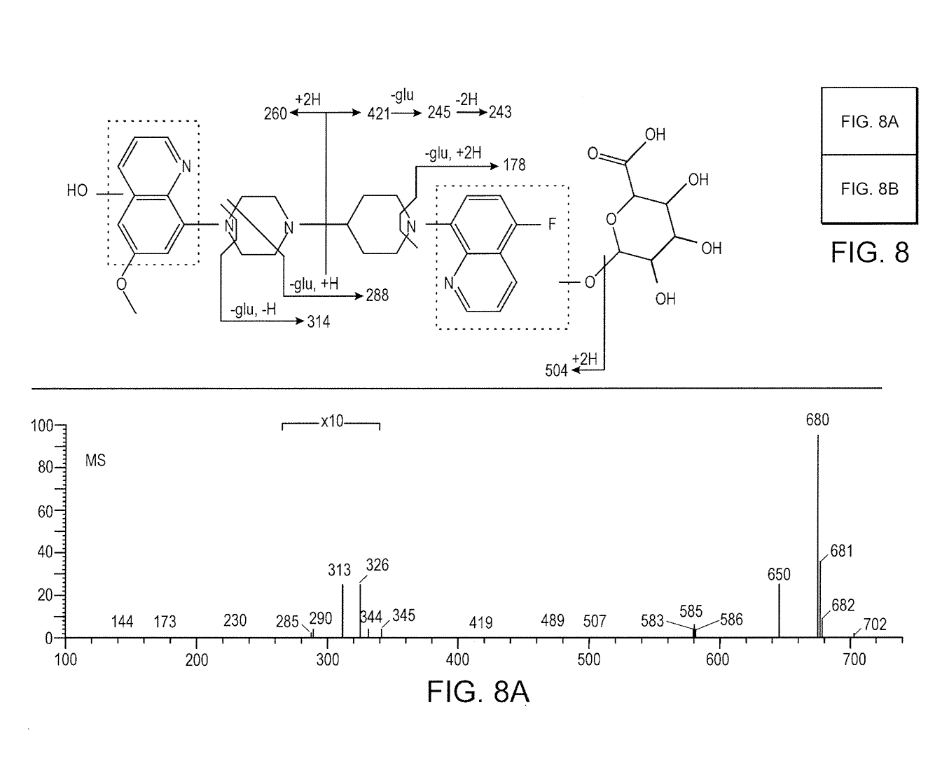

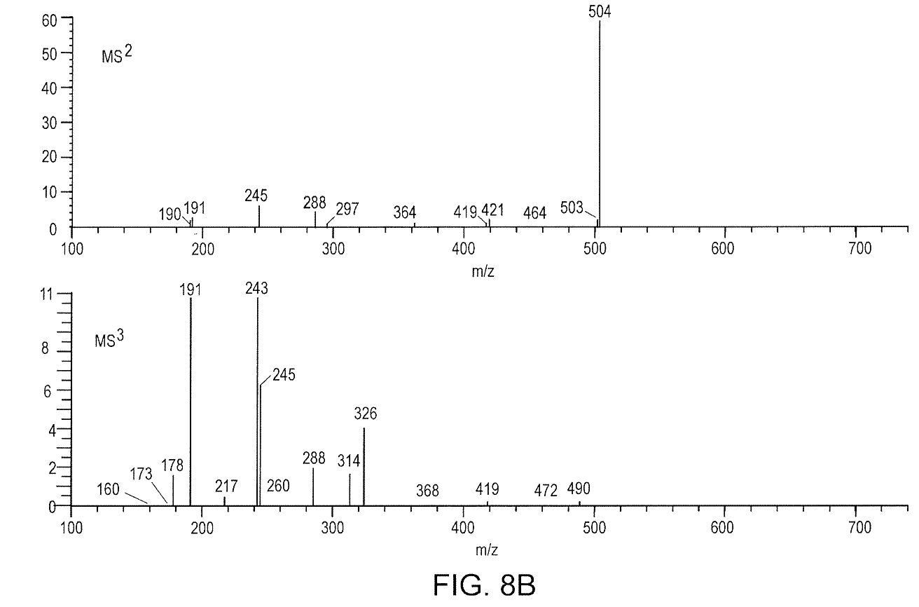

Metabolite M3 produced a [M+H]⁺ at m/z 680, which was 208 Da larger than COMPOUND I. LC/MS with D₂O substituted for H₂O in the mobile phase generated [M+D]⁺ at m/z 686, which indicated five exchangeable hydrogens. The proposed fragmentation scheme and product ions of m/z 680 mass spectra for M3 are shown in FIG. 8. Loss of 176 Da from [M+H]⁺ yielded m/z 504, which was 32 Da larger than COMPOUND I and indicated that M3 was a glucuronide of a dihydroxy COMPOUND I. Product ions at m/z 245, 243 and 178 were 16 Da larger than the corresponding ions at m/z 229, 227 and 162, respectively, for COMPOUND I, which indicated hydroxylation of the fluoroquinoline. The product ion at m/z 421 was 176 Da larger than m/z 245, which indicated that the hydroxylated fluoroquinoline was the site of glucuronidation. Product ions at m/z 314, 288 and 260, of which m/z 314 and 288 were 16 Da larger than the corresponding ions at m/z 298 and 272, respectively, for COMPOUND I and m/z 260 was 16 Da larger than the corresponding ion at m/z 244 for COMPOUND I indicated hydroxylation of the methoxyquinoline moiety. Therefore, M3 was identified as a dihydroxy COMPOUND I glucuronide.

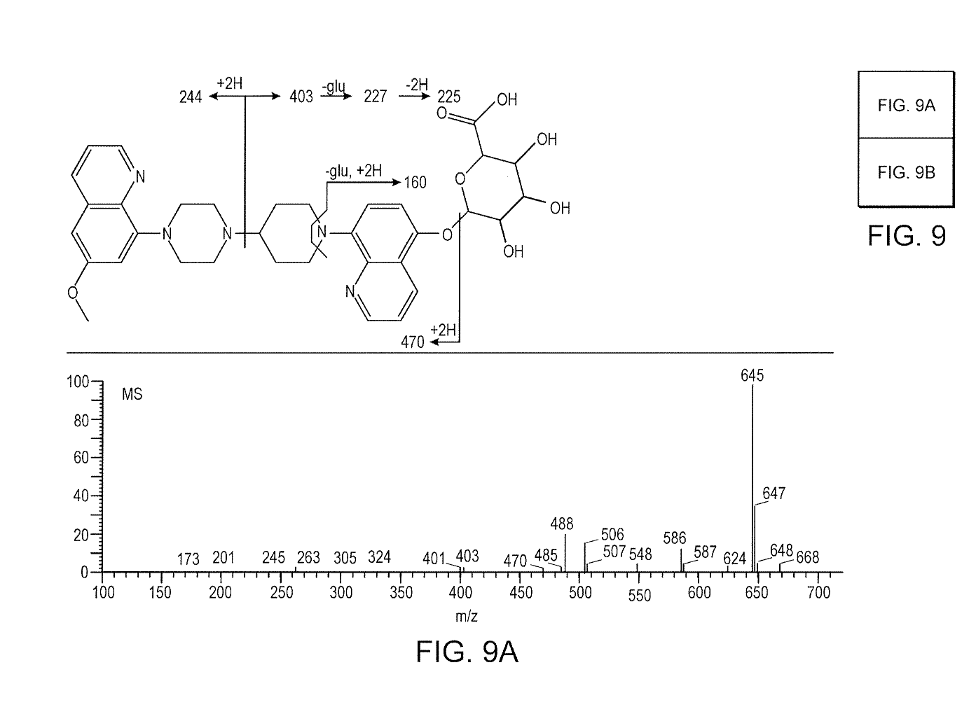

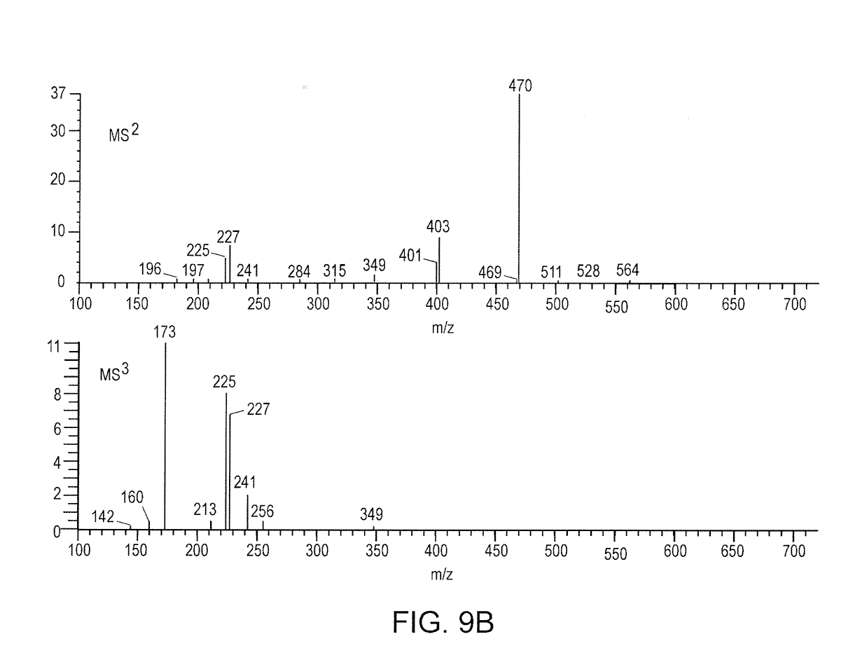

The [M+H]⁺ for M5 was observed at m/z 646, which was 174 Da larger than COMPOUND I. LC/MS with D₂O substituted for H₂O in the mobile phase generated [M+D]⁺ at m/z 651, which indicated four exchangeable hydrogens, consistent with a glucuronide. The proposed fragmentation scheme and product ions of m/z 646 mass spectra for M5 are shown in FIG. 9. Loss of 176 Da from [M+H]⁺ yielded m/z 470, which indicated that M3 was a glucuronide. Product ions at m/z 227, 225 and 160 were 2 Da less than the corresponding ions at m/z 229, 227 and 162, respectively, for COMPOUND I, which indicated oxidative defluorination of the fluoroquinoline. The product ion at m/z 403 was 176 Da larger than m/z 227, which indicated that the hydroxyl group resulting from the oxidative defluorination was the site of glucuronidation. The product ion at m/z 244 was also observed for COMPOUND I, which indicated an unchanged methoxyquinolinylpiperazine moiety. Therefore, M5 was identified as hydroxy desfluoro COMPOUND I glucuronide.

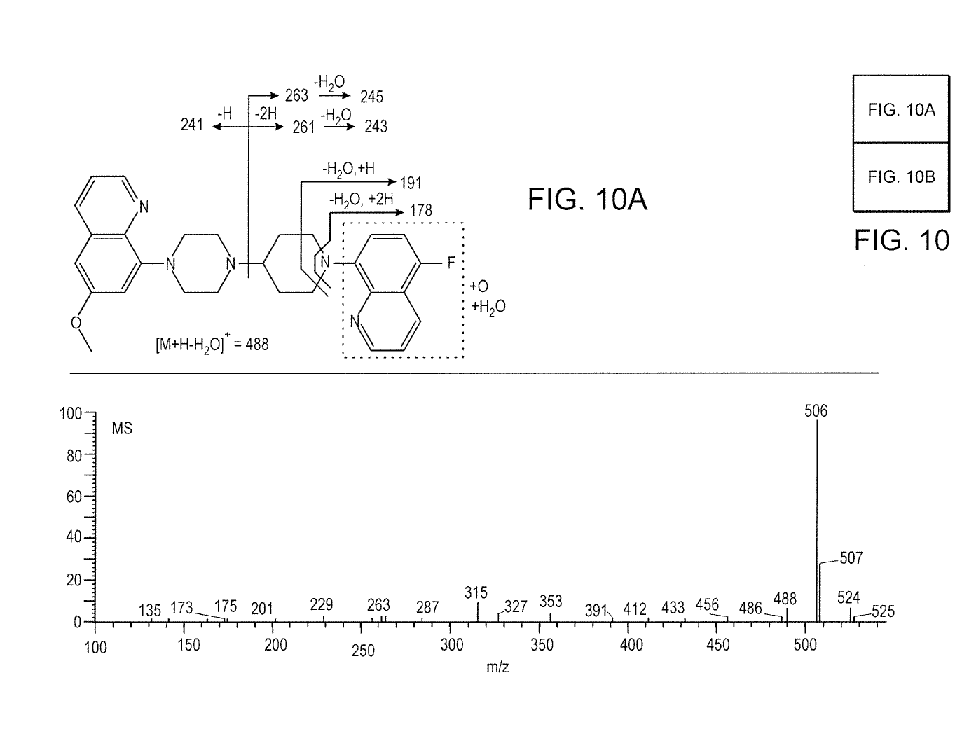

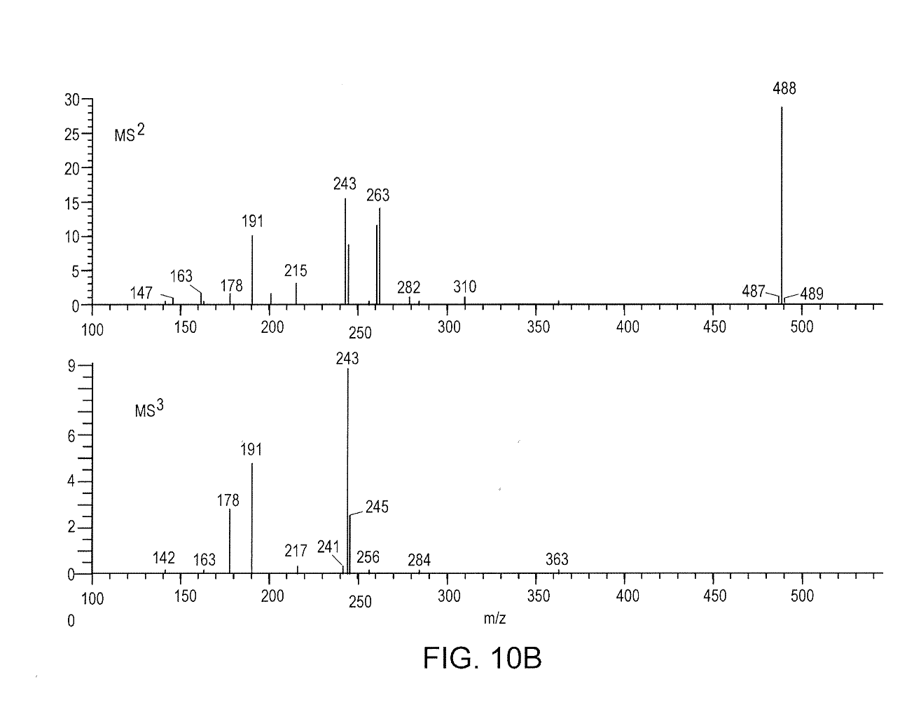

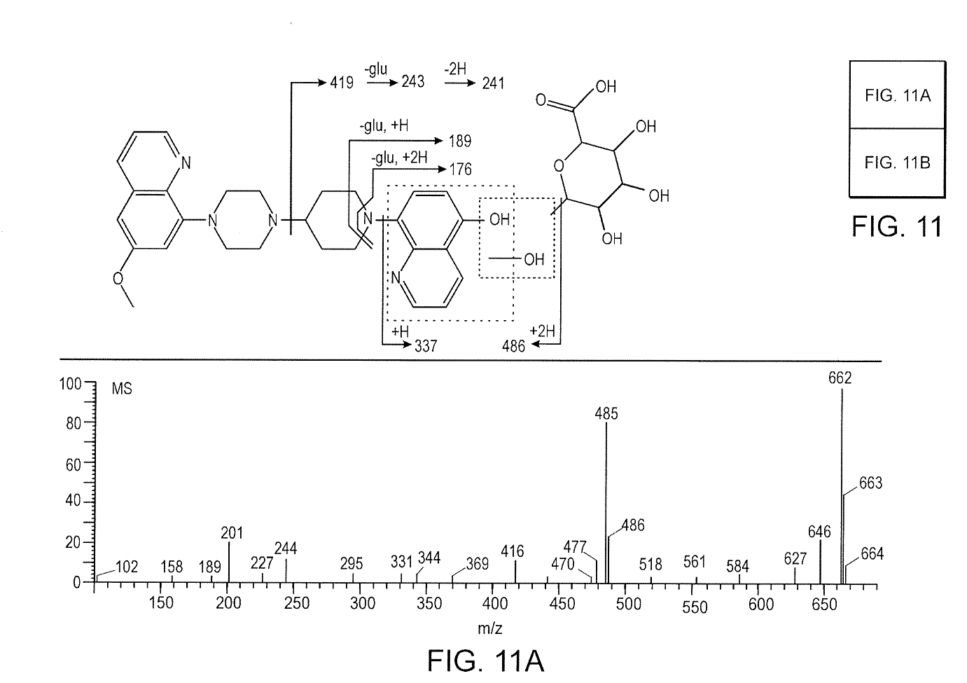

The [M+H]⁺ for metabolites M6, M13 and M15 were observed at m/z 506, which was 34 Da larger than COMPOUND I. Mass spectral data for M6, M13 and M15 were similar. LC/MS with D₂O substituted for H₂O in the mobile phase generated [M+D]⁺ at m/z 509. These data indicated two exchangeable hydrogens, which was two more than COMPOUND I and consistent with the presence of two hydroxyl groups. The product ions of m/z 506 mass spectra and the proposed fragmentation scheme for M15 are presented in FIG. 10. Loss of H₂O from [M+H]⁺ yielded m/z 488 as the base peak in the MS² spectrum, which indicated the presence of an aliphatic hydroxyl group. The product ion at m/z 241 was also observed for COMPOUND I, which indicated an unchanged methoxyquinoline moiety. Product ions at m/z 263 and 261 were 34 Da larger than the corresponding ions at m/z 229 and 227, respectively, for COMPOUND I, which indicated metabolism of the fluoroquinolinyl-piperidine moiety. Subsequent losses of water from m/z 263 and 261 yielded m/z 245 and 243, respectively, also consistent with an aliphatic hydroxyl group. Product ions at m/z 191 and 178, generated after losses of H₂O, were 16 Da larger than the corresponding ions at m/z 175 and 162, respectively, for COMPOUND I. These data and the molecular weight difference between M6, M13 and M15 versus COMPOUND I were consistent with dihydrodiol metabolites. Therefore, M6, M13 and M15 were proposed to be COMPOUND I dihydrodiol metabolites.

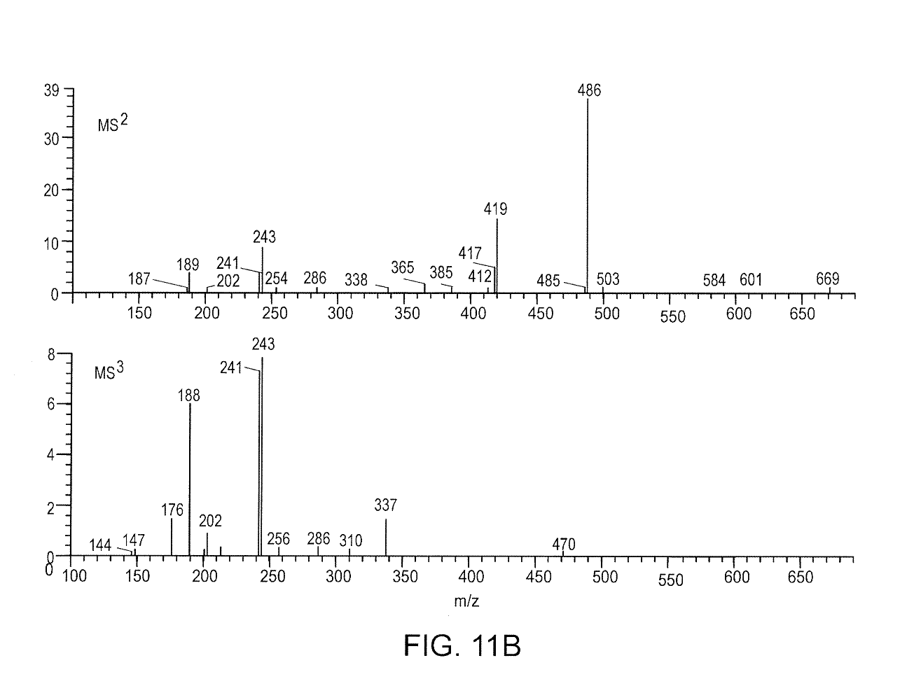

Metabolite M7 produced a [M+H]⁺ at m/z 662, which was 190 Da larger than COMPOUND I. LC/MS with D₂O substituted for H₂O in the mobile phase generated [M+D]⁺ at m/z 668, which indicated five exchangeable hydrogens. The proposed fragmentation scheme and product ions of m/z 662 mass spectra for M7 are shown in FIG. 11. Loss of 176 Da from [M+H]⁺ yielded m/z 504, which was 32 Da larger than COMPOUND I and indicated that M3 was a glucuronide of a dihydroxy COMPOUND I. Product ions at m/z 243, 241, 189 and 176 were 14 Da larger than the corresponding ions at m/z 229, 227, 175 and 162, respectively, for COMPOUND I, which indicated both hydroxylation and oxidative defluorination of the fluoroquinoline. The product ion at m/z 419 was 176 Da larger than m/z 243, which indicated that one of the hydroxyl groups on the defluorinated fluoroquinoline was the site of glucuronidation. This was also consistent with the product ion at m/z 337, generated by fragmentation of the piperidine-quinoline bond. Therefore, M7 was identified as a dihydroxy desfluoro COMPOUND I glucuronide.

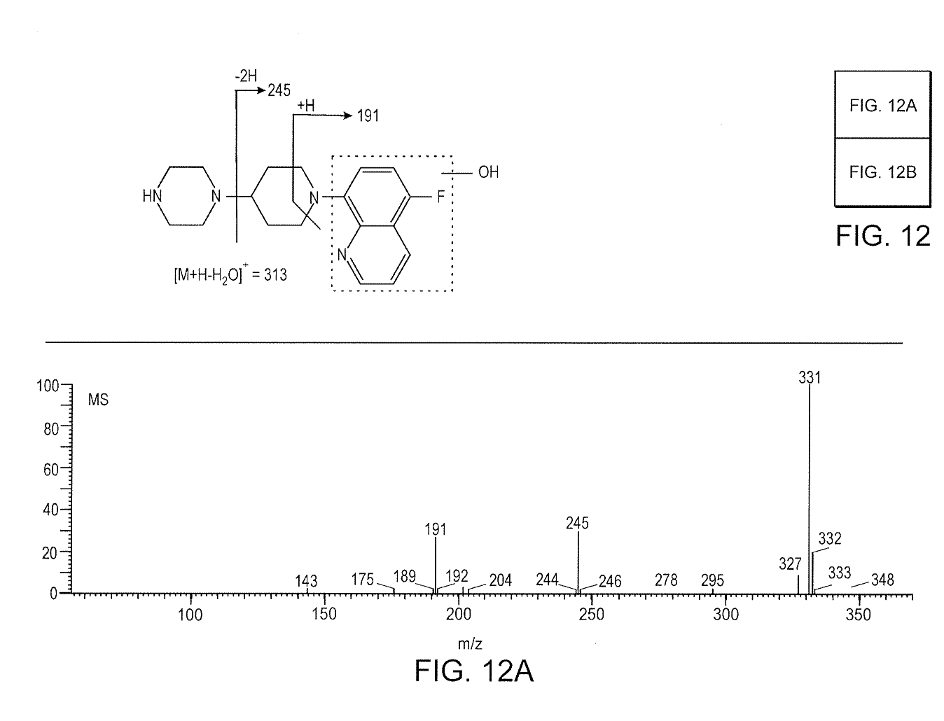

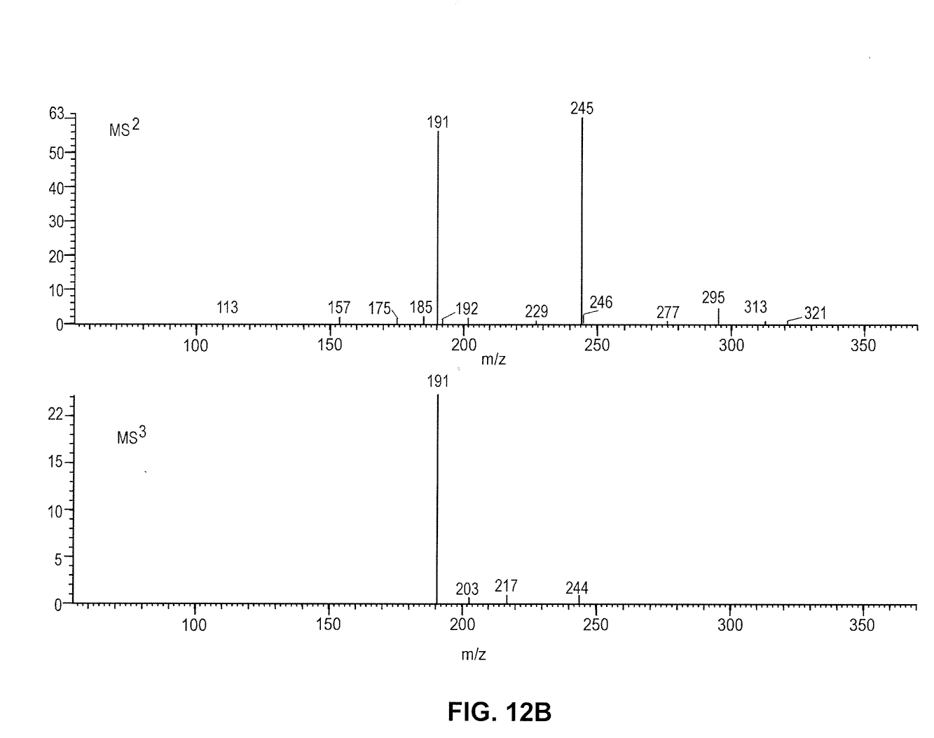

The [M+H]⁺ for M8 was observed at m/z 331, which was 141 Da less than COMPOUND I and 16 Da larger than M14. LC/MS with D₂O substituted for H₂O in the mobile phase generated [M+D]⁺ at m/z 334. These data indicated two exchangeable hydrogens, which was two more than COMPOUND I and consistent with N-dealkylation of the COMPOUND I molecule. The product ions of m/z 331 mass spectra and the proposed fragmentation scheme for M8 are presented in FIG. 12. Loss of H₂O from [M+H]⁺ generated a weak product ion at m/z 313, which was consistent with an aromatic hydroxyl group. The product ions at m/z 245 and 191 were 16 Da larger than the corresponding ions at m/z 229 and 175, respectively, for COMPOUND I, which indicated hydroxylation of the fluoroquinolinyl-aminomethylene moiety. These data and the molecular weight difference between M8 and COMPOUND I indicated that the methoxyquinoline moiety of COMPOUND I was not present in M8. Therefore, M8 was identified as a hydroxyl N-desmethoxyquinolinyl COMPOUND I.

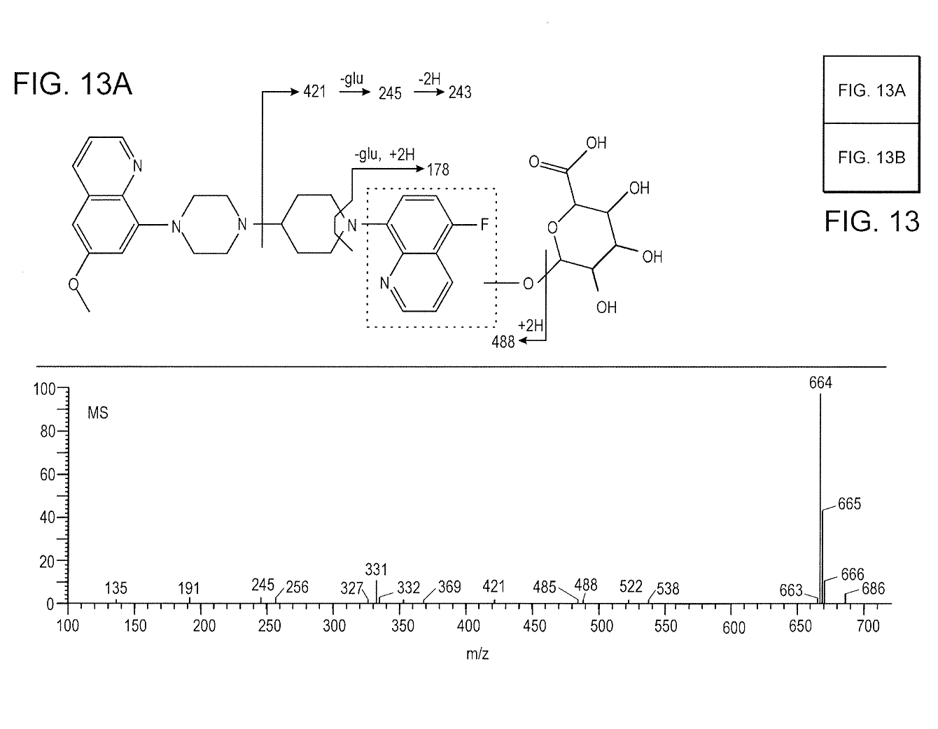

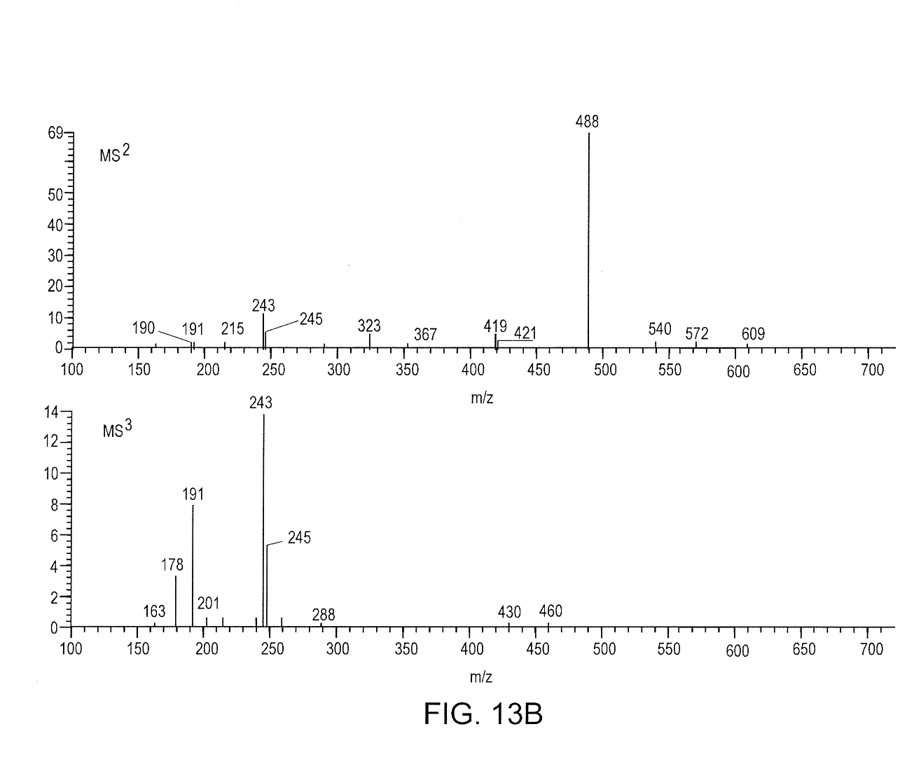

Metabolite M9 produced a [M+H]⁺ at m/z 664, which was 192 Da larger than COMPOUND I. LC/MS with D₂O substituted for H₂O in the mobile phase generated [M+D]⁺ at m/z 669, which indicated four exchangeable hydrogens. The proposed fragmentation scheme and product ions of m/z 664 mass spectra for M9 are shown in FIG. 13. Loss of 176 Da from [M+H]⁺ yielded m/z 488, which was 16 Da larger than COMPOUND I and indicated that M9 was a glucuronide of a hydroxyl COMPOUND I. Product ions at m/z 245, 243 and 178 were 16 Da larger than the corresponding ions at m/z 229, 227 and 162, respectively, for COMPOUND I, which indicated hydroxylation of the fluoroquinoline. The product ion at m/z 421 was 176 Da larger than m/z 245, which indicated that the hydroxylated fluoroquinoline was the site of glucuronidation. Therefore, M9 was identified as a hydroxy COMPOUND I glucuronide.

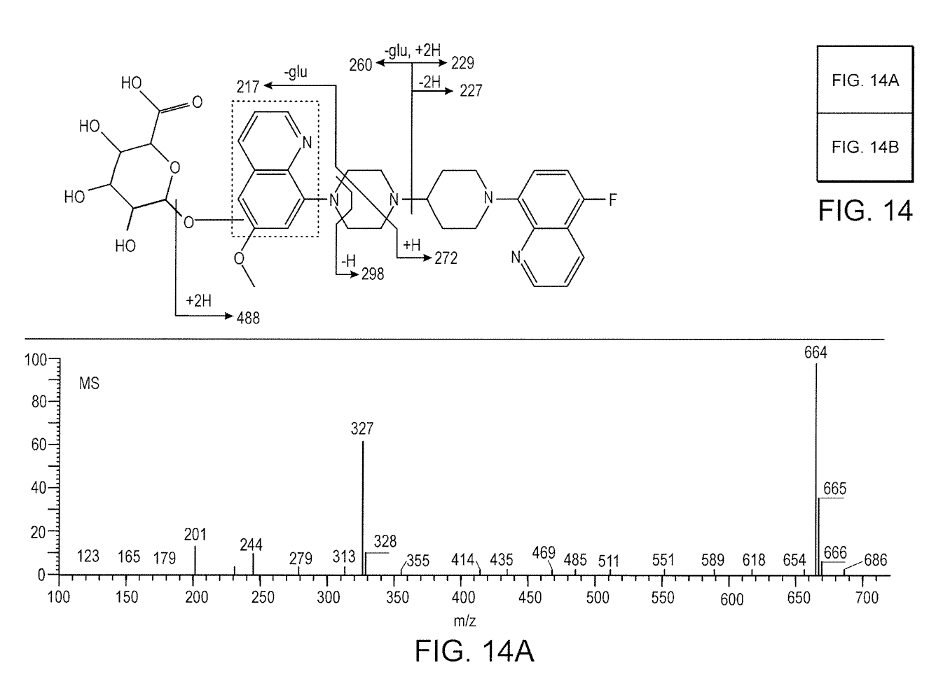

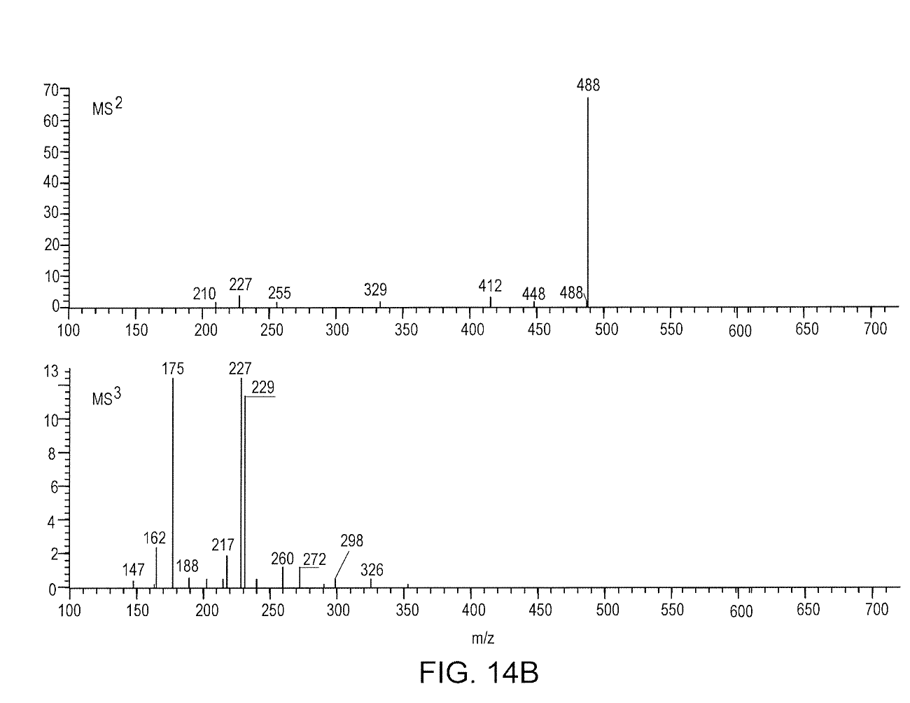

The [M+H]⁺ for M10 was observed at m/z 664, which was 192 Da larger than COMPOUND I. LC/MS with D₂O substituted for H₂O in the mobile phase generated [M+D]⁺ at m/z 669, which indicated four exchangeable hydrogens. The proposed fragmentation scheme and product ions of m/z 664 mass spectra for M10 are shown in FIG. 14. Loss of 176 Da from [M+H]⁺ yielded m/z 488, which was 16 Da larger than COMPOUND I and indicated that M10 was a glucuronide of a hydroxy COMPOUND I. Product ions at m/z 298, 272, 229 and 227 were also observed for COMPOUND I, which indicated unchanged fluoroquinoline, piperidine and piperazine rings. Product ions at m/z 260 and 217 were 16 Da larger than the corresponding ions at m/z 244 and 201, respectively, for COMPOUND I, which in combination with m/z 298 and 272 indicated hydroxylation of the methoxyquinoline. Consequently, the hydroxylated methoxyquinoline was the site of glucuronidation. Therefore, M10 was identified as a hydroxy COMPOUND I glucuronide.

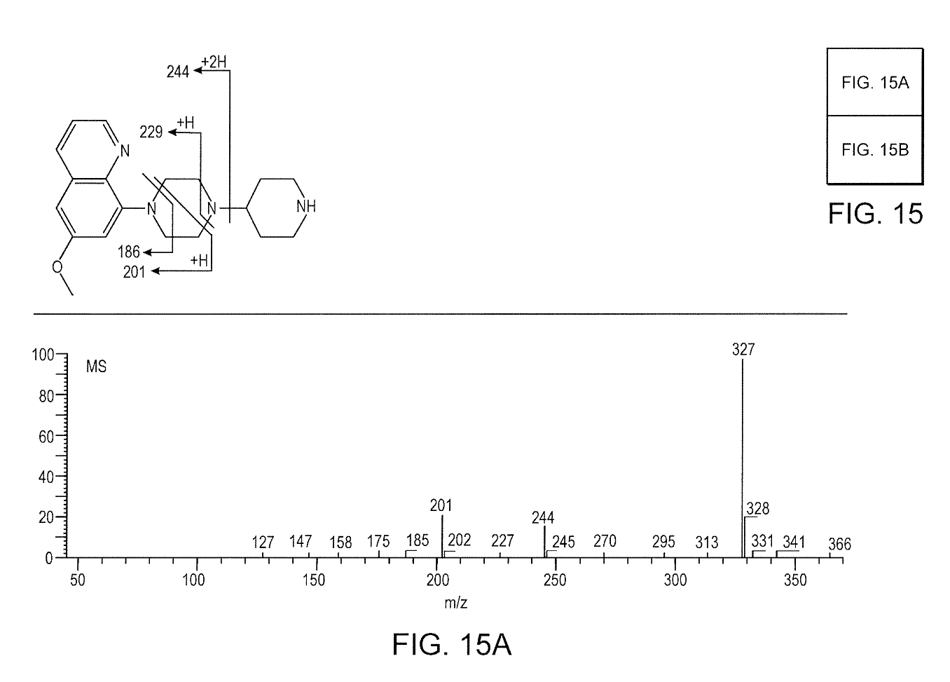

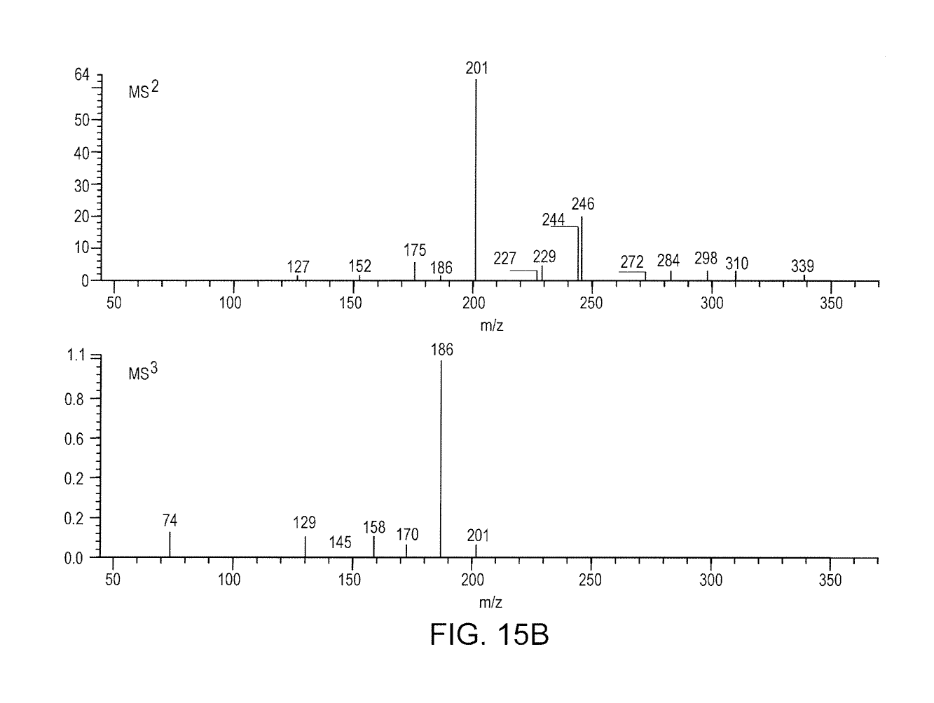

Metabolite M12 produced a [M+H]⁺ at m/z 327, which was 145 Da less than COMPOUND I. LC/MS with D₂O substituted for H₂O in the mobile phase generated [M+D]⁺ at m/z 329. These data indicated one exchangeable hydrogen, which was one more than COMPOUND I and consistent with N-dealkylation of the COMPOUND I molecule. The product ions of m/z 327 mass spectra and the proposed fragmentation scheme for M12 are presented in FIG. 15. Product ions at m/z 244 and 201 were also observed for COMPOUND I. Fragmentation of the piperazine ring generated m/z 229 and 186. These data indicated an intact methoxyquinolinyl-piperazine moiety, which in combination with the molecular weight difference between M12 and COMPOUND I indicated that the fluoroquinoline moiety of COMPOUND I was not present in M12. Therefore, M12 was identified as N-desfluoroquinolinyl COMPOUND I.

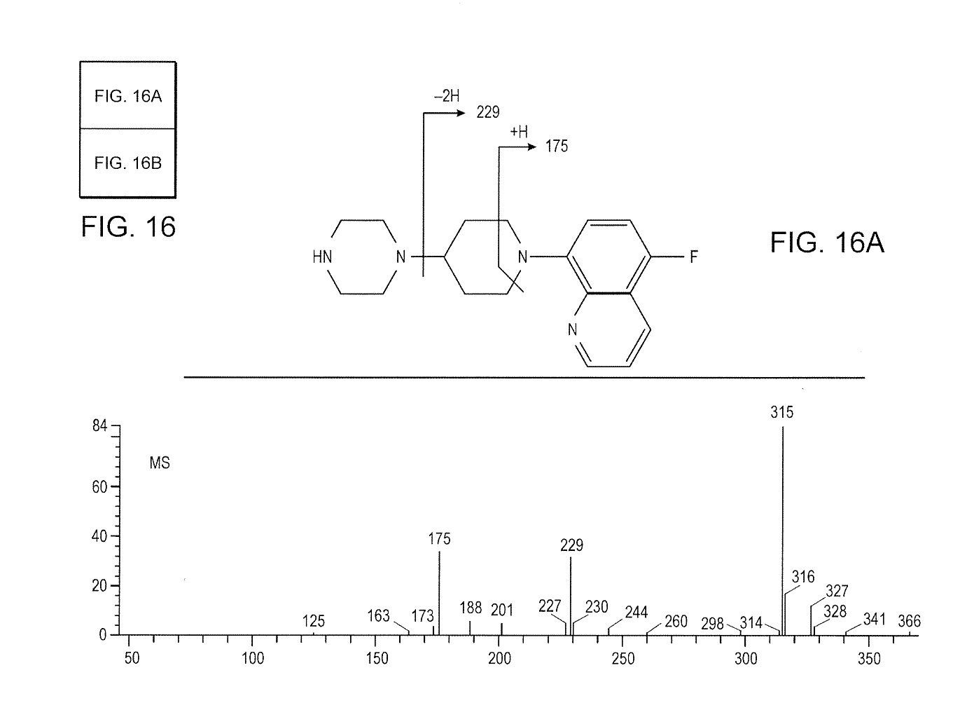

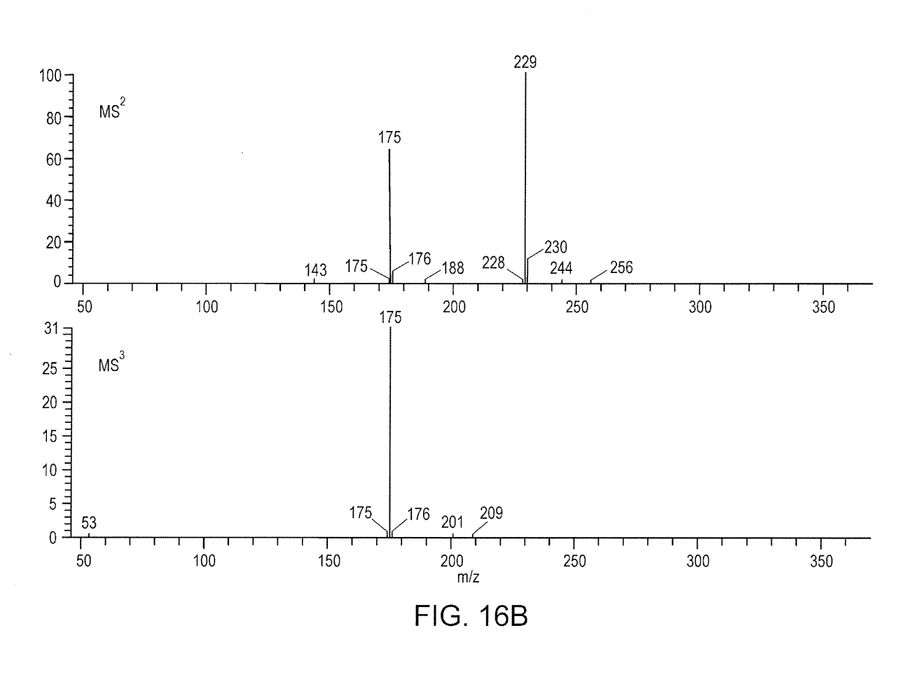

The [M+H]⁺ for M14 was observed at m/z 315, which was 157 Da less than COMPOUND I. LC/MS with D₂O substituted for H₂O in the mobile phase generated [M+D]⁺ at m/z 317. These data indicated one exchangeable hydrogen, which was one more than COMPOUND I and consistent with N-dealkylation of the COMPOUND I molecule. The productions of m/z 315 mass spectra and the proposed fragmentation scheme for M14 are presented in FIG. 16. The product ions at m/z 229 and 175 were also observed for COMPOUND I, which indicated unchanged fluoroquinoline and piperidine rings. These data and the molecular weight difference between M14 and COMPOUND I indicated that the methoxyquinoline moiety of COMPOUND I was not present in M14. Therefore, M14 was identified as N-desmethoxyquinolinyl COMPOUND I.

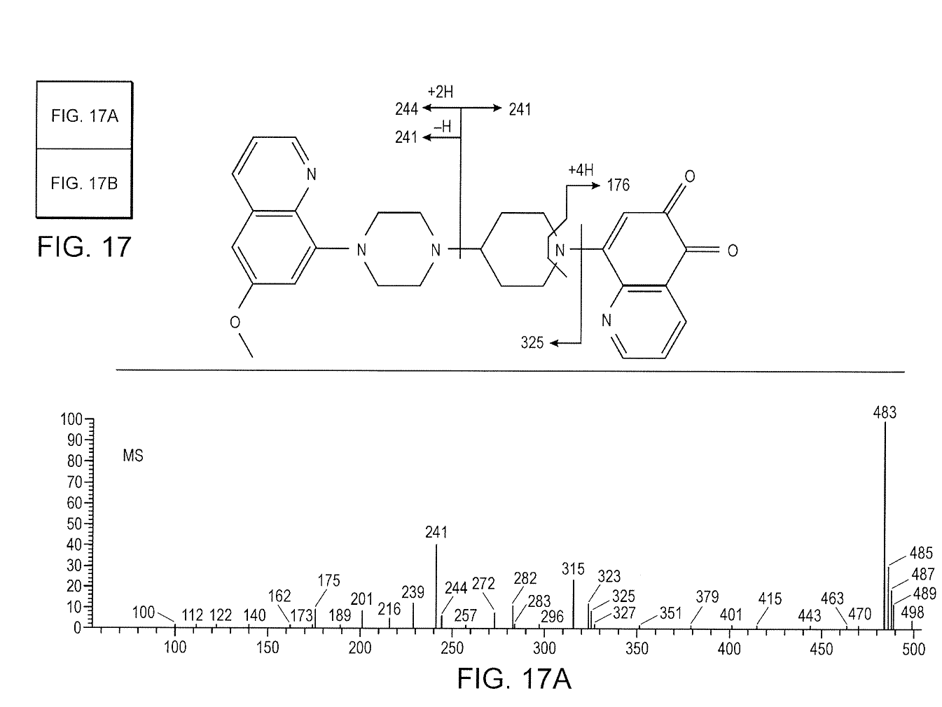

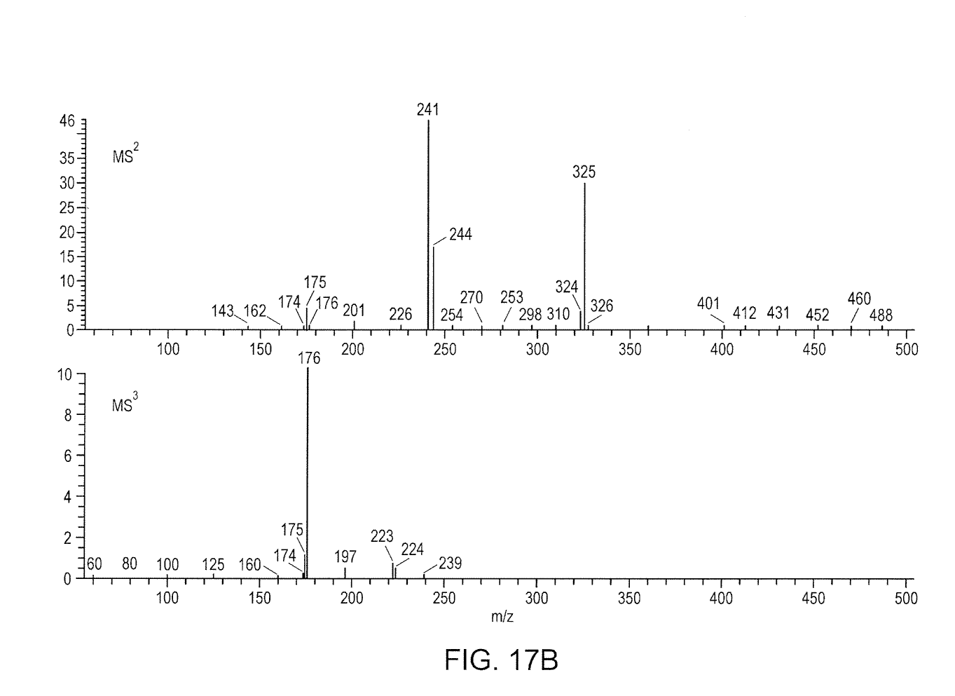

Metabolite M116 produced a [M+H]⁺ at m/z 484, which was 12 Da larger than COMPOUND I. LC/MS with D₂O substituted for H₂O in the mobile phase generated [M+D]⁺ at m/z 485, which indicated no exchangeable hydrogens, the same as for COMPOUND I. The product ions of m/z 484 mass spectra and the proposed fragmentation scheme for M16 are presented in FIG. 17. Product ions at m/z 244 and 241, also observed for COMPOUND I, suggested an unchanged methoxyquinolinyl-piperazine moiety. The product ion at m/z 241 was also 12 Da larger than the m/z 229 product ion of COMPOUND I, which was consistent with metabolism of the fluoroquinolinyl-piperidine moiety. Fragmentation of the piperidine-quinoline bond generated m/z 325, not observed for COMPOUND I, which was consistent with unchanged methoxyquinoline, piperazine and piperidine rings, and consequently indicated metabolism of the fluoroquinoline ring. Fragmentation of the piperidine ring with charge retention on the fragment containing the metabolized fluoroquinoline ring yielded m/z 176. These data and the molecular weight difference between M16 and COMPOUND I were consistent with the keto metabolite of a keto desfluoro COMPOUND I formed by oxidative defluorination and subsequent oxidation. Therefore, M16 was proposed to be a desfluoro COMPOUND I quinone.

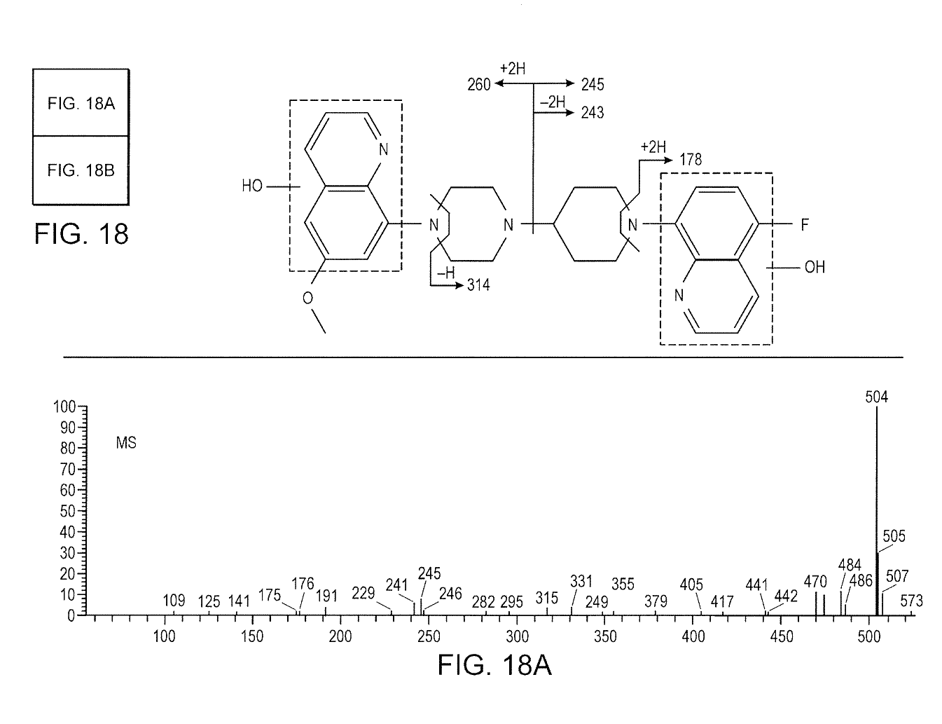

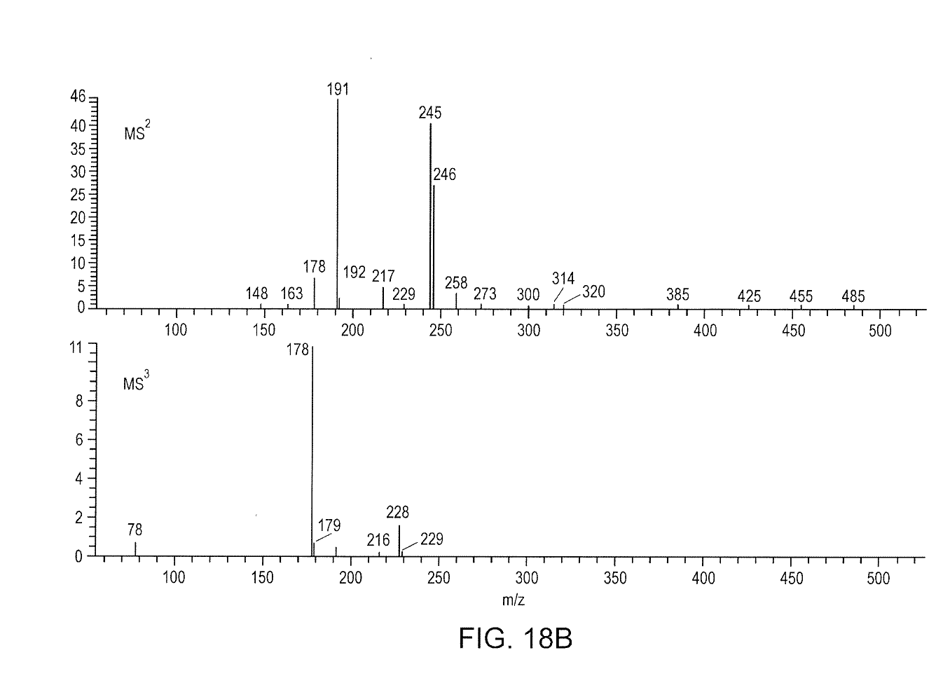

Metabolite M17 produced a [M+H]⁺ at m/z 504, which was 32 Da larger than COMPOUND I. LC/MS with D₂O substituted for H₂O in the mobile phase generated [M+D]⁺ at m/z 507. These data indicated two exchangeable hydrogens, which was two more than COMPOUND I and consistent with the presence of two hydroxyl groups. The product ions of m/z 504 mass spectra and the proposed fragmentation scheme for M17 are presented in FIG. 18. Product ions atm/z 314, 245 and 243 were 16 Da larger than the corresponding ions at m/z 298, 229, 227 and 162, respectively, for COMPOUND I, which indicated hydroxylation of the fluoroquinoline ring. The product ion at m/z 260 was 16 Da larger than the corresponding ion at m/z 244 for COMPOUND I, which in combination with m/z 314 indicated hydroxylation of the methoxyquinoline ring. Therefore, M17 was identified as a dihydroxy COMPOUND I.

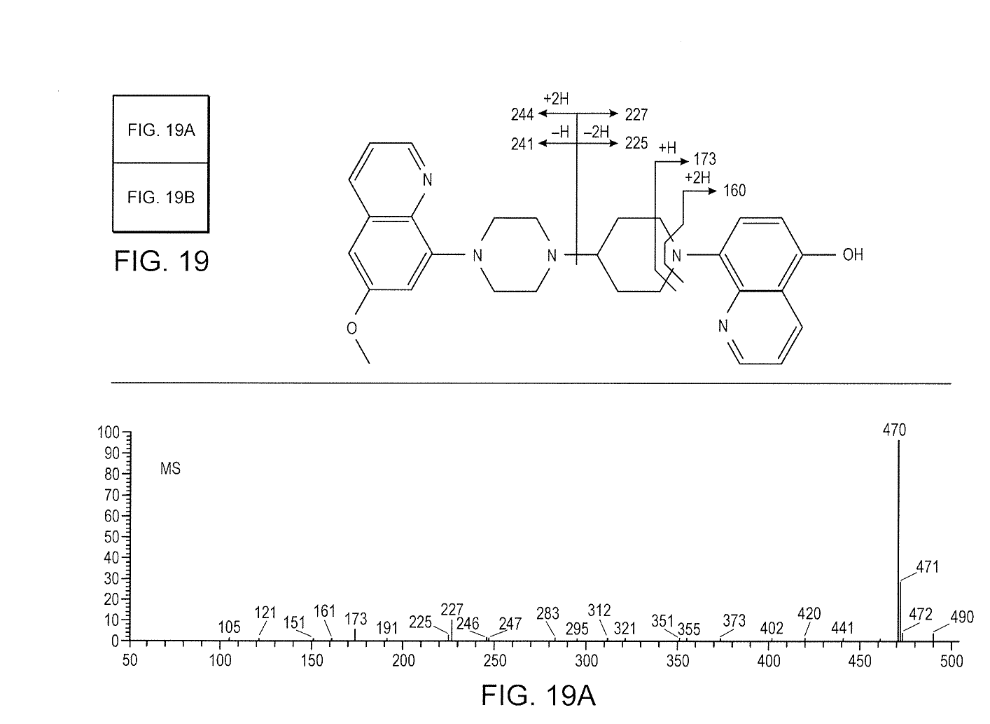

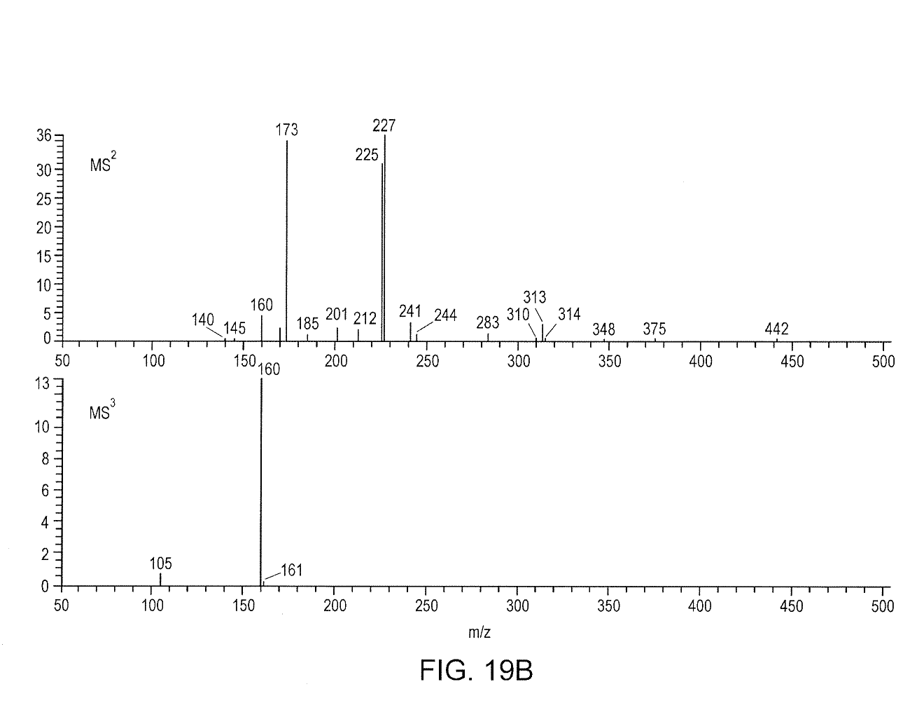

Metabolite M18 produced a [M+H]⁺ at m/z 470, which was 2 Da less than COMPOUND I. LC/MS with D₂O substituted for H₂O in the mobile phase generated [M+D]⁺ at m/z 472. These data indicated one exchangeable hydrogen, which was one more than COMPOUND I and consistent with the presence of an —OH or —NH group not present in COMPOUND I. The product ions of m/z 470 mass spectra and the proposed fragmentation scheme for M18 are presented in FIG. 19. Product ions at m/z 244 and 241 were also observed for COMPOUND I, which indicated an unchanged methoxyquinolinyl-piperazine moiety. Product ions at m/z 227, 225, 173 and 160 were 2 Da less than the corresponding ions at m/z 229, 227, 175 and 162, respectively, for COMPOUND I, which indicated that the fluoroquinoline ring was the site of metabolism. The molecular weight difference and additional exchangeable hydrogen for M18 compared to COMPOUND I indicated oxidative defluorination of the fluoroquinoline. Therefore, M18 was identified as hydroxyl desfluoro COMPOUND I.

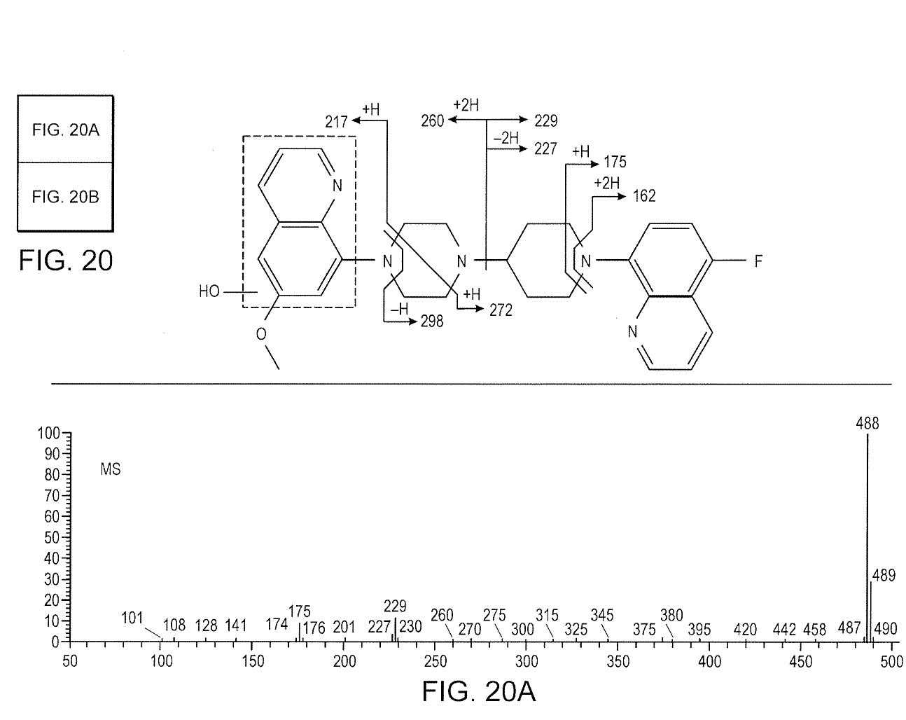

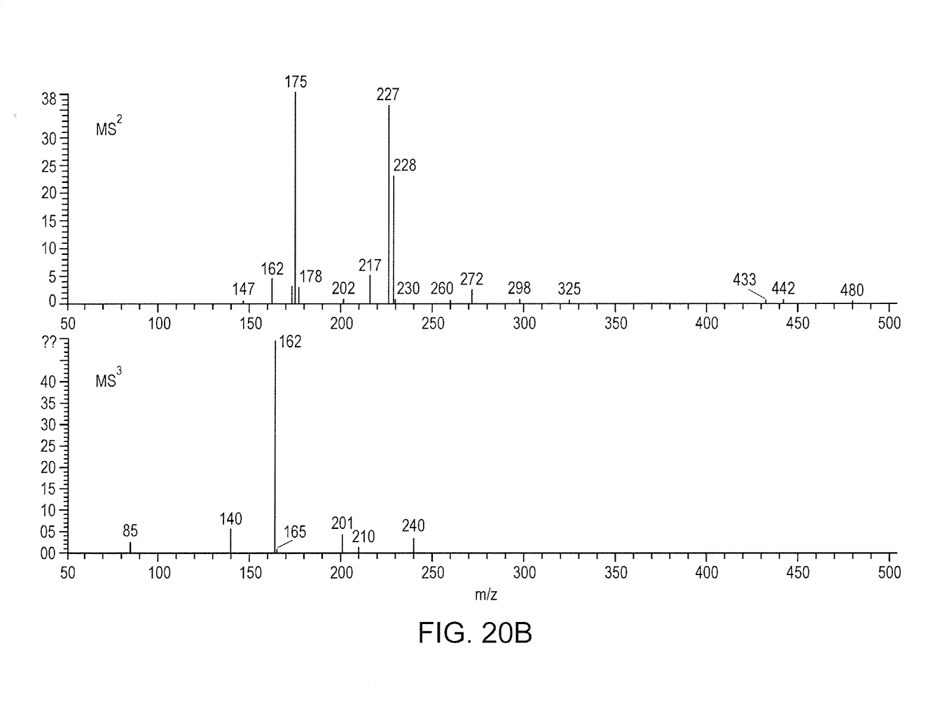

Metabolite M20 produced a [M+H]⁺ at m/z 488, which was 16 Da larger than COMPOUND I. LC/MS with D₂O substituted for H₂O in the mobile phase generated [M+D]⁺ at m/z 490. These data indicated one exchangeable hydrogen, which was one more than COMPOUND I and consistent with hydroxylation rather than N-oxidation. The product ions of m/z 488 mass spectra and the proposed fragmentation scheme for M20 are presented in FIG. 20. Product ions at m/z 298, 272, 229 and 227 were also observed for COMPOUND I, which indicated unchanged fluoroquinoline, piperidine and piperazine rings. Product ions at m/z 260 and 217 were 16 Da larger than the corresponding ions at m/z 244 and 201, respectively, for COMPOUND I, which in combination with m/z 298 and 272 indicated hydroxylation of the methoxyquinoline. Therefore, M20 was identified as a hydroxy COMPOUND I.

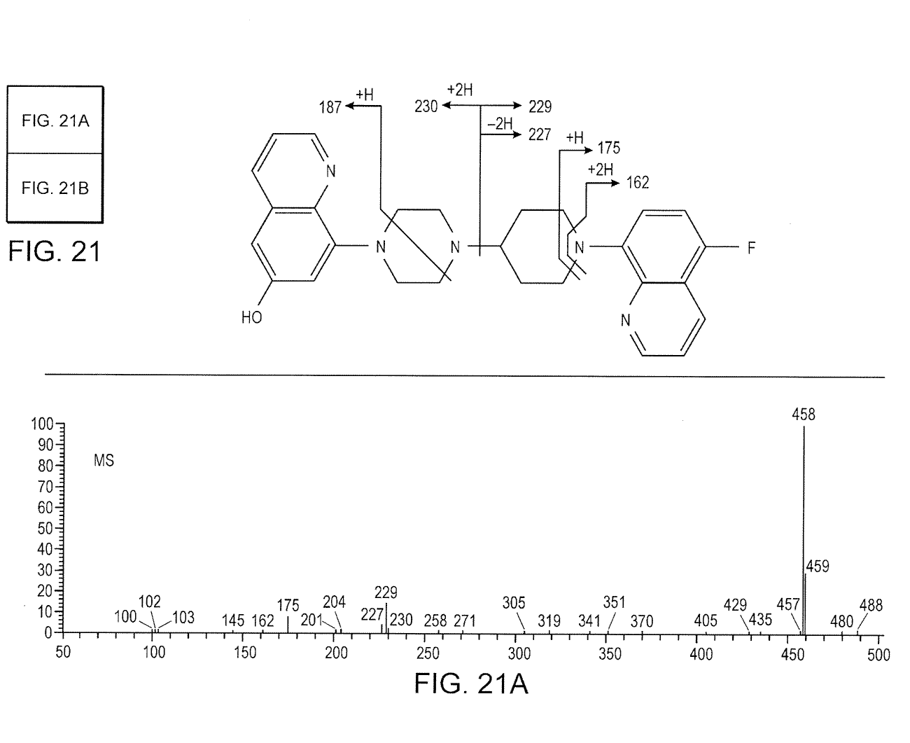

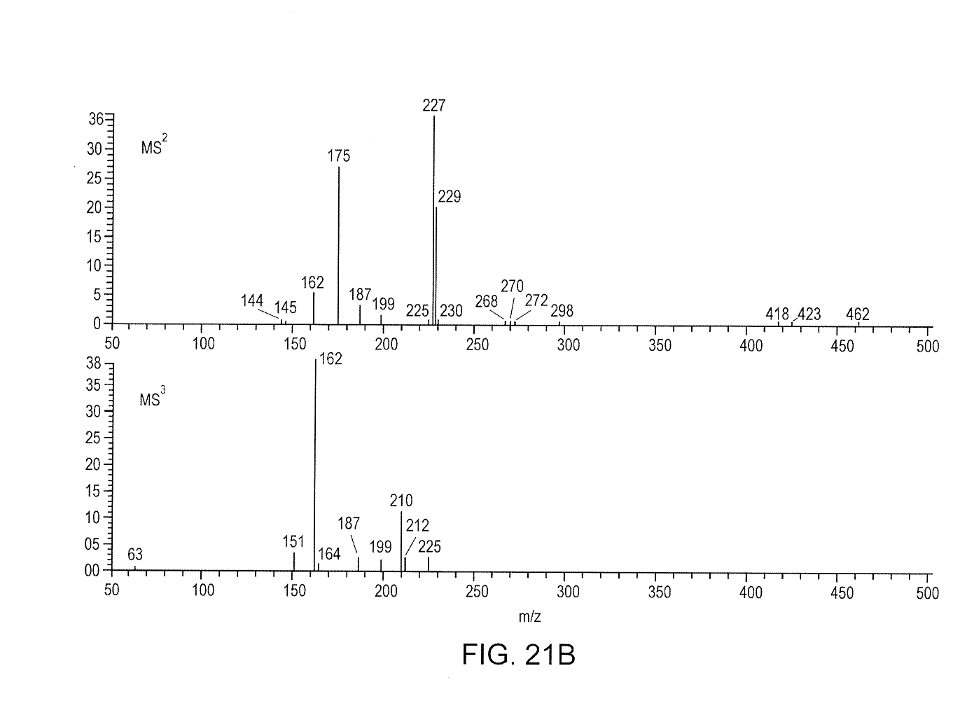

The [M+H]⁺ for M21 was observed at m/z 458, which was 14 Da less than COMPOUND I. LC/MS with D₂O substituted for H₂O in the mobile phase generated [M+D]⁺ at m/z 460 (data not shown). These data indicated one exchangeable hydrogen, which was one more than COMPOUND I and consistent with the presence of a hydroxyl group. The product ions of m/z 458 mass spectra and the proposed fragmentation scheme for M21 are presented in FIG. 21. Product ions at m/z 229, 227, 175 and 162 were also observed for COMPOUND I, which indicated an unchanged fluoroquinolinyl-piperidine moiety. The product ion at m/z 187 was 14 Da less than the corresponding methoxyquionolinyl ion at m/z 201 for COMPOUND I, which indicated demethylation of the methoxy group. Therefore, M21 was identified as O-desmethyl COMPOUND I.

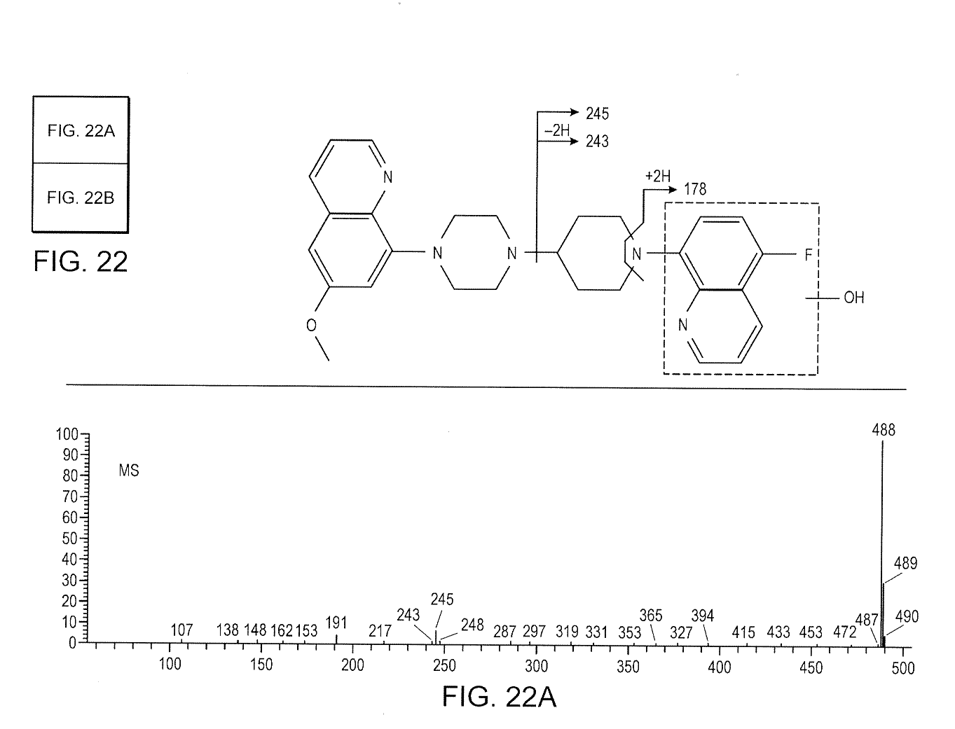

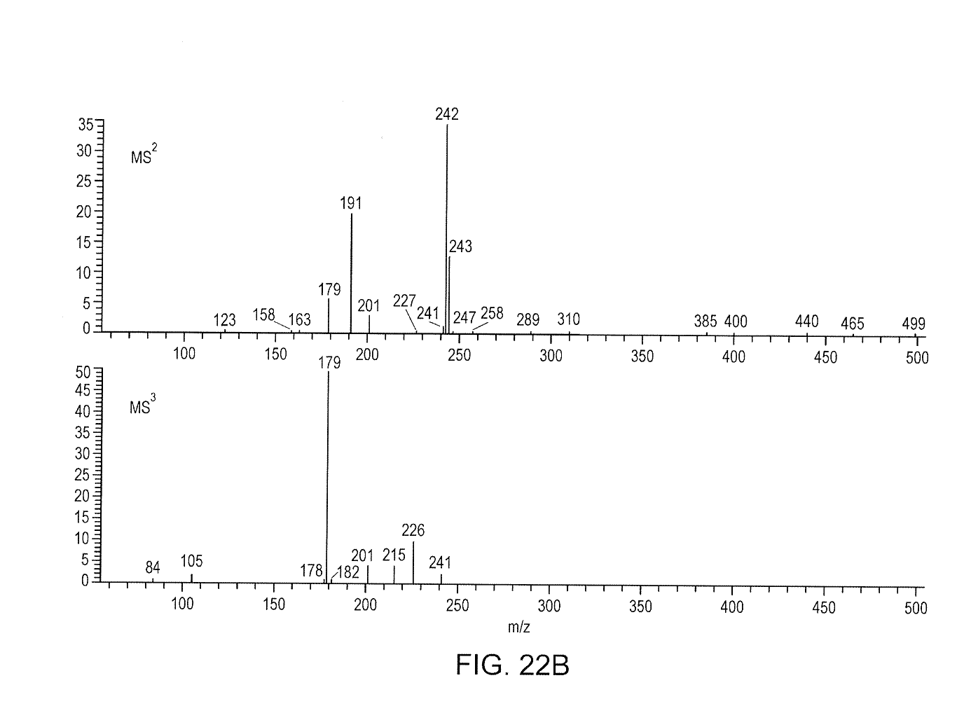

Metabolite M22 produced a [M+H]⁺ at m/z 488, which was 16 Da larger than COMPOUND I. LC/MS with D₂O substituted for H₂O in the mobile phase generated [M+D]⁺ at m/z 490. These data indicated one exchangeable hydrogen, which was one more than COMPOUND I and consistent with hydroxylation rather than N-oxidation. The product ions of m/z 488 mass spectra and the proposed fragmentation scheme for M22 are presented in FIG. 22. Product ions at m/z 245, 243, 191 and 178 were 16 Da larger than the corresponding ions at m/z 229, 227, 175 and 162, respectively, for COMPOUND I, which indicated hydroxylation of the fluoroquinoline. Therefore, M22 was identified as a hydroxy COMPOUND I.

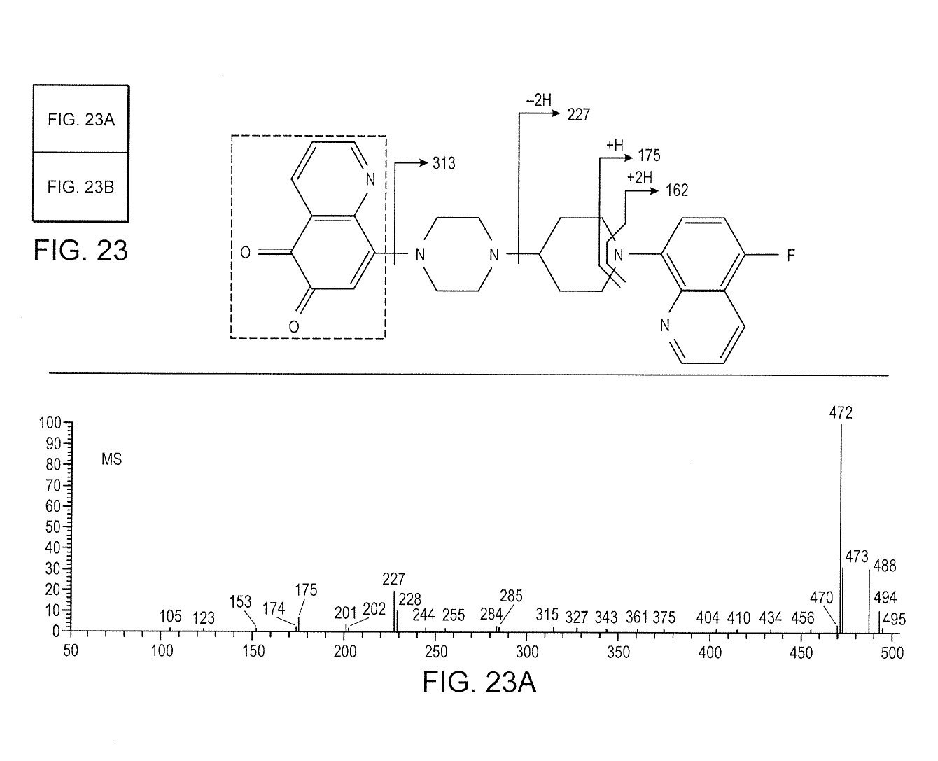

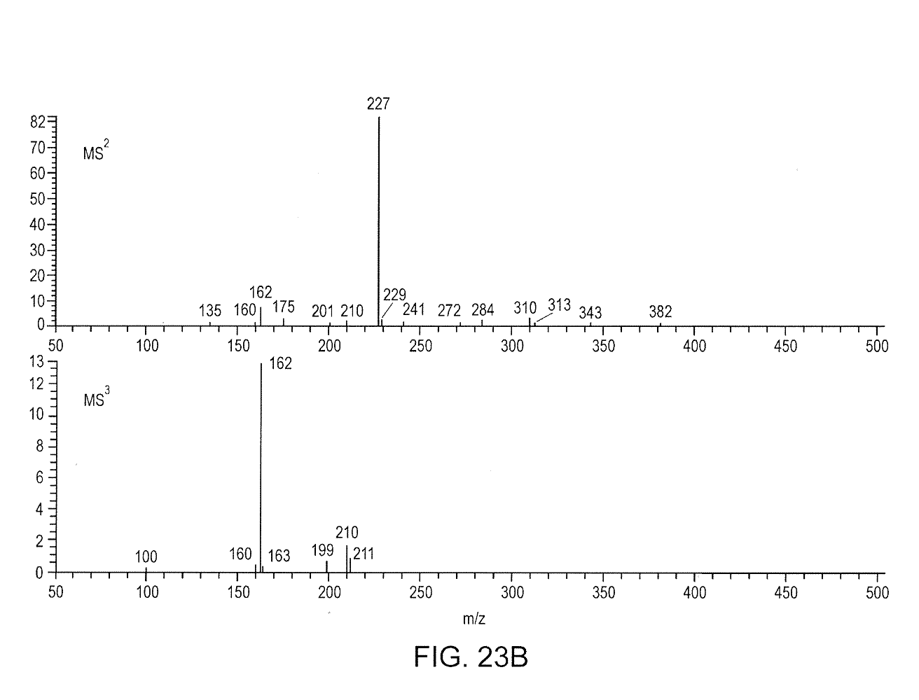

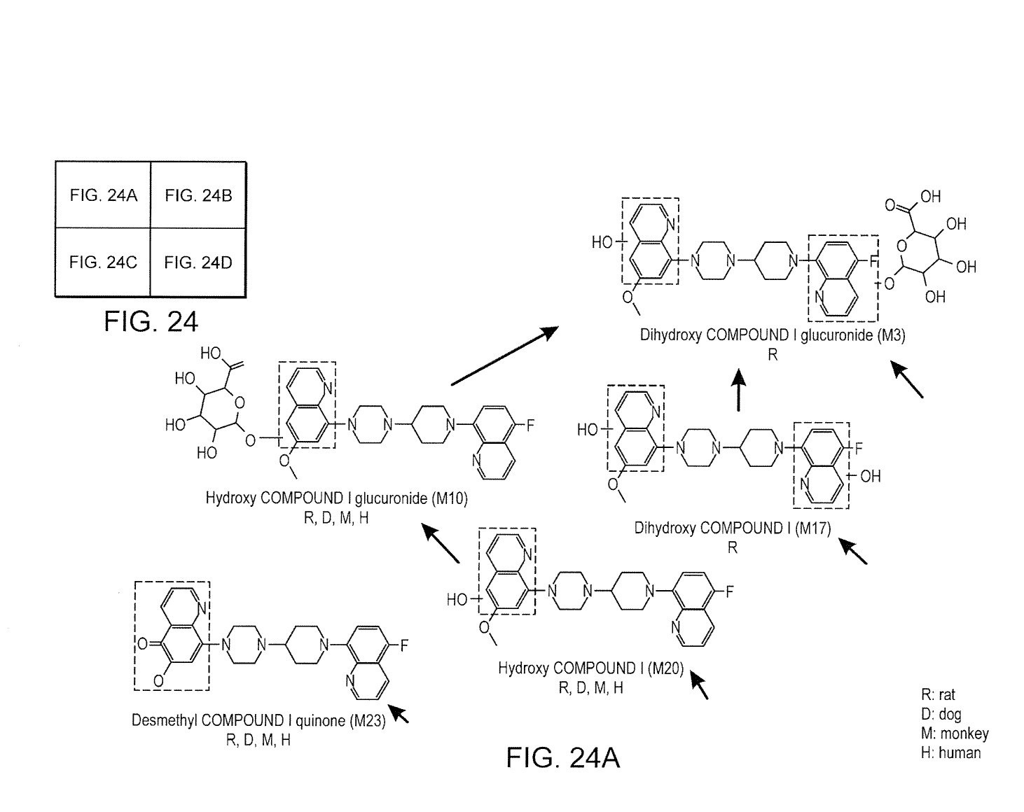

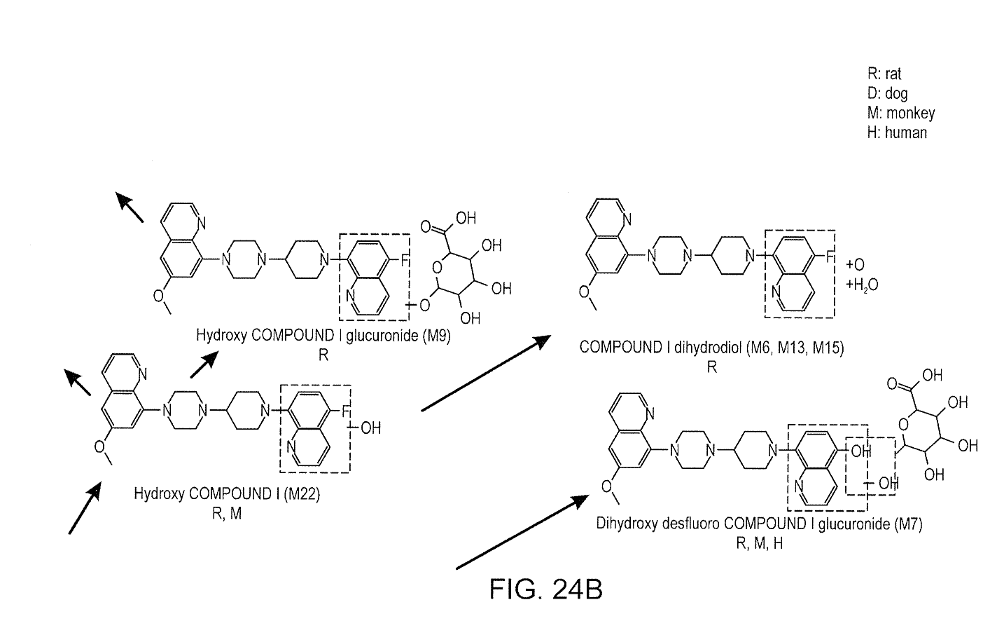

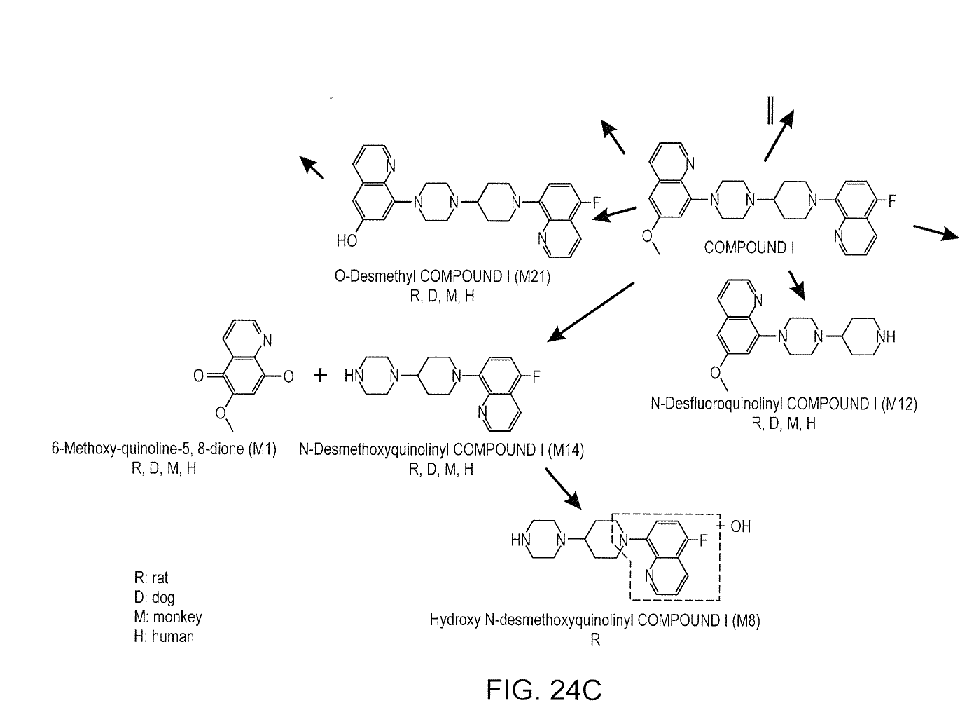

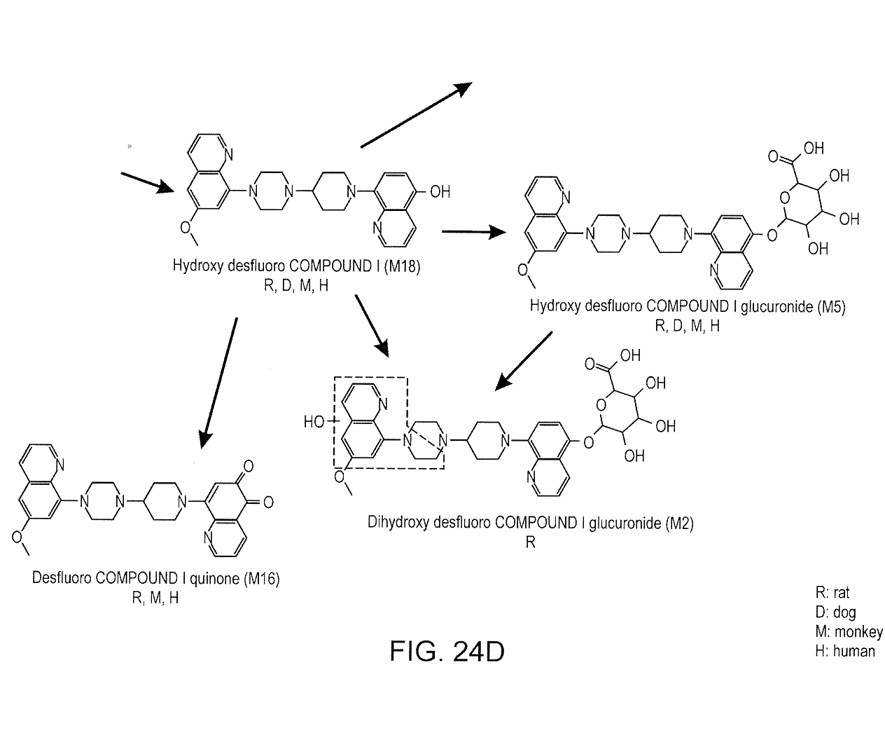

Metabolite M23 produced a [M+H]⁺ at m/z 472, which the same as for COMPOUND I. LC/MS with D₂O substituted for H₂O in the mobile phase generated [M+D]⁺ at m/z 473, which indicated no exchangeable hydrogens, the same as for COMPOUND I. The product ions of m/z 472 mass spectra and the proposed fragmentation scheme for M23 are presented in FIG. 23. Product ions at m/z 227 and 162 were also observed for COMPOUND I, which indicated an unchanged fluoroquinolinyl-piperidine moiety. Fragmentation of the piperazine-quinoline bond generated m/z 313, not observed for COMPOUND I, which was consistent with unchanged fluoroquinoline, piperidine and piperazine rings, and consequently indicated metabolism of the methoxyquinoline. These data and the molecular weight difference between M23 and COMPOUND I were consistent with the keto metabolite of a keto desmethyl COMPOUND I formed by O-demethylation and subsequent oxidation. Therefore, M23 was proposed to be a desmethyl COMPOUND I quinone.

Metabolite M25 produced a [M+H]⁺ at m/z 524. Metabolite M26 produced a [M+H]⁺ at m/z 506. Both M25 and M26 were identified as the tetrahydro triols of COMPOUND I.

### In Vitro Metabolism of [¹⁴C]COMPOUND I in Cryopreserved Rat, Dog, and Human Hepatocytes

The in vitro biotransformation of [¹⁴C]COMPOUND I in pooled cryopreserved rat, dog, and human hepatocytes was investigated. Metabolite profiles were determined by HPLC with UV and radioactivity detection and metabolites were identified by LC/MS.

[¹⁴C]COMPOUND I trisuccinate salt was prepared as described in Example 3. The structure of [¹⁴C]COMPOUND I with the positions of the ¹⁴C labels is shown below:

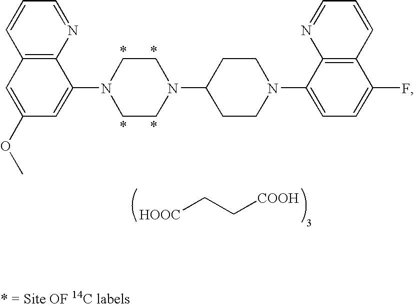

Pooled cryopreserved male rat (n=5), male dog (n=2), and mixed male and female human (n=10) hepatocytes used in this study and their characteristics are described in Table 12. Hepatocyte thawing and incubation media were purchased from In Vitro Technologies (Baltimore, Md.). [¹⁴C]7-Ethoxycoumarin (64.5 mCi/mmol, purity 98%) was purchased from New England Nuclear (Boston, Mass.). Deuterium oxide (D₂O) was obtained from Cambridge Isotope Laboratories (Andover, Mass.). Scintillation cocktails, Ultima Gold and Ultima Flo M, were purchased from Perkin Elmer Life Sciences (Boston, Mass.). The solvents used for extraction and for chromatographic analysis were HPLC grade or ACS reagent grade (Mallinckrodt Baker, Phillipsburg, N.J.).

Pooled male rat, dog, and mixed male and female human cryopreserved hepatocytes were used in this study. The cryopreserved hepatocytes (two vials from each species; three to five million viable cells per vial) were thawed in a 37° C. water bath with gentle shaking until the ice was almost melted. The suspensions from the two individual vials of the same species were immediately transferred to a 50 mL centrifuge tube containing pre-warmed thawing media at 37° C. with gentle handshaking to prevent the cells from settling. The cell suspension was centrifuged at 50 g for 5 min at 4° C. Supernatants were discarded and the pellets were resuspended in pre-warmed incubation media (8 mL) at 37° C. The percentages of viable cells in the suspension were 57%, 75%, and 90% for rat, dog, and human hepatocytes, respectively, which was determined using the Trypan Blue stain method. [¹⁴C]COMPOUND I (20 μM) was incubated in a 12-well plate containing hepatocyte suspensions at 1.0 mL per well in duplicate (≧1 million viable cells/well) at 37° C. for 1 or 4 hr in the presence of 5% CO₂:95% O₂. Control incubations without hepatocytes were also run under the same conditions. Positive controls containing [¹⁴C]7-ethoxycoumarin (100 μM) were also incubated under the same conditions, but only for 1 hr. At the end of the incubations, reactions were stopped by adding acetonitrile with 2% acetic acid (1 mL), mixed for 10 min, followed by centrifugation at 4000 rpm for 10 min at 4° C. Aliquots (50 μL) of supernatants were analyzed by HPLC with UV and radioactivity flow detection for metabolite profiling, and by LC/MS for metabolite identification as described in below. To determine extraction recovery, aliquots (20 μL) of supernatants were analyzed for radioactivity content utilizing a Packard Tri-Carb Model 3100 TR liquid scintillation counter and 5 mL of Ultima Gold.

Chromatographic analyses were performed with a Waters Alliance model 2695 HPLC system (Waters Corp., Milford, Mass.) that was equipped with a built-in autosampler. The column eluent was monitored with a model 996 diode array UV detector, set to monitor 250 nm, and a FloOne β Model A525 radioactivity flow detector (Perkin Elmer) with a 250 μL flow cell. Separation of the parent compound from metabolites was accomplished using a Discovery C18 column, 250×4.6 mm, 5 μm (Supelco, Bellefonte, Pa.) at an ambient temperature of approximately 20° C. Mobile phase A was 10 mM ammonium acetate (pH 4.5) and mobile phase B was acetonitrile and they were delivered at 1 mL/min using the gradient described in Table 10. The Ultima Flo M scintillant flow rate was 3 mL/min.

TABLE 10
HPLC Gradient

| Time (min) | A (%) | B (%) |
|---|---|---|
| 0 | 95 | 5 |
| 10 | 95 | 5 |
| 20 | 82 | 18 |
| 27 | 82 | 18 |
| 30 | 79 | 21 |
| 50 | 72 | 28 |
| 60 | 55 | 45 |
| 65 | 25 | 75 |
| 70 | 25 | 75 |
| 80 | 10 | 90 |
| 81 | 95 | 5 |

The HPLC system used for mass spectrometric analysis was a Waters Alliance model 2695 HPLC system (Waters Corp). It was equipped with a built-in autosampler and a model 996 diode array UV detector. The UV detector was set to monitor 210-400 nm. The HPLC conditions were the same as those described hereinabove with the following exceptions. The internal diameter of the HPLC column was 2.1 mm and the flow rate was 0.2 mL/min. The column re-equilibration time was 14 min (95 min total run time). For H-D exchange experiments, D₂O was substituted for water in mobile phase A. During LC/MS sample analysis, up to the first 5 min of flow was diverted away from the mass spectrometer prior to evaluation of metabolites.

The mass spectrometer used for metabolite characterization was a Micromass Quattro Micro triple quadrupole mass spectrometer (Waters Corp.). It was equipped with an electrospray ionization (ESI) interface and operated in the positive ionization mode. Settings for the mass spectrometer are listed below in Table 11.

TABLE 11
Micromass Mass Spectrometer Settings

| | |
|---|---|
| ESI spray | 2.5 kV |
| Cone | 45 V |
| Mass resolution of scanning mass analyzer | 0.7 Da ± 0.2 Da |
| | width at half height |
| Mass resolution of non-scanning mass | 1-2 Da width at half height |
| analyzer for MS/MS experiments | |
| Desolvation gas flow | 950-1100 L/hr |
| Source block temp | 80° C. |
| Desolvation gas temp | 250° C. |
| Collision gas pressure | 1.0-1.2 × 10⁻³ mbar |
| Collision offset | 30 eV |

Flo-One analytical software (version 3.65) was utilized to integrate the radioactive peaks. Means and standard deviations were calculated using Microsoft Excel® 2000. Micromass MassLynx software (version 4.0, Waters Corp.) was used for control of LC/MS equipment and recording of data from LC/MS analyses.

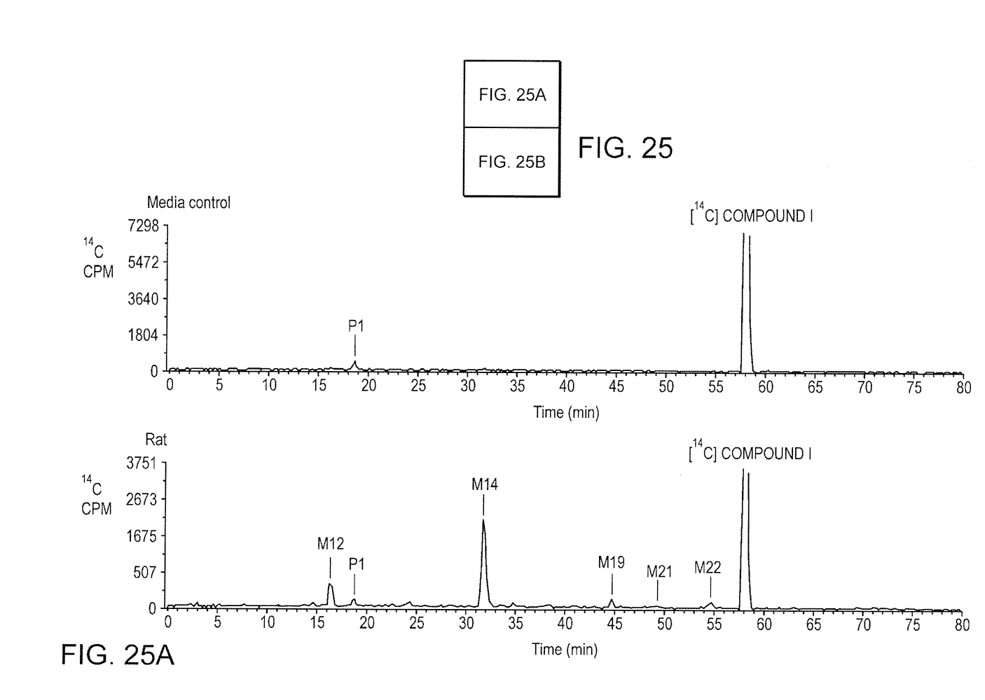

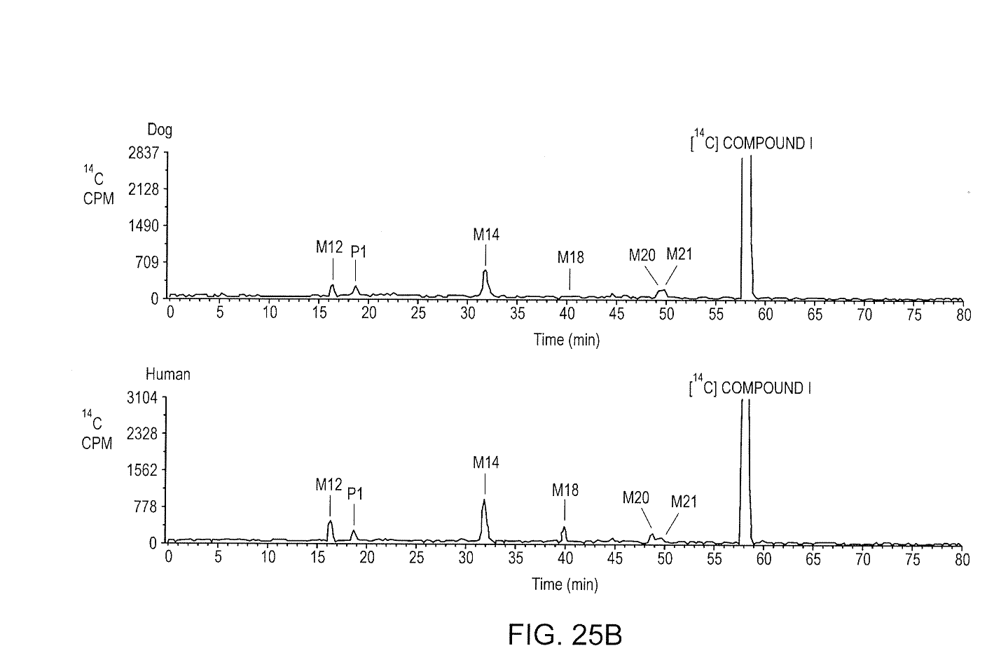

The average extraction recoveries of radioactivity in all incubations were ≧90%. Representative radiochromatograms of incubations with 20 μM of [¹⁴C]COMPOUND I in cryopreserved rat, dog, and human hepatocytes at 1 hr are shown in FIG. 25. Metabolite profiles after incubations for 1 or 4 hr were qualitatively similar (data not shown for 4 hr incubations). Under the conditions used in this study, the average turnover of [¹⁴C]COMPOUND I to metabolites in rat, dog, and human hepatocytes was 32, 8, and 13% for the 1 hr incubations and 38, 12, and 18% for the 4 hr incubations, respectively. Six phase I and one phase II metabolites present in the hepatocyte incubations were identified as N-desfluoroquinolinyl COMPOUND I (M12), N-desmethoxyquinolinyl COMPOUND I (M14), hydroxy desfluoro COMPOUND I (M18), hydroxy COMPOUND I sulfate (M19), hydroxy COMPOUND I (M20), O-desmethyl COMPOUND I (M21), and hydroxy COMPOUND I (M22). N-Desfluoroquinolinyl COMPOUND I (M12), N-desmethoxyquinolinyl COMPOUND I (M14), and O-desmethyl COMPOUND I (M21) metabolites were observed in all species. Hydroxy desfluoro COMPOUND I (M18) and hydroxy COMPOUND I (M20) metabolites were observed in dog and human hepatocytes, while hydroxy COMPOUND I sulfate (M19) and hydroxy COMPOUND I (M22) metabolites were only observed in rat hepatocytes. The most prominent metabolite in all species was N-desmethoxyquinolinyl COMPOUND I (M14); all other metabolites were present in small or trace amounts. A decomposition product of [¹⁴C]COMPOUND I (P1) was observed in a control incubation (without hepatocytes) and present in all hepatocyte incubations in small amounts. P1 was identified as methoxyquinolinyl-piperazine by LC/MS. The positive control, [¹⁴C]7-ethoxycoumarin, had a turnover of 10, 9, and 6% in rat, dog, and human hepatocytes, respectively. The UGT activity in the hepatocytes was established by the formation of 7-ethoxycoumarin glucuronide at a rate of ≧40 μmol/10⁶/min, which was comparable with the data reported by the vendor for 7-hydroxycoumarin (Table 12).

TABLE 12
Cryopreserved Rat, Dog, and Human Hepatocytes Utilized for Incubations with [¹⁴C]COMPOUND I

| | | | | | Metabolizing Enzyme Activity (pmole/10⁶/min)^{a} | | | |
|---|---|---|---|---|---|---|---|---|
| | | | | # in | | | 7-OH-Coumarin | 7-OH-Coumarin |
| Species | Lot # | Sex | Prep Date | pool | 6β-OH-Testosterone | 7-OH-Coumarin | glucuronide | sulfate |
| Rat | 44047 | M | January 2006 | 5 | 750 | 78 | 33 | 260 |
| Dog^{b} | P1 | M | July 2003 | 1 | 150 | NA^{c} | 210 | 14 |
| Dog^{b} | FPA | M | June 2004 | 1 | NA^{c} | 318 | 98 | 200 |
| Human | DRF | Mixed sexes | March 2005 | 10 | 138 | 50 | 170 | 24 |

^{a}The cryopreserved rat and dog (lot #P1) hepatocytes were purchased from BD Biosciences (San Jose, CA) and the cryopreserved dog (lot #FPA) and human hepatocytes were purchased from In Vitro Technologies (Baltimore, MD); the metabolizing enzyme activities were determined by the vendors.
^{b}Hepatocytes from the two dogs were pooled before incubations.
^{c}Not Available.

Mass spectra were obtained by LC/MS and LC/MS/MS analysis for COMPOUND I and its metabolites in samples of rat, dog, and human hepatocytes. Structural characterization of these compounds is summarized in Table 13. The mass spectral characterization of COMPOUND I and its metabolites is discussed below. In LC/MS experiments conducted with D₂O substituted for H₂O in the mobile phase to determine number of exchangeable hydrogens, the mass difference between [M+D]⁺ and [M+H]⁺ was 1 Da larger than the number of exchangeable hydrogens on COMPOUND I and its metabolites due to exchange of the proton required for ionization to generate [M+H]⁺.

TABLE 13
Summary of COMPOUND I Metabolite Structural Characterization in
Cryopreserved Rat, Dog and Human Hepatocytes

| | Ret. | | | | |
|---|---|---|---|---|---|
| | Time | | Site of | | |
| Metabolite | (min)^{a} | [M + H]⁺ | Metabolism | Name | Source^{b} |
| P1 | 18.8 | 244 | Piperidine ring | Methoxyquinolinyl-piperazine | Media^{c} |
| M12 | 16.5 | 327 | Fluoroquinoline | N-Desfluoroquinolinyl | R, D, H |
| | | | | COMPOUND I | |
| M14 | 31.9 | 315 | Methoxyquinoline | N-Desmethoxyquinolinyl | R, D, H |
| | | | | COMPOUND I | |
| M18 | 40.0 | 470 | Fluoroquinoline | Hydroxy desfluoro COMPOUND I | D (trace), H |
| M19 | 44.5 | 568 | Methoxyquinoline | Hydroxy COMPOUND I sulfate | R |
| | | | or piperazine ring | | |
| M20 | 48.7 | 488 | Methoxyquinoline | Hydroxy COMPOUND I | D (trace), H |
| M21 | 49.5 | 458 | Methoxyquinoline | O-Desmethyl COMPOUND I | R (trace), D, H |
| M22 | 54.6 | 488 | Fluoroquinoline | Hydroxy COMPOUND I | R |
| COMPOUND I | 58.2 | 472 | None | COMPOUND I | All |

^{a}LC retention time taken from radiochromatograms and may differ from LC/MS retention times
^{b}R, rat; D, dog; H, human
^{c}Decomposition product, observed in media control

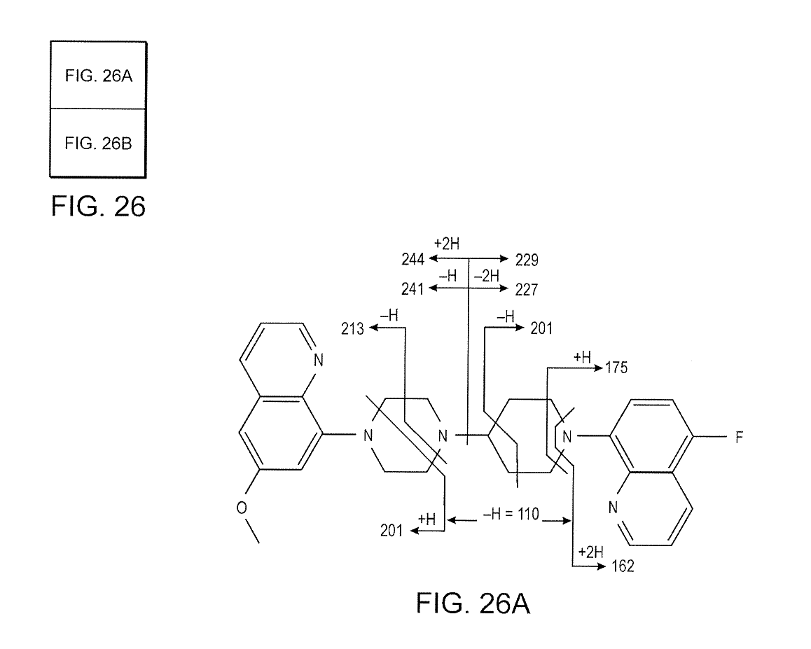

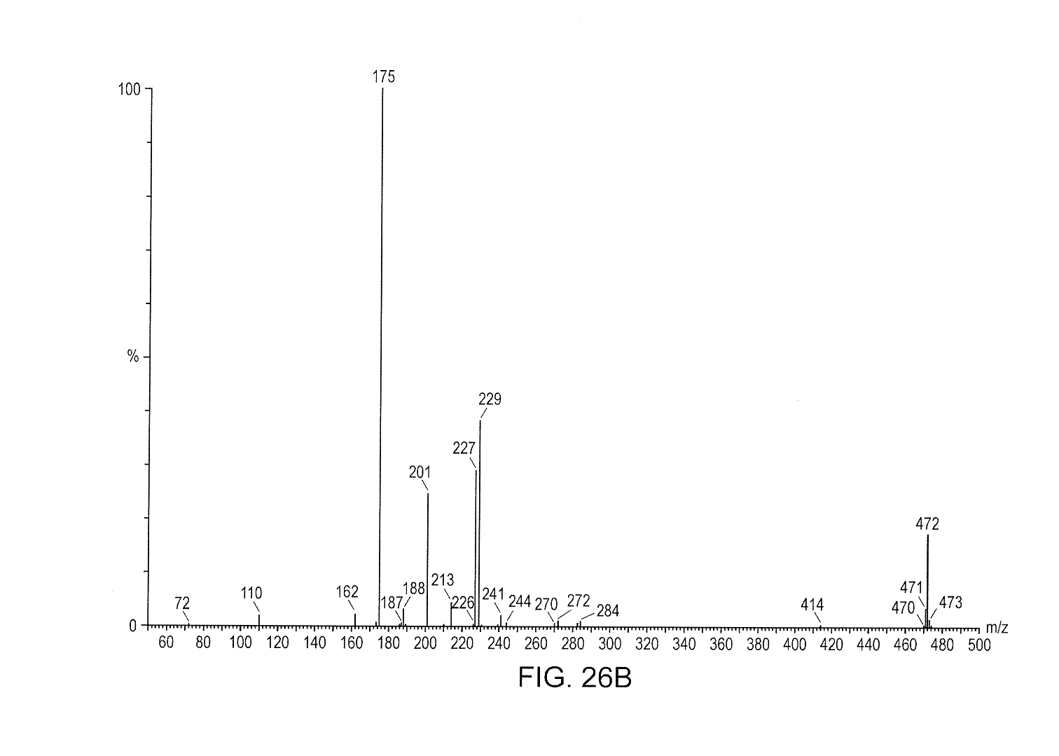

The mass spectral characteristics of synthetic COMPOUND I were examined for comparison with metabolites. In the LC/MS spectrum of COMPOUND I, the protonated molecular ion, [M+H]⁺ was observed at m/z 472. LC/MS with D₂O substituted for H₂O in the mobile phase generated [M+D]⁺ at m/z 473 (data not shown), consistent with COMPOUND I having no exchangeable hydrogens. The MS/MS spectrum obtained from collision activated dissociation of m/z 472 from COMPOUND I and the proposed fragmentation scheme are shown in FIG. 26. Fragmentation of the piperazine-piperidine bond with charge retention on the methoxyquinoline half of the molecule yielded m/z 244 and 241. The same fragmentation with charge retention on the fluoroquinoline half of the molecule yielded m/z 229 and 227. Fragmentation of the piperazine ring generated a methoxyquinoline-containing ion at m/z 213. Fragmentation of the piperidine ring generated fluoroquinoline-containing ions at m/z 175 and 162. Fragmentation of the piperazine and piperidine rings yielded m/z 110. Two assignments for the m/z 201 product ion were made. One m/z 201 product ion originated from cleavage of the piperidine ring with charge retention on the moiety containing the fluoroquinoline ring. The other m/z 201 product ion originated from cleavage of the piperazine ring. These assignments were confirmed by the product ions of m/z 474 (¹⁴C[M+H]⁺) and m/z 476 (¹⁴C₂[M+H]⁺) mass spectral data for radiolabeled COMPOUND I (data not shown).

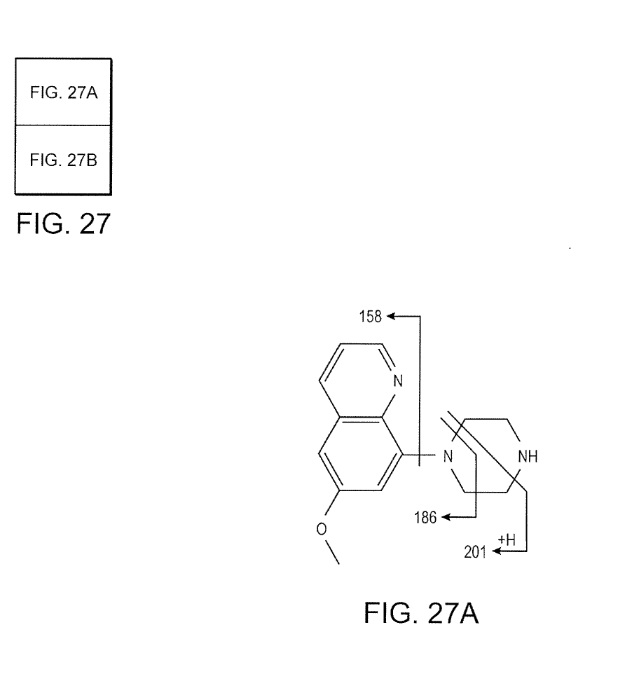

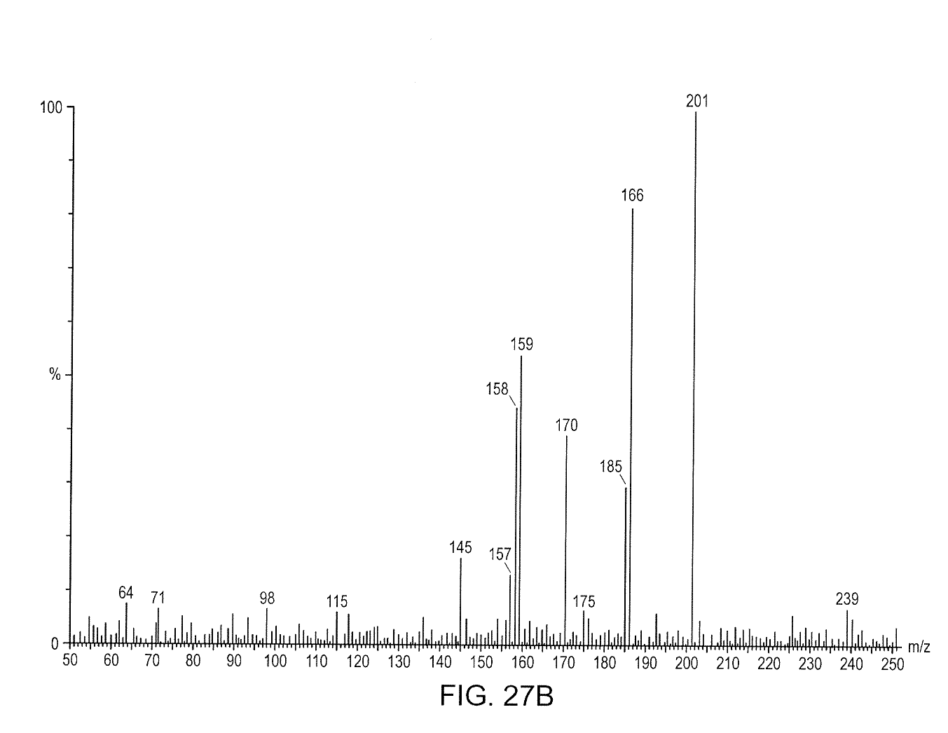

The [M+H]⁺ for P1 was observed at m/z 244, which was 228 Da less than COMPOUND I. LC/MS with D₂O substituted for H₂O in the mobile phase generated [M+D]⁺ at m/z 246 (data not shown). These data indicated one exchangeable hydrogen, which was one more than COMPOUND I and consistent with N-dealkylation of the COMPOUND I molecule. The product ions of m/z 244 mass spectrum and the proposed fragmentation scheme for P1 are presented in FIG. 27. The product ion at m/z 201 was also observed for COMPOUND I, which suggested an intact methoxyquinolinyl-piperazine moiety. Fragmentation of the piperazine ring and quinoline-piperazine bond generated m/z 186 and 158, respectively, which were consistent with methoxyquinolinyl-piperazine. Therefore, P1 was identified as methoxyquinolinyl-piperazine.

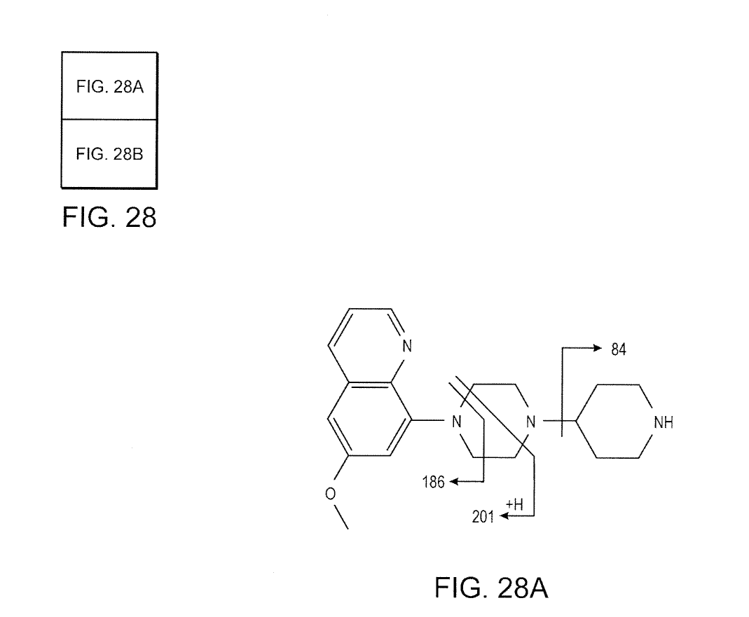

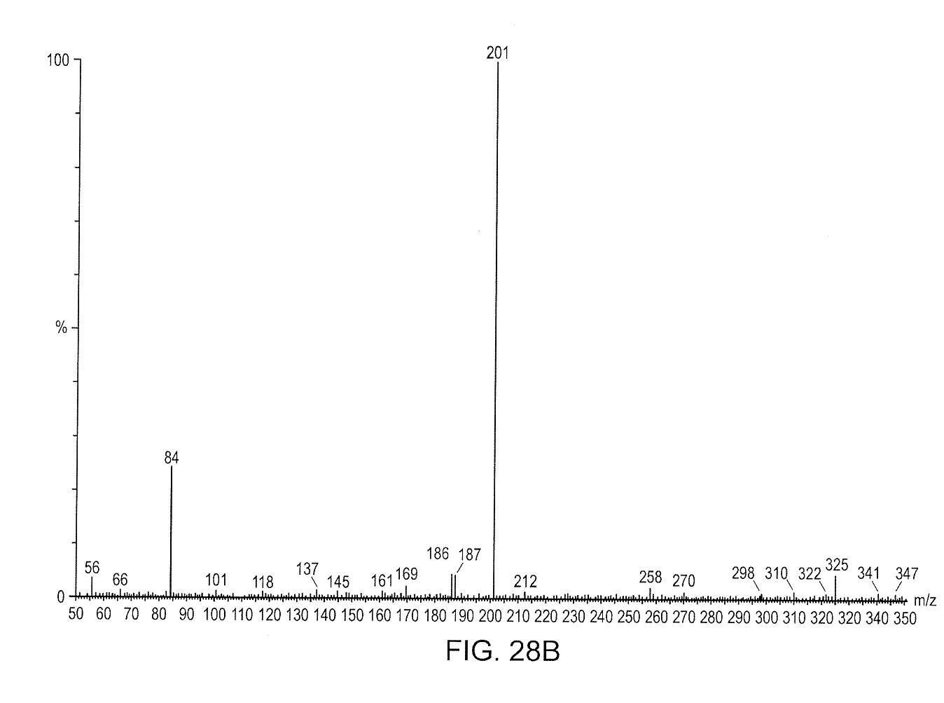

Metabolite M12 produced a [M+H]⁺ at m/z 327, which was 145 Da less than COMPOUND I. LC/MS with D₂O substituted for H₂O in the mobile phase generated [M+D]⁺ at m/z 329 (data not shown). These data indicated one exchangeable hydrogen, which was one more than COMPOUND I and consistent with N-dealkylation of the COMPOUND I molecule. The product ions of m/z 327 mass spectrum and the proposed fragmentation scheme for M12 are presented in FIG. 28. The product ion at m/z 201 was also observed for COMPOUND I. Fragmentation of the piperazine ring generated m/z 186. The product ion at m/z 84 represented a piperidinyl ion. These data indicated intact methoxyquinolinyl-piperazine and piperidine moieties, which in combination with the molecular weight difference between M12 and COMPOUND I indicated that the fluoroquinoline moiety of COMPOUND I was not present in M12. Therefore, M12 was identified as N-desfluoroquinolinyl COMPOUND I.

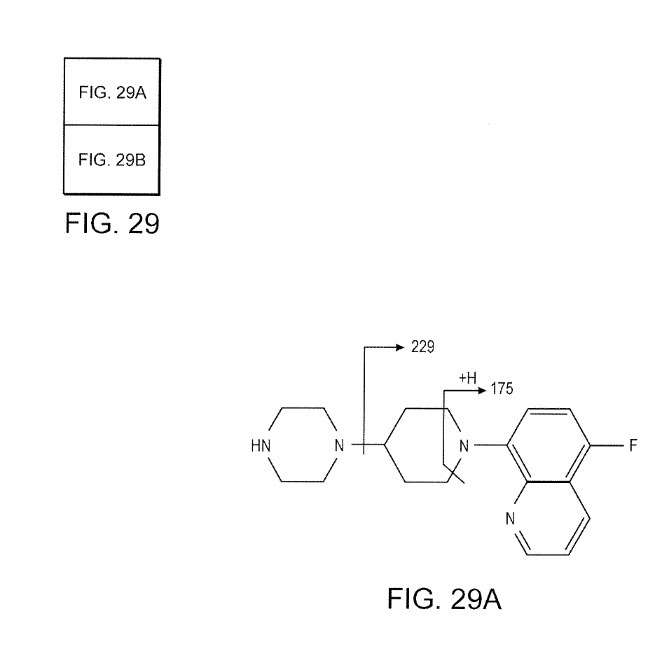

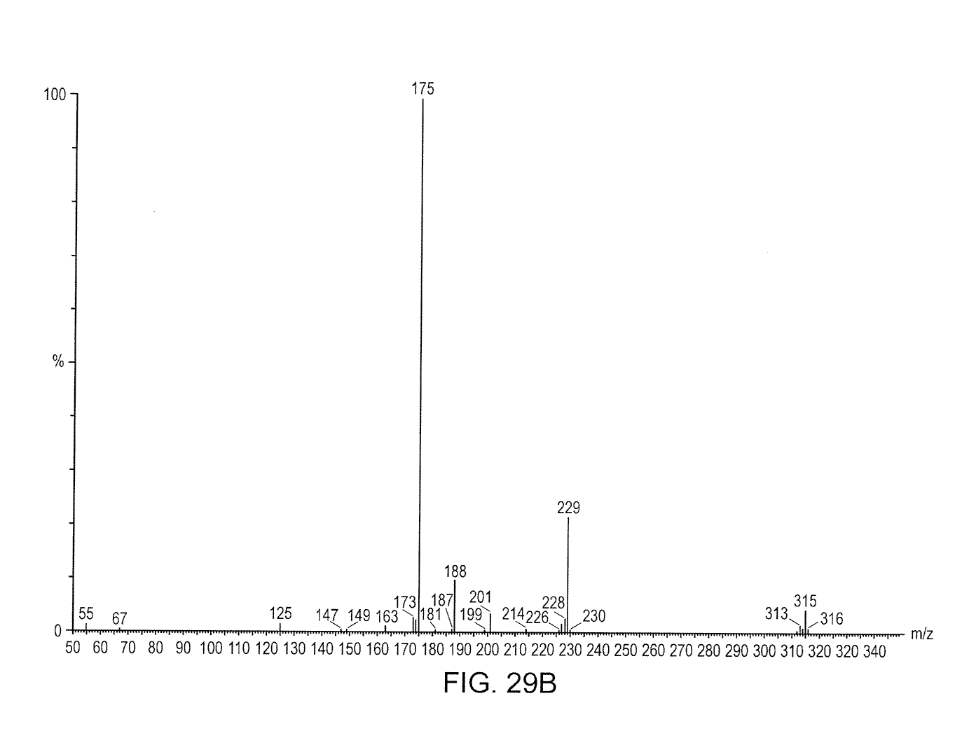

The [M+H]⁺ for M14 was observed at m/z 315, which was 157 Da less than COMPOUND I. LC/MS with D₂O substituted for H₂O in the mobile phase generated [M+D]⁺ at m/z 317 (data not shown). These data indicated one exchangeable hydrogen, which was one more than COMPOUND I and consistent with N-dealkylation of the COMPOUND I molecule. The product ions of m/z 315 mass spectrum and the proposed fragmentation scheme for M14 are presented in FIG. 29. The product ions at m/z 229 and 175 were also observed for COMPOUND I, which indicated unchanged piperidine and fluoroquinoline rings. These data and the molecular weight difference between M14 and COMPOUND I indicated that the methoxyquinoline moiety of COMPOUND I was not present in M14. Therefore, M14 was identified as N-desmethoxyquinolinyl COMPOUND I.

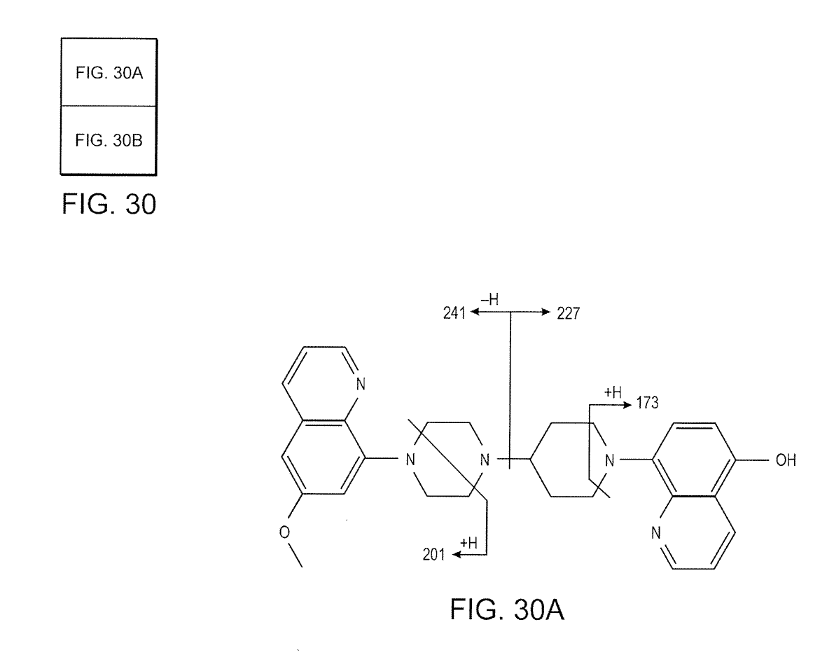

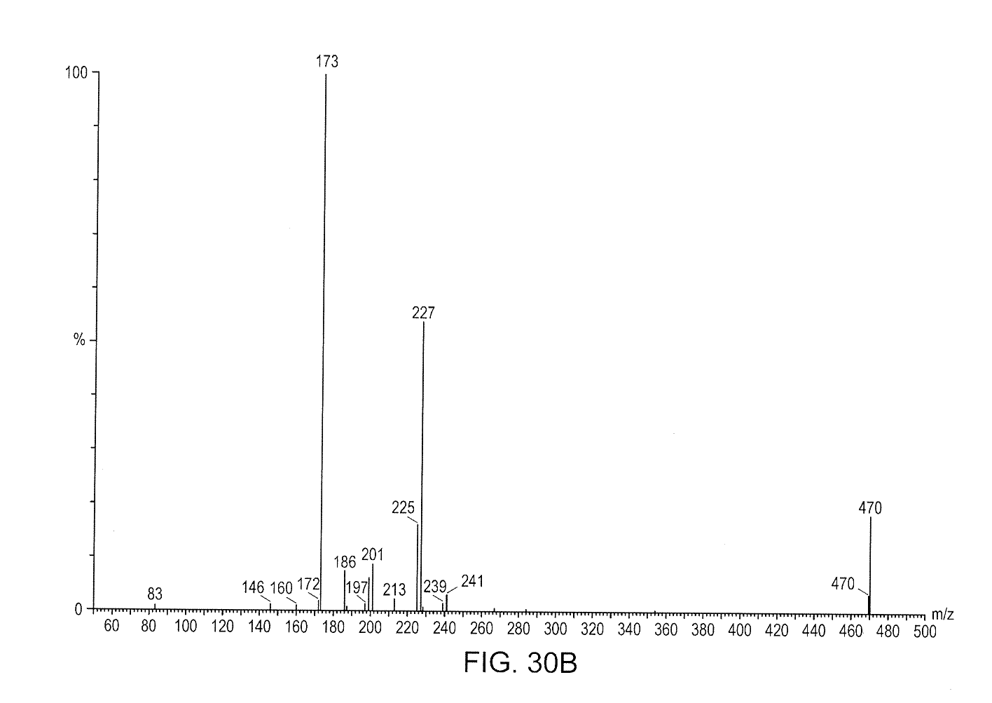

Metabolite M18 produced a [M+H]⁺ at m/z 470, which was 2 Da less than COMPOUND I. LC/MS with D₂O substituted for H₂O in the mobile phase generated [M+D]⁺ at m/z 472 (data not shown). These data indicated one exchangeable hydrogen, which was one more than COMPOUND I and consistent with the presence of a hydroxyl group not present in COMPOUND I. The product ions of m/z 470 mass spectrum and the proposed fragmentation scheme for M18 are presented in FIG. 30. Product ions at m/z 241 and 201 were also observed for COMPOUND I, which indicated an unchanged methoxyquinolinyl-piperazine moiety. Product ions at m/z 227 and 173 were 2 Da less than the corresponding ions at m/z 229 and 175, respectively, for COMPOUND I, which indicated that the fluoroquinoline ring was the site of metabolism. The molecular weight difference and additional exchangeable hydrogen for M18 compared to COMPOUND I indicated oxidative defluorination of the fluoroquinoline. Therefore, M18 was identified as hydroxy desfluoro COMPOUND I.

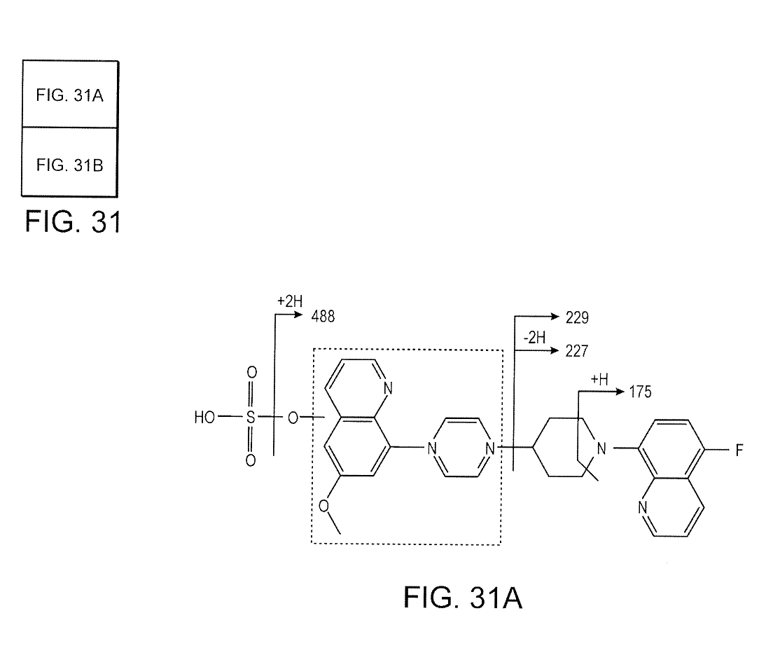

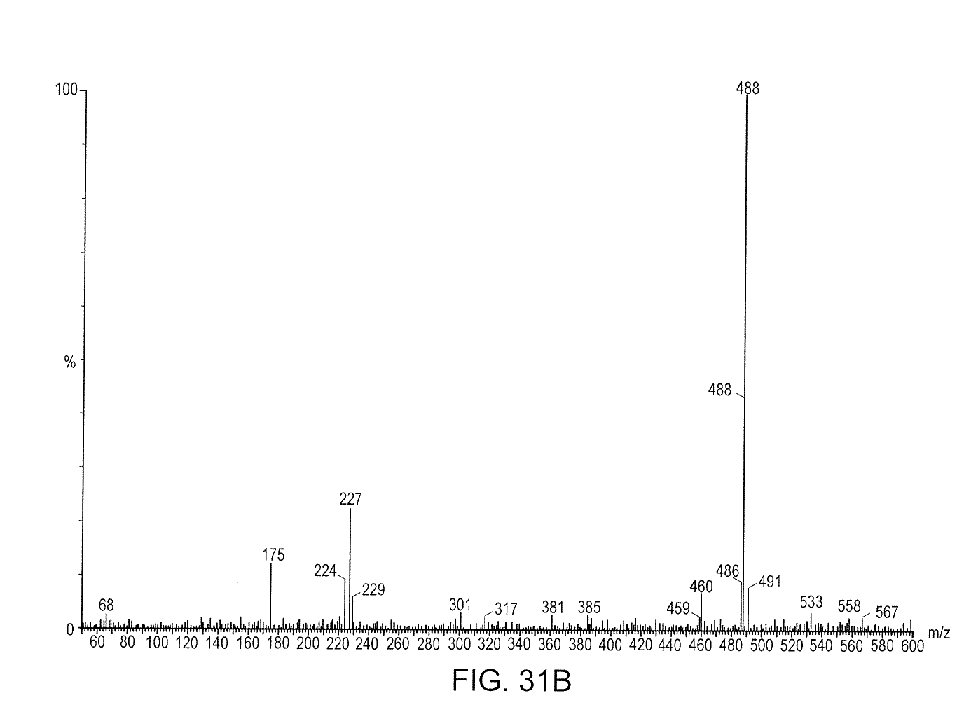

The [M+H]⁺ for M19 was observed at m/z 568, which was 96 Da larger than COMPOUND I. LC/MS with D₂O substituted for H₂O in the mobile phase generated [M+D]⁺ at m/z 570 (data not shown). These data indicated one exchangeable hydrogen, which was one more than COMPOUND I. The product ions of m/z 568 mass spectrum and the proposed fragmentation scheme for M19 are presented in FIG. 31. Neutral loss of 80 Da from [M+H]⁺ yielded m/z 488, which was 16 Da larger than COMPOUND I and indicated that M19 was a sulfate of a hydroxy COMPOUND I. Product ions at m/z 229, 227 and 175 were also observed for COMPOUND I and indicated an unchanged fluoroquinolinyl-piperidine moiety. The methoxyquinolinyl-piperazine moiety was consequently the site of hydroxylation and subsequent sulfation. Therefore, M19 was identified as a hydroxy COMPOUND I sulfate.

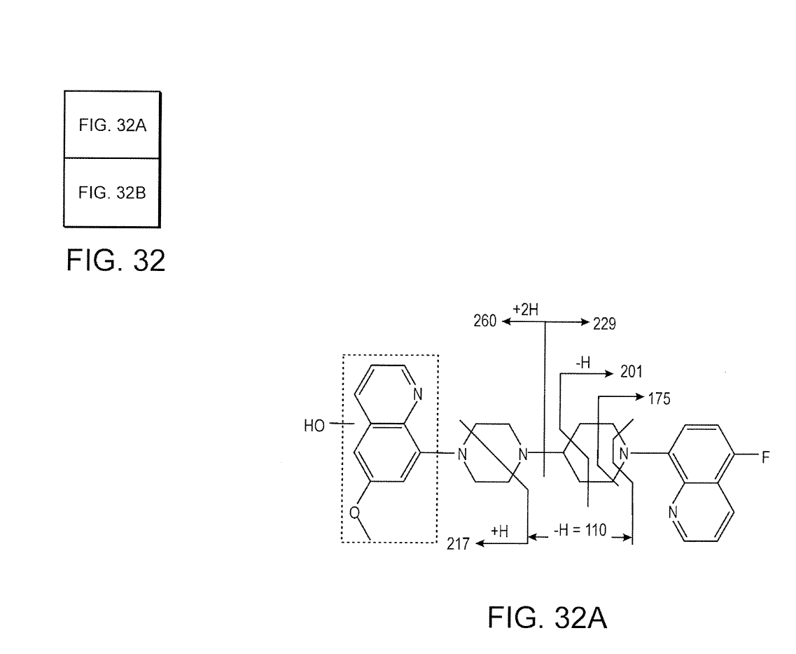

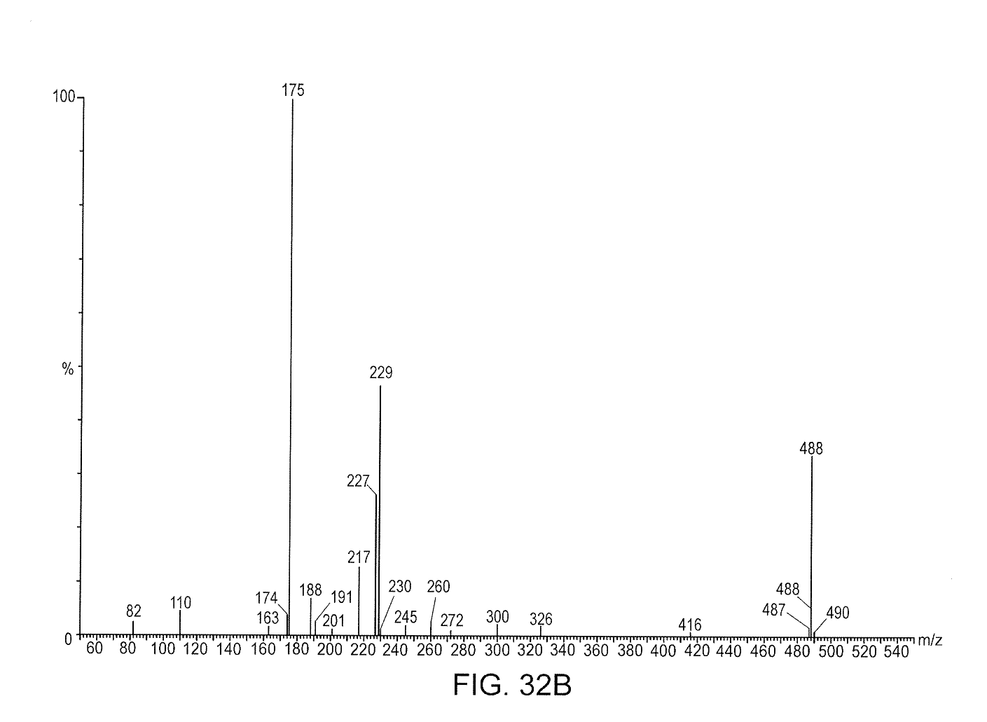

Metabolite M20 produced a [M+H]⁺ at m/z 488, which was 16 Da larger than COMPOUND I. LC/MS with D₂O substituted for H₂O in the mobile phase generated [M+D]⁺ at m/z 490 (data not shown). These data indicated one exchangeable hydrogen, which was one more than COMPOUND I and consistent with hydroxylation rather than N-oxidation. The product ions of m/z 488 mass spectrum and the proposed fragmentation scheme for M20 are presented in FIG. 32. No loss of H₂O from [M+H]⁺ was observed, which was consistent with an aromatic hydroxyl group. Product ions at m/z 229, 201, 175 and 110 were also observed for COMPOUND I, which indicated an unchanged fluoroquinolinyl-piperidine moiety. Product ions at m/z 260 and 217 were 16 Da larger than the corresponding methoxyquinolinyl ions at m/z 244 and 201, respectively, for COMPOUND I, which indicated hydroxylation of the methoxyquinoline. Therefore, M20 was identified as a hydroxy COMPOUND I.

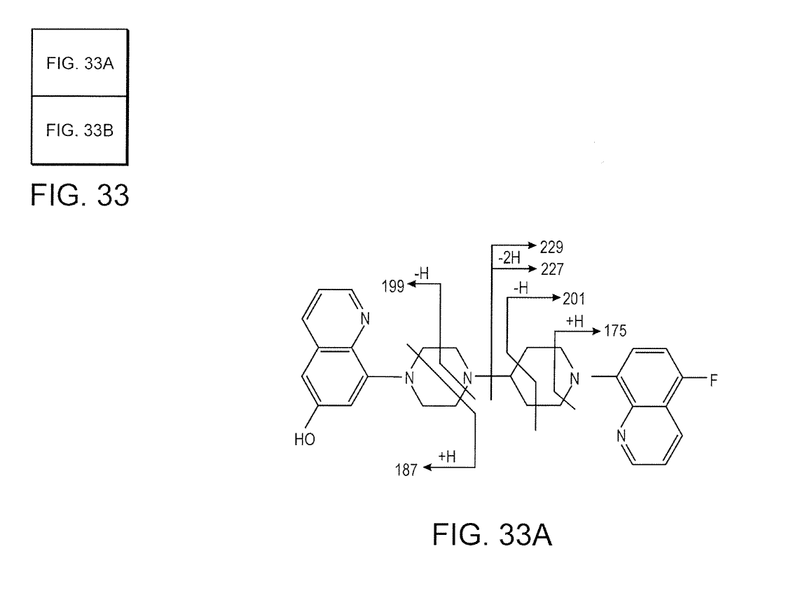

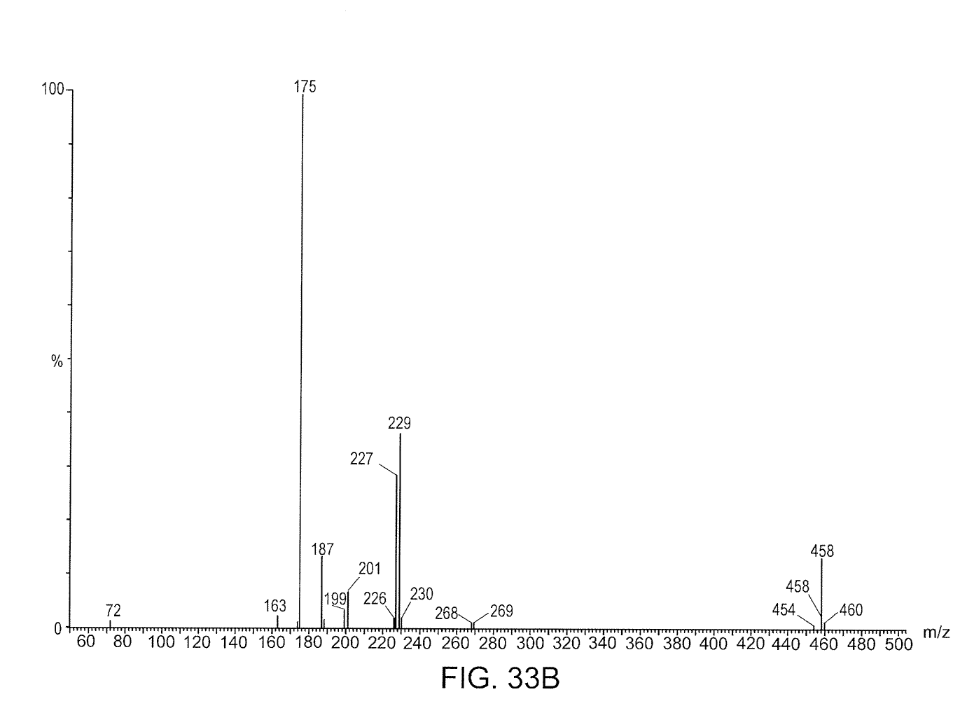

The [M+H]⁺ for M21 was observed at m/z 458, which was 14 Da less than COMPOUND I. LC/MS with D₂O substituted for H₂O in the mobile phase generated [M+D]⁺ at m/z 460 (data not shown). These data indicated one exchangeable hydrogen, which was one more than COMPOUND I and consistent with the presence of a hydroxyl group. The product ions of m/z 458 mass spectrum and the proposed fragmentation scheme for M21 are presented in FIG. 33. Product ions at m/z 229, 227, 201 and 175 were also observed for COMPOUND I, which indicated an unchanged fluoroquinolinyl-piperidine moiety. Product ions at m/z 199 and 187 were 14 Da less than the corresponding methoxyquinolinyl ions at m/z 213 and 201, respectively, for COMPOUND I, which indicated demethylation of the methoxy group. Therefore, M21 was identified as O-desmethyl COMPOUND I.

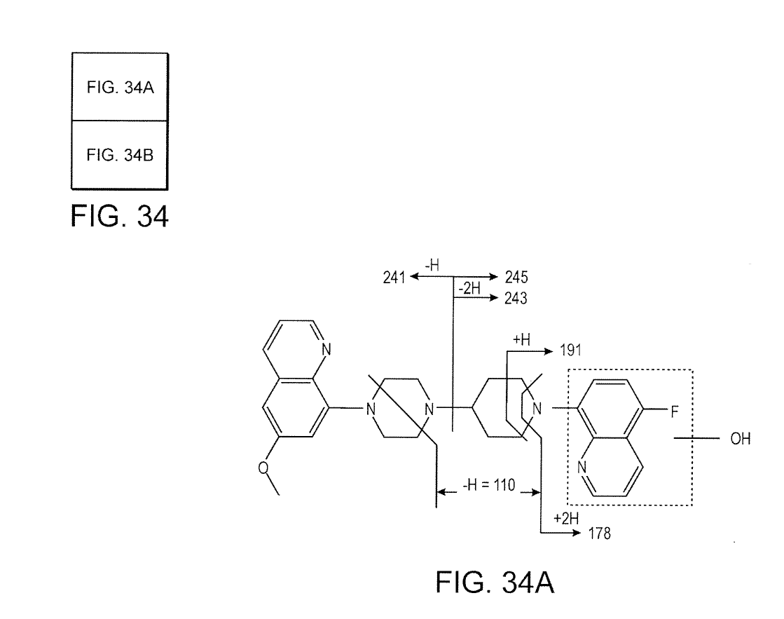

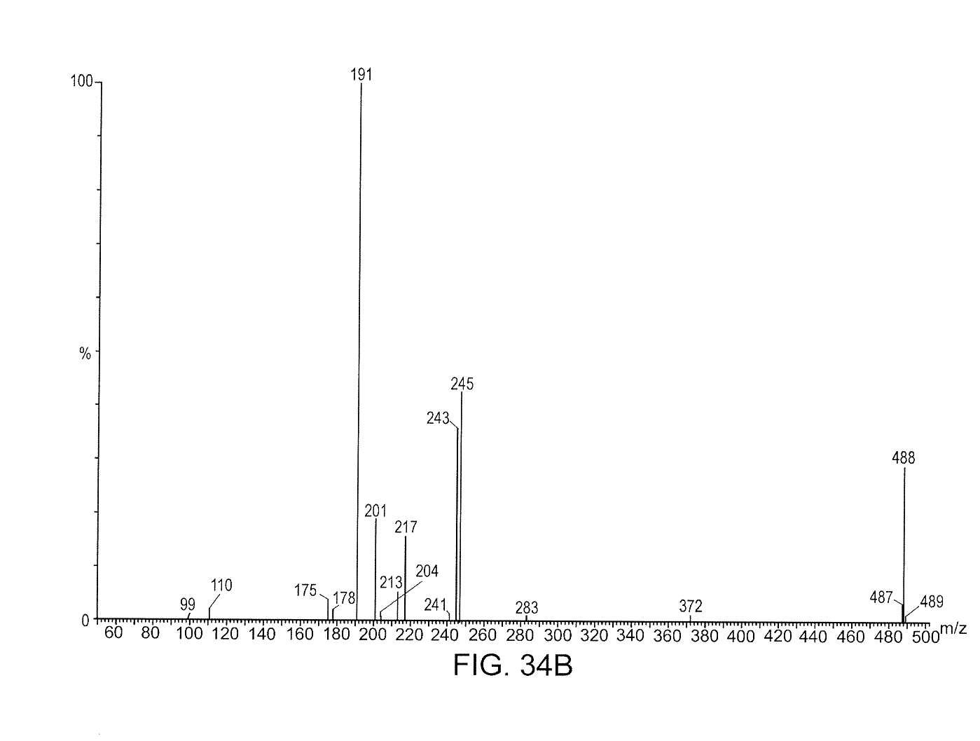

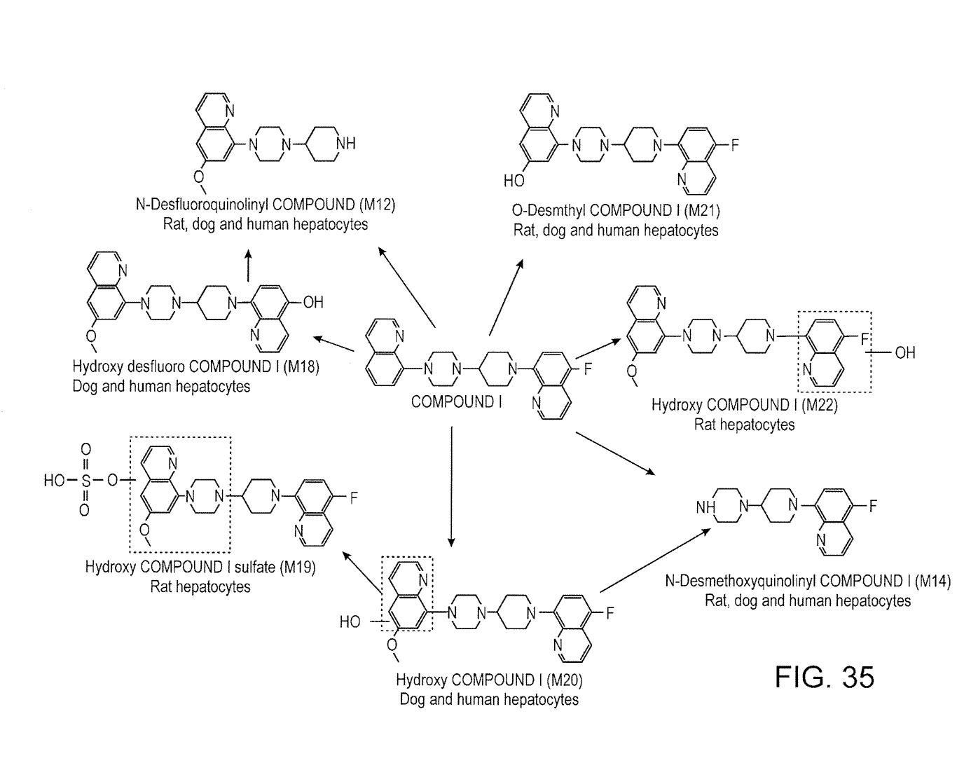

Metabolite M22 produced a [M+H]⁺ at m/z 488, which was 16 Da larger than COMPOUND I. LC/MS with D₂O substituted for H₂O in the mobile phase generated [M+D]⁺ at m/z 490 (data not shown). These data indicated one exchangeable hydrogen, which was one more than COMPOUND I and consistent with hydroxylation rather than N-oxidation. The product ions of m/z 488 mass spectrum and the proposed fragmentation scheme for M22 are presented in FIG. 34. Product ions at m/z 241 and 110 were also observed for COMPOUND I, which indicated unchanged methoxyquinoline, piperazine and piperidine rings. Product ions at m/z 245, 243, 191 and 178 were 16 Da larger than the corresponding ions at m/z 229, 227, 175 and 162, respectively, for COMPOUND I, which indicated hydroxylation of the fluoroquinoline. Therefore, M22 was identified as a hydroxy COMPOUND I.

### In Vivo Metabolism of [¹⁴C]COMPOUND I IN Sprague-Dawley Rats Following a Single Oral 5 Mg/Kg Dose of [¹⁴C]COMPOUND I

The in vivo metabolism of [¹⁴C]COMPOUND I was investigated in male and female rats following a single 5 mg/kg oral dose and the metabolites were characterized by LC/MS. [¹⁴C]COMPOUND I trisuccinate salt was prepared as described above. The chemical structure of COMPOUND I with the positions of ¹⁴C labels is shown below:

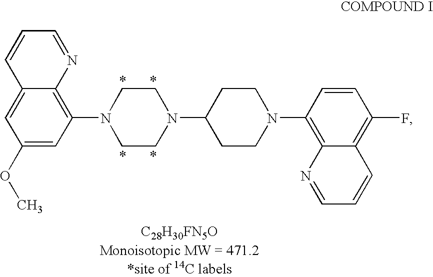

Ultima Gold, Ultima Flo M, Permafluor® E⁺ scintillation cocktails, and Carbo-Sorb E carbon dioxide absorber were purchased from Perkin Elmer Life Sciences (Boston, Mass.). Polysorbate 80 was obtained from Mallinckrodt Baker (Phillipsburg, N.J.). Methylcellulose was from Sigma-Aldrich (Milwaukee, Wis.). Solvents used for extraction and for chromatographic analysis were HPLC or ACS reagent grade and were purchased from EMD Chemicals (Gibbstown, N.J.). Deuterium oxide (D₂O) was obtained from Cambridge Isotope Laboratories (Andover, Mass.).

Dose preparation, animal dosing, and specimen collection were performed at Wyeth Research, Collegeville, Pa. The vehicle contained 2% (w/v) polysorbate 80, NF and 0.5% (w/v) methylcellulose (4000 cps) in water. [¹⁴C]COMPOUND I trisuccinate salt (36.6 mg) and non-labeled COMPOUND I trisuccinate salt (26.7 mg) were dissolved in 1 mL of ethanol and suspended in 35 mL of the vehicle with stirring. The target [¹⁴C]COMPOUND I concentration was approximately 1 mg/mL as free base and 70 μCi/mL with a target specific activity of 70 μCi/mg. Three pre- and post-dose aliquots (100 μL) were taken for the determination of radiochemical purity, drug and radioactivity concentrations and specific activity (see below).

Male rats weighing from 323 to 354 g and female rats weighing from 270 to 317 g at the time of dosing were purchased from Charles River Laboratories (Wilmington, Mass.). Non-fasted rats were given a single 5 mg/kg (˜350 μCi/kg) dose of [¹⁴C]COMPOUND I at a volume of 5.0 mL/kg via intragastric gavage. Three rats were dosed for each time point. Animals were provided Purina rat chow and water ad libitum, and were kept individually in metabolism cages.

Blood samples were collected at 1, 3, 6 and 24 hr from male rats and at 1 and 3 hr from female rats after dose administration by cardiac puncture into tubes containing EDTA as the anticoagulant and placing them on ice. Triplicate aliquots (50 μL) of whole blood were removed and plasma was immediately obtained from the remaining blood by centrifugation at 4° C. Whole brain samples were collected after saline perfusion at 1, 3, 6 and 24 hr post-dose from male rats and at 1 and 3 hr post-dose from female rats. Urine samples were collected on dry ice from male rats at intervals of 0-6 and 6-24 hr post-dose. Feces were collected from male rats at 0-24 hr post-dose at room temperature. The biological specimens and aliquots of the dose suspension were stored at approximately −70° C. until analysis.

Aliquots of the pre- and post-dose suspension were dissolved in 25% methanol in water and analyzed for radioactivity concentrations as described in below. Approximately 80,000 dpm in 40 μL was analyzed by HPLC for radiochemical purity and chemical purity (see below). To determine the specific activity of the dose suspension, non-radiolabeled COMPOUND I was dissolved in 25% methanol in water to give five different concentrations ranging from 4.9 to 98 μg/mL and concurrently analyzed by HPLC to generate a standard curve. Aliquots (40 μL) of the diluted [¹⁴C]COMPOUND I dose suspension were injected onto the HPLC column and fractions were collected at 60 second intervals after UV detection. Radioactivity in each fraction was determined as described in section 2.2.3.1. Fractions were also collected from a blank injection to obtain the background level of radioactivity. The UV peak associated with [¹⁴C]COMPOUND I was integrated to calculate the drug concentration. The specific activity of [¹⁴C]COMPOUND I was derived from the amount of drug in the peak and the total radioactivity in the fractions associated with the drug peak.

Triplicate aliquots of diluted dose (20 μL), dose fractions (10 μL), and plasma (50 μL) and urine (100 μL) from individual rats were analyzed for radioactivity concentrations. Radioactivity determinations were made with a Tri-Carb Model 3100 TR liquid scintillation counter (LSC) (Perkin Elmer) using 5 mL of Ultima Gold as the scintillation fluid.

Brain and fecal samples from individual rats were weighed and homogenized in water at a volume-to-weight ratio of about 3:1 and 5:1, respectively, with a Polytron PT homogenizer at ice-cold temperature. Duplicate aliquots of blood (50 μL), brain homogenates (0.2 g) and fecal homogenates (0.2 g) were placed on Combusto-cones with Combusto-pads and allowed to dry overnight. Samples were combusted with a model 307 Tri-Carb Sample Oxidizer, equipped with an Oximate-80 Robotic Automatic Sampler (Perkin Elmer). The liberated ¹⁴CO₂ was trapped with Carbo-Sorb E carbon dioxide absorber, mixed with PermaFluor® E⁺ liquid scintillation cocktail, and counted in a Tri-Carb Model 3100 TR/LL liquid scintillation counter (Perkin Elmer). The oxidation efficiency of the oxidizer was 97.9%.

Plasma samples were pooled by equal volumes from three animals at each time point (1, 3, 6 and 24 hr for males; 1 and 3 hr for females). Aliquots of 2 mL of pooled plasma were mixed with 2 mL of acetonitrile, placed on ice for about 10 min, and then centrifuged at 4° C. The supernatant was transferred to a clean tube. The protein pellets were extracted two more times with 4 mL of acetonitrile. The supernatants from precipitation and extraction of each sample were pooled, mixed, and evaporated at 22° C. under nitrogen in a TurboVap LV evaporator (Caliper Life Sciences, Hopkinton, Mass.) to about 1.0 mL. The concentrated extract was centrifuged, the supernatant volume measured and the extraction efficiency was determined by analyzing duplicate 10 μL aliquots for radioactivity concentrations. For the 1, 3 and 6 hr plasma samples, an aliquot of the supernatant (200 μL) was injected onto the HPLC column as described in below and a radioactivity flow detector was used for data acquisition. The 24 hr sample from male rats was not analyzed for profiles due to low radioactivity concentration and low extraction recovery. Plasma extracts were also analyzed by LC/MS for metabolite characterization as described in below.

Brain homogenates were pooled proportionally to their total weight by time point (1, 3 and 6 hr for males; 1 and 3 hr for females) and analyzed for metabolite profiles. Aliquots of 3.0 g of brain homogenates were mixed with 6.0 mL of acetonitrile, placed on ice for about 10 min and centrifuged. The supernatant was transferred to a clean tube. The residue was extracted two more times with 6.0 mL of acetonitrile. The supernatants of each sample were combined, evaporated to about 1.5 mL and centrifuged. Extraction efficiency was determined by analyzing aliquots of 20 μL of the supernatant for radioactivity. For metabolite profiling, an aliquot (500 μL) of the supernatant was injected onto the HPLC column as described hereinbelow and HPLC fractions were collected at 20 second intervals into 96-well Lumaplates (Perkin Elmer). The plates were dried overnight in an oven at 40° C. and analyzed by a TopCount NXT radiometric microplate reader (Perkin Elmer). Brain extracts were also analyzed by LC/MS for metabolite characterization as described hereinbelow. The 24 hr samples were not analyzed for profiles due to low radioactivity content.

Fecal homogenates (0-24 hr) were pooled proportionally to their total weight and analyzed for metabolite profiles. An aliquot of 2.0 g of the pooled fecal homogenate was mixed with 6.0 mL of acetonitrile, placed on ice for about 10 min and centrifuged at 4° C. The supernatant was transferred to a clean tube. The residue was extracted two more times with 6.0 mL of acetonitrile. The supernatants were combined and evaporated to a volume of about 2.0 mL. Extraction efficiency was determined by analyzing aliquots of 10 μL of the supernatant for radioactivity. For metabolite profiling, an aliquot (40 μL) of the supernatant was analyzed by HPLC with radioactivity flow detection (section 2.2.5). The sample was also analyzed by LC/MS to characterize the radioactive peaks (see below).

Since only 3.6% of dose was excreted in 0-24 hr urine, metabolite profiles and metabolite characterization in urine are not reported.

A Waters model 2695 HPLC system (Waters Corp., Milford, Mass.) with a built-in autosampler was used for analysis. Separations were accomplished on a Luna C18(2) column (150×2.0 mm, 5 μm) (Phenomenex, Torrance, Calif.) for dose analysis and on a Synergi Hydro-RP column (250×2.0 mm, 4 μm) (Phenomenex) for metabolite profiling. A C18 guard cartridge (4×2 mm) was coupled to the columns. The sample chamber in the autosampler was maintained at 4° C., while the columns were at ambient temperature of about 20° C. For brain samples, fractions were collected and analyzed by TopCount as described hereinabove. For the plasma and fecal extracts, a Flo-One β Model A525 radioactivity flow detector (Perkin Elmer) with a 250 μL LQTR flow cell and a Waters model 996 photodiode array UV detector set to monitor at 250 nm were used for data acquisition. The flow rate of Ultima Flo M scintillation fluid was 1.0 mL/min, providing a mixing ratio of scintillation cocktail to mobile phase of about 5:1. The mobile phase consisted of 10 mM ammonium acetate, pH 4.5 (A) and acetonitrile (B), and was delivered at 0.2 mL/min. The linear gradient conditions for dose analysis and metabolite profiles are summarized in Table 14 and Table 15, respectively.

TABLE 14
HPLC Linear Elution Gradient for Dose Analysis

| Time (min) | A (%) | B (%) |
|---|---|---|
| 0 | 95 | 5 |
| 5 | 95 | 5 |
| 45 | 55 | 45 |
| 50 | 55 | 45 |

TABLE 15
HPLC Linear Elution Gradient for Metabolite Profiles

| Time (min) | A (%) | B (%) |
|---|---|---|
| 0 | 98 | 2 |
| 5 | 98 | 2 |
| 15 | 92 | 8 |
| 40 | 80 | 20 |
| 55 | 78 | 22 |
| 65 | 78 | 22 |
| 66 | 70 | 30 |
| 75 | 50 | 50 |
| 85 | 50 | 50 |
| 90 | 40 | 60 |
| 95 | 30 | 70 |

The HPLC system used for mass spectrometric analysis was a Waters Alliance Model 2695 HPLC system (Waters Corp.). It was equipped with a built-in autosampler and a Model 996 diode array UV detector (Waters Corp.). The UV detector was set to monitor 210-400 nm. The HPLC conditions were as described hereinabove for metabolite profiling. The column re-equilibration time was 15 min (110 min total run time). For H-D exchange experiments, D₂O was substituted for H₂O in mobile phase A. The first 5 min of flow was diverted away from the mass spectrometer prior to evaluation of metabolites.

The mass spectrometer used for metabolite characterization was a Micromass Quattro Ultima triple quadrupole mass spectrometer (Waters Corp). It was equipped with an electrospray ionization (ESI) interface and operated in the positive ionization mode. Settings for the mass spectrometer are listed in Table 16.

TABLE 16
Mass Spectrometer Settings

| | |
|---|---|
| ESI spray | 2.5 kV |
| Cone | 45 V |
| Mass resolution of scanning | 0.7 Da ± 0.2 Da width at half height |
| mass analyzer | |
| Mass resolution of non- | 1-2 Da width at half height |
| scanning mass analyzer for | |
| MS/MS experiments | |
| Desolvation gas flow | 950-1100 L/hr |
| Source block temp. | 80° C. |
| Desolvation gas temp. | 250° C. |
| Collision gas pressure | 0.9-1.1 × 10⁻³ mbar |
| Collision offset | 35 eV |

Flo-One analytical software (Perkin Elmer, version 3.65) was utilized to integrate the radioactive peaks. DataFlo software utility (Perkin Elmer, beta version 0.55) was used to convert ASCII files from the TopCount NXT microplate counter into CR format for processing in Flo-One analysis software. Micromass MassLynx software (version 4.0, Waters Corp.) was used for analysis of LC/MS data. Microsoft Excel® 2000 was used to calculate means and standard deviations.

The radiochemical purity and chemical purity of [¹⁴C]COMPOUND I in the dose suspension were greater than 99%. The pre- and post-dose aliquots had similar purity. The specific activity of the [¹⁴C]COMPOUND I dosing suspension was 69.5 μCi/mg. The average drug concentration was 1.07 mg/mL (74.2 μCi/mL). The actual dose of COMPOUND I administered averaged 5.3 mg/kg. Dose concentration and specific activity were within 10% of theoretical values.

The concentrations of radioactivity in whole blood and plasma, and whole blood to plasma ratios of radioactivity after a single oral dose of [¹⁴C]COMPOUND I are summarized in Table 17. The mean plasma radioactivity concentrations were 516, 191, 130 and 24.7 ng equivalents/mL at 1, 3, 6 and 24 hr post-dose, respectively, in male rats, and were 837 and 412 ng equivalents/mL at 1 and 3 hr post-dose, respectively, in female rats. The mean radioactivity concentrations were higher in female than male rats; the difference in radioactivity concentrations between male and female rats was statistically significant at 3 hr post-dose. The average whole blood-to-plasma radioactivity ratios ranged from 0.49 to 0.89 from 1 to 6 hr post-dose, but increased to 1.04 in male rats at 24 hr post-dose, indicating some partitioning of COMPOUND I and its metabolites into red blood cells (Table 17).

Brain radioactivity concentrations in male rats were 51, 25, 16 and 6.2 ng equivalents/g at 1, 3, 6 and 24 hr post-dose, respectively, while brain radioactivity concentrations in female rats were 133 and 49 ng equivalents/g at 1 and 3 hr post-dose, respectively (Table 18). As in plasma, the mean radioactivity concentrations were higher in female than male rats; the difference in radioactivity concentrations between male and female rats was statistically significant at 1 hr post-dose. The brain-to-plasma ratios of total radioactivity were 0.12 to 0.25 over 24 hr post-dose. Based on the total radioactivity concentrations and the chromatographic distribution of the radioactivity, the estimated brain COMPOUND I concentrations in male rats were 40.9, 15.9 and 10.0 ng equivalents/g at 1, 3 and 6 hr post-dose, respectively, while the estimated brain COMPOUND I concentrations in female rats were 106 and 34.7 ng equivalents/g at 1 and 3 hr post-dose, respectively (Table 18). The brain-to-plasma ratios of COMPOUND I concentrations were 0.47 to 0.85, indicating uptake of COMPOUND I into rat brain.

TABLE 17
Concentrations (ng equivalents/mL) of Total Radioactivity in
Whole Blood and Plasma and Whole Blood to Plasma Ratios
of Radioactivity in Rats Following a Single 5 mg/kg Oral
Dose of [¹⁴C]COMPOUND I

| | | | | |
|---|---|---|---|---|
| Time (hr) | Rat 1 | Rat 2 | Rat 3 | Mean ± SD |

Whole Blood
Male

| | | | | |
|---|---|---|---|---|
| 1 | 228 | 169 | 251 | 216 ± 42.3^{a} |
| 3 | 115 | 166 | 98.6 | 127 ± 35.1^{a} |
| 6 | 77.2 | 75.5 | 118 | 90.2 ± 24.1 |
| 24 | 22.8 | 26.2 | 28.0 | 25.7 ± 2.64 |

Female

| | | | | |
|---|---|---|---|---|
| 1 | 815 | 549 | 536 | 633 ± 157 |
| 3 | 326 | 206 | 287 | 273 ± 61.2 |

Plasma
Male

| | | | | |
|---|---|---|---|---|
| 1 | 366 | 295 | 887 | 516 ± 323 |
| 3 | 172 | 256 | 144 | 191 ± 58.3^{a} |
| 6 | 112 | 114 | 163 | 130 ± 28.9 |
| 24 | 23.3 | 24.2 | 26.7 | 24.7 ± 1.76 |

Female

| | | | | |
|---|---|---|---|---|
| 1 | 1274 | 854 | 383 | 837 ± 446 |
| 3 | 504 | 298 | 436 | 412 ± 105 |

Whole Blood/Plasma Ratio
Male

| | | | | |
|---|---|---|---|---|
| 1 | 0.62 | 0.57 | 0.28 | 0.49 ± 0.18 |
| 3 | 0.67 | 0.65 | 0.68 | 0.67 ± 0.02 |
| 6 | 0.69 | 0.66 | 0.72 | 0.69 ± 0.03 |
| 24 | 0.98 | 1.08 | 1.05 | 1.04 ± 0.06 |

Female

| | | | | |
|---|---|---|---|---|
| 1 | 0.64 | 0.64 | 1.40 | 0.89 ± 0.44 |
| 3 | 0.65 | 0.69 | 0.66 | 0.67 ± 0.02 |

^{a}Significantly lower than female, p < 0.05.

TABLE 18
Brain Concentrations (ng equivalents/g) of Radioactivity and COMPOUND I and
Mean Brain-to-Plasma Ratios in Rats Following a Single Oral Administration of
5 mg/kg of [¹⁴C]COMPOUND I

| | | | | Brain | Brain/Plasma |
|---|---|---|---|---|---|
| Time | Radioactivity Concentrations | | Brain/Plasma | COMPOUND I | COMPOUND I |
| (hr) | Individual | Mean ± SD | Radioactivity Ratio^{a} | Concentrations^{b} | Ratio |

Male

| | | | | | | | |
|---|---|---|---|---|---|---|---|
| 1 | 48 | 42 | 63 | 51 ± 11^{c} | 0.12 ± 0.04 | 40.9 | 0.47 |
| 3 | 22 | 31 | 23 | 25 ± 4.9 | 0.14 ± 0.02 | 15.9 | 0.54 |
| 6 | 15 | 13 | 19 | 16 ± 3.2 | 0.12 ± 0.01 | 10.0 | 0.85 |
| 24 | 7.1 | 5.3 | 6.1 | 6.2 ± 0.9 | 0.25 ± 0.05 | NA^{d} | NA |

Female

| | | | | | | | |
|---|---|---|---|---|---|---|---|
| 1 | 149 | 121 | 128 | 133 ± 15 | 0.20 ± 0.12 | 106 | 0.77 |
| 3 | 54 | 30 | 61 | 49 ± 16 | 0.12 ± 0.02 | 34.7 | 0.58 |

^{a}Data are presented as mean ± SD (n = 3).
^{b}Calculated by multiplying the mean brain radioactivity concentration by the percentage of COMPOUND I (section 3.3.2) in the pooled sample for each time point.
^{c}Significantly lower than female, p < 0.01.
^{d}NA: not available (metabolite profiles were not generated at 24 hr).

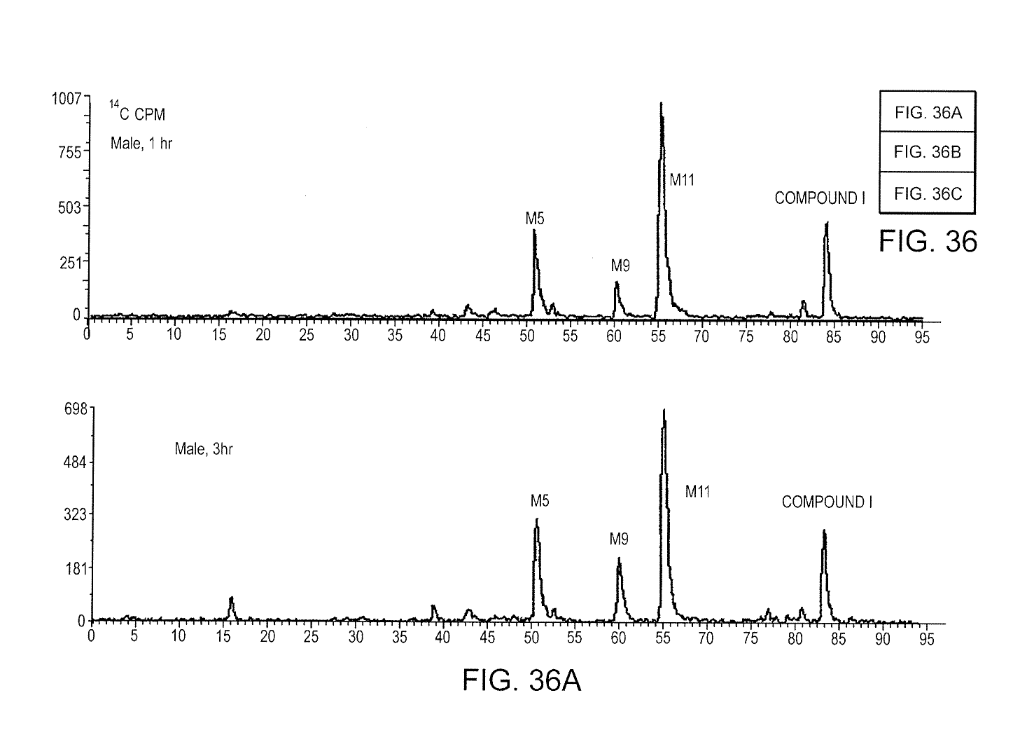

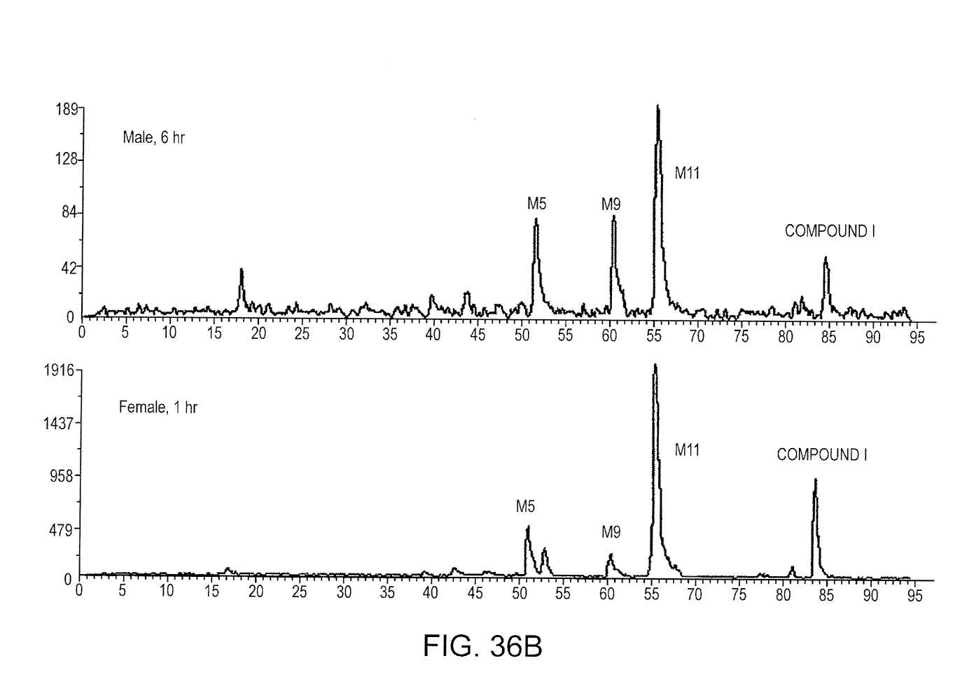

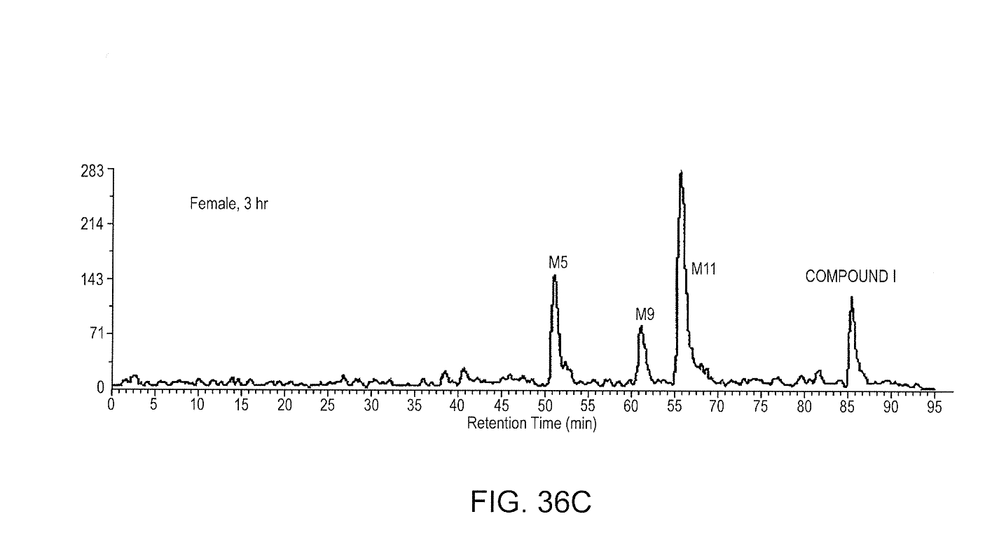

The extraction recovery of radioactivity from the 1, 3 and 6 hr pooled plasma samples was 77-95%. In male rats, COMPOUND I represented 8.7-16.9% of the total plasma radioactivity from 1 to 6 hr post-dose, decreasing with time (Table 19 and FIG. 36). In female rats, COMPOUND I represented 16.4 and 14.6% of total plasma radioactivity at 1 and 3 hr post-dose, respectively. Male and female rats had similar metabolite profiles (Table 19). O-Desmethyl COMPOUND I glucuronide (M11, 41.3-56.8% of total plasma radioactivity), hydroxy desfluoro COMPOUND I glucuronide (M5, 9.6-17.1%) and hydroxy COMPOUND I glucuronide (M9, 5.6-16.9%) were the major metabolites in plasma of male and female rats. Several minor radioactive peaks observed in plasma were not characterized due to low concentrations. The pooled 24 hr sample was not analyzed due to low radioactivity concentration and low extraction recovery.

TABLE 19
Chromatographic Distribution (%) of Radioactivity in Pooled
Plasma Samples from Rats Following a Single Oral
Administration of 5 mg/kg of [¹⁴C]COMPOUND I

| | | | | | |
|---|---|---|---|---|---|
| Time (hr) | M5 | M9 | M11 | COMPOUND I | Others^{a} |

Male

| | | | | | |
|---|---|---|---|---|---|
| 1 | 15.2 | 6.5 | 52.9 | 16.9 | 8.6 |
| 3 | 17.1 | 12.4 | 43.1 | 15.5 | 11.9 |
| 6 | 16.5 | 16.9 | 41.3 | 8.7 | 16.6 |

Female

| | | | | | |
|---|---|---|---|---|---|
| 1 | 9.6 | 5.6 | 56.8 | 16.4 | 11.5 |
| 3 | 16.2 | 9.9 | 48.2 | 14.6 | 11.1 |

^{a}Includes uncharacterized minor peaks (each represented less than 5% of plasma radioactivity).

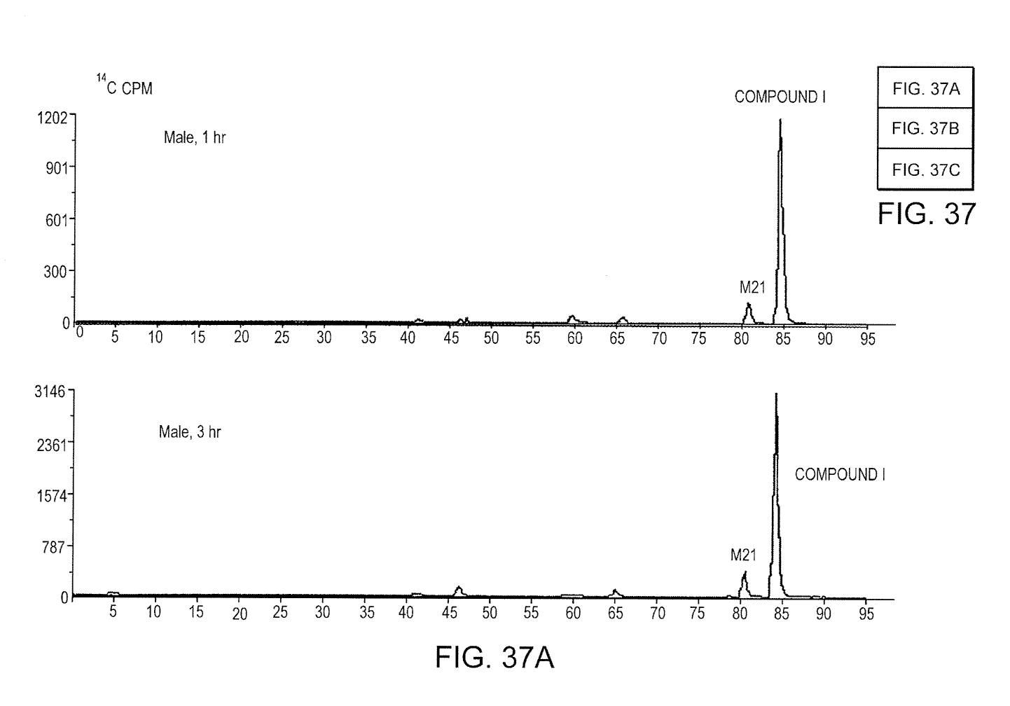

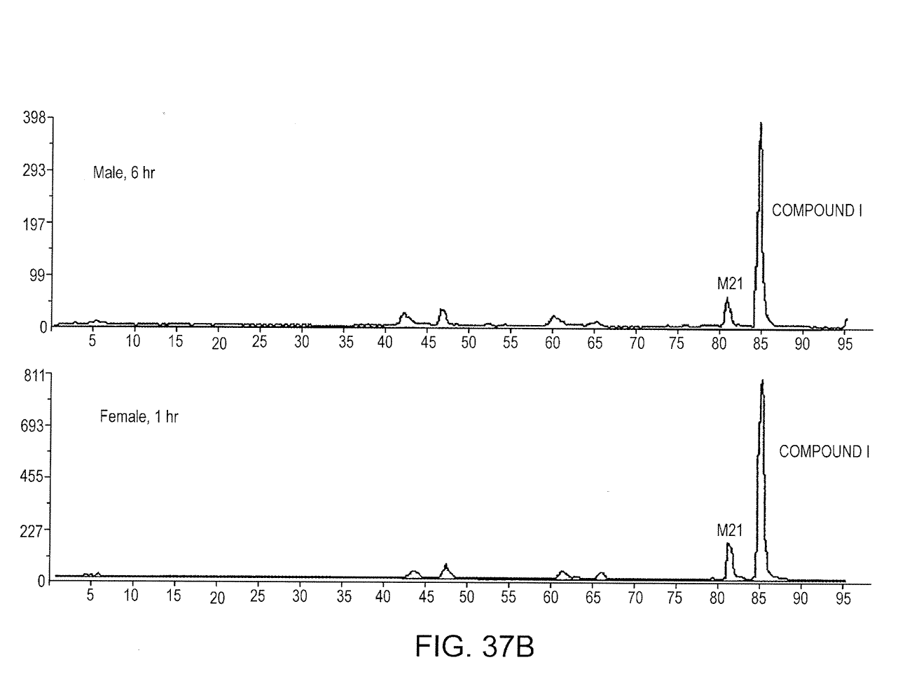

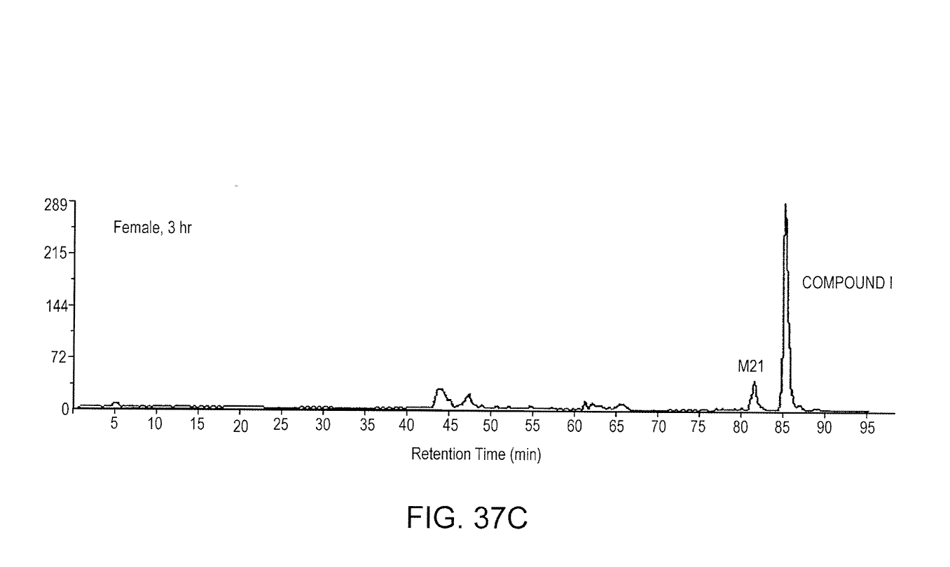

An average of 92% of the radioactivity in pooled brain samples was extracted. COMPOUND I was the major radioactive component in male and female rat brain. In male rats, COMPOUND I represented 80.3% of the total brain radioactivity at 1 hr, 63.8% at 3 hr and 60.5% at 6 hr post-dose. In female rats, COMPOUND I represented 79.5 and 70.7% of the total brain radioactivity at 1 and 3 hr post-dose, respectively. O-Desmethyl COMPOUND I (M21) was the major metabolite in brain, representing 9.0-9.4% of total radioactivity in male rats between 1 and 6 hr post-dose and 11.3-13.6% of total radioactivity in female rats between 1 and 3 hr post-dose (FIG. 37). Several minor radioactive peaks observed in brain extract were not characterized due to low concentrations. The 24 hr brain samples collected from male rats were not analyzed due to low radioactivity concentrations.

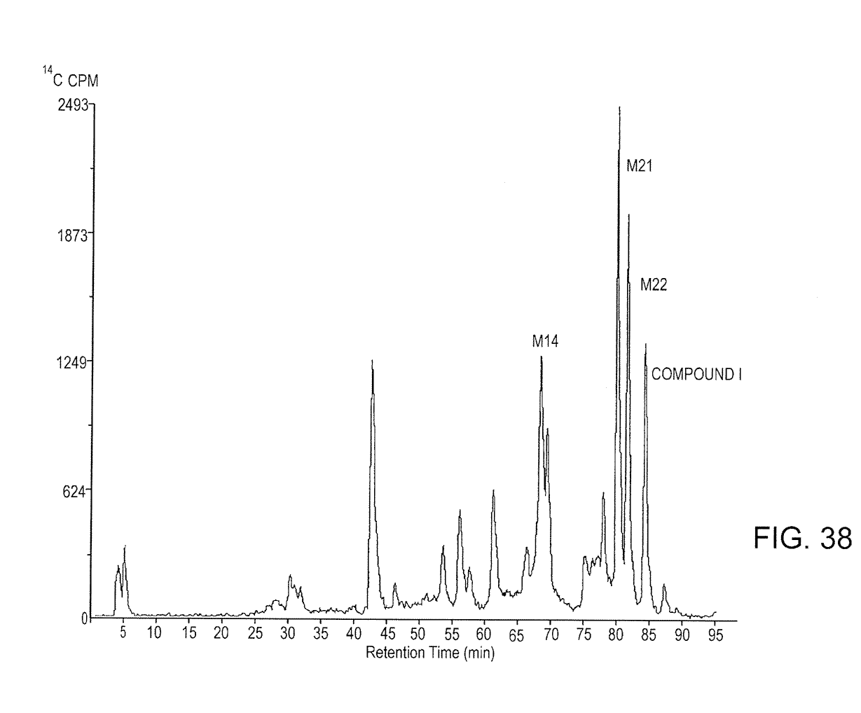

An average of 81.9% of administered radioactivity was recovered in feces within the first 24 hr. The extraction recovery of radioactivity from the pooled 0-24 hr fecal homogenate was 70%. COMPOUND I represented 8.1% of total radioactivity in the fecal extract. Major fecal metabolites included N-desmethoxyquinolinyl COMPOUND I (M14, 11.8%), O-desmethyl COMPOUND I (M21, 15.1%) and hydroxy COMPOUND I (M22, 10.9%) (FIG. 38). A number of other smaller radioactive peaks observed in rat feces were not characterized due to matrix interference.

Mass spectra for COMPOUND I and its metabolites in rat plasma, brain and feces were obtained by LC/MS and LC/MS/MS analysis. Structural characterization of these compounds is summarized in Table 20. The mass spectral characterization of COMPOUND I and its metabolites is discussed below. In LC/MS experiments conducted with D₂O substituted for H₂O in the mobile phase to determine number of exchangeable hydrogens, the mass difference between [M+D]⁺ and [M+H]⁺ was 1 Da larger than the number of exchangeable hydrogens on COMPOUND I and its metabolites due to exchange of the proton required for ionization to generate [M+H]⁺.

TABLE 20
[¹⁴C]COMPOUND I and Metabolites Characterized by LC/MS

| | t_{R} | | Site of | | |
|---|---|---|---|---|---|
| Peak | (min)^{a} | [M + H]⁺ | Metabolism | Metabolite Name | Source^{b} |
| M5 | 51 | 646 | Fluoroquinoline | Hydroxy desfluoro | P |
| | | | | COMPOUND I | |
| | | | | glucuronide | |
| M9 | 60 | 664 | Fluoroquinoline | Hydroxy COMPOUND | P |
| | | | | I glucuronide | |
| M11 | 66 | 634 | Methoxyquinoline | O-Desmethyl | P |
| | | | | COMPOUND I | |
| | | | | glucuronide | |
| M14 | 68 | 315 | Methoxyquinoline | N- | F |
| | | | | Desmethoxyquinolinyl | |
| | | | | COMPOUND I | |
| M21 | 81 | 458 | Methoxyquinoline | O-Desmethyl | B, F |
| | | | | COMPOUND I | |
| M22 | 83 | 488 | Fluoroquinoline | Hydroxy COMPOUND I | F |
| COMPOUND I | 85 | 472 | | | P, B, F |

^{a}Approximate HPLC retention times were taken from radiochromatograms and may differ from LC/MS retention times.
^{b}P, plasma; F, feces; B, brain. Bold face indicates major components in the matrix.

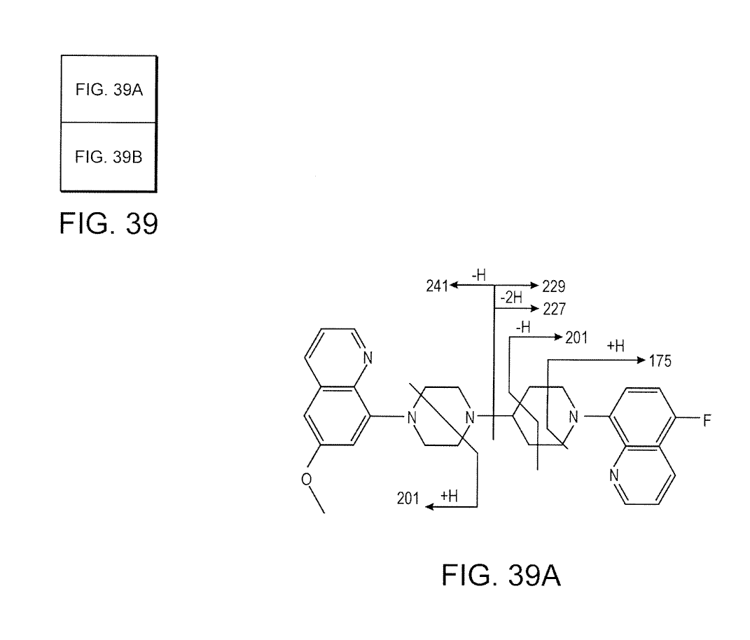

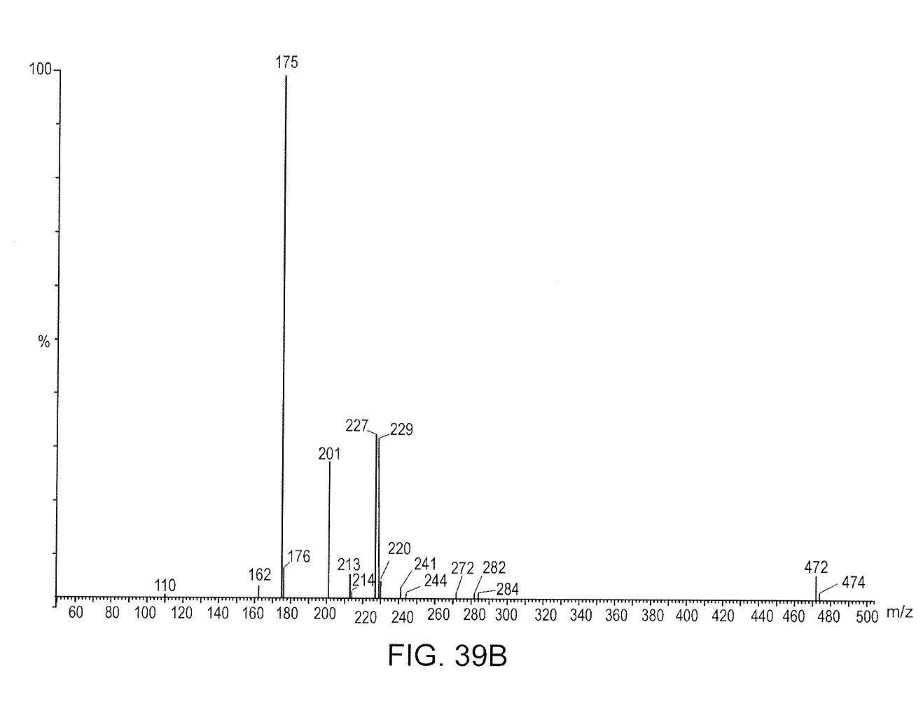

The mass spectral characteristics of synthetic COMPOUND I were examined for comparison with metabolites. In the LC/MS spectrum of COMPOUND I, the protonated molecular ion, [M+H]⁺ was observed at m/z 472. LC/MS with D₂O substituted for H₂O in the mobile phase generated [M+D]⁺ at m/z 473 (data not shown), consistent with COMPOUND I having no exchangeable hydrogens. The MS/MS spectrum obtained from collision activated dissociation of m/z 472 from COMPOUND I and the proposed fragmentation scheme are shown in FIG. 39. Fragmentation of the piperazine-piperidine bond with charge retention on the methoxyquinoline half of the molecule yielded m/z 241. The same fragmentation with charge retention on the fluoroquinoline half of the molecule yielded m/z 229 and 227. Fragmentation of the piperidine ring generated a fluoroquinoline-containing ion at m/z 175. Two assignments for the m/z 201 product ion were made. One m/z 201 product ion originated from cleavage of the piperidine ring with charge retention on the moiety containing the fluoroquinoline. The other m/z 201 product ion originated from cleavage of the piperazine ring. These assignments were confirmed by the product ions of m/z 474 (¹⁴C[M+H]⁺) and m/z 476 (¹⁴ C₂[M+H]⁺) mass spectral data for radiolabeled COMPOUND I (data not shown).

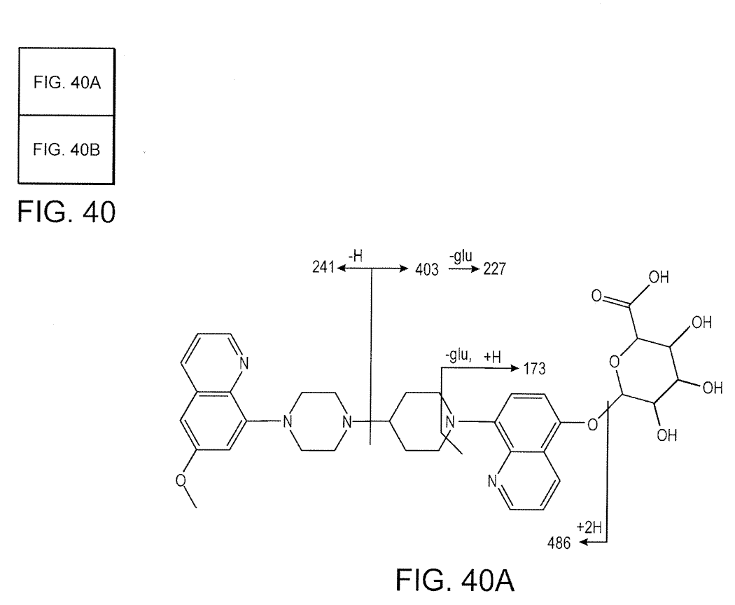

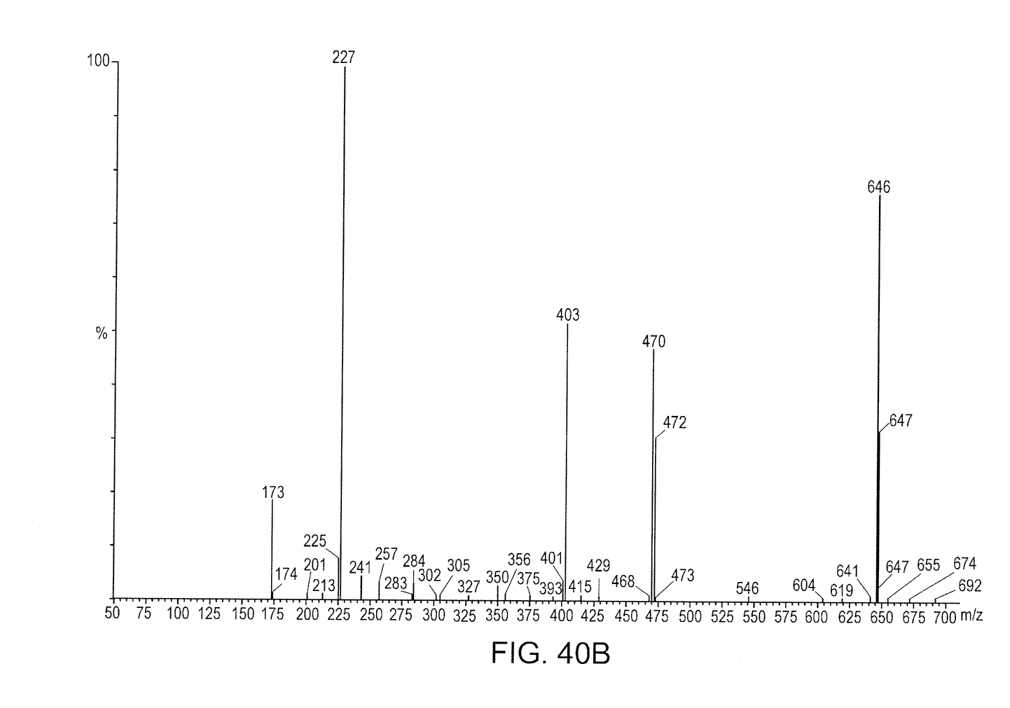

The [M+H]⁺ for M5 was observed at m/z 646, which was 174 Da larger than COMPOUND I. LC/MS with D₂O substituted for H₂O in the mobile phase generated [M+D]⁺ at m/z 651, which indicated four exchangeable hydrogens. The proposed fragmentation scheme and product ions of m/z 646 mass spectrum for M5 are shown in FIG. 40. Loss of 176 Da from [M+H]⁺ yielded m/z 470, which indicated that M5 was a glucuronide. The product ion at m/z 241 was also observed for COMPOUND I, which indicated an unchanged methoxyquinolinyl-piperazine moiety. Product ions at m/z 227 and 173 were 2 Da less than the corresponding ions at m/z 229 and 175, respectively, for COMPOUND I, which indicated oxidative defluorination of the fluoroquinoline. The product ion at m/z 403 was 176 Da larger than m/z 227, which indicated that the hydroxyl group resulting from the oxidative defluorination was the site of glucuronidation. Therefore, M5 was identified as hydroxy desfluoro COMPOUND I glucuronide.

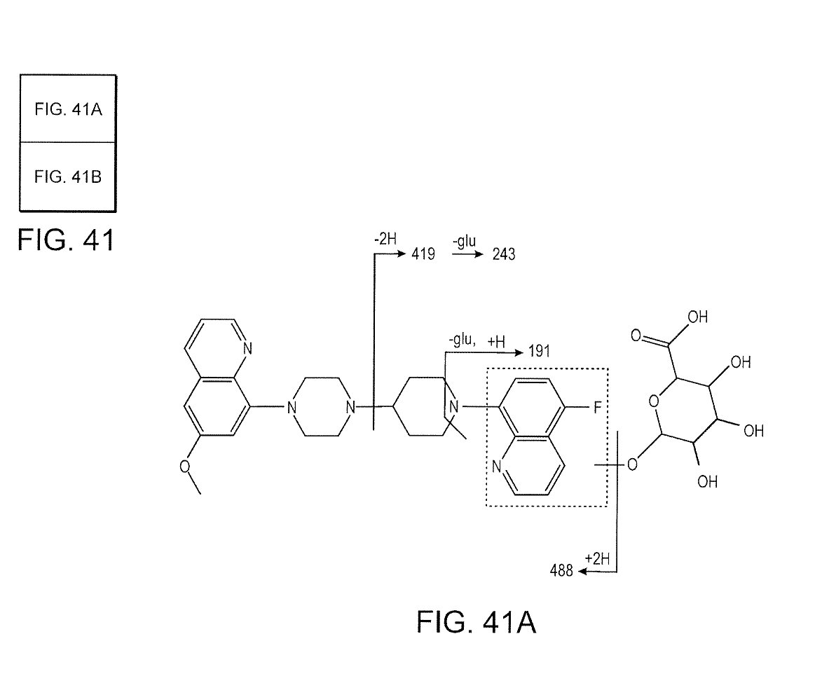

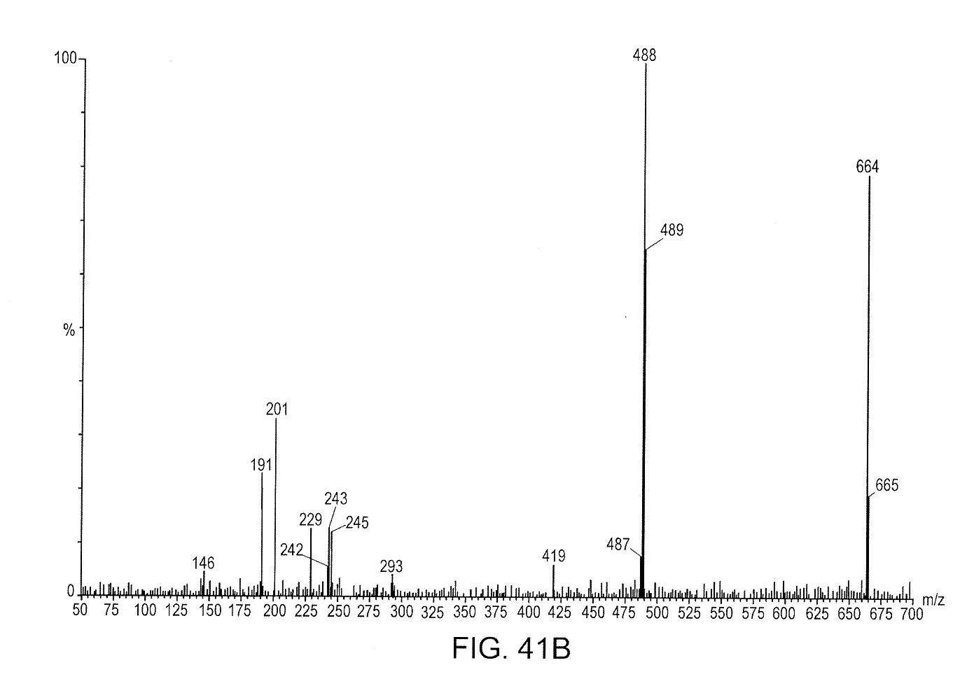

Metabolite M9 produced a [M+H]⁺ at m/z 664, which was 192 Da larger than COMPOUND I. LC/MS with D₂O substituted for H₂O in the mobile phase generated [M+D]⁺ at m/z 669, which indicated four exchangeable hydrogens. The proposed fragmentation scheme and product ions of m/z 664 mass spectrum for M9 are shown in FIG. 41. Loss of 176 Da from [M+H]⁺ yielded m/z 488, which was 16 Da larger than COMPOUND I and indicated that M9 was a glucuronide of a hydroxy COMPOUND I. Product ions at m/z 243 and 191 were 16 Da larger than the corresponding ions at m/z 227 and 175, respectively, for COMPOUND I, which was consistent with hydroxylation of the fluoroquinoline. The product ion at m/z 419 was 176 Da larger than m/z 243, which indicated that the hydroxylated fluoroquinoline was the site of glucuronidation. Therefore, M9 was identified as a hydroxy COMPOUND I glucuronide.

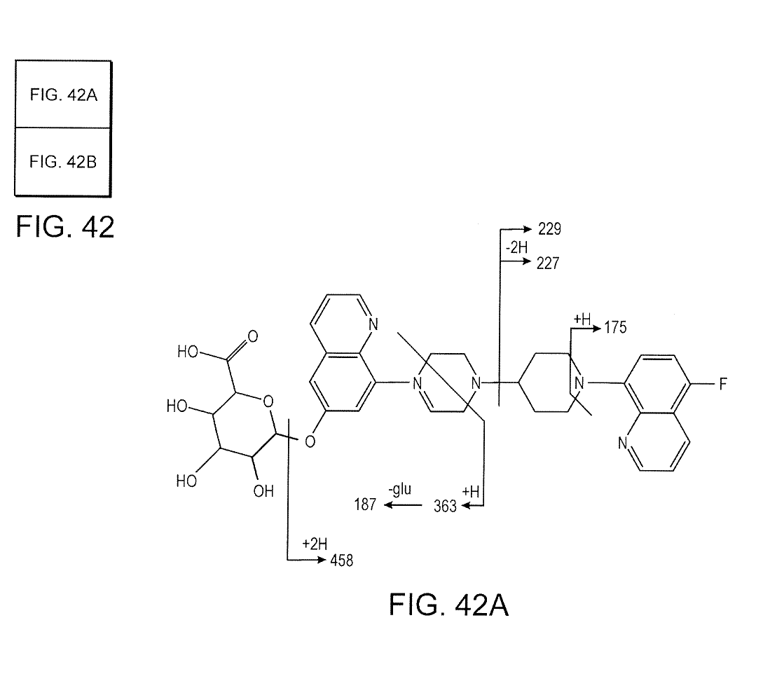

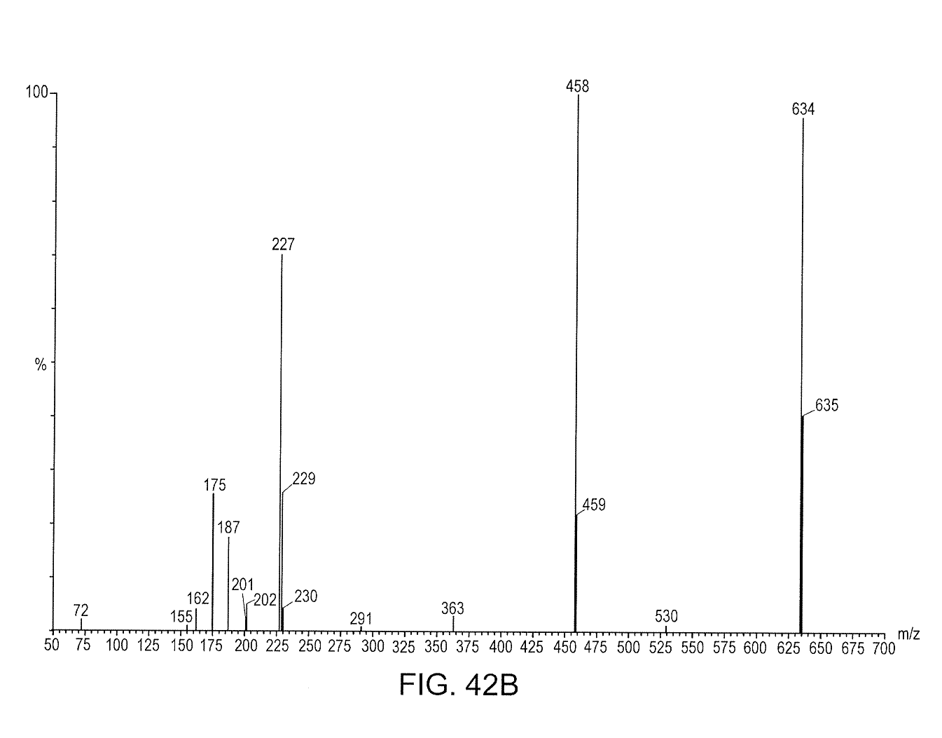

The [M+H]⁺ for M11 was observed at m/z 634, which was 162 Da larger than COMPOUND I. LC/MS with D₂O substituted for H₂O in the mobile phase generated [M+D]⁺ at m/z 639 (data not shown). These data indicated four exchangeable hydrogens, which was four more than COMPOUND I. The product ions of m/z 634 mass spectrum and the proposed fragmentation scheme for M11 are presented in FIG. 42. Neutral loss of 176 Da from [M+H]⁺ yielded m/z 458, which was 14 Da less than COMPOUND I. These data indicated that M11 was a glucuronide of a desmethyl COMPOUND I. Product ions at m/z 229, 227 and 175 were also observed for COMPOUND I, which indicated an unchanged fluoroquinolinyl-piperidine moiety. The product ion at m/z 187 was 14 Da less than the corresponding methoxyquinolinyl ion at m/z 201 for COMPOUND I, which indicated demethylation of the methoxy group. The product ion at m/z 363 was 176 Da larger than m/z 187, which indicated that the hydroxyl group formed from O-demethylation was the site of glucuronidation. Therefore, M11 was identified as O-desmethyl COMPOUND I glucuronide.

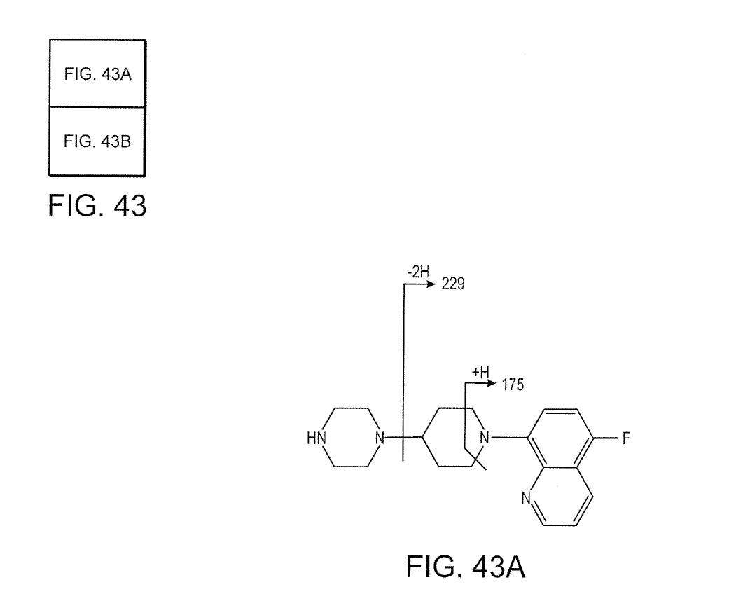

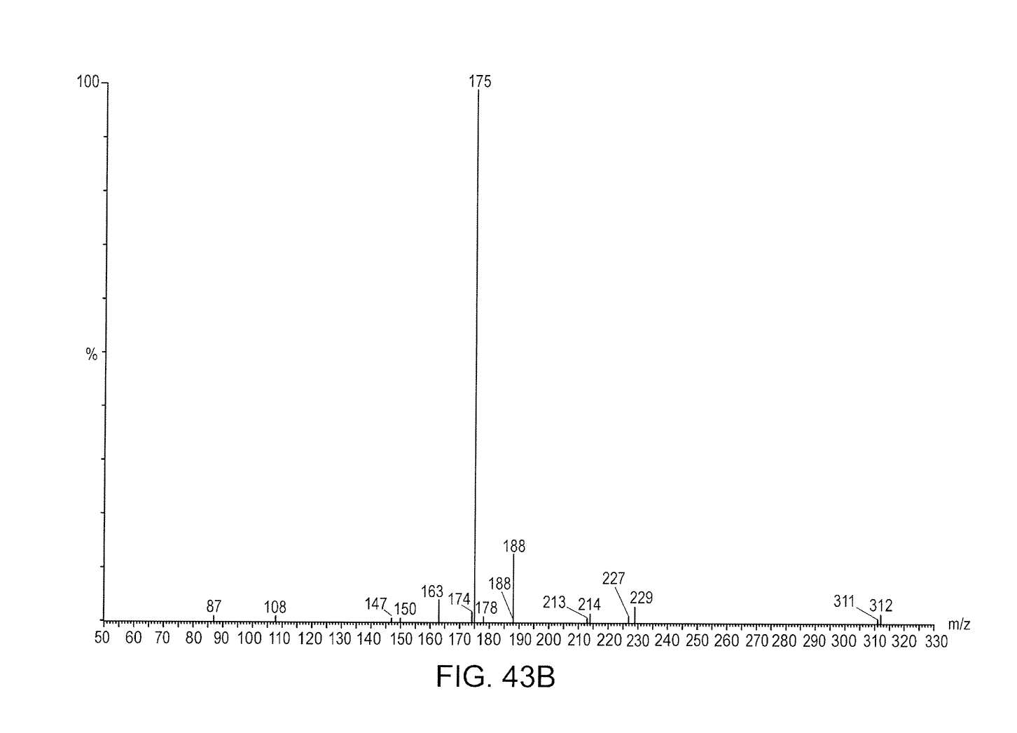

The [M+H]⁺ for M14 was observed at m/z 315, which was 157 Da less than COMPOUND I. LC/MS with D₂O substituted for H₂O in the mobile phase generated [M+D]⁺ at m/z 317 (data not shown). These data indicated one exchangeable hydrogen, which was one more than COMPOUND I and consistent with N-dealkylation of the COMPOUND I molecule. The product ions of m/z 315 mass spectrum and the proposed fragmentation scheme for M14 are presented in FIG. 43. Product ions at m/z 229 and 175 were also observed for COMPOUND I, which indicated unchanged piperidine and fluoroquinoline rings. These data and the molecular weight difference between M14 and COMPOUND I indicated that the methoxyquinoline moiety of COMPOUND I was not present in M14. Therefore, M14 was identified as N-desmethoxyquinolinyl COMPOUND I.

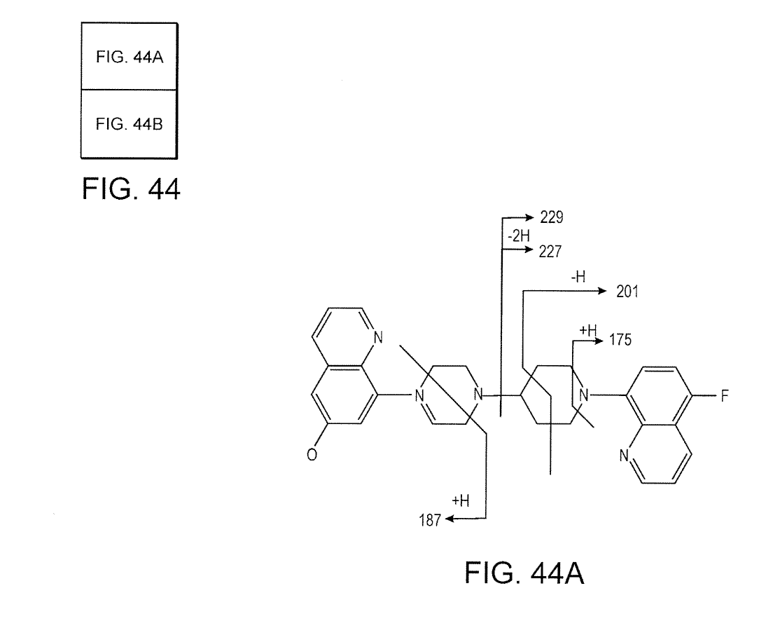

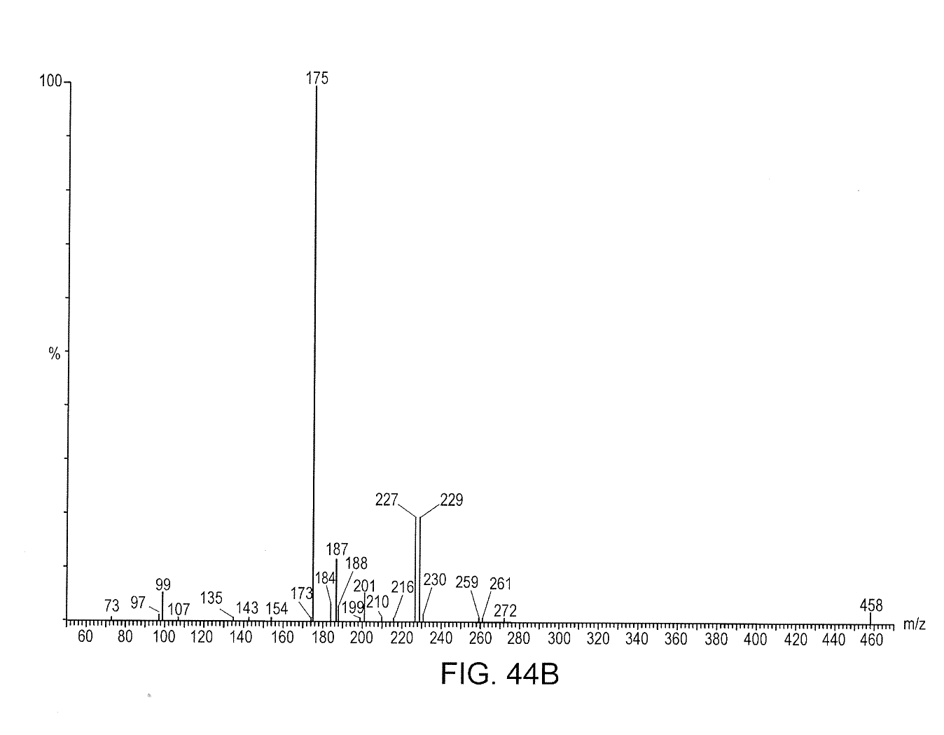

The [M+H]⁺ for M21 was observed at m/z 458, which was 14 Da less than COMPOUND I. LC/MS with D₂O substituted for H₂O in the mobile phase generated [M+D]⁺ at m/z 460 (data not shown). These data indicated one exchangeable hydrogen, which was one more than COMPOUND I and consistent with the presence of a hydroxyl group. The product ions of m/z 458 mass spectrum and the proposed fragmentation scheme for M21 are presented in FIG. 44. Product ions at m/z 229, 227, 201 and 175 were also observed for COMPOUND I, which indicated an unchanged fluoroquinolinyl-piperidine moiety. The product ion at m/z 187 was 14 Da less than the corresponding methoxyquinolinyl ion at m/z 201 for COMPOUND I, which indicated demethylation of the methoxy group. Therefore, M21 was identified as O-desmethyl COMPOUND I.

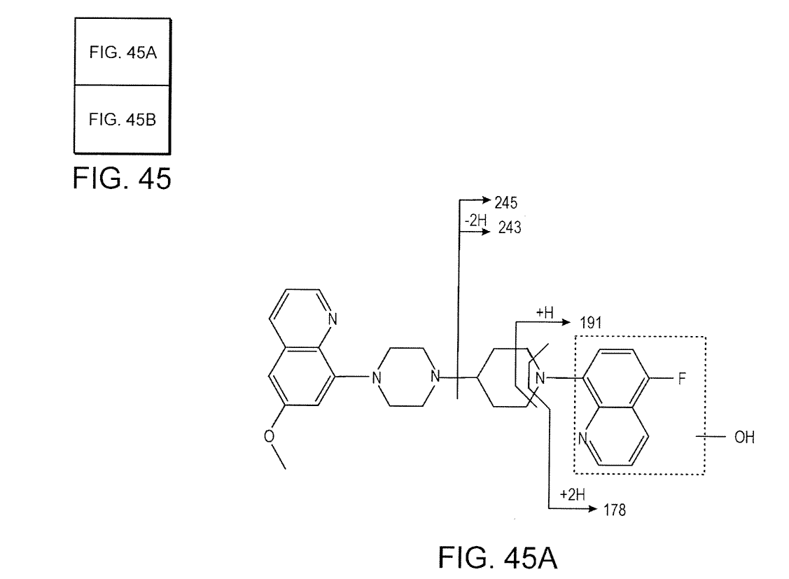

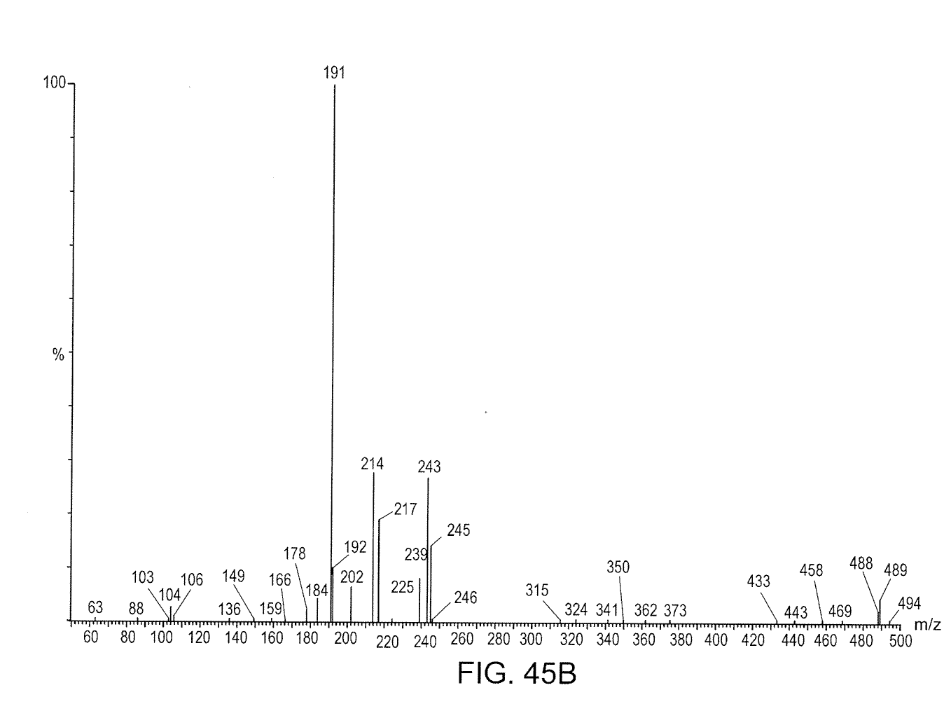

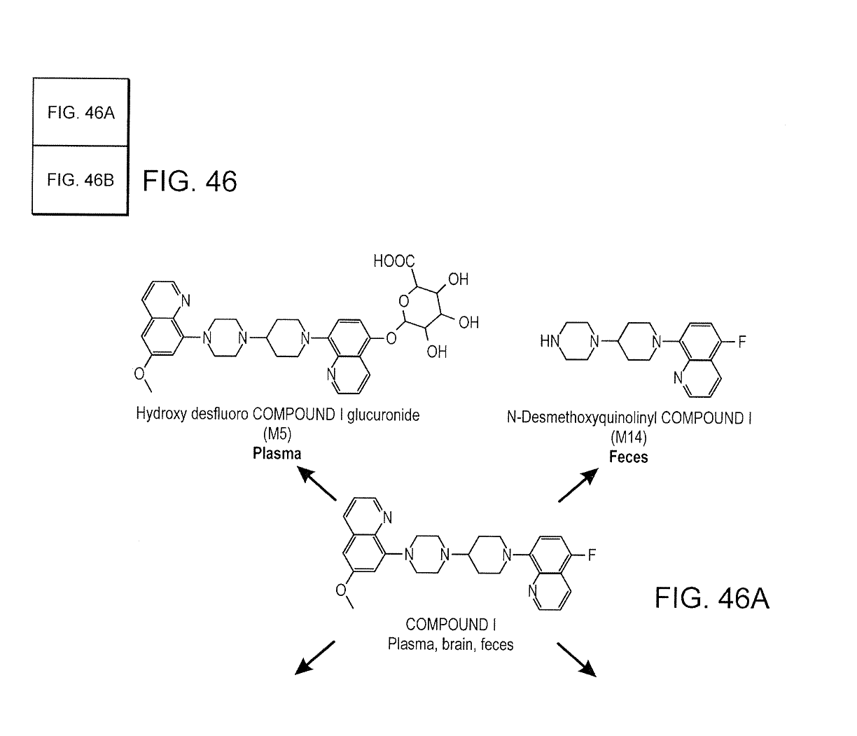

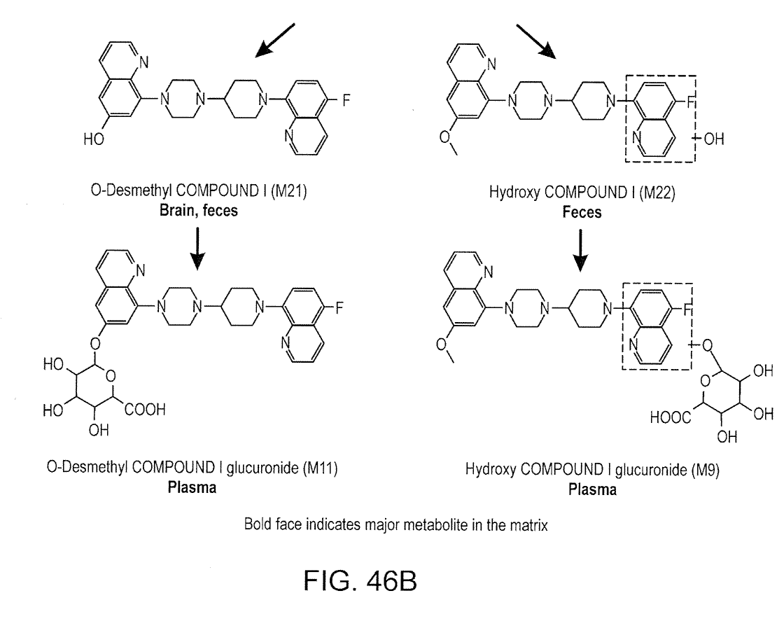

Metabolite M22 produced a [M+H]⁺ at m/z 488, which was 16 Da larger than COMPOUND I. LC/MS with D₂O substituted for H₂O in the mobile phase generated [M+D]⁺ at m/z 490 (data not shown). These data indicated one exchangeable hydrogen, which was one more than COMPOUND I and consistent with hydroxylation rather than N-oxidation. The product ions of m/z 488 mass spectrum and the proposed fragmentation scheme for M22 are presented in FIG. 45. Product ions at m/z 245, 243 and 191 were 16 Da larger than the corresponding ions at m/z 229, 227 and 175, respectively, for COMPOUND I, which indicated hydroxylation of the fluoroquinoline. Therefore, M22 was identified as a hydroxy COMPOUND I.

### In Vivo Metabolism of [¹⁴C]COMPOUND I in Male Beagle Dogs Following a Single Oral 3 Mg/Kg Dose of [¹⁴C]COMPOUND I

The in vivo metabolism of [¹⁴C]COMPOUND I was investigated in male beagle dogs following a single 3 mg/kg oral dose and the metabolites were characterized by LC/MS. The synthesis of [¹⁴C]COMPOUND I trisuccinate salt was prepared as described above. Ultima Gold, Ultima Flo M, Permafluor® E⁺ scintillation cocktails, and Carbo-Sorb E carbon dioxide absorber were purchased from Perkin Elmer Life Sciences (Boston, Mass.). Polysorbate 80 was obtained from Mallinckrodt Baker (Phillipsburg, N.J.). Methylcellulose was from Sigma-Aldrich (Milwaukee, Wis.). Solvents used for extraction and for chromatographic analysis were HPLC or ACS reagent grade and were purchased from EMD Chemicals (Gibbstown, N.J.). Deuterium oxide (D₂O) was obtained from Cambridge Isotope Laboratories (Andover, Mass.).

Dose preparation, animal dosing, and specimen collection were performed at Wyeth Research, Collegeville, Pa. The vehicle contained 2% (w/v) polysorbate 80, NF and 0.5% (w/v) methylcellulose (4000 cps) in water. [¹⁴C]COMPOUND I trisuccinate salt (50.25 mg) and non-labeled COMPOUND I trisuccinate salt (331.9 mg) were suspended in 72 mL of the vehicle by grinding with a mortar and a pestle. The target [¹⁴C]COMPOUND I concentration was approximately 3 mg/mL as free base and 48 μCi/mL with a target specific activity of 16 μCi/mg. Three pre- and post-dose aliquots (100 μL) were taken for the determination of radiochemical purity, drug and radioactivity concentrations and specific activity (see below).

Four male beagle dogs, weighing from 9.4 to 11.5 kg at the time of dosing, were from an in-house colony. Non-fasted dogs were given a single 3 mg/kg (approximately 48 μCi/kg) dose of [¹⁴C]COMPOUND I at a volume of 1 mL/kg via intragastric gavage. Animals were provided Purina dog chow and water ad libitum, and were housed individually in metabolic cages.

Blood samples were collected from the jugular vein at 1, 4, 8, 24, 48, 72 and 120 hr after dose administration into tubes containing potassium EDTA as the anticoagulant and then placed on ice. Aliquots of 200 μL were removed and plasma was harvested immediately from the remaining blood by centrifugation at 4° C. Urine samples were collected into tubes on dry ice from 0-24 and 24-48 hr, and then at ambient temperature at 24 hr intervals for 7 days post-dose. Fecal samples were collected at 24 hr intervals for 7 days post-dose at room temperature. Cage rinses were collected daily by rinsing each cage with approximately 500 mL of 30% ethanol in water. The dose aliquots and biological specimens were stored at approximately −70° C. until analysis.

Aliquots of the pre- and post-dose suspension were dissolved in 25% methanol in water and analyzed for radioactivity concentrations as described hereinbelow. Approximately 80,000 dpm in 40 μL was analyzed by HPLC for radiochemical purity and chemical purity (see below). To determine the specific activity of the dose suspension, non-radiolabeled COMPOUND I was dissolved in 25% methanol in water to give five different concentrations ranging from 4.9 to 98 μg/mL and concurrently analyzed by HPLC to generate a standard curve. Aliquots (40 μL) of the diluted [¹⁴C]COMPOUND I dose suspension were injected onto the HPLC column and fractions were collected at 60 second intervals after UV detection. Radioactivity in each fraction was determined as described hereinbelow. Fractions were also collected from a blank injection to obtain the background level of radioactivity. The UV peak associated with [¹⁴C]COMPOUND I was integrated to calculate the drug concentration. The specific activity of [¹⁴C]COMPOUND I was derived from the amount of drug in the peak and the total radioactivity in the fractions associated with the drug peak.

Triplicate aliquots of diluted dose (20 μL), dose fractions (10 μL) and plasma (50 μL), urine (200 μL) and cage wash (500 μL) from individual dogs were analyzed for radioactivity content. The samples were assayed in a Tri-Carb Model 3100TR liquid scintillation counter (Perkin Elmer) using 5 mL of Ultima Gold scintillation cocktail.

Fecal samples from individual dogs were weighed and homogenized in water at a volume-to-weight ratio of about 4:1 with a Silverson sealed-unit homogenizer at ambient temperature. Triplicate aliquots of whole blood (50 μL) and fecal homogenates (approximately 0.2 g) were placed into Combusto-cones with Combusto-pads and allowed to dry overnight. Samples were combusted with a model 307 Tri-Carb Sample Oxidizer, equipped with an Oximate-80 Robotic Automatic Sampler (Perkin Elmer). The liberated ¹⁴CO₂ was trapped with Carbo-Sorb® E carbon dioxide absorber, mixed with PermaFluor® E+liquid scintillation cocktail, and counted in a Tri-Carb Model 3100 TR/LL liquid scintillation counter. The oxidation efficiency of the oxidizer was 98.0%.

Plasma samples were pooled by mixing equal volumes from four animals at each time point (1, 4, 8 and 24 hr). Aliquots of 2.0 mL of pooled plasma were mixed with 2.0 mL of acetonitrile, placed on ice for about 10 min, and then centrifuged at 4° C. The supernatant was transferred to a clean tube. The protein pellets were extracted two more times with 2.0 mL of acetonitrile. The supernatants from precipitation and extraction of each sample were pooled, mixed, and evaporated at 22° C. under nitrogen in a TurboVap LV evaporator (Caliper Life Sciences, Hopkinton, Mass.) to about 0.8 mL. The concentrated extract was centrifuged, the supernatant volume measured and the extraction efficiency was determined by analyzing duplicate 10 μL aliquots for radioactivity concentrations. For metabolite profiling, an aliquot of the supernatant (200 μL) was injected onto the HPLC column as described hereinbelow, and fractions were collected at 20 second intervals into 96-well Lumaplates (Perkin Elmer). The plates were dried overnight in an oven at 40° C. and analyzed by a TopCount NXT radiometric microplate reader (Perkin Elmer). Plasma extracts were also analyzed by LC/MS for metabolite characterization as described hereinbelow.

Fecal homogenates (0-24 and 24-48 hr) were pooled proportionally to their total weight and analyzed for metabolite profiles. Aliquots of 2.0 g of the pooled fecal homogenates were mixed with 4.0 mL of acetonitrile, placed on ice for about 10 min and centrifuged at 4° C. The supernatant was transferred to a clean tube. The residue was extracted two more times with 4.0 mL of acetonitrile. The supernatants were combined and evaporated to a volume of about 2.0 mL. Extraction efficiency was determined by analyzing aliquots of 10 μL of the supernatant for radioactivity. For metabolite profiling, an aliquot (50 μL) of the supernatant was analyzed by HPLC with radioactivity flow detection (see below). Samples were also analyzed by LC/MS to characterize the radioactive peaks (see below).

Since less than 7% of dose was excreted in urine (see below), urine samples were not analyzed for metabolite profiles.

A Waters Model 2695 HPLC system (Waters Corp., Milford, Mass.) with a built-in autosampler was used for analysis. Separations were accomplished on a Luna C18(2) column (150×2.0 mm, 5 μm) (Phenomenex, Torrance, Calif.) for dose analysis and on a Synergi Hydro-RP column (150×2.0, 4 μm) (Phenomenex) for metabolite profiling. A Phenomenex Securiguard cartridge (4×2 mm) was coupled to the columns. The sample chamber in the autosampler was maintained at 4° C., while the columns were at ambient temperature of about 20° C. For plasma samples, fractions were collected and analyzed by TopCount as described hereinabove. For fecal extracts, a Flo-One β Model A525 radioactivity flow detector (Perkin Elmer) with a 250 μL LQTR flow cell and a Waters Model 996 photodiode array UV detector set to monitor at 250 nm were used for data acquisition. The flow rate of Ultima Flo M scintillation fluid was 1.2 mL/min, providing a mixing ratio of scintillation cocktail to mobile phase of about 6:1. The mobile phase consisted of 10 mM ammonium acetate, pH 4.5 (A) and acetonitrile (B), and was delivered at 0.2 mL/min. The linear gradient conditions for dose analysis and metabolite profiles are summarized in Table 21 and Table 22, respectively.

TABLE 21
HPLC Linear Gradient Conditions for Dose Analysis

| Time (min) | A (%) | B (%) |
|---|---|---|
| 0 | 95 | 5 |
| 5 | 95 | 5 |
| 45 | 55 | 45 |
| 50 | 55 | 45 |

TABLE 22
HPLC Linear Gradient Conditions for Metabolite Profiles

| Time (min) | A (%) | B (%) |
|---|---|---|
| 0 | 95 | 5 |
| 5 | 95 | 5 |
| 5.1 | 90 | 10 |
| 55 | 65 | 35 |
| 60 | 40 | 60 |
| 70 | 40 | 60 |

The HPLC system used for mass spectrometric analysis was a Waters Alliance Model 2695 HPLC system (Waters Corp.). It was equipped with a built-in autosampler and a Model 996 diode array UV detector (Waters Corp.). The UV detector was set to monitor 210-400 nm. The HPLC conditions were as described hereinabove for metabolite profiling. The column re-equilibration time was 16 min (86 min total run time). For H-D exchange experiments, D₂O was substituted for H₂O in mobile phase A. During LC/MS analysis, up to the first 5 min of flow was diverted away from the mass spectrometer prior to evaluation of metabolites.

The mass spectrometer used for metabolite characterization was a Micromass Quattro Ultima triple quadrupole mass spectrometer (Waters Corp). It was equipped with an electrospray ionization (ESI) interface and operated in the positive ionization mode. Settings for the mass spectrometer are listed in Table 23.

TABLE 23
Micromass Mass Spectrometer Settings

| | |
|---|---|
| ESI spray | 2.5 kV |
| Cone | 45 V |
| Mass resolution of scanning | 0.7 Da ± 0.2 Da width at half height |
| mass analyzer | |
| Mass resolution of non- | 1-2 Da width at half height |
| scanning mass analyzer for | |
| MS/MS experiments | |
| Desolvation gas flow | 950-1100 L/hr |
| Source block temp. | 80° C. |
| Desolvation gas temp. | 250° C. |
| Collision gas pressure | 0.9-1.1 × 10⁻³ mbar |
| Collision offset | 35 eV |

Flo-One analytical software (Perkin Elmer, version 3.65) was utilized to integrate the radioactive peaks. DataFlo software utility (Perkin Elmer, beta version 0.55) was used to convert ASCII files from the TopCount NXT microplate counter into CR format for processing in Flo-One analysis software. Micromass MassLynx software (version 4.0, Waters Corp.) was used for analysis of LC/MS data. Microsoft Excel® 2000 was used to calculate means and standard deviations. Radioactivity in urine samples collected after the study completion was estimated by multiplying the radioactivity concentrations with the approximate urine volumes.

The radiochemical purity and chemical purity of [¹⁴C]COMPOUND I in the dose suspension were greater than 99%. The pre- and post-dose aliquots had similar purity. The specific activity of the [¹⁴C]COMPOUND I dosing suspension was 15.3 μCi/mg. The average drug concentration was 3.38 mg/mL (51.7 μCi/mL). The actual administered dose of [¹⁴C]COMPOUND I was 3.37 mg/kg. Dose concentration and specific activity were within 15% of theoretical values.

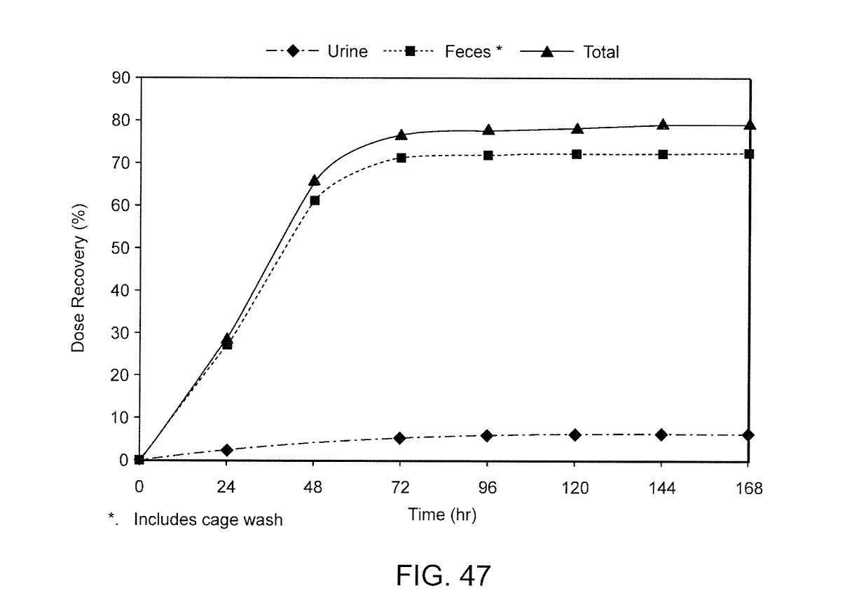

The mean (±SD) and individual cumulative and daily excretion of radioactivity by male dogs after a single oral administration of [¹⁴C]COMPOUND I are presented in Tables 24, 25 and 26, and FIG. 47. The mean recoveries in feces (including cage wash) and urine by 48 hr post-dose were 61.2% and 4.31%, respectively (Table 24). By 168 hr post-dose, 72.8% was recovered in feces and 6.56% was recovered in urine (Table 24). The overall total (mean ±SD) recovery was 79.4±5.87% and ranged from 72.5% to 84.9% at 168 hr post-dose. By day 7, a mean of 0.44% of the dose was present in the excreta. Based on the amounts of radioactivity in urine collected for several additional weeks (data not shown), it was estimated that about 5% of the administered dose was excreted after the completion of the study.

TABLE 24
Mean (±SD) Cumulative Percent Excretion of Radioactivity
Following a Single 3 mg/kg Oral Dose of
[¹⁴C]COMPOUND I in Male Dogs

| | % Dose (0-48 hr) | % Dose (0-168 hr) |
|---|---|---|
| Parameters | (n = 4) | (n = 4) |
| Urine | 4.31 ± 2.51 | 6.56 ± 3.56 |
| Feces^{a} | 61.2 ± 14.1 | 72.8 ± 3.28 |
| Total | 65.5 ± 15.6 | 79.4 ± 5.87 |

^{a}Includes cage rinse.

TABLE 25
Recovery of Radioactivity (Cumulative Percent of Dose)
in Excreta of Male Dogs Following a Single 3 mg/kg Oral
Dose of [¹⁴C]COMPOUND I

| | | | | | |
|---|---|---|---|---|---|
| Time (hr) | Dog 1 | Dog 2 | Dog 3 | Dog 4 | Mean ± SD |

Urine

| | | | | | |
|---|---|---|---|---|---|
| 0-24 | 1.63 | 3.32 | 1.12 | 1.63 | 1.93 ± 0.96 |
| 0-48 | 2.63 | 7.90 | 2.55 | 4.15 | 4.31 ± 2.51 |
| 0-72 | 3.33 | 9.15 | 2.46 | 5.45 | 5.10 ± 2.98 |
| 0-96 | 3.49 | 9.57 | 2.91 | 6.16 | 5.53 ± 3.04 |
| 0-120 | 4.05 | 10.3 | 3.01 | 6.62 | 6.00 ± 3.25 |
| 0-144 | 4.18 | 10.6 | 3.17 | 7.27 | 6.31 ± 3.35 |
| 0-168 | 4.26 | 11.1 | 3.24 | 7.63 | 6.56 ± 3.56 |

Feces

| | | | | | |
|---|---|---|---|---|---|
| 0-24 | 0.00 | 31.3 | 39.0 | 32.4 | 25.7 ± 17.5 |
| 0-48 | 40.0 | 65.4 | 61.9 | 70.2 | 59.4 ± 13.4 |
| 0-72 | 66.7 | 69.4 | 69.2 | 72.2 | 69.4 ± 2.25 |
| 0-96 | 66.8 | 69.8 | 69.9 | 72.9 | 69.9 ± 2.49 |
| 0-120 | 67.2 | 70.0 | 70.1 | 73.5 | 70.2 ± 2.58 |
| 0-144 | 67.3 | 70.1 | 70.3 | 73.6 | 70.3 ± 2.58 |
| 0-168 | 67.4 | 70.2 | 70.5 | 73.8 | 70.5 ± 2.62 |

Cage Rinse

| | | | | | |
|---|---|---|---|---|---|
| 0-24 | 0.18 | 1.64 | 1.74 | 0.78 | 1.09 ± 0.74 |
| 0-48 | 0.46 | 2.57 | 2.20 | 1.75 | 1.75 ± 0.92 |
| 0-72 | 0.65 | 3.00 | 2.57 | 1.97 | 2.05 ± 1.02 |
| 0-96 | 0.72 | 3.27 | 2.66 | 2.05 | 2.18 ± 1.09 |
| 0-120 | 0.75 | 3.39 | 2.73 | 2.12 | 2.25 ± 1.13 |
| 0-144 | 0.79 | 3.49 | 2.77 | 2.17 | 2.31 ± 1.15 |
| 0-168 | 0.84 | 3.58 | 2.83 | 2.20 | 2.36 ± 1.16 |

Total

| | | | | | |
|---|---|---|---|---|---|
| 0-24 | 1.81 | 36.3 | 41.9 | 34.8 | 28.7 ± 18.2 |
| 0-48 | 43.1 | 75.9 | 66.7 | 76.2 | 65.5 ± 15.6 |
| 0-72 | 70.6 | 81.5 | 74.5 | 79.7 | 76.6 ± 4.97 |
| 0-96 | 71.0 | 82.6 | 75.4 | 81.1 | 77.5 ± 5.34 |
| 0-120 | 72.0 | 83.6 | 75.9 | 82.2 | 78.4 ± 5.44 |
| 0-144 | 72.3 | 84.3 | 76.3 | 83.0 | 79.0 ± 5.66 |
| 0-168 | 72.5 | 84.9 | 76.6 | 83.6 | 79.4 ± 5.87 |

TABLE 26
Recovery of Radioactivity (Percent of Dose) in Excreta of Male Dogs
Following a Single 3 mg/kg Oral Dose of [¹⁴C]COMPOUND I

| | | | | | |
|---|---|---|---|---|---|
| Time (hr) | Dog 1 | Dog 2 | Dog 3 | Dog 4 | Mean ± SD |

Urine

| | | | | | |
|---|---|---|---|---|---|
| 0-24 | 1.63 | 3.32 | 1.12 | 1.63 | 1.93 ± 0.96 |
| 24-48 | 1.00 | 4.58 | 1.43 | 2.52 | 2.38 ± 1.60 |
| 48-72 | 0.70 | 1.25 | 0.21 | 1.30 | 0.87 ± 0.51 |
| 72-96 | 0.16 | 0.42 | 0.15 | 0.71 | 0.36 ± 0.26 |
| 96-120 | 0.56 | 0.69 | 0.16 | 0.46 | 0.47 ± 0.23 |
| 120-144 | 0.13 | 0.39 | 0.10 | 0.65 | 0.32 ± 0.26 |
| 144-168 | 0.08 | 0.46 | 0.07 | 0.36 | 0.24 ± 0.20 |

Feces

| | | | | | |
|---|---|---|---|---|---|
| 0-24 | 0.00 | 31.3 | 39.0 | 32.4 | 25.7 ± 17.5 |
| 24-48 | 40.0 | 34.1 | 22.9 | 37.9 | 33.7 ± 7.62 |
| 48-72 | 26.7 | 3.97 | 7.26 | 1.98 | 10.0 ± 11.4 |
| 72-96 | 0.14 | 0.41 | 0.68 | 0.70 | 0.48 ± 0.26 |
| 96-120 | 0.37 | 0.21 | 0.22 | 0.52 | 0.33 ± 0.15 |
| 120-144 | 0.15 | 0.12 | 0.26 | 0.14 | 0.17 ± 0.06 |
| 144-168 | 0.06 | 0.13 | 0.20 | 0.16 | 0.14 ± 0.06 |

Cage Rinse

| | | | | | |
|---|---|---|---|---|---|
| 0-24 | 0.18 | 1.64 | 1.74 | 0.78 | 1.09 ± 0.74 |
| 24-48 | 0.28 | 0.93 | 0.46 | 0.97 | 0.66 ± 0.34 |
| 48-72 | 0.19 | 0.43 | 0.37 | 0.22 | 0.30 ± 0.12 |
| 72-96 | 0.07 | 0.27 | 0.09 | 0.08 | 0.13 ± 0.10 |
| 96-120 | 0.03 | 0.12 | 0.07 | 0.07 | 0.07 ± 0.04 |
| 120-144 | 0.04 | 0.10 | 0.04 | 0.05 | 0.06 ± 0.03 |
| 144-168 | 0.05 | 0.09 | 0.06 | 0.03 | 0.06 ± 0.03 |

Total

| | | | | | |
|---|---|---|---|---|---|
| 0-24 | 1.81 | 36.3 | 41.9 | 34.8 | 28.7 ± 18.2 |
| 24-48 | 41.3 | 39.6 | 24.8 | 41.4 | 36.8 ± 8.03 |
| 48-72 | 27.5 | 5.65 | 7.84 | 3.50 | 11.1 ± 11.1 |
| 72-96 | 0.37 | 1.10 | 0.92 | 1.49 | 0.97 ± 0.47 |
| 96-120 | 0.96 | 1.02 | 0.45 | 1.05 | 0.87 ± 0.28 |
| 120-144 | 0.32 | 0.61 | 0.40 | 0.84 | 0.54 ± 0.23 |
| 144-168 | 0.19 | 0.68 | 0.33 | 0.55 | 0.44 ± 0.22 |

The concentrations of radioactivity in whole blood and plasma, and whole blood-to-plasma ratios of radioactivity following administration of a single oral dose of 3 mg/kg of [¹⁴C]COMPOUND I to male beagle dogs are summarized in Table 27. The mean (±SD) plasma concentrations of total radioactivity quickly reached a maximum of 424±66.1 ng equivalents/mL at 1 hr post-dose (the first time point taken) and declined fairly rapidly to 90.8±45.9 ng equivalents/mL at 24 hr post-dose. The average whole blood-to-plasma ratios of radioactivity were 0.79 to 0.97 over 72 hr post-dose, indicating some partitioning of COMPOUND I and its metabolites into blood cells.

TABLE 27
Concentrations (ng equivalents/mL) of Total Radioactivity in
Whole Blood and Plasma and Whole Blood to Plasma Ratios
of Radioactivity in Male Dogs Following a Single 3 mg/kg Oral
Dose of [¹⁴C]COMPOUND I

| | | | | | |
|---|---|---|---|---|---|
| Sampling | | | | | |
| Time (hr) | Dog 1 | Dog 2 | Dog 3 | Dog 4 | Mean ± SD |

Whole Blood

| | | | | | |
|---|---|---|---|---|---|
| 1 | 270 | 284 | 404 | 375 | 333 ± 66.3 |
| 4 | 138 | 201 | 191 | 281 | 203 ± 59.0 |
| 8 | 93.5 | 131 | 160 | 126 | 128 ± 27.2 |
| 24 | 41.2 | 63.3 | 98.5 | 82.9 | 71.5 ± 24.8 |
| 48 | 22.2 | 30.2 | 40.8 | 42.2 | 33.9 ± 9.44 |
| 72 | 16.8 | 22.6 | 25.2 | 31.6 | 24.1 ± 6.14 |
| 120 | 16.6 | 18.4 | 18.0 | 24.0 | 19.3 ± 3.26 |

Plasma

| | | | | | |
|---|---|---|---|---|---|
| 1 | 336 | 426 | 496 | 437 | 424 ± 66.1 |
| 4 | 166 | 228 | 326 | 181 | 225 ± 72.2 |
| 8 | 102 | 135 | 171 | 121 | 132 ± 29.2 |
| 24 | 49.1 | 69.3 | 155 | 89.7 | 90.8 ± 45.9 |
| 48 | 25.5 | 28.2 | 58.5 | 40.7 | 38.2 ± 15.1 |
| 72 | 17.6 | 21.4 | 73.6 | 39.0 | 37.9 ± 25.6 |
| 120 | 45.1 | 46.0 | 45.1 | 46.7 | 45.7 ± 0.78 |

Whole Blood/Plasma Ratio

| | | | | | |
|---|---|---|---|---|---|
| 1 | 0.80 | 0.67 | 0.82 | 0.86 | 0.79 ± 0.08 |
| 4 | 0.83 | 0.88 | 0.59 | 1.55 | 0.96 ± 0.41 |
| 8 | 0.92 | 0.97 | 0.93 | 1.04 | 0.97 ± 0.06 |
| 24 | 0.84 | 0.91 | 0.63 | 0.92 | 0.83 ± 0.13 |
| 48 | 0.87 | 1.07 | 0.70 | 1.04 | 0.92 ± 0.17 |
| 72 | 0.95 | 1.06 | 0.34 | 0.81 | 0.79 ± 0.32 |

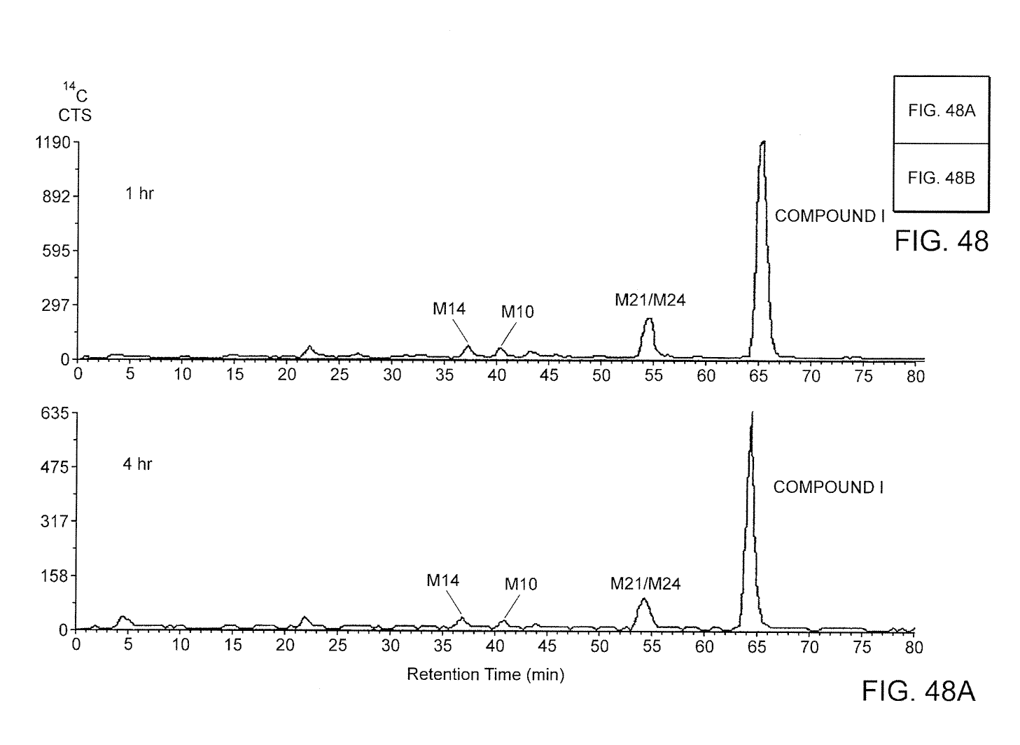

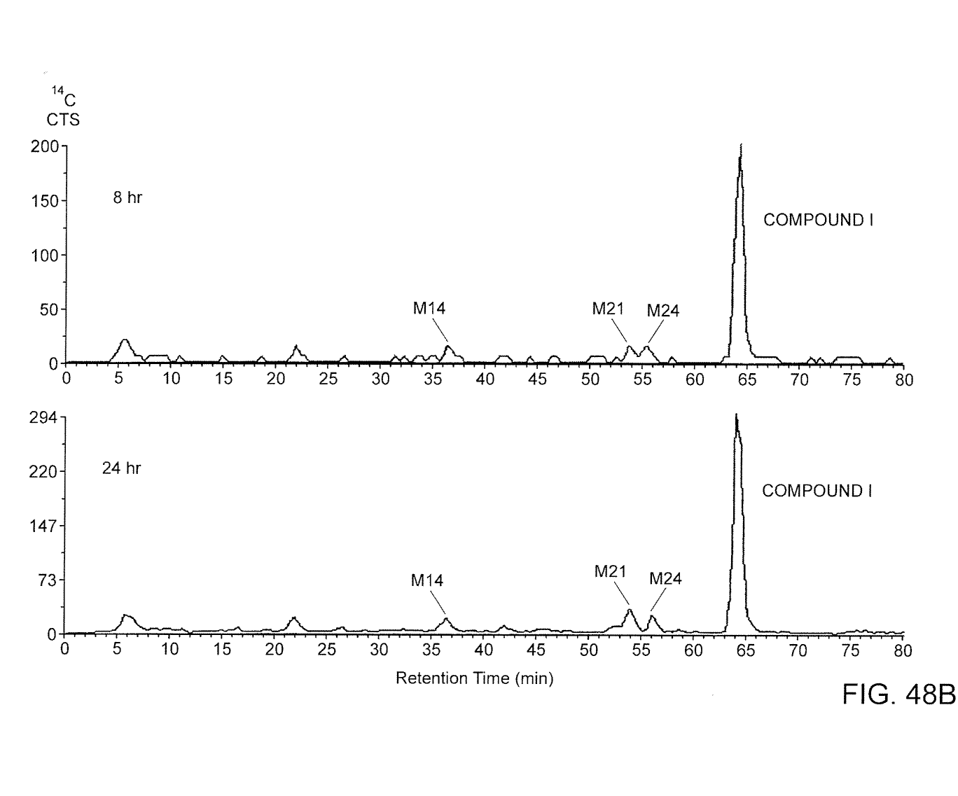

The extraction recovery of radioactivity from plasma samples was greater than 82%. Radiochromatograms of pooled plasma samples are shown in FIG. 48. COMPOUND I represented 71-78% of the total plasma radioactivity over 24 hr post-dose. O-Desmethyl COMPOUND I (M21) and O-desmethyl COMPOUND I sulfate (M24) were the major metabolites in plasma, together representing up to 11% of total radioactivity. N-Desmethoxyquinolinyl COMPOUND I (M14) and hydroxy COMPOUND I glucuronide (M10) were also observed in plasma, each representing less than 5% of plasma radioactivity. Several additional minor metabolites (each less than 5%) were observed in plasma but were not characterized due to low concentrations.

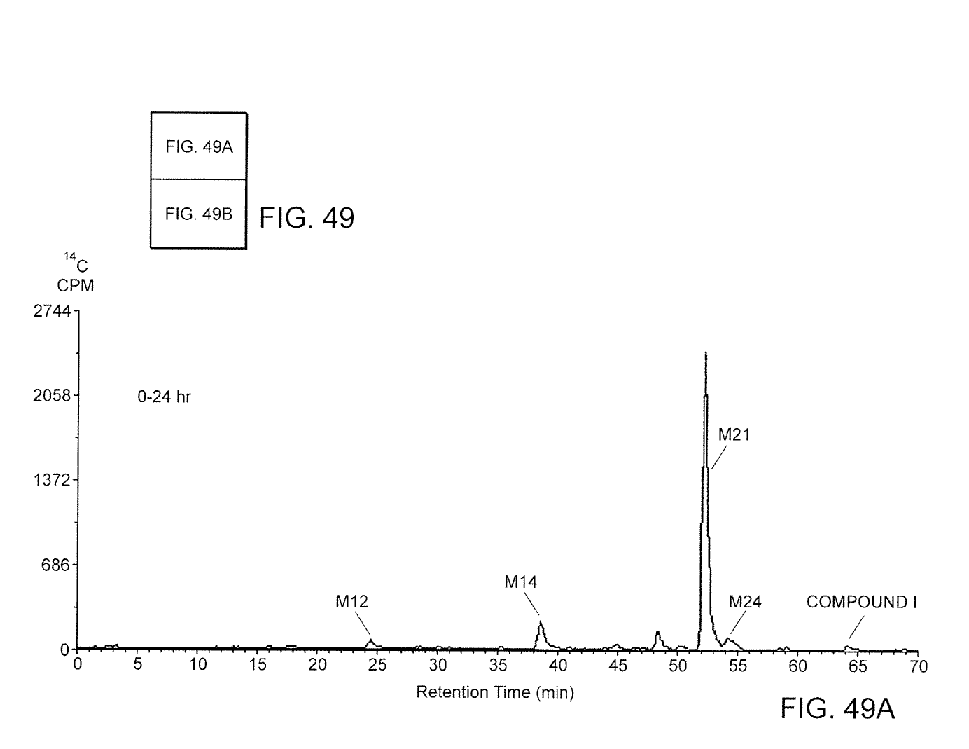

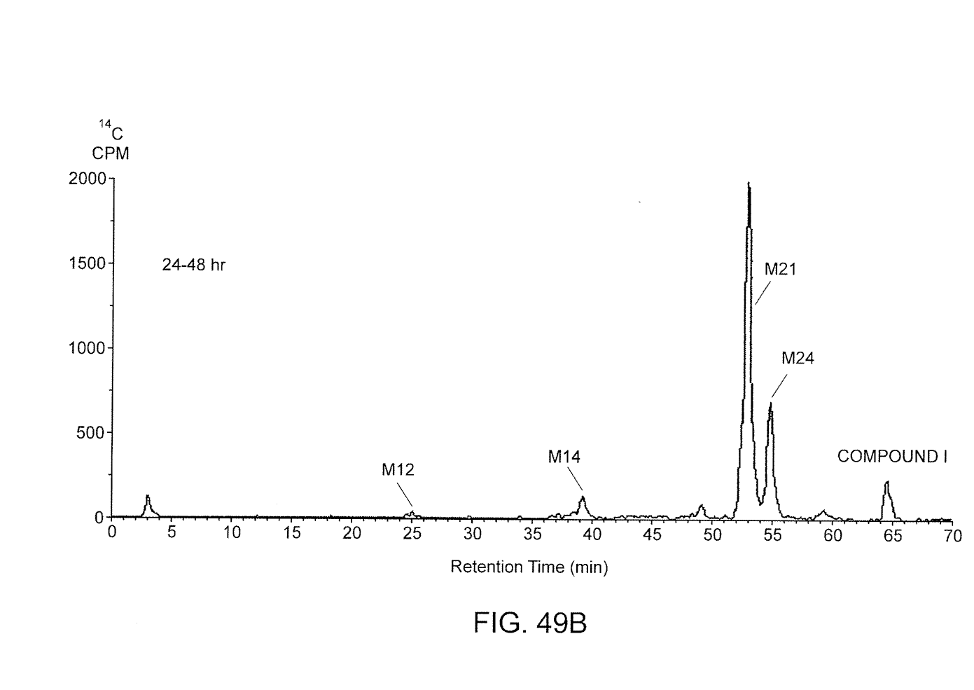

An average of 25.7% and 33.7% of the administered radioactivity was excreted in the 0-24 and 24-48 hr feces, respectively. The extraction recovery of radioactivity from the pooled 0-24 and 24-48 hr fecal homogenates was greater than 80%. COMPOUND I represented 1.2% and 6.7% of total fecal radioactivity in the 0-24 and 24-48 hr fecal samples, respectively (FIG. 49). O-Desmethyl COMPOUND I (M21), which represented 79.3 and 60.9% of total radioactivity in the 0-24 and 24-48 hr samples, respectively, was the predominant metabolite in fecal extracts. Another major metabolite in fecal extracts was O-desmethyl COMPOUND I sulfate (M24), representing 4.6% and 19.3% of total radioactivity in the 0-24 and 24-48 hr fecal samples, respectively. N-Desfluoroquinolinyl COMPOUND I (M12) and N-desmethoxyquinolinyl COMPOUND I (M14) were observed as minor metabolites (each less than 8%) in fecal extracts. Several other smaller radioactive peaks present in fecal extracts were not characterized.

Mass spectra were obtained by LC/MS and LC/MS/MS analysis for COMPOUND I and its metabolites in the samples of dog plasma and feces. Structural characterization of these compounds is summarized in Table 28. The mass spectral characterization of COMPOUND I and its metabolites is discussed below. In LC/MS experiments conducted with D₂O substituted for H₂O in the mobile phase to determine number of exchangeable hydrogens, the mass difference between [M+D]⁺ and [M+H]⁺ was 1 Da larger than the number of exchangeable hydrogens on COMPOUND I and its metabolites due to exchange of the proton required for ionization to generate [M+H]⁺.

TABLE 28
[¹⁴C]COMPOUND I and Its Metabolites Characterized in Dogs

| | Retention | | | | |
|---|---|---|---|---|---|
| | Time | | | | |
| Peak | (min)^{a} | [M + H]⁺ | Site of Metabolism | Name | Source^{b} |
| M10 | 40.0 | 664 | Methoxyquinoline | Hydroxy COMPOUND I | P |
| | | | | glucuronide | |
| M12 | 24.5 | 327 | Fluoroquinoline | N-Desfluoroquinolinyl | F |
| | | | | COMPOUND I | |
| M14 | 36.8 | 315 | Methoxyquinoline | N-Desmethoxyquinolinyl | P, F |
| | | | | COMPOUND I | |
| M21 | 53.9 | 458 | Methoxyquinoline | O-Desmethyl | P, F |
| | | | | COMPOUND I | |
| M24 | 55.7 | 538 | Methoxyquinoline | O-Desmethyl | P, F |
| | | | | COMPOUND I sulfate | |
| COMPOUND I | 64.0 | 472 | None | COMPOUND I | P, F |

^{a}LC retention time taken from radiochromatograms and may differ from LC/MS retention times.
^{b}P, plasma; F, feces. Bold face indicates major drug-related components in the matrix.

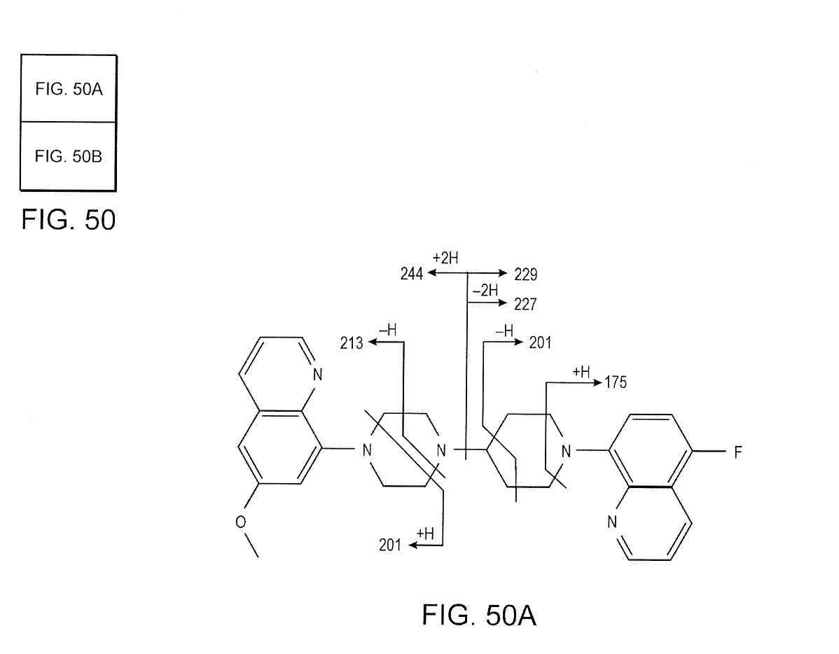

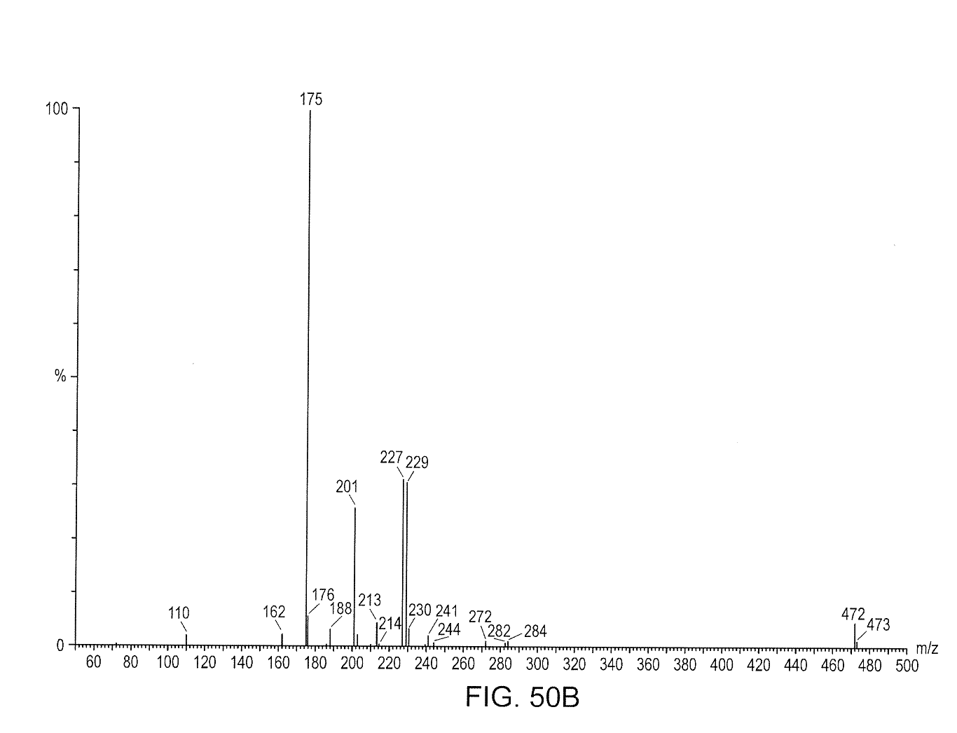

The mass spectral characteristics of synthetic COMPOUND I were examined for comparison with metabolites. In the LC/MS spectrum of COMPOUND I, the protonated molecular ion, [M+H]⁺ was observed at m/z 472. LC/MS with D₂O substituted for H₂O in the mobile phase generated [M+D]⁺ at m/z 473 (data not shown), consistent with COMPOUND I having no exchangeable hydrogens. The MS/MS spectrum obtained from collision activated dissociation of m/z 472 from COMPOUND I and the proposed fragmentation scheme are shown in FIG. 50. Fragmentation of the piperazine-piperidine bond with charge retention on the methoxyquinoline half of the molecule yielded m/z 244. The same fragmentation with charge retention on the fluoroquinoline half of the molecule yielded m/z 229 and 227. Fragmentation of the piperazine ring generated a methoxyquinoline-containing ion at m/z 213. Fragmentation of the piperidine ring generated a fluoroquinoline-containing ion at m/z 175. Two assignments for the m/z 201 product ion were made. One m/z 201 product ion originated from cleavage of the piperidine ring with charge retention on the moiety containing the fluoroquinoline. The other m/z 201 product ion originated from cleavage of the piperazine ring. These assignments were confirmed by the product ions of m/z 474 (¹⁴C[M+H]⁺) and m/z 476 (¹⁴C₂[M+H]⁺) mass spectral data for radiolabeled COMPOUND I (data not shown).

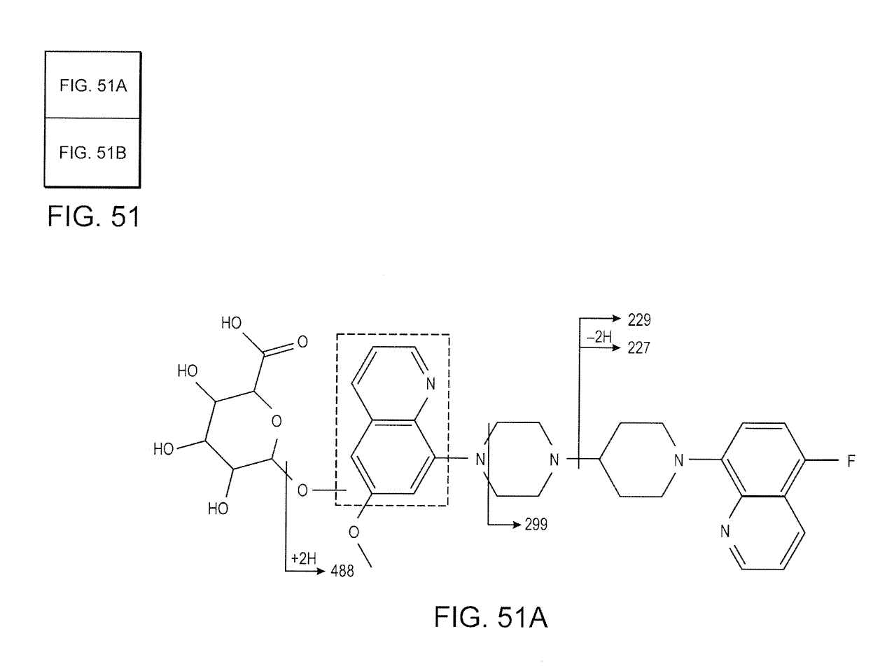

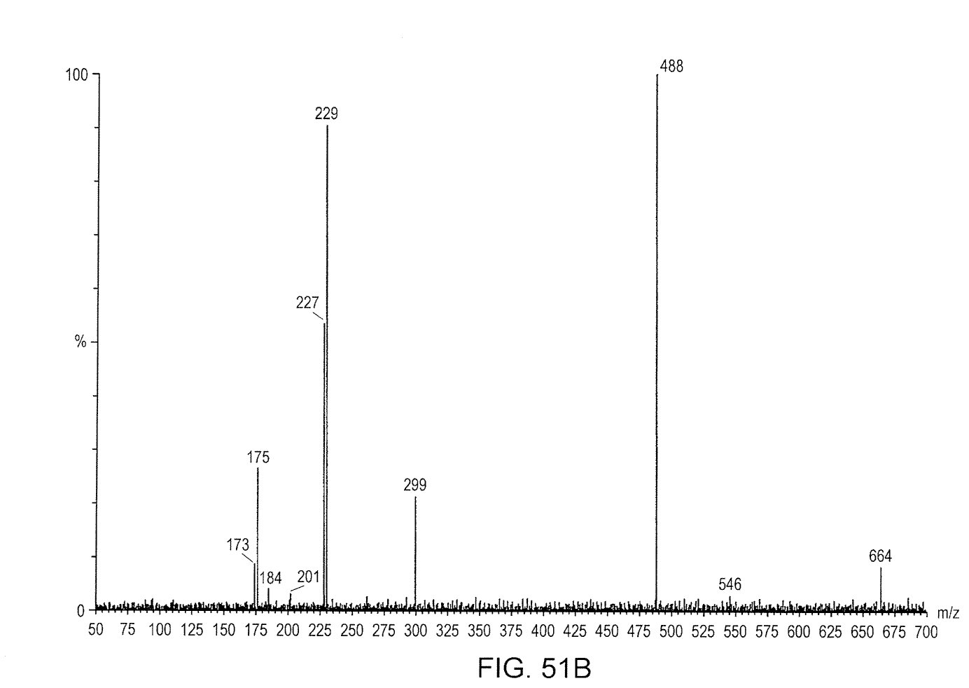

The [M+H]⁺ for M10 was observed at m/z 664, which was 192 Da larger than COMPOUND I. LC/MS with D₂O substituted for H₂O in the mobile phase generated [M+D]⁺ at m/z 669 (data not shown). These data indicated four exchangeable hydrogens, which was four more than COMPOUND I. The product ions of m/z 664 mass spectrum and the proposed fragmentation scheme for M10 are presented in FIG. 51. Neutral loss of 176 Da from [M+H]⁺ yielded m/z 488, which was 16 Da larger than COMPOUND I. These data indicated that M10 was a glucuronide of a hydroxy COMPOUND I. Product ions at m/z 229 and 227 were also observed for COMPOUND I, which indicated an unchanged fluoroquinolinyl-piperidine moiety. Fragmentation of the piperazine ring generated m/z 299, which indicated that the piperazine ring was also unchanged and consequently the methoxyquinoline was the site of hydroxylation and subsequent glucuronidation. Therefore, M10 was identified as a hydroxy COMPOUND I glucuronide.

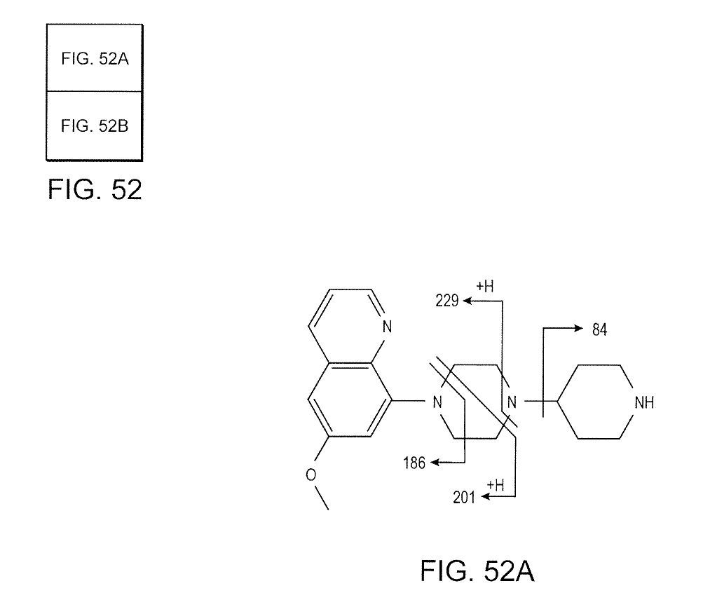

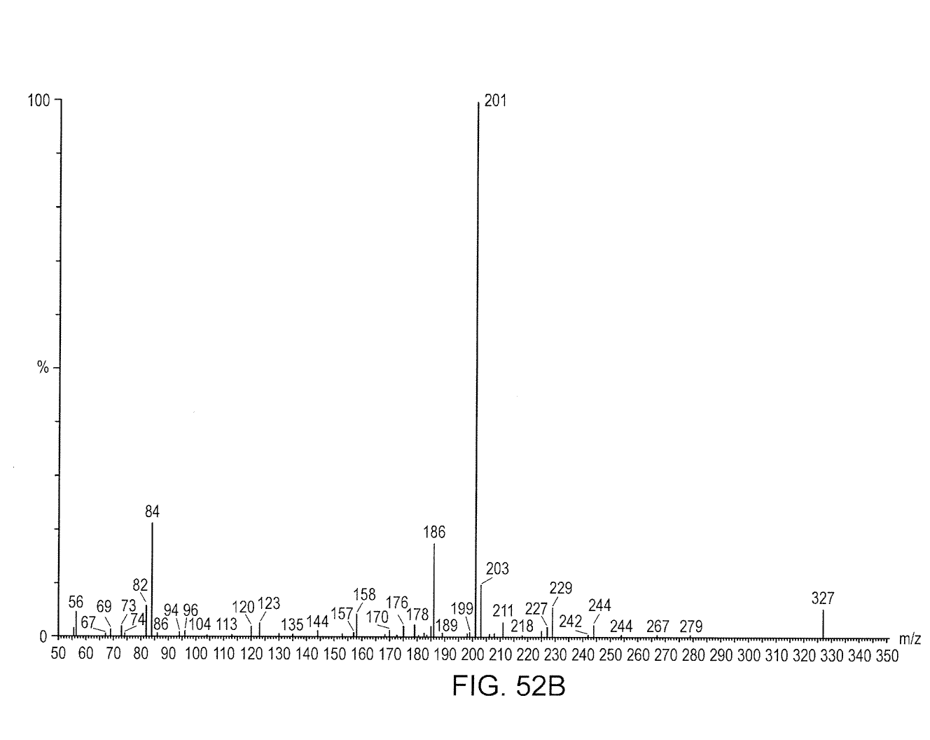

Metabolite M12 produced a [M+H]⁺ at m/z 327, which was 145 Da less than COMPOUND I. LC/MS with D₂O substituted for H₂O in the mobile phase generated [M+D]⁺ at m/z 329 (data not shown). These data indicated one exchangeable hydrogen, which was one more than COMPOUND I and consistent with N-dealkylation of the COMPOUND I molecule. The product ions of m/z 327 mass spectrum and the proposed fragmentation scheme for M12 are presented in FIG. 52. The product ion at m/z 201 was also observed for COMPOUND I. Fragmentation of the piperazine ring generated m/z 229 and 186. The product ion at m/z 84 represented a piperidinyl ion. These data indicated intact methoxyquinolinyl-piperazine and piperidine moieties, which in combination with the molecular weight difference between M12 and COMPOUND I indicated that the fluoroquinoline moiety of COMPOUND I was not present in M12. Therefore, M12 was identified as N-desfluoroquinolinyl COMPOUND I.

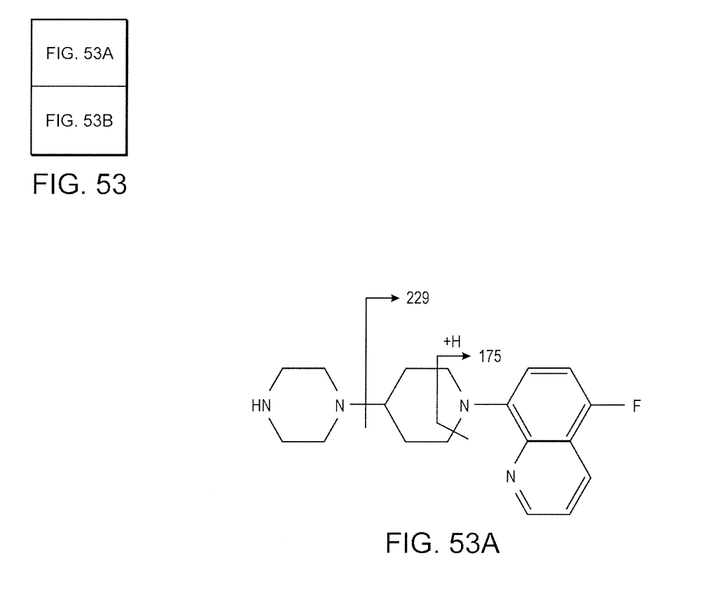

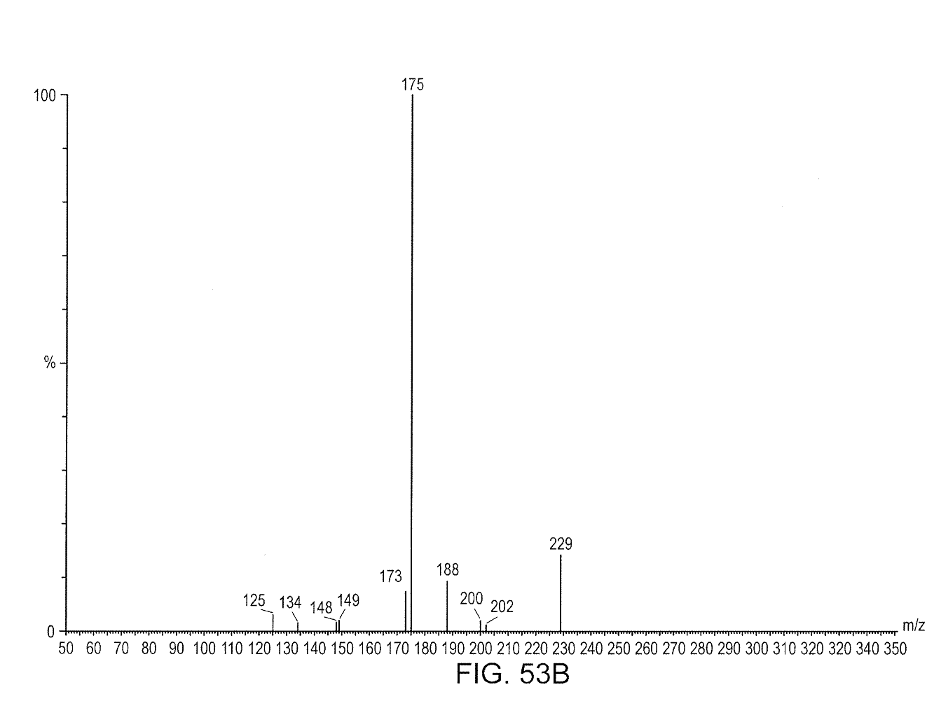

The [M+H]⁺ for M14 was observed at m/z 315, which was 157 Da less than COMPOUND I. LC/MS with D₂O substituted for H₂O in the mobile phase generated [M+D]⁺ at m/z 317 (data not shown). These data indicated one exchangeable hydrogen, which was one more than COMPOUND I and consistent with N-dealkylation of the COMPOUND I molecule. The product ions of m/z 315 mass spectrum and the proposed fragmentation scheme for M14 are presented in FIG. 53. Product ions at m/z 229 and 175 were also observed for COMPOUND I, which indicated unchanged piperidine and fluoroquinoline rings. These data and the molecular weight difference between M14 and COMPOUND I indicated that the methoxyquinoline moiety of COMPOUND I was not present in M14. Therefore, M14 was identified as N-desmethoxyquinolinyl COMPOUND I.

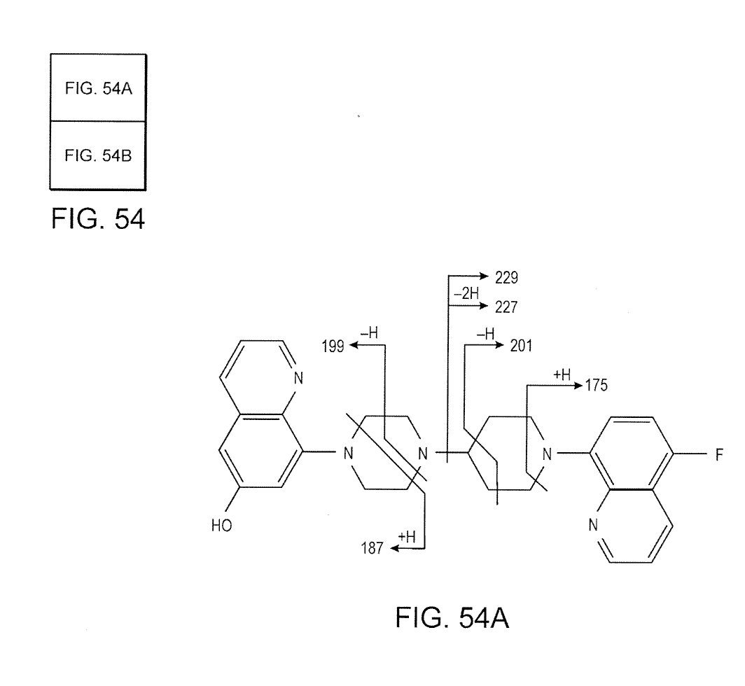

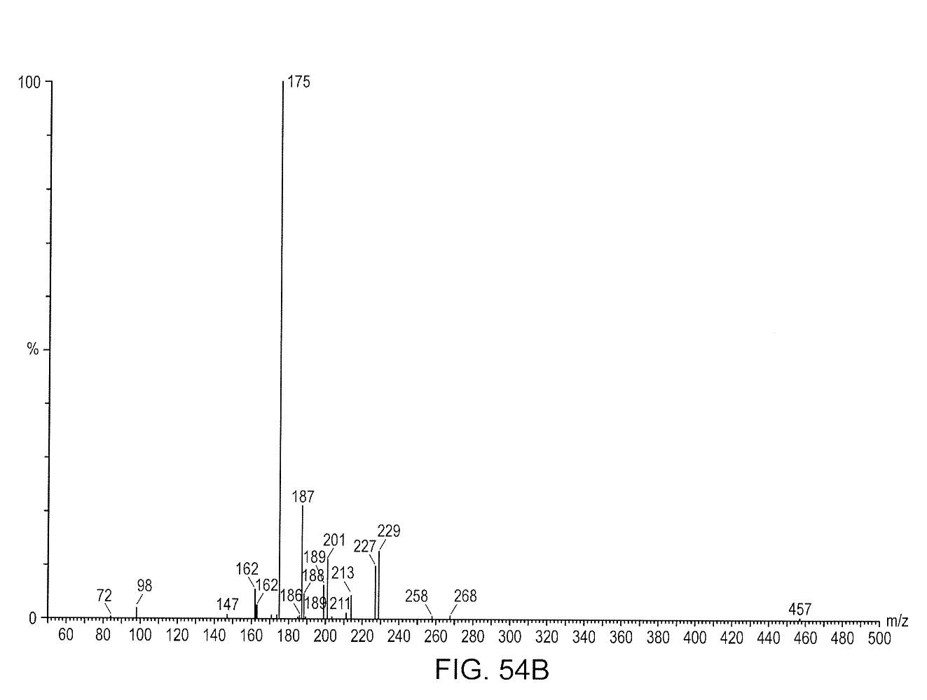

The [M+H]⁺ for M21 was observed at m/z 458, which was 14 Da less than COMPOUND I. LC/MS with D₂O substituted for H₂O in the mobile phase generated [M+D]⁺ at m/z 460 (data not shown). These data indicated one exchangeable hydrogen, which was one more than COMPOUND I and consistent with the presence of a hydroxyl group. The product ions of m/z 458 mass spectrum and the proposed fragmentation scheme for M21 are presented in FIG. 54. Product ions at m/z 229, 227, 201 and 175 were also observed for COMPOUND I, which indicated an unchanged fluoroquinolinyl-piperidine moiety. Product ions at m/z 199 and 187 were 14 Da less than the corresponding methoxyquionolinyl ions at m/z 213 and 201, respectively, for COMPOUND I, which indicated demethylation of the methoxy group. Therefore, M21 was identified as O-desmethyl COMPOUND I.

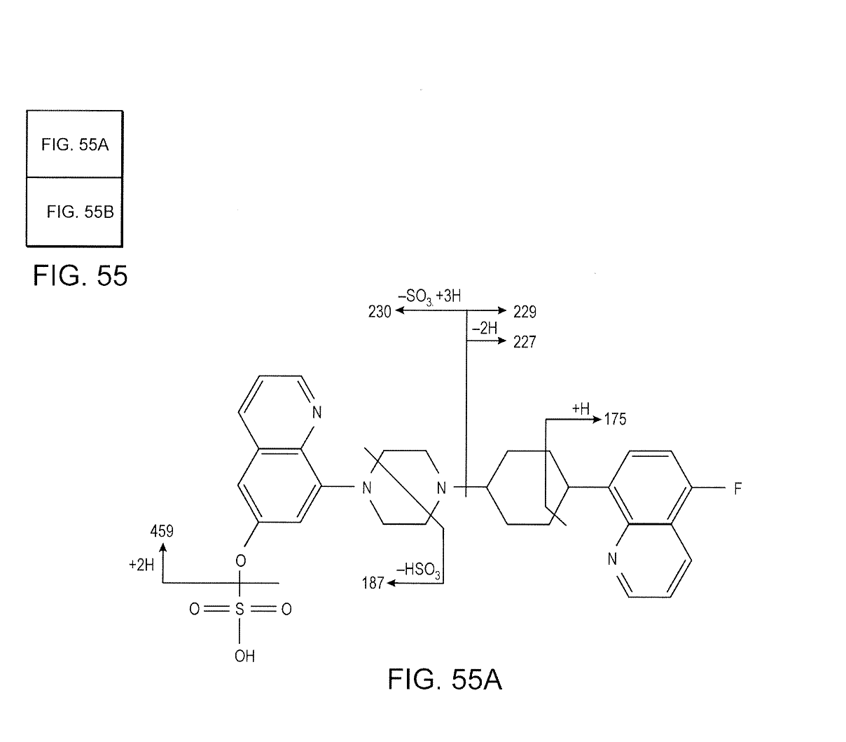

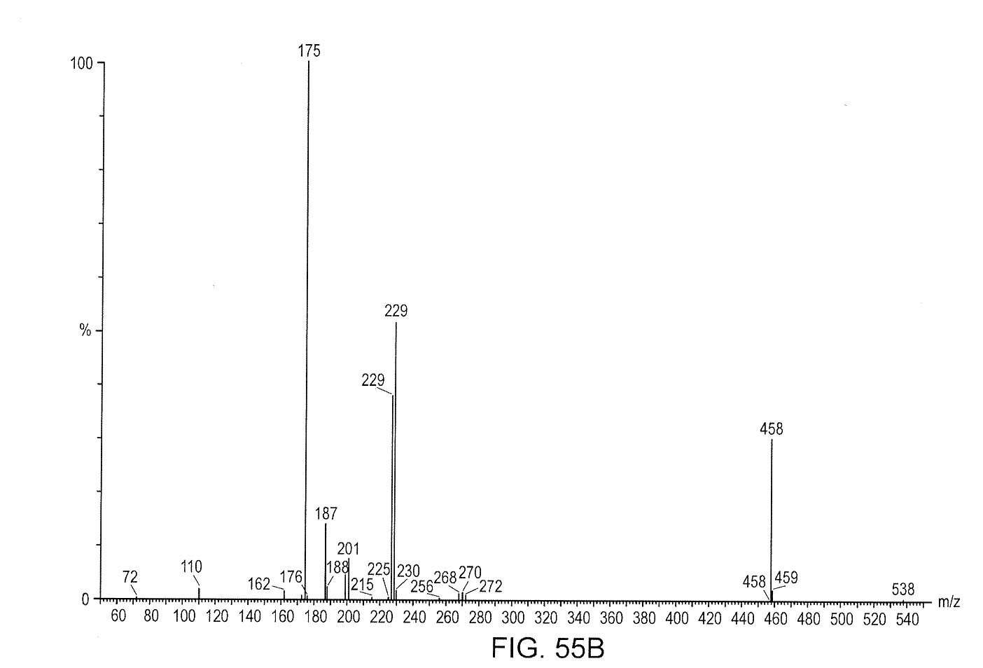

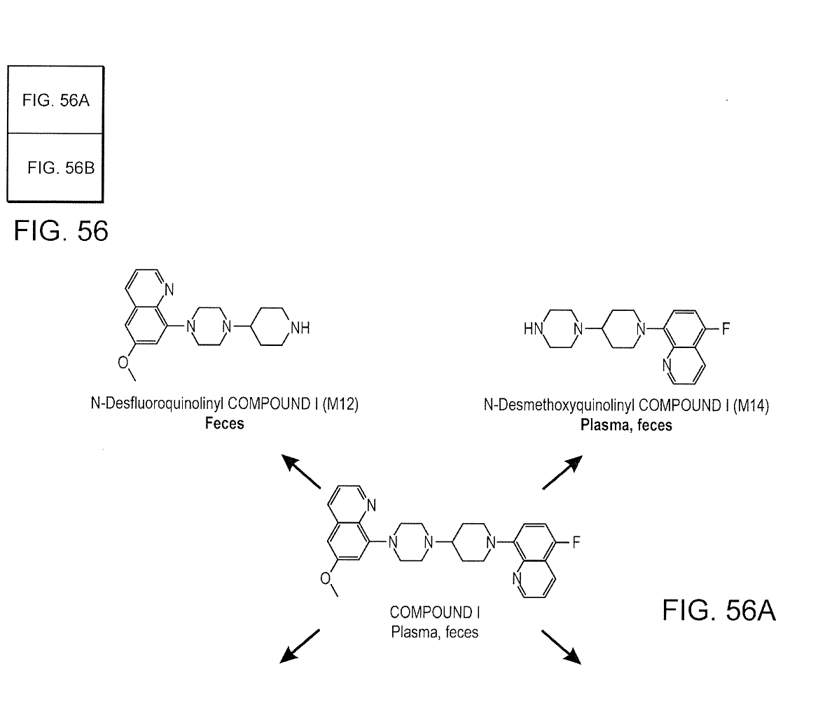

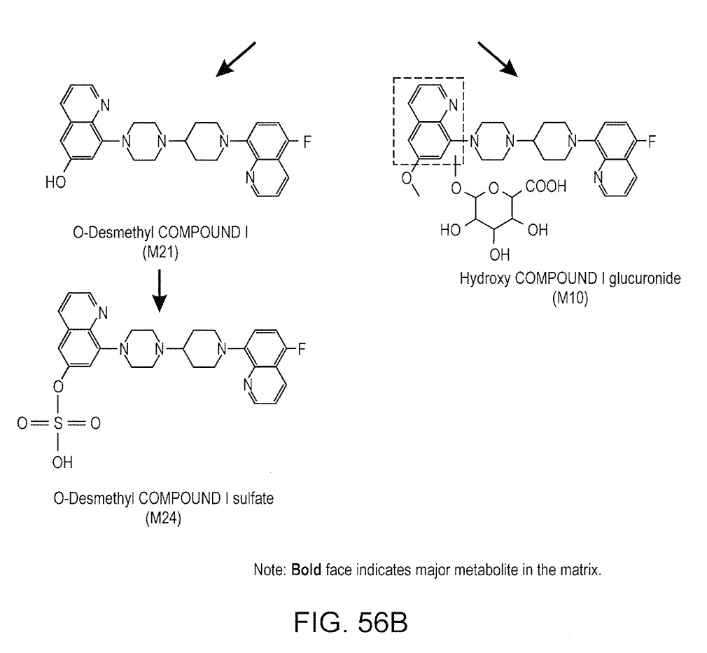

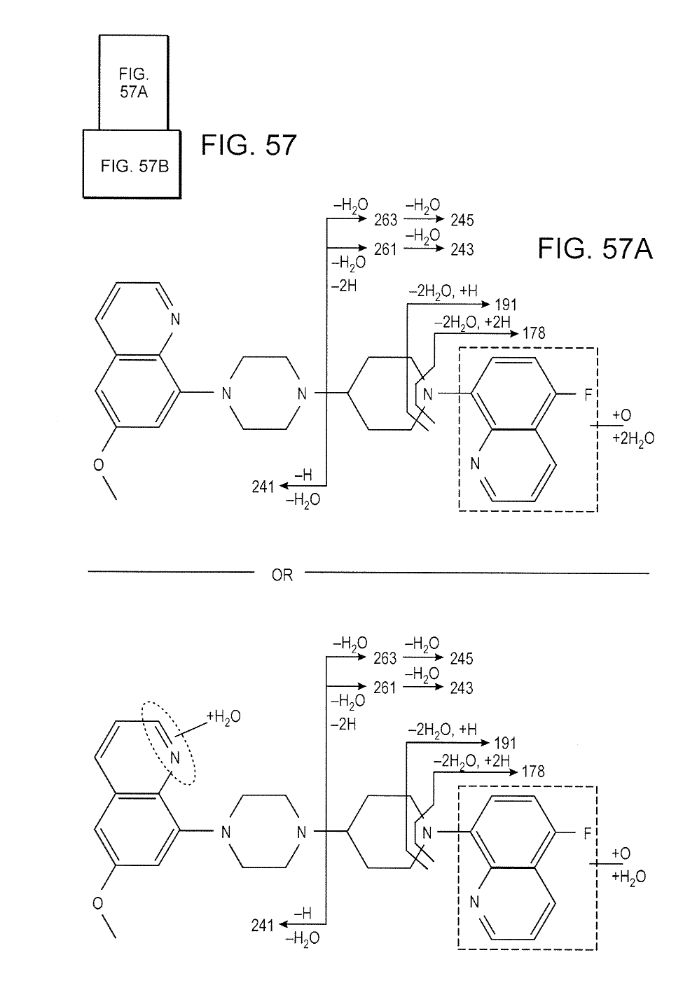

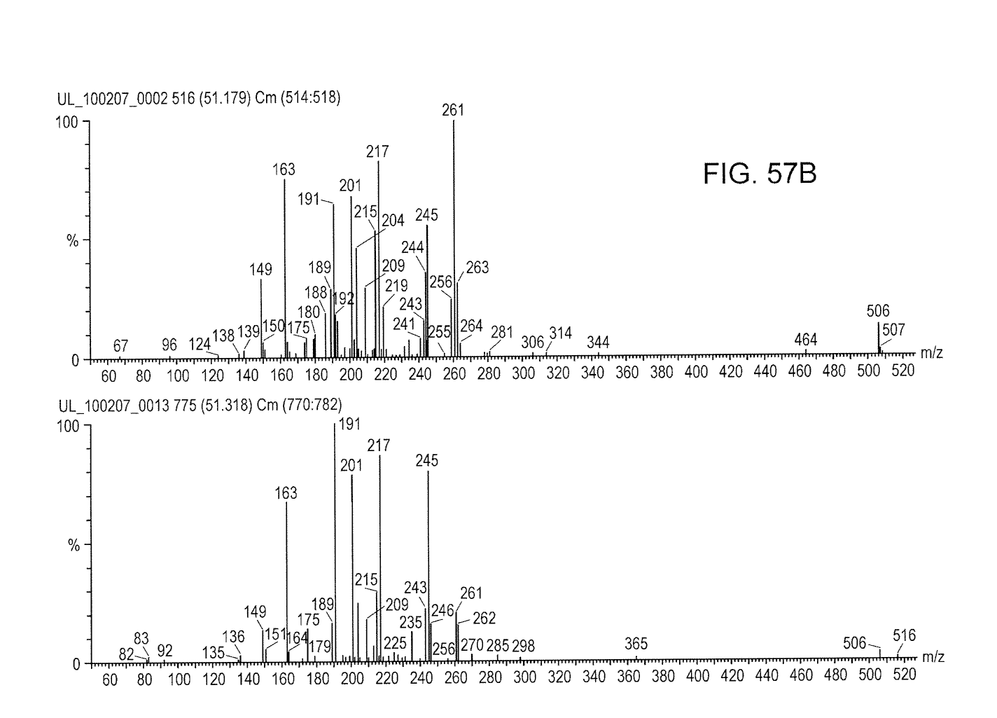

Metabolite M24 produced a [M+H]⁺ at m/z 538, which was 66 Da larger than COMPOUND I. LC/MS with D₂O substituted for H₂O in the mobile phase generated [M+D]⁺ at m/z 540 (data not shown). These data indicated one exchangeable hydrogen, which was one more than COMPOUND I. The product ions of m/z 538 mass spectrum and the proposed fragmentation scheme for M24 are presented in FIG. 55. Neutral loss of 80 Da from [M+H]⁺ yielded m/z 458, which indicated that M24 was a sulfate. Product ions at m/z 229, 227 and 175 were also observed for COMPOUND I, which indicated unchanged fluoroquinoline and piperidine rings. Product ions at m/z 230 and 187 were 14 Da less than the corresponding ions at m/z 244 and 201, respectively, for COMPOUND I, which indicated demethylation of the methoxyquinolinyl moiety. Therefore, M24 was identified as O-desmethyl COMPOUND I sulfate.

### Synthesis of M21

M21 can also be synthesized according to Scheme 1, below. The methoxy group of COMPOUND I can be demethylated by treating COMPOUND I with an acid, such as Me₃SiI, BBr₃, BF₃.Et₂, MeSSiMe₃, PhSSiMe₃, AlCl₃, AlBr₃, t-BuCOCl, AcCl, Ac₂O & FeCl₃, Me₂BBr, BI₃-Et₂NPh, TMSCl, and RuCl₃ etc., to provide M21.

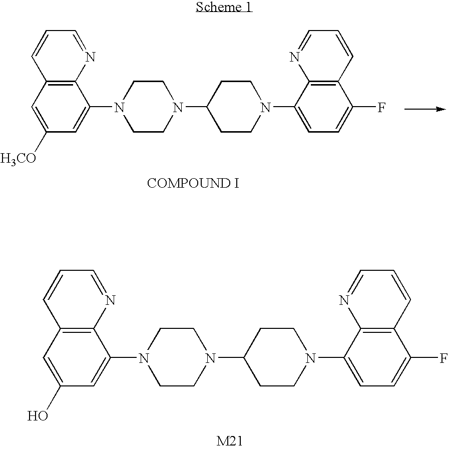

Alternatively, M21 can be prepared according to Scheme 2, below. Two intermediates, A and B, can be synthesized from known starting materials. For example, Intermediate A can be prepared by protecting the hydroxy group of 4-hydroxyaniline with a suitable hydroxyl protecting group (R₁), followed by ring formation using Skraup conditions (see, e.g., Mundy et al., Name Reactions and Reagents in Organic Synthesis, John Wiley & Sons, (1988), pages 196-197), Buchwald-Hartwig condensation (see, e.g., Wolfe et al., J. Am. Chem. Soc. 1996, 118, 7215-7216; and Driver et al., J. Am. Chem. Soc. 1996, 118, 7217-7218) with an appropriately protected piperazine derivative (R₂=amine protecting group) and de-protection. The group X denotes a suitable leaving group such as chloro or bromo. Intermediate B can be prepared in a similar sequence from 3-fluoroanilines by employing Skraup ring formation conditions, Buchwald/Hartig condensation with an appropriately protected piperidinone (i.e., protected by a suitable ketone protecting group) and de-protection of the ketone. Again, the group X denotes a suitable leaving group such as chloro or bromo. The two intermediates A and B are reacted together under reductive ammination conditions to yield compound C, which is converted to the compound of this invention M21 by de-protection of the hydroxy group. M21 may be further converted to a suitable salt form, such as a hydrochloride or trisuccinate salt.

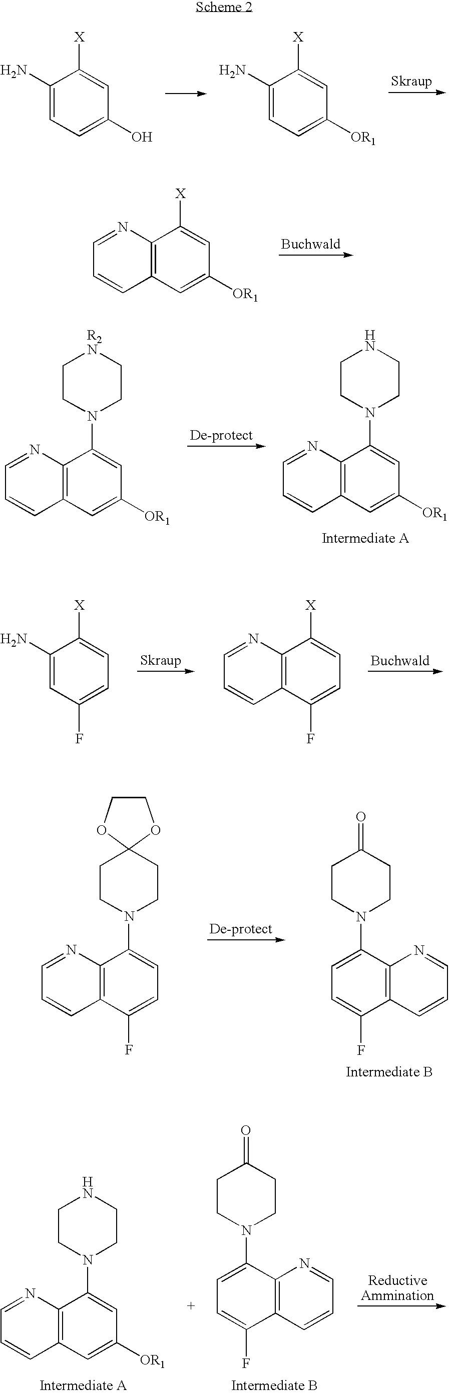

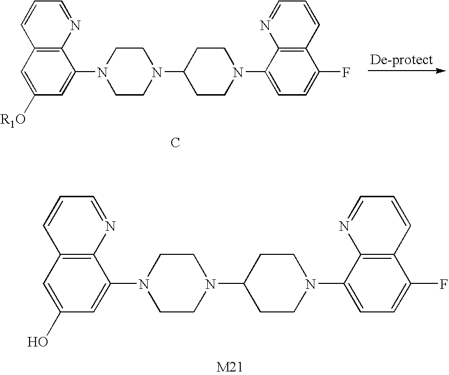

The 5-HT_{1A} affinity of the compounds of this invention can be assessed by measuring the ability of the compound to displace [³H]-8-OH-DPAT from its binding site on the human 5-HT_{1A} receptor stably transfected in Chinese hamster ovary (CHO) cells as described below. A Ki value is reported. The in vitro functional activity of the compounds of this invention is determined by measuring the effect of the compound on forskolin-induced adenylate cyclase activity in the same cell line. 5-HT_{1A} agonists, such as the full agonist 8-OH-DPAT, inhibit forskolin-induced adenylate cyclase activity, as measured by a reduction in cAMP levels. The compounds of this invention display agonist activity as shown by its ability to induce a decrease in cAMP levels. An EC₅₀ value is reported and the maximum response of the test compound is reported as the percent of a full agonist response (the maximum response obtained with the full agonist 8-OH-DPAT=100%). This percent is expressed as an Emax value.

The PCR cloning of the human 5-HT_{1A} receptor subtype from a human genomic library has been described previously (Chanda et al., Mol. Pharmacol., 43:516 (1993)). A stable Chinese hamster ovary cell line expressing the human 5-HT_{1A} receptor subtype (h5-HT_{1A}.CHO cells) was employed throughout this study. Cells were maintained in DMEM supplemented with 10% fetal calf serum, non-essential amino acids and penicillin/streptomycin.

Radioligand binding assays can be performed as described in Dunlop, J. et al., J. Pharmacol. and Toxicol. Methods 40: 47-55 (1998). Cells were grown to 95-100% confluency as a monolayer before membranes were harvested for binding studies. Cells are gently scraped from the culture plates, transferred to centrifuge tubes, and washed twice by centrifugation (2000 rpm for 10 min., 4° C.) in buffer (50 mM Tris; pH 7.5). The resulting pellets are aliquoted and placed at −80° C. On the day of assay, the cells are thawed on ice, and re-suspended in buffer. Studies are conducted using [³H]8-OH-DPAT as the radioligand. The binding assay is performed in 96 well microtiter plates in a final total volume of 250 μL of buffer. Competition experiments are performed by using seven different concentrations of unlabelled drug and a final ligand concentration of 1.5 nM. Non-specific binding is determined in the presence of 10 μM 5HT. Saturation analysis is conducted by using [³H]8-OH-DPAT at concentrations ranging from 0.3-30 nM. Following a 30 min. incubation at room temperature, the reaction is terminated by the addition of ice cold buffer and rapid filtration using a M-96 Brandel Cell Harvester (Gaithersburg, Md.) through a GF/B filter presoaked for 30 min. in 0.5% polyethyleneimine.

Measurements can be performed as described in Dunlop, J. et al., supra. Assays are performed by incubating the cells with DMEM containing 25 mM HEPES, 5 mM theophylline and 10 μM pargyline for a period of 20 min. at 37° C. Functional activity is assessed by treating the cells with forskolin (1 uM final concentration) followed immediately by test compound (6 different concentrations) for an additional 10 min. at 37° C. The reaction is terminated by removal of the media and addition of 0.5 mL ice cold assay buffer. Plates are stored at −20° C. prior to assessment of cAMP formation by a cAMP SPA assay (Amersham).

When administered to an animal, the compounds or pharmaceutically acceptable salts of the compounds can be administered neat or as a component of a composition that comprises a physiologically acceptable carrier or vehicle. A pharmaceutical composition of the invention can be prepared using a method comprising admixing the compound or a pharmaceutically acceptable salt of the compound and a physiologically acceptable carrier, excipient, or diluent. Admixing can be accomplished using methods well known for admixing a compound or a pharmaceutically acceptable salt of the compound and a physiologically acceptable carrier, excipient, or diluent.

The present pharmaceutical compositions, comprising compounds or pharmaceutically acceptable salts of the compounds of the invention, can be administered orally. The compound of the invention can also be administered by any other convenient route, for example, by infusion or bolus injection, by absorption through epithelial or mucocutaneous linings (e.g., oral, rectal, vaginal, and intestinal mucosa, etc.) and can be administered together with another therapeutic agent. Administration can be systemic or local. Various known delivery systems, including encapsulation in liposomes, microparticles, microcapsules, and capsules, can be used.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result of release of the compound or a pharmaceutically acceptable salt of the compound into the bloodstream. The mode of administration is left to the discretion of the practitioner.

In one embodiment, the compound of the invention is administered orally.

In another embodiment, the compound of the invention is administered intravenously.

In another embodiment, it may be desirable to administer the compound of the invention locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, it can be desirable to introduce the compound of the invention into the CNS, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compound or a pharmaceutically acceptable salt of the compound can be formulated as a suppository, with traditional binders and excipients such as triglycerides.

In another embodiment, the compound of the invention can be delivered in a vesicle, in particular a liposome (see Langer, Science 249:1527-1533 (1990) and Treat et al., Liposomes in the Therapy of Infectious Disease and Cancer 317-327 and 353-365 (1989)).

In yet another embodiment, the compound of the invention can be delivered in a controlled-release system or sustained-release system (see, e.g., Goodson, in Medical Applications of Controlled Release, vol. 2, pp. 115-138 (1984)). Other controlled or sustained-release systems discussed in the review by Langer, Science 249:1527-1533 (1990) can be used. In one embodiment, a pump can be used (Langer, Science 249:1527-1533 (1990); Sefton, CRC Crit. Ref Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

The present compositions can optionally comprise a suitable amount of a physiologically acceptable excipient.

Such physiologically acceptable excipients can be liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment the physiologically acceptable excipients are sterile when administered to an animal. The physiologically acceptable excipient should be stable under the conditions of manufacture and storage and should be preserved against the contaminating action of microorganisms. Water is a particularly useful excipient when the compound or a pharmaceutically acceptable salt of the compound is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Liquid carriers may be used in preparing solutions, suspensions, emulsions, syrups, and elixirs. The compound or pharmaceutically acceptable salt of the compound of this invention can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule. Other examples of suitable physiologically acceptable excipients are described in Remington's Pharmaceutical Sciences 1447-1676 (Alfonso R. Gennaro, ed., 19th ed. 1995).

In one embodiment, the compound or a pharmaceutically acceptable salt of the compound is formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided compound or pharmaceutically acceptable salt of the compound. In tablets, the compound or pharmaceutically acceptable salt of the compound is mixed with a carrier having the necessary compression properties in suitable proportions and compacted in the shape and size desired. The powders and tablets can contain up to about 99% of the compound or pharmaceutically acceptable salt of the compound.

Capsules may contain mixtures of the compounds or pharmaceutically acceptable salts of the compounds with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins). Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the compound or a pharmaceutically acceptable salt of the compound can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the compound or a pharmaceutically acceptable salt of the compound is to be administered by infusion, it can be dispensed, for example, with an infusion bottle containing sterile pharmaceutical grade water or saline. Where the compound or a pharmaceutically acceptable salt of the compound is administered by injection, an ampule of sterile water for injection or saline can be provided so that the ingredients can be mixed prior to administration.

In another embodiment, the compound or pharmaceutically acceptable salt of the compound can be administered transdermally through the use of a transdermal patch. Transdermal administrations include administrations across the surface of the body and the inner linings of the bodily passages including epithelial and mucosal tissues. Such administrations can be carried out using the present compounds or pharmaceutically acceptable salts of the compounds, in lotions, creams, foams, patches, suspensions, solutions, and suppositories (e.g., rectal or vaginal).

Transdermal administration can be accomplished through the use of a transdermal patch containing the compound or pharmaceutically acceptable salt of the compound and a carrier that is inert to the compound or pharmaceutically acceptable salt of the compound, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the compound or pharmaceutically acceptable salt of the compound into the blood stream, such as a semi-permeable membrane covering a reservoir containing the compound or pharmaceutically acceptable salt of the compound with or without a carrier, or a matrix containing the active ingredient.

The compounds or pharmaceutically acceptable salts of the compounds of the invention may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

The compound or a pharmaceutically acceptable salt of the compound can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of the compound or a pharmaceutically acceptable salt of the compound to treat or prevent a 5-HT_{1A}-related disorder in a minimal amount of time. Advantages of controlled- or sustained-release compositions include extended activity of the drug, reduced dosage frequency, and increased compliance by the animal being treated. In addition, controlled- or sustained-release compositions can favorably affect the time of onset of action or other characteristics, such as blood levels of the compound or a pharmaceutically acceptable salt of the compound, and can thus reduce the occurrence of adverse side effects.

Controlled- or sustained-release compositions can initially release an amount of the compound or a pharmaceutically acceptable salt of the compound that promptly produces the desired therapeutic or prophylactic effect, and gradually and continually release other amounts of the compound or a pharmaceutically acceptable salt of the compound to maintain this level of therapeutic or prophylactic effect over an extended period of time. To maintain a constant level of the compound or a pharmaceutically acceptable salt of the compound in the body, the compound or a pharmaceutically acceptable salt of the compound can be released from the dosage form at a rate that will replace the amount of the compound or a pharmaceutically acceptable salt of the compound being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

In certain embodiments, the present invention is directed to prodrugs of the compounds or pharmaceutically acceptable salts of compounds of the present invention. Various forms of prodrugs are known in the art, for example as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975). In some embodiments, the glucuronide derivatives of the COMPOUND I metabolites, including M2, M3, M5, M7, M9, M10, and M11, can be administered as prodrugs which can be cleaved by glucuronidase in vivo. In one embodiment, administration is by the oral route to maximize the advantages of glucuronidase activity in the gut.

The amount of the compound or a pharmaceutically acceptable salt of the compound delivered is an amount that is effective for treating or preventing a 5-HT_{1A}-related disorder. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound or a pharmaceutically acceptable salt of the compound is administered, the effective dosage amounts correspond to the total amount administered.

The amount of the compound or a pharmaceutically acceptable salt of the compound that is effective for treating or preventing a 5-HT_{1A}-related disorder will typically range from about 0.001 mg/kg to about 600 mg/kg of body weight per day, in one embodiment, from about 1 mg/kg to about 600 mg/kg body weight per day, in another embodiment, from about 10 mg/kg to about 400 mg/kg body weight per day, in another embodiment, from about 10 mg/kg to about 200 mg/kg of body weight per day, in another embodiment, from about 10 mg/kg to about 100 mg/kg of body weight per day, in another embodiment, from about 1 mg/kg to about 10 mg/kg body weight per day, in another embodiment, from about 0.001 mg/kg to about 100 mg/kg of body weight per day, in another embodiment, from about 0.001 mg/kg to about 10 mg/kg of body weight per day, and in another embodiment, from about 0.001 mg/kg to about 1 mg/kg of body weight per day.

In one embodiment, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 0.01 mg/kg to about 250 mg/kg, and may be given in a single dose or in two or more divided doses. Variations in the dosage will necessarily occur depending upon the species, weight and condition of the patient being treated and the patient's individual response to the medicament.

In one embodiment, the unit dosage form is about 0.01 to about 1000 mg. In another embodiment, the unit dosage form is about 0.01 to about 500 mg; in another embodiment, the unit dosage form is about 0.01 to about 250 mg; in another embodiment, the unit dosage form is about 0.01 to about 100 mg; in another embodiment, the unit dosage form is about 0.01 to about 50 mg; in another embodiment, the unit dosage form is about 0.01 to about 25 mg; in another embodiment, the unit dosage form is about 0.01 to about 10 mg; in another embodiment, the unit dosage form is about 0.01 to about 5 mg; and in another embodiment, the unit dosage form is about 0.01 to about 10 mg.

The compound or a pharmaceutically acceptable salt of the compound can be assayed in vitro or in vivo for the desired therapeutic or prophylactic activity prior to use in humans. Animal model systems can be used to demonstrate safety and efficacy.

The present methods for treating or preventing a 5-HT_{1A}-related disorder can further comprise administering another therapeutic agent to the animal being administered the compound or a pharmaceutically acceptable salt of the compound. In one embodiment the other therapeutic agent is administered in an effective amount.

Effective amounts of the other therapeutic agents are well known to those skilled in the art. However, it is well within the skilled artisan's purview to determine the other therapeutic agent's optimal effective amount range. The compound or a pharmaceutically acceptable salt of the compound and the other therapeutic agent can act additively or, in one embodiment, synergistically. In one embodiment of the invention, where another therapeutic agent is administered to an animal, the effective amount of the compound or a pharmaceutically acceptable salt of the compound is less than its effective amount would be where the other therapeutic agent is not administered. In this case, without being bound by theory, it is believed that the compound or a pharmaceutically acceptable salt of the compound and the other therapeutic agent act synergistically. In some cases, the patient in need of treatment is being treated with one or more other therapeutic agents. In some cases, the patient in need of treatment is being treated with at least two other therapeutic agents.

In one embodiment, the other therapeutic agent is selected from the group consisting of one or more anti-depressant agents, anti-anxiety agents, anti-psychotic agents, or cognitive enhancers. Examples of classes of antidepressants that can be used in combination with the active compounds of this invention include norepinephrine reuptake inhibitors, selective serotonin reuptake inhibitors (SSRIs), NK-1 receptor antagonists, monoamine oxidase inhibitors (MAOs), reversible inhibitors of monoamine oxidase (RIMAs), serotonin and noradrenaline reuptake inhibitors (SNRIs), corticotropin releasing factor (CRF) antagonists, α-adrenoreceptor antagonists, and atypical antidepressants. Suitable norepinephrine reuptake inhibitors include tertiary amine tricyclics and secondary amine tricyclics. Suitable tertiary amine tricyclics and secondary amine tricyclics include amitriptyline, clomipramine, doxepin, imipramine, trimipramine, dothiepin, butriptyline, iprindole, lofepramine, nortriptyline, protriptyline, amoxapine, desipramine and maprotiline. Suitable selective serotonin reuptake inhibitors include fluoxetine, citolopram, escitalopram, fluvoxamine, paroxetine and sertraline. Examples of monoamine oxidase inhibitors include isocarboxazid, phenelzine, and tranylcypromine. Suitable reversible inhibitors of monoamine oxidase include moclobemide. Suitable serotonin and noradrenaline reuptake inhibitors of use in the present invention include venlafaxine, nefazodone, milnacipran, and duloxetine. Suitable CRF antagonists include those compounds described in International Patent Publication Nos. WO 94/13643, WO 94/13644, WO 94/13661, WO 94/13676 and WO 94/13677. Suitable atypical anti-depressants include bupropion, lithium, nefazodone, trazodone and viloxazine. Suitable NK-1 receptor antagonists include those referred to in International Patent Publication WO 01/77100.

Anti-anxiety agents that can be used in combination with the active compounds of this invention include without limitation benzodiazepines and serotonin 1A (5-HT_{1A}) agonists or antagonists, especially 5-HT_{1A} partial agonists, and corticotropin releasing factor (CRF) antagonists. Exemplary suitable benzodiazepines include alprazolam, chlordiazepoxide, clonazepam, chlorazepate, diazepam, halazepam, lorazepam, oxazepam, and prazepam. Exemplary suitable 5-HT_{1A} receptor agonists or antagonists include buspirone, flesinoxan, gepirone and ipsapirone.

Anti-psychotic agents that are used in combination with the active compounds of this invention include without limitation aliphatic phethiazine, a piperazine phenothiazine, a butyrophenone, a substituted benzamide, and a thioxanthine. Additional examples of such drugs include without limitation haloperidol, olanzapine, clozapine, risperidone, pimozide, aripiprazol, and ziprasidone. In some cases, the drug is an anticonvulsant, e.g., phenobarbital, phenyloin, primidone, or carbamazepine.

Cognitive enhancers that are co-administered with the 5-HT_{1A} antagonist compounds of this invention include, without limitation, drugs that modulate neurotransmitter levels (e.g., acetylcholinesterase or cholinesterase inhibitors, cholinergic receptor agonists or serotonin receptor antagonists), drugs that modulate the level of soluble Aβ, amyloid fibril formation, or amyloid plaque burden (e.g., γ-secretase inhibitors, β-secretase inhibitors, antibody therapies, and degradative enzymes), and drugs that protect neuronal integrity (e.g., antioxidants, kinase inhibitors, caspase inhibitors, and hormones). Other representative candidate drugs that are co-administered with the compounds of the invention include cholinesterase inhibitors, (e.g., tacrine (COGNEX®), donepezil (ARICEPT®), rivastigmine (EXELON®) galantamine (REMINYL®), metrifonate, physostigmine, and Huperzine A), N-methyl-D-aspartate (NMDA) antagonists and agonists (e.g., dextromethorphan, memantine, dizocilpine maleate (MK-801), xenon, remacemide, eliprodil, amantadine, D-cycloserine, felbamate, ifenprodil, CP-101606 (Pfizer), Delucemine, and compounds described in U.S. Pat. Nos. 6,821,985 and 6,635,270), ampakines (e.g., cyclothiazide, aniracetam, CX-516 (Ampalex®), CX-717, CX-516, CX-614, and CX-691 (Cortex Pharmaceuticals, Inc. Irvine, Calif.), 7-chloro-3-methyl-3-4-dihydro-2H-1,2,4-benzothiadiazine S,S-dioxide (see Zivkovic et al., 1995, J. Pharmacol. Exp. Therap., 272:300-309; Thompson et al., 1995, Proc. Natl. Acad. Sci. USA, 92:7667-7671), 3-bicyclo[2,2,1]hept-5-en-2-yl-6-chloro-3,4-dihydro-2H-1,2,4-benzothiadiazine-7-sulfonamide-1,1-dioxide (Yamada, et al., 1993, J. Neurosc. 13:3904-3915); 7-fluoro-3-methyl-5-ethyl-1,2,4-benzothiadiazine-S,S-dioxide; and compounds described in U.S. Pat. No. 6,620,808 and International Patent Application Nos. WO 94/02475, WO 96/38414, WO 97/36907, WO 99/51240, and WO 99/42456), benzodiazepine (BZD)/GABA receptor complex modulators (e.g., progabide, gengabine, zaleplon, and compounds described in U.S. Pat. Nos. 5,538,956, 5,260,331, and 5,422,355); serotonin antagonists (e.g., 5-HT receptor modulators, including other 5-HT_{1A} antagonist compounds and 5-HT6 antagonists (including without limitation compounds described in U.S. Pat. Nos. 6,727,236, 6,825,212, 6,995,176, and 7,041,695)); nicotinics (e.g., niacin); muscarinics (e.g., xanomeline, CDD-0102, cevimeline, talsaclidine, oxybutin, tolterodine, propiverine, tropsium chloride and darifenacin); monoamine oxidase type B (MAO B) inhibitors (e.g., rasagiline, selegiline, deprenyl, lazabemide, safinamide, clorgyline, pargyline, N-(2-aminoethyl)-4-chlorobenzamide hydrochloride, and N-(2-aminoethyl)-5(3-fluorophenyl)-4-thiazolecarboxamide hydrochloride); phosphodiesterase (PDE) inhibitors (e.g., PDE IV inhibitors, roflumilast, arofylline, cilomilast, rolipram, RO-20-1724, theophylline, denbufylline, ARIFLO, CDP-840 (a tri-aryl ethane) CP80633 (a pyrimidone), RP 73401 (Rhone-Poulenc Rorer), denbufylline (SmithKline Beecham), arofylline (Almirall), CP-77,059 (Pfizer), pyrid[2,3d]pyridazin-5-ones (Syntex), EP-685479 (Bayer), T-440 (Tanabe Seiyaku), and SDZ-ISQ-844 (Novartis)); G proteins; channel modulators; immunotherapeutics (e.g., compounds described in U.S. Patent Application Publication No. US 2005/0197356 and US 2005/0197379); anti-amyloid or amyloid lowering agents (e.g., bapineuzumab and compounds described in U.S. Pat. No. 6,878,742 or U.S. Patent Application Publication Nos. US 2005/0282825 or U.S. 2005/0282826); statins and peroxisome proliferators activated receptor (PPARS) modulators (e.g., gemfibrozil (LOPID®), fenofibrate (TRICOR®), rosiglitazone maleate (AVANDIA®), pioglitazone (Actos™), rosiglitazone (Avandia™), clofibrate and bezafibrate); cysteinyl protease inhibitors; an inhibitor of receptor for advanced glycation endproduct (RAGE) (e.g., aminoguanidine, pyridoxaminem carnosine, phenazinediamine, OPB-9195, and tenilsetam); direct or indirect neurotropic agents (e.g., Cerebrolysin®, piracetam, oxiracetam, AIT-082 (Emilieu, 2000, Arch. Neurol. 57:454)); beta-secretase (BACE) inhibitors, α-secretase, immunophilins, caspase-3 inhibitors, Src kinase inhibitors, tissue plasminogen activator (TPA) activators, AMPA (alpha-amino-3-hydroxy-5-methyl-4-isoxazolepropionic acid) modulators, M4 agonists, JNK3 inhibitors, LXR agonists, H3 antagonists, and angiotensin IV antagonists. Other cognition enhancers include, without limitation, acetyl-1-carnitine, citicholine, huperzine, DMAE (dimethylaminoethanol), Bacopa monneiri extract, Sage extract, L-alpha glyceryl phosphoryl choline, Ginko biloba and Ginko biloba extract, Vinpocetine, DHA, nootropics including Phenyltropin, Pikatropin (from Creative Compounds, LLC, Scott City, Mo.), besipirdine, linopirdine, sibopirdine, estrogen and estrogenic compounds, idebenone, T-588 (Toyama Chemical, Japan), and FK960 (Fujisawa Pharmaceutical Co. Ltd.). Compounds described in U.S. Pat. Nos. 5,219,857, 4,904,658, 4,624,954 and 4,665,183 are also useful as cognitive enhancers as described herein. Cognitive enhancers that act through one or more of the above mechanisms are also within the scope of this invention.

In one embodiment, the compound or a pharmaceutically acceptable salt of the compound of the invention and cognitive enhancer act additively or, in one embodiment, synergistically. In one embodiment, where a cognitive enhancer and a compound or a pharmaceutically acceptable salt of the compound of the invention are co-administered to an animal, the effective amount of the compound or pharmaceutically acceptable salt of the compound of the invention is less than its effective amount would be where the cognitive enhancer agent is not administered. In one embodiment, where a cognitive enhancer and a compound or a pharmaceutically acceptable salt of the compound of the invention are co-administered to an animal, the effective amount of the cognitive enhancer is less than its effective amount would be where the compound or pharmaceutically acceptable salt of the invention is not administered. In one embodiment, a cognitive enhancer and a compound or a pharmaceutically acceptable salt of the compound of the invention are co-administered to an animal in doses that are less than their effective amounts would be where they were no co-administered. In these cases, without being bound by theory, it is believed that the compound or a pharmaceutically acceptable salt of the compound and the cognitive enhancer act synergistically.

In one embodiment, the other therapeutic agent is an agent useful for treating Alzheimer's disease or conditions associate with Alzheimer's disease, such as dementia. Exemplary agents useful for treating Alzheimer's disease include, without limitation, donepezil, rivastigmine, galantamine, memantine, and tacrine.

In one embodiment, the compound or a pharmaceutically acceptable salt of the compound is administered concurrently with another therapeutic agent.

In one embodiment, a composition comprising an effective amount of the compound or a pharmaceutically acceptable salt of the compound and an effective amount of another therapeutic agent within the same composition can be administered.

In another embodiment, a composition comprising an effective amount of the compound or a pharmaceutically acceptable salt of the compound and a separate composition comprising an effective amount of another therapeutic agent can be concurrently administered. In another embodiment, an effective amount of the compound or a pharmaceutically acceptable salt of the compound is administered prior to or subsequent to administration of an effective amount of another therapeutic agent. In this embodiment, the compound or a pharmaceutically acceptable salt of the compound is administered while the other therapeutic agent exerts its therapeutic effect, or the other therapeutic agent is administered while the compound or a pharmaceutically acceptable salt of the compound exerts its preventative or therapeutic effect for treating or preventing a 5-HT_{1A}-related disorder.

Thus, in one embodiment, the invention provides a composition comprising an effective amount of the compound or a pharmaceutically acceptable salt of the compound of the present invention and a pharmaceutically acceptable carrier. In another embodiment, the composition further comprises a second therapeutic agent.

In another embodiment, the composition further comprises a therapeutic agent selected from the group consisting of one or more other antidepressants, anti-anxiety agents, anti-psychotic agents or cognitive enhancers. Antidepressants, anti-anxiety agents, anti-psychotic agents and cognitive enhancers suitable for use in the composition include the antidepressants, anti-anxiety agents, anti-psychotic agents and cognitive enhancers provided above.

In another embodiment, the pharmaceutically acceptable carrier is suitable for oral administration and the composition comprises an oral dosage form.

In one embodiment, the compounds or pharmaceutically acceptable salts of the compounds of the present invention are useful as 5-HT_{1A} receptor antagonists and/or agonists. Compounds that are 5-HT_{1A} antagonists and/or agonists can readily be identified by those skilled in the art using numerous art-recognized methods, including standard pharmacological test procedures such as those described herein. Accordingly, the compounds and pharmaceutically acceptable salts of the compounds of the present invention are useful for treating a mammal with a 5-HT_{1A}-related disorder. One non-limiting example of a disorder that 5-HT_{1A} receptor antagonists are useful for treating is cognition-related disorder, while a non-limiting example of a disorder that 5-HT_{1A} receptor agonists are useful for treating is anxiety-related disorder. In some embodiments, the compounds and pharmaceutical salts of the invention are useful for improving cognitive function or cognitive deficits. Examples of improvements in cognitive function include, without limitation, memory improvement and retention of learned information. Accordingly, the compounds and pharmaceutical salts of the invention are useful for slowing the loss of memory and cognition and for maintaining independent function for patients afflicted with a cognition-related disorder. Thus, in one embodiment, the compounds and pharmaceutically acceptable salts of the compounds of the present invention that act as 5-HT_{1A} receptor antagonists are useful for treating a mammal with a cognition-related disorder. In one embodiment, the compounds and pharmaceutically acceptable salts of the compounds of the present invention that act as 5-HT_{1A} receptor antagonists are useful for improving the cognitive function of a mammal. Similarly, in one embodiment, the compounds and pharmaceutically acceptable salts of the compounds of the present invention that act as 5-HT_{1A} receptor agonists are useful for treating a mammal with an anxiety-related disorder.

In one embodiment, the invention provides a method for treating a 5-HT_{1A}-related disorder, comprising administering to a mammal in need thereof at least one purified and isolated metabolite of COMPOUND I (e.g., M2, M3, M5, M6, M7, M9, M10, M11, M16, M17, M18, M20, M21, M22, M23, M25 and M26) or a pharmaceutically acceptable salt thereof in an amount effective to treat a 5-HT_{1A}-related disorder. In one embodiment, the invention provides a method for treating a cognition-related disorder, comprising administering to a mammal in need thereof at least one purified and isolated metabolite of COMPOUND I (e.g., M2, M3, M5, M6, M7, M9, M10, M11, M16, M17, M18, M20, M21, M22, M23, M25 and M26) or a pharmaceutically acceptable salt thereof in an amount effective to treat a cognition-related disorder. In one embodiment, the invention provides a method for treating an anxiety-related disorder, comprising administering to a mammal in need thereof at least one purified and isolated metabolite of COMPOUND I (e.g., M2, M3, M5, M6, M7, M9, M10, M11, M16, M17, M18, M20, M21, M22, M23, M25 and M26) or a pharmaceutically acceptable salt thereof in an amount effective to treat an anxiety-related disorder.

In one embodiment, the invention provides a method for treating Alzheimer's disease, comprising administering to a mammal in need thereof at least one purified and isolated metabolite of COMPOUND I (e.g., M2, M3, M5, M6, M7, M9, M10, M11, M16, M17, M18, M20, M21, M22, M23, M25 and M26) or a pharmaceutically acceptable salt thereof in an amount effective to treat Alzheimer's disease. In one embodiment, the method for treating Alzheimer's disease includes administering a second therapeutic agent. In some embodiments, the second therapeutic agent is an anti-depressant agent, an anti-anxiety agent, an anti-psychotic agent, or a cognitive enhancer.

In one embodiment, the invention provides a method for treating mild cognitive impairment (MCI), comprising administering to a mammal in need thereof at least one purified and isolated metabolite of COMPOUND I (e.g., M2, M3, M5, M6, M7, M9, M10, M11, M16, M17, M18, M20, M21, M22, M23, M25 and M26) or a pharmaceutically acceptable salt thereof in an amount effective to treat mild cognitive impairment (MCI). In one embodiment, the method for treating MCI includes administering a second therapeutic agent. In some embodiments, the second therapeutic agent is an anti-depressant agent, an anti-anxiety agent, an anti-psychotic agent, or a cognitive enhancer.

In one embodiment, the invention provides a method for treating depression, comprising administering to a mammal in need thereof at least one purified and isolated metabolite of COMPOUND I (e.g., M2, M3, M5, M6, M7, M9, M10, M1, M16, M17, M18, M20, M21, M22, M23, M25 and M26) or a pharmaceutically acceptable salt thereof in an amount effective to treat depression. In one embodiment, the method for treating depression includes administering a second therapeutic agent. In some embodiments, the second therapeutic agent is an anti-depressant agent, an anti-anxiety agent, an anti-psychotic agent, or a cognitive enhancer.

In some embodiments, the invention provides a pharmaceutical composition for treating a 5-HT_{1A}-related disorder, the composition including at least one purified and isolated metabolite of COMPOUND I (e.g., M2, M3, M5, M6, M7, M9, M0, M11, M16, M17, M18, M20, M21, M22, M23, M25 and M26) or a pharmaceutically acceptable salt thereof. In some embodiments, the invention provides a pharmaceutical composition for treating a cognition-related disorder, the composition including at least one purified and isolated metabolite of COMPOUND I (e.g., M2, M3, M5, M6, M7, M9, M10, M1, M16, M17, M18, M20, M21, M22, M23, M25 and M26) or a pharmaceutically acceptable salt thereof. In some embodiments, the invention provides a pharmaceutical composition for treating an anxiety-related disorder, the composition including at least one purified and isolated metabolite of COMPOUND I (e.g., M2, M3, M5, M6, M7, M9, M0, M11, M16, M17, M18, M20, M21, M22, M23, M25 and M26) or a pharmaceutically acceptable salt thereof.

In one embodiment, the invention provides a pharmaceutical composition for treating Alzheimer's disease, the composition including at least one purified and isolated metabolite of COMPOUND I (e.g., M2, M3, M5, M6, M7, M9, M10, M11, M16, M17, M18, M20, M21, M22, M23, M25 and M26) or a pharmaceutically acceptable salt thereof.

In one embodiment, the invention provides a pharmaceutical composition for treating mild cognitive impairment (MCI), the composition including at least one purified and isolated metabolite of COMPOUND I (e.g., M2, M3, M5, M6, M7, M9, M1, M11, M16, M17, M18, M20, M21, M22, M23, M25 and M26) or a pharmaceutically acceptable salt thereof.

In one embodiment, the invention provides a pharmaceutical composition for treating depression, the composition including at least one purified and isolated metabolite of COMPOUND I (e.g., M2, M3, M5, M6, M7, M9, M10, M11, M16, M17, M18, M20, M21, M22, M23, M25 and M26) or a pharmaceutically acceptable salt thereof.

In one embodiment, the compounds or pharmaceutically acceptable salts of the compounds of the present invention are useful for treating sexual dysfunction, e.g., sexual dysfunction associated with drug treatment such as drug treatment with an antidepressant, an antipsychotic, or an anticonvulsant. Accordingly, in one embodiment, the invention provides a method for treating sexual dysfunction associated with drug treatment in a patient in need thereof. The method includes administering to the patient an effective amount of one or more of the compounds disclosed herein. In some embodiments, the drug treatment is antidepressant drug treatment, antipsychotic drug treatment, or anticonvulsant drug treatment. The compound can be at least one purified and isolated metabolite of COMPOUND I (e.g., M2, M3, M5, M6, M7, M9, M0, M11, M16, M17, M18, M20, M21, M22, M23, M25 and M26) or a pharmaceutically acceptable salt thereof.

In certain embodiments, the drug associated with sexual dysfunction is a selective serotonin reuptake inhibitor (SSRI) (for example, fluoxetine, citalopram, escitalopram oxalate, fluvoxamine maleate, paroxetine, or sertraline), a tricyclic antidepressant (for example, desipramine, amitriptyline, amoxipine, clomipramine, doxepin, imipramine, nortriptyline, protriptyline, trimipramine, dothiepin, butriptyline, iprindole, or lofepramine), an aminoketone class compound (for example, bupropion). In some embodiments, the drug is a monoamine oxidase inhibitor (MAOI) (for example, phenelzine, isocarboxazid, or tranylcypromine), a serotonin and norepinepherine reuptake inhibitor (SNRI) (for example, venlafaxine, nefazodone, milnacipran, duloxetine), a norepinephrine reuptake inhibitor (NRI) (for example, reboxetine), a partial 5-HT_{1A} agonist (for example, buspirone), a 5-HT_{2A} receptor antagonist (for example, nefazodone), a typical antipsychotic drug, or an atypical antipsychotic drug. Examples of such antipsychotic drugs include aliphatic phethiazine, a piperazine phenothiazine, a butyrophenone, a substituted benzamide, and a thioxanthine. Additional examples of such drugs include haloperidol, olanzapine, clozapine, risperidone, pimozide, aripiprazol, and ziprasidone. In some cases, the drug is an anticonvulsant, e.g., phenobarbital, phenyloin, primidone, or carbamazepine. In some cases, the patient in need of treatment for sexual dysfunction is being treated with at least two drugs that are antidepressant drugs, antipsychotic drugs, anticonvulsant drugs, or a combination thereof.

In some embodiments of the invention, the sexual dysfunction comprises a deficiency in penile erection.

The invention also provides a method of improving sexual function in a patient in need thereof. The method includes administering to the patient a pharmaceutically effective amount of one or more of the compounds disclosed herein. The compound can be at least one purified and isolated metabolite of COMPOUND I (e.g., M2, M3, M5, M6, M7, M9, M10, M11, M116, M17, M18, M20, M21, M22, M23, M25 and M26) or a pharmaceutically acceptable salt thereof.

In another embodiment, the invention provides a pharmaceutical composition for treating sexual dysfunction associated with drug treatment, the composition including at least one purified and isolated metabolite of COMPOUND I (e.g., M2, M3, M5, M6, M7, M9, M10, M11, M116, M17, M18, M20, M21, M22, M23, M25 and M26) or a pharmaceutically acceptable salt thereof. In some embodiments, the drug is an antidepressant, an antipsychotic, or an anticonvulsant. In other embodiments, the compound or pharmaceutically acceptable salt of the compound is effective for ameliorating sexual dysfunction in an animal model of sexual dysfunction associated with drug treatment, for example, in an animal model of sexual dysfunction that is an antidepressant drug-induced model of sexual dysfunction.

The compounds and pharmaceutically acceptable salts of the purified and isolated metabolite of COMPOUND I (e.g., M2, M3, M5, M6, M7, M9, M10, M11, M16, M17, M18, M20, M21, M22, M23, M25 and M26) are also useful in the manufacture of medicaments for treating a 5-HT_{1A}-related disorder in a mammal. Similarly, the compounds and pharmaceutically acceptable salts of the purified and isolated metabolite of COMPOUND I (e.g., M2, M3, M5, M6, M7, M9, M10, M11, M16, M17, M18, M20, M21, M22, M23, M25 and M26) are also useful in the manufacture of medicaments for treating a cognition-related disorder in a mammal. Also, the compounds and pharmaceutically acceptable salts of the purified and isolated metabolite of COMPOUND I (e.g., M2, M3, M5, M6, M7, M9, M10, M11, M16, M17, M18, M20, M21, M22, M23, M25 and M26) are also useful in the manufacture of medicaments for treating an anxiety-related disorder in a mammal.

### EXAMPLES

### Example 1

### Preparation of 5-Fluoro-8-(4-(4-(6-methoxyquinolin-8-yl)piperazin-1-yl)piperidin-1-yl)-quinoline (COMPOUND I)

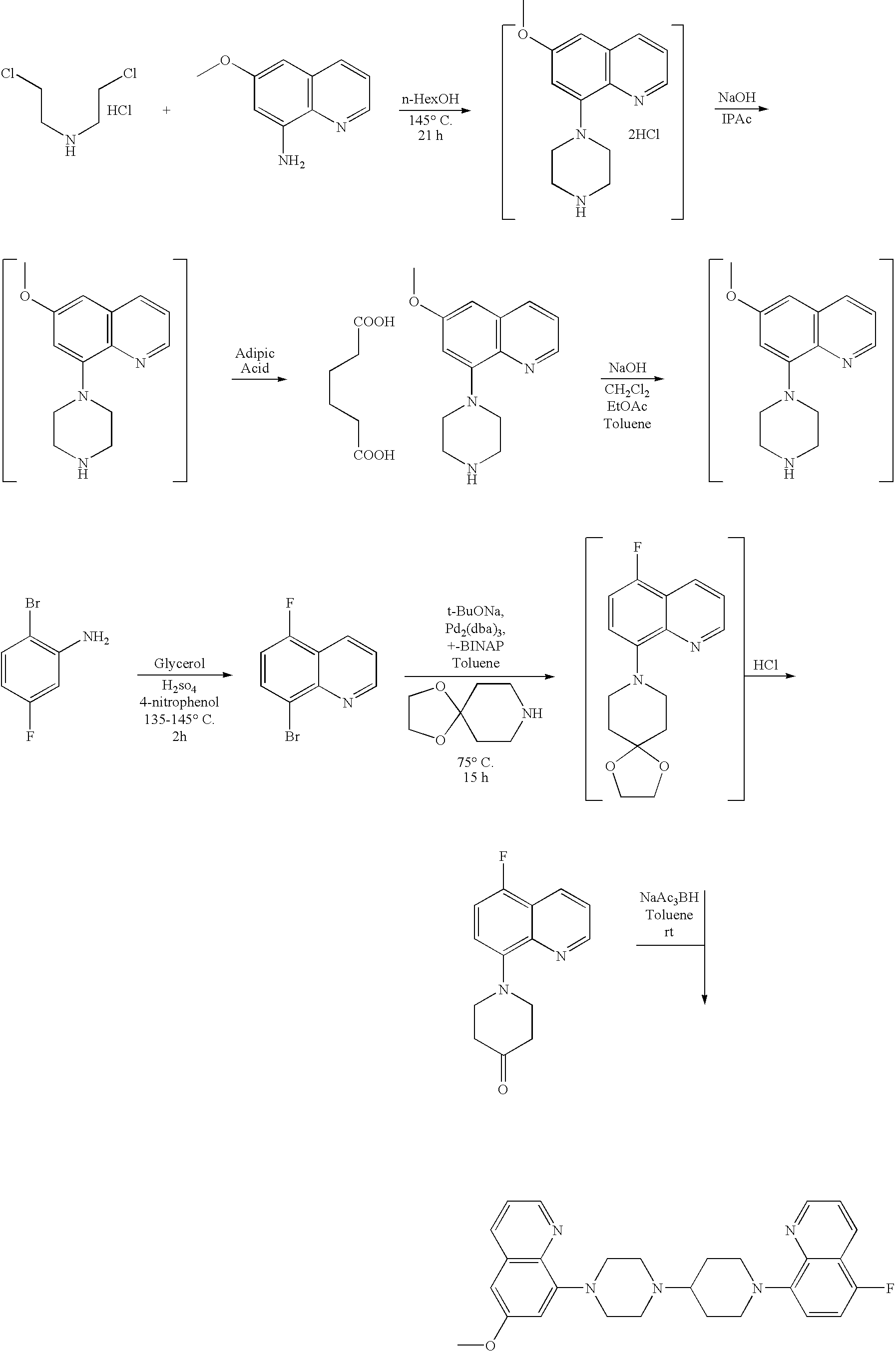

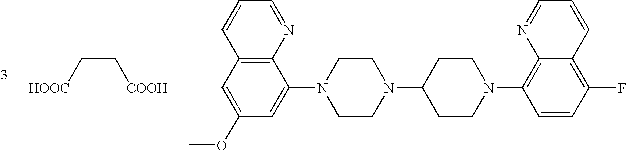

### 1) 6-methoxy-8-(1-piperazinyl)quinoline

A mixture of 8-amino-6-methoxyquinoline (150.0 g, 0.862 mol) and bis(2-chloroethyl)amine (219 g, 1.23 mol) in 6 parts (volume of hexanol vs weight of 8-amino-6-methoxyquinoline) of 1-hexanol (900 mL) was heated to 145° C. and stirred for 21 hours. Upon completion, the reaction mixture was cooled 50-60° C., and 507 g of aqueous NaOH solution was added slowly. The reaction mixture was cooled to 25-30° C. and isopropyl acetate (750 mL) was added. The mixture was clarified through a celite pad. The aqueous phase was then split off. The organic solution was treated with a slurry of adipic acid (126 g, 0.862 mol) in isopropyl acetate (250 ml). The resulting mixture was stirred for 16 hours to form 6-methoxy-8-(1-piperazinyl)quinoline adipate salt. The adipate salt was filtered and washed with isopropyl acetate (2×150 ml) and dried by nitrogen flow to give adipate of 6-Methoxy-8-piperazin-1-yl-quinoline (186 g, 55% yield) with ˜97% HPLC area, 88% strength purity in 51% yield.

The salt can be recrystallized from a mixture of methanol and isopropyl acetate if further purification is required. To purify the adipate salt, 580 g of the crude adipate salt and 2.8 liter of methanol were mixed and heated to 65° C. and a dark solution was obtained. To this solution was charged slowly 1.1 liter of isopropyl acetate over 40 min at about 63° C. The mixture was stirred at about 63° C. for about 1 h and cooled to 0-5° C. After stirring at 0-5° C. for 2 hours, the mixture was filtered and washed with 300 ml of isopropyl acetate and dried with airflow. Yield, 395 g, 68.1% recovery yield.

To liberate 6-methoxy-8-(1-piperazinyl)quinoline from its adipate salt, 100 g (0.257 mol) of the adipate salt was added into a 2-L reactor followed by the addition of 500 ml of dichloromethane. To this mixture was added 100 g of water followed by the slow (in about 15 min) addition of 41 g of 50% sodium hydroxide solution to maintain the pH in the 13-14 range, adding sodium hydroxide solution as necessary if the pH is below 10. The organic bottom layer was separated and filtered through a pad of activated basic aluminum oxide (100 g, 6.5 cm diameter×3 cm depth). The pad was washed with 100 ml of isopropyl acetate twice. The dichloromethane was replaced by toluene by distillation under vacuum (450 to 500 mm Hg) while 3×150 ml of toluene was added into the reactor until the final volume was about 135 ml. Some white solid precipitated after distillation, the solid was removed by filtration, the filter cake was washed with 50 ml of toluene. Final volume, 185 ml, purity 97.56%, solution strength 27.4%)

### 2) 8-bromo-5-fluoroquinoline

To a 2-L reactor equipped with a mechanic agitator, a condenser, a thermocouple, a baffle, and nitrogen inlet were charged 228 g of water, 200 g of 2-bromo-5-fluoroaniline and 80 g of 4-nitrophenol. To this mixture was charged 96% sulfuric acid in 10-30 min at 20-120° C. The mixture was heated to 135-140° C. and 194 g of glycerol was charged into the reactor over two hours at 135-145° C. The mixture was held at 135-145° C. for 1 hour after the addition. The reaction mixture was cooled to below 20-50° C. and slowly transferred to a 5-L reactor containing 1100 g of water and 1210 g of toluene. The 2-L reactor was washed with 300 g of water and the wash was combined into the 5-L reactor. The pH of the contents in the 5-L reactor was adjusted to pH 8-10 by adding approximately 1233 g (1370 mL) ammonium hydroxide (28-30% NH₃) at 20-40° C. The mixture was stirred at room temperature for 15 min and the solid by-product was filtered off while the filtrate was retained. The filter cake was washed with 400 ml of toluene and the all the filtrate was combined and charged a 3-L reactor. About 500 ml of 8.5% KOH solution was charged into the 3-L reactor and stirred for 10 min and bottom aqueous layer was spit off. A second portion of 500 ml of 8.5% KOH solution was added and the mixture was stirred for 15 min and the bottom aqueous layer was split off. Water 500 ml was added and stirred for 15 min before the bottom aqueous layer was split off. The organic layer was heated to distill off about 100-200 ml of toluene to azeotropically remove water. A clear solution will be obtained. Typical yield 178 g real 8-bromo-5-fluoroquinoline, ˜75%.

Alternatively, 8-bromo-5-fluoroquinoline was prepared by adding a warm mixture containing 2-bromo-5-fluoroaniline (100 g, 1.0 eq), 4-nitrophenol (40 g, 0.54 eq), and glycerol (97 g, 2.0 eq) over 1.5 hours to sulfuric acid (267 ml) and water (114 mL) at 140-150° C. The initial mixture showed 37.8% 4-nitrophenol by relative HPLC area %. Samples showed 4.7% 4-nitrophenol immediately after adding 50% of mixed starting materials and 5.0% immediately after adding all of the materials. The yield upon workup was 87.5%, with total impurities 0.29%. Addition of less (0.46 eq, 34 g) 4-nitrophenol also successfully produced the intermediate of interest at acceptable yield.

### 3) 1-(5-fluoroquinolin-8-yl)piperidin-4-one

To a 5-L jacketed cylindrical reactor equipped with an impeller-style agitator, condenser, thermocouple, and vacuum/nitrogen inlet was charged 2-L, 15% toluene solution of 8-bromo-5-fluoroquinoline, 209 g of 1,4-Dioxa-8-azaspiro[4.5]decane. Meanwhile in a 500-mL Erlenmeyer flask, a suspension of 16.5 g (26.5 mmol)±[1,1′-binaphthalene]-2,2′-diylbis[diphenyl-Phosphine, and 6.08 g (6.64 mmol) tris[μ-[(1,2-η:4,5-η)-(1E,4E)-1,5-diphenyl-1,4-pentadien-3-one]]dipalladium in 260 g of toluene was prepared. This freshly made suspension was charged into the 5-L reactor followed by a rinse of 170 g of toluene. 166 g sodium tert-butoxide was then charged into the reactor followed by a rinse with 430 g of toluene. The reactor was degassed by vacuum to less than 125 mmHg and then filled with nitrogen to atmosphere three times. The mixture was then heated to 50-60° C. and stirred for 1 h and then heat to 65-75° and stirred at this temperature for about 10 hours. The mixture was cooled to 40-50° C. and then quenched with 800 g of water. The lower aqueous layer was split off and the volume of the organic layer was reduced to about 1.5 L by vacuum distillation. To this residual was charged 2.28 kg of 20% sulfuric acid at 25-30° C. The mixture was stirred for an hour and was clarified by filtration and a bi-phase filtrate was obtained. The aqueous phase was split and retained. Toluene 870 g was added to the aqueous solution and the mixture was neutralized by slowly adding 770 g 50% sodium hydroxide solution. The lower aqueous layer was split off and extracted with 600 g of toluene. The organic layers were combined and the volume of the reaction was reduced to about 1 L by vacuum distillation. The residue was cooled to room temperature and 480 g of toluene was charged. The mixture was heated to 45-55° C. to form a clear solution, which was filtered through a celite/charcoal pad to remove palladium. The filtrate was concentrated by vacuum distillation to about 0.7 L and diluted with 620 g heptane, cooled to −15 to −5° C. to form a slurry. The solid was collected by filtration. The product was dried by air flow at room temperature. Typical yield is about 70%.

### 4) 5-fluoro-8-[4-[4-(6-methoxyquinolin-8-yl)piperazin-1-yl]piperidin-1-yl]quinoline

Toluene (118 g), sodium triacetoxyborohydride (44.5 g) were mixed at 0° C. to room temperature. To this mixture was charged a premixed toluene solution of 6-methoxy-8-(1-piperazinyl)quinoline (Step 1, 160 g, 27.4 wt % in toluene) and 1-(5-fluoroquinolin-8-yl)piperidin-4-one (Step 3, 41 g). The resulting mixture was stirred for 2 to 3 hours at about 30° C. KOH solution (443 g 9% in water) was charged to quench the residual sodium triacetoxyborohydride. Heptane (118 g) was added to further precipitate the product. The product was then filtered and washed with ethanol (2×100 ml). Yield 68 g, 86%. This crude product (67 g) was dissolved in 586 g dichloromethane and passed through a charcoal/celite pad to remove palladium. The dichloromethane was distilled off while 400 g of ethanol was slowly added at the same time. The resulting slurry was filtered and washed with ethanol twice (65 g+100 g). The product was dried in oven at 55° C. overnight. Purification recovery yield 59.9 g, 89.4%.

### Example 2

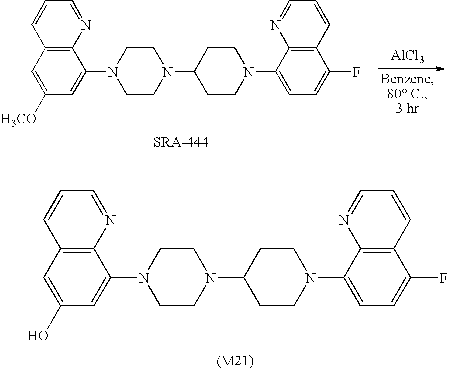

### Preparation of 8-{4-[1-(5-fluoroquinolin-8-yl)piperidin-4-yl]piperazin-1-yl}quinolin-6-ol trihydrochloride (M21)

To a solution of 5-fluoro-8-{4-[4-(6-methoxyquinolin-8-yl)piperazin-1-yl]piperidin-1-yl}quinoline (508 mg, 1.08 mmol) in anhydrous benzene (30 ml) was added anhydrous aluminum chloride (430 mg, 3.24 mmol). The reaction mixture was stirred at 80° C. for 3 hours. The solvent was removed on a rotary evaporator and the residue was dissolved in dichloromethane (150 ml), washed with saturated aq. NaHCO₃ and then washed with saturated aqueous sodium chloride. The organic layer was dried over anhydrous Na₂SO₄ and concentrated on a rotary evaporator to give a mixture of starting material and the desired product. Purification using preparative HPLC (Luna CN 5×15 cm column, 80:16:4 hexane/dichloromethane/methanol, containing 0.1% diethylamine) yielded the title compound as a yellow solid (240 mg, 49%), which was converted to the trihydrochloride salt with HCl/dichloromethane; MS (ES+) m/z=458 [M+H]⁺.

### Example 3

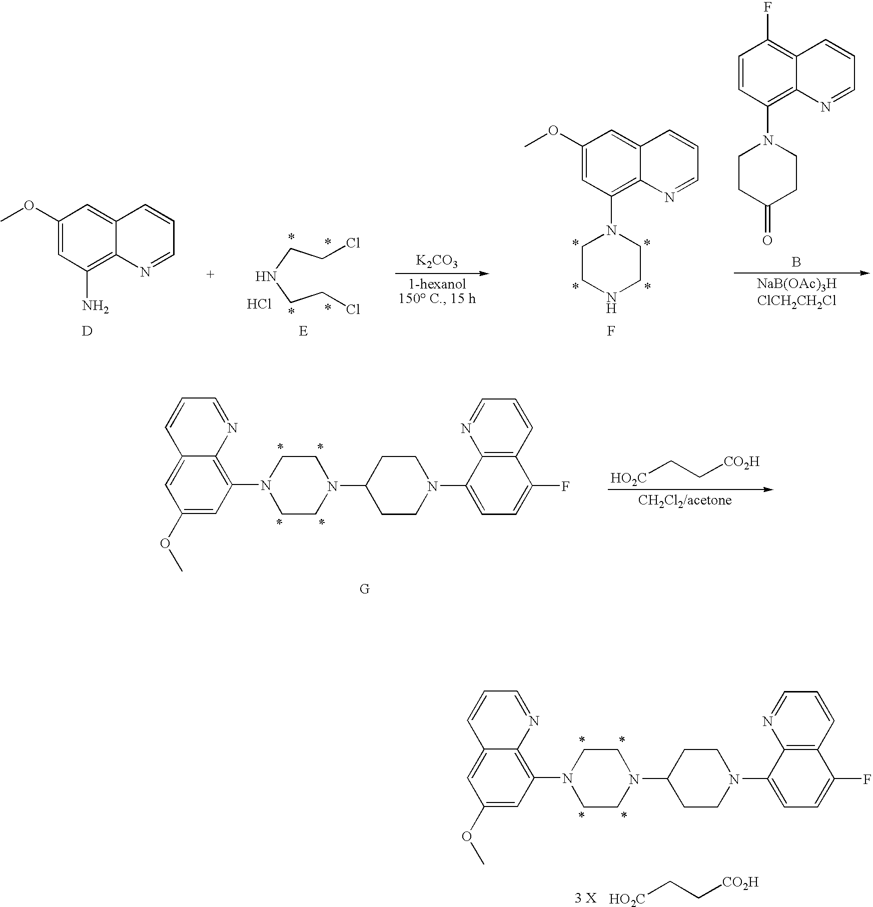

### Preparation of [Piperazine-¹⁴C(U)]-5-fluoro-8-(4-(4-(6-methoxyquinolin-8-yl)piperazin-1-yl)piperidin-1-yl)-quinoline (COMPOUND I)

### 1) [piperazine-¹⁴C(U)]-6-methoxy-8-(piperazin-1-yl)quinoline

To a mixture of 6-methoxyquinolin-8-amine (D, 589 mg, 3.38 mmol), K₂CO₃ (1.40 g, 10.14 mmol) and bis(2-chloro[U-¹⁴C]ethyl)amine hydrochloride (E, commercially available; 200 mCi, 59.2 mCi/mmol), 1-hexanol (8.5 mL) was added. The reaction mixture was stirred and heated at 150° C. for 17 hours. After cooling to room temperature, the reaction mixture was added H₂O (40 mL), NaOH (50% w/w 10 mL) and EtOAc (50 mL). Organic layer was separated. The aqueous layer was extracted with EtOAc (40 mL×3). Combined organic layers were washed with brine (30 mL) and dried over Na₂SO₄. The mixture was filtered through a celite pad. The celite pad was washed with EtOAc. The filtrate was concentrated in vacco to less than 3 mL in volume. The resulting viscous oil was diluted with EtOAc (13 mL) and added adipic acid (493 mg, 3.38 mmol). After stirring at room temperature overnight, the mixture was cooled down to 0° C. for 1 h. The precipitate was filtered, washed with EtOAc and dried under vacuum to give [U-¹⁴C]-6-methoxy-8-(piperazin-1-yl)quinoline (F) adipic acid salt (670 mg). The adipic acid salt (670 mg) was added H₂O (8 mL), NaOH (50% w/w, 2 mL) and CH₂Cl₂ (10 mL). CH₂Cl₂ layer was separated. The aqueous layer was extracted with CH₂Cl₂ (10 mL×3). Combined CH₂Cl₂ layers were concentrated to afford [piperazine-¹⁴C(U)]-6-methoxy-8-(piperazin-1-yl)quinoline (F, 459 mg, 55%) as a dark brown syrup, which was used for the next reaction without further purification.

### 2) [piperazine-¹⁴C(U)]-5-fluoro-8-(4-(4-(6-methoxyquinolin-8-yl)piperazin-1-yl)piperidin-1-yl)quinoline

To a solution of [piperazine-¹⁴C(U)]-6-methoxy-8-(piperazin-1-yl)quinoline (F, 459 mg, 1.87 mmol, 110.7 mCi) and 1-(5-fluoroquinolin-8-yl)piperidin-4-one (Intermediate B in Scheme 2, 457 mg, 1.87 mmol) in 1,2-dichloroethane (19 mL), NaBH(OAc)₃ (793 mg, 3.74 mmol) was added. After stirring at room temperature overnight, the reaction mixture was quenched with H₂O (25 mL) and NaOH (50% w/w, 5 mL), then extracted with CH₂Cl₂ (30 mL×3). CH₂Cl₂ layers were dried over Na₂SO₄. The crude product was purified by semi-prep. HPLC (Column: Luna C18 (2) 100 A 5 μm, 250×21.2 mm; Mobile phase A: 1900 mL H₂O/100 mL MeCN/1 mL TFA; Mobile phase B: 1900 mL H₂O/100 mL MeCN/1 mL TFA; 0-2 min: 100% A; 20 min: 60% A and 40% B; 25 min: 100% B; Retention time: 13.6 min) to afford [piperazine-¹⁴C(U)]-5-fluoro-8-(4-(4-(6-methoxyquinolin-8-yl)piperazin-1-yl)piperidin-1-yl)quinoline (G, 247 mg, 27%) as a yellow foam.

### 3) [piperazine-¹⁴C(U)]-5-fluoro-8-(4-(4-(6-methoxyquinolin-8-yl)piperazin-1-yl)piperidin-1-yl)quinoline trisuccinate

To a solution of [piperazine-¹⁴C(U)]-5-fluoro-8-(4-(4-(6-methoxyquinolin-8-yl)piperazin-1-yl)piperidin-1-yl)quinoline (247 mg, 0.522 mmol) in CH₂Cl₂ (5 mL), a solution of succinic acid (191 mg, 1.618 mmol) in acetone (7.5 mL) was added. After stirring at room temperature for 20 hours, the while precipitate was filtered, washed w/acetone and dried under vacuum to afford [piperazine-¹⁴C(U)]-5-fluoro-8-(4-(4-(6-methoxyquinolin-8-yl)piperazin-1-yl)piperidin-1-yl)quinoline trisuccinate (326 mg, 22.4 mCi, 75%) as a white solid. The specific activity was determined to be 56.9 mCi/mmol by gravimetric analysis for the trisuccinate salt. Chemical purity and radiochemical purity were found to be >99%.

### Example 4

### Biological Assays

Compounds of the invention can be tested according to the protocol described. The data demonstrate the protocol is effective for identifying compounds that have 5-HT_{1A} agonist activity and 5-HT_{1A} antagonist activity. 5-HT_{1A} agonist activity is demonstrated by inhibiting the forskolin-induced increase in cAMP levels and the results reported as EC₅₀ values. Compounds having 5-HT_{1A} antagonist activity show no effect on forskolin-induced increases in cAMP levels on their own, but block the 8-OH-DPAT-induced inhibition of forskolin-stimulated increases in cAMP levels. Results are required as IC₅₀ values. Using this protocol, M21 was found to be a potent 5-HT_{1A} receptor agonist having a Ki value of 0.47 nM for 5-HT_{1A} affinity and an EC₅₀ value of 0.39 nM (Emax=92%) for in vitro agonist activity.

All references cited herein are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supercede and/or take precedence over any such contradictory material.

All numbers expressing quantities of ingredients, reaction conditions, analytical results and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

We claim:

1. A metabolite of 5-Fluoro-8-{4-[4-(6-methoxyquinolin-8-yl)piperazin-1-yl]piperidin-1-yl}quinoline, or an enantiomer, diastereomer, tautomer, or pharmaceutically acceptable salt or solvate thereof.

2. The metabolite of claim 1 made by treating 5-Fluoro-8-{4-[4-(6-methoxyquinolin-8-yl)piperazin-1-yl]piperidin-1-yl}quinoline or its pharmaceutically acceptable salt with:
(a) rat, mouse, dog, monkey or human liver microsomes;
(b) rat, mouse, dog, monkey or human liver S9 fractions; or
(c) cryopreserved rat, dog, or human hepatocytes.

3. The metabolite of claim 1 made by administering 5-Fluoro-8-{4-[4-(6-methoxyquinolin-8-yl)piperazin-1-yl]piperidin-1-yl}quinoline or its pharmaceutically acceptable salt in a mammal.

4. The metabolite of claim 1, wherein the metabolite is not isolated.

5. A metabolite of 5-Fluoro-8-{4-[4-(6-methoxyquinolin-8-yl)piperazin-1-yl]piperidin-1-yl}quinoline, or an enantiomer, diastereomer, tautomer, or pharmaceutically acceptable salt or solvate thereof, wherein the metabolite is purified and isolated.

6. The metabolite of claim 5 made by treating 5-Fluoro-8-{4-[4-(6-methoxyquinolin-8-yl)piperazin-1-yl]piperidin-1-yl}quinoline or its pharmaceutically acceptable salt with:
(a) rat, mouse, dog, monkey or human liver microsomes;
(b) rat, mouse, dog, monkey or human liver S9 fractions; or
(c) cryopreserved rat, dog, or human hepatocytes.

7. The metabolite of claim 5 made by administering 5-Fluoro-8-{4-[4-(6-methoxyquinolin-8-yl)piperazin-1-yl]piperidin-1-yl}quinoline or its pharmaceutically acceptable salt in a mammal.

8. The metabolite of claim 5, exhibiting a mass spectral peak [M+H]⁺ at an m/z selected from the group consisting of:
(a) m/z 244;
(b) m/z 662;
(c) m/z 680;
(d) m/z 646;
(e) m/z 506;
(f) m/z 664;
(g) m/z 484;
(h) m/z 504;
(i) m/z 470;
(j) m/z 488;
(k) m/z 458;
(l) m/z 472;
(m) m/z 568;
(n) m/z 634;
(o) m/z 538; and
(p) m/z 524.

9. The metabolite of claim 5 selected for the group consisting of:

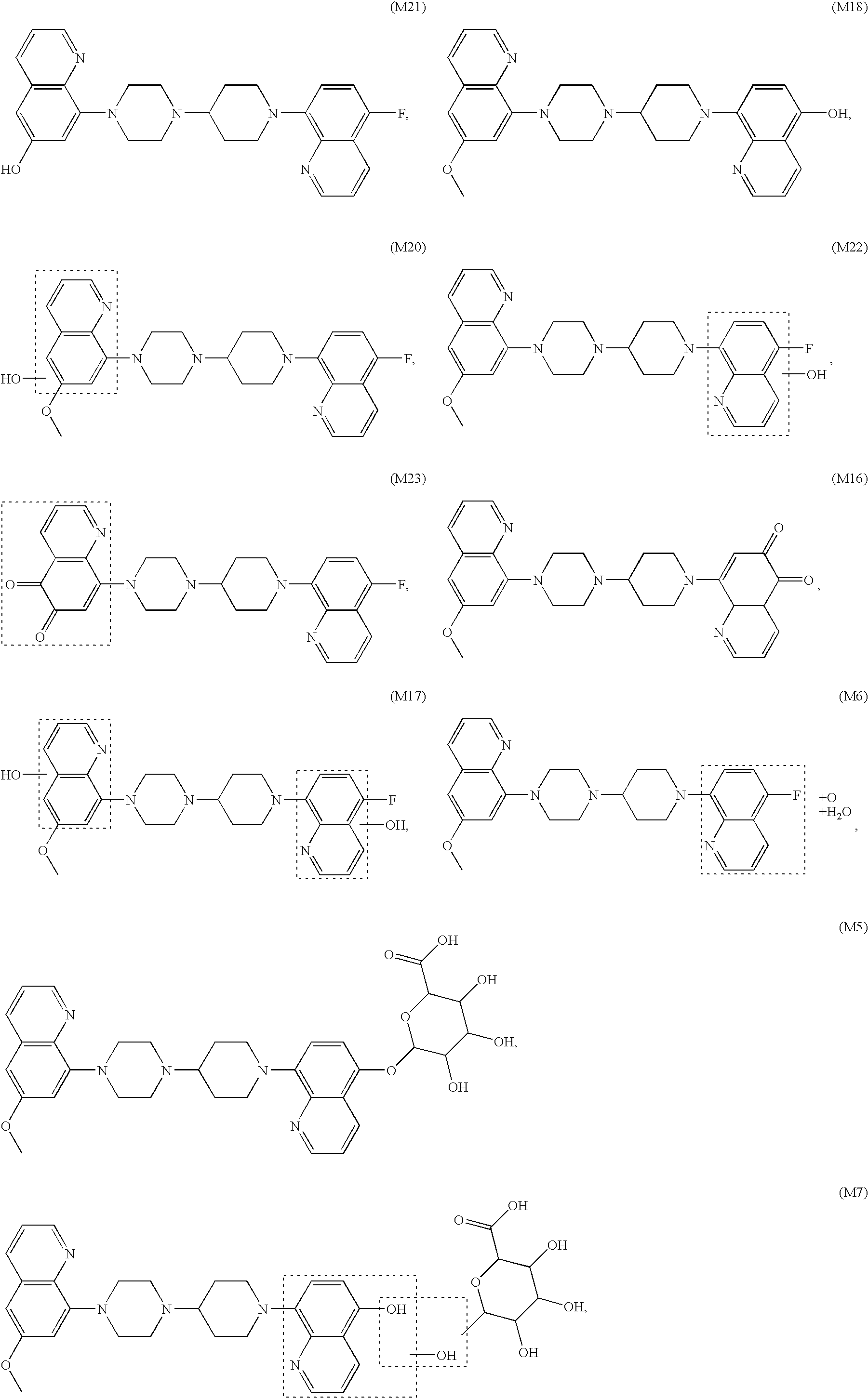

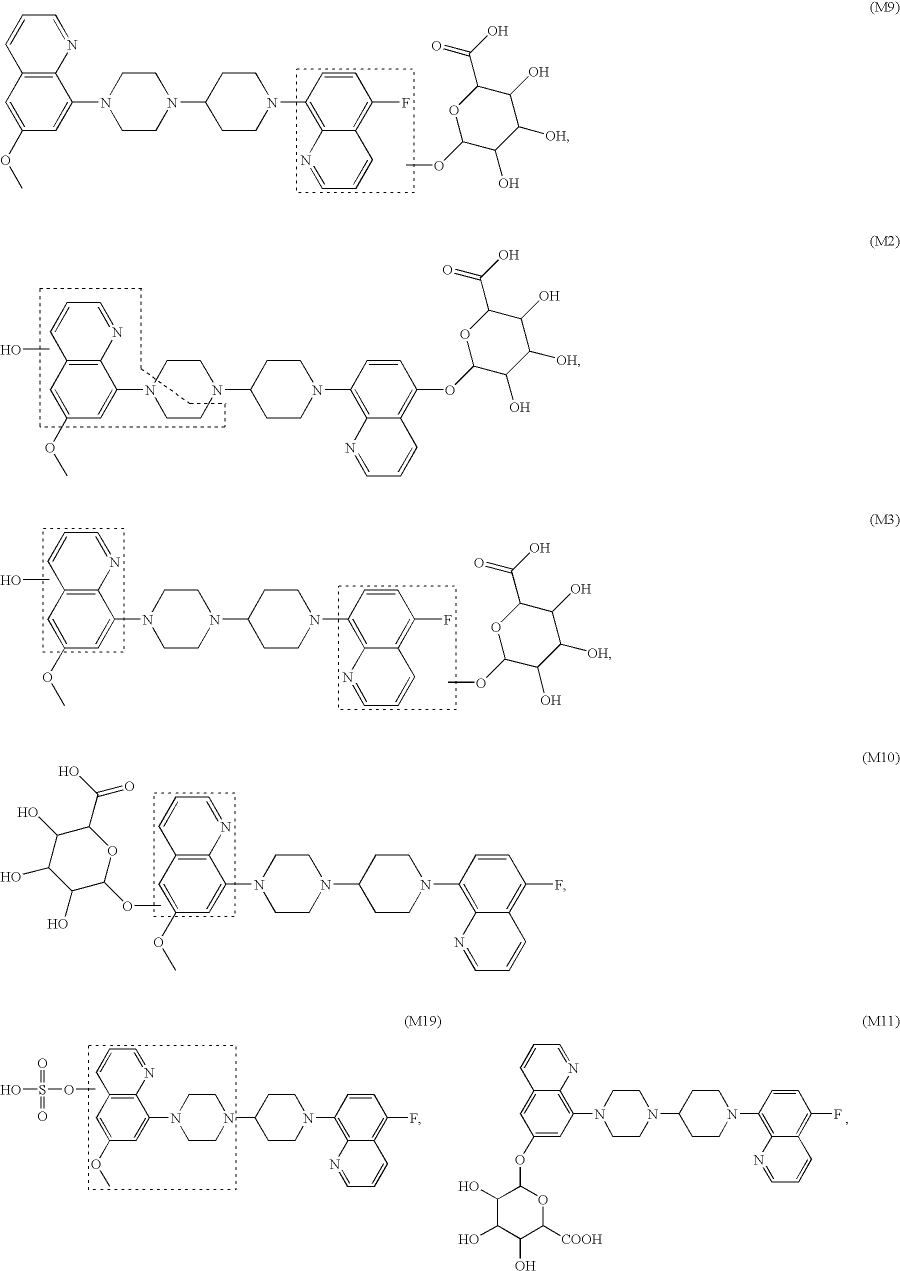

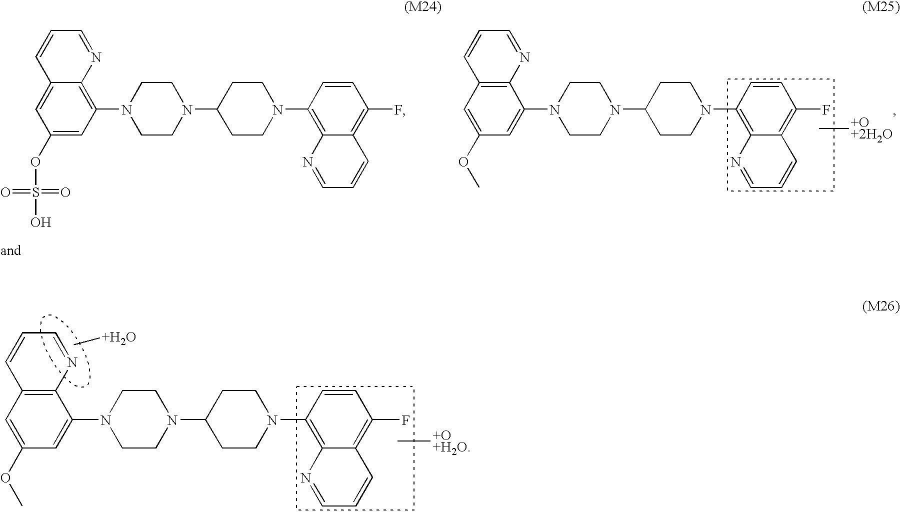

10. The metabolite of claim 5 in substantially pure form.

11. A pharmaceutical composition comprising at least one metabolite of claim 5 and a pharmaceutically acceptable carrier, diluent, or excipient.

12. A method of preparing a purified and isolated metabolite of 5-Fluoro-8-{4-[4-(6-methoxyquinolin-8-yl)piperazin-1-yl]piperidin-1-yl}quinoline, comprising:
(i) treating 5-Fluoro-8-{4-[4-(6-methoxyquinolin-8-yl)piperazin-1-yl]piperidin-1-yl}quinoline or its pharmaceutically acceptable salt with rat, mouse, dog, monkey or human liver microsomes;
(ii) treating 5-Fluoro-8-{4-[4-(6-methoxyquinolin-8-yl)piperazin-1-yl]piperidin-1-yl}quinoline or its pharmaceutically acceptable salt with rat, mouse, dog, monkey or human liver S9 fractions; or
(iii) treating 5-Fluoro-8-{4-[4-(6-methoxyquinolin-8-yl)piperazin-1-yl]piperidin-1-yl}quinoline or its pharmaceutically acceptable salt with cryopreserved rat, dog, or human hepatocytes.

13. The method of claim 12, further comprising isolating said metabolite.

14. A method for preparing a compound of formula (M21), 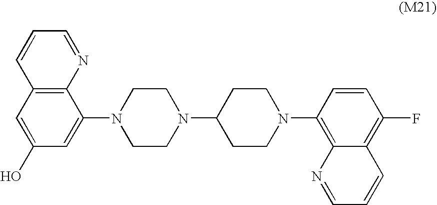
comprising demethylating the methoxy group of COMPOUND I,

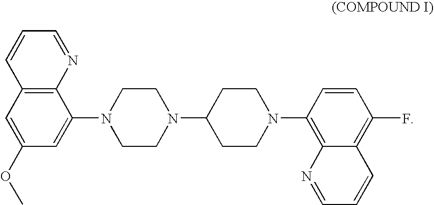

15. The method of claim 14, wherein said demethylating is achieved by contacting COMPOUND I with an acid.

16. The method of claim 15, wherein said acid is Me₃SiI, BBr₃, BF₃-Et₂, MeSSiMe₃, PhSSiMe₃, AlCl₃, AlBr₃, t-BuCOCl, AcCl, Ac₂O & FeCl₃, Me₂BBr, BI₃-Et₂NPh, TMSCl, or RuCl₃.

17. The method of claim 15, wherein said acid is AlCl₃.

18. A compound of formula (M21), 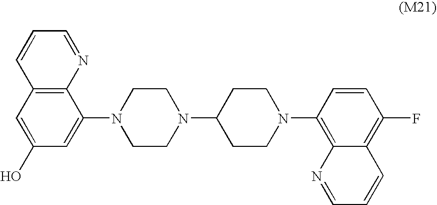
prepared by the method of claim 14.

19. A method for preparing a compound of formula (M21), 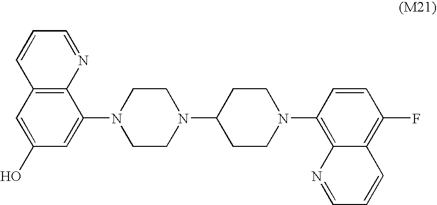
comprising:
(i) contacting a compound of formula (A), 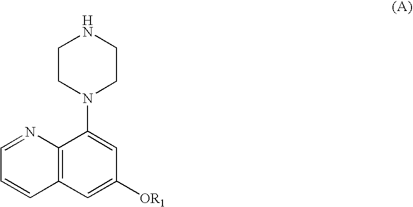
wherein R₁ is a hydroxyl protecting group;
with a compound of formula (B), 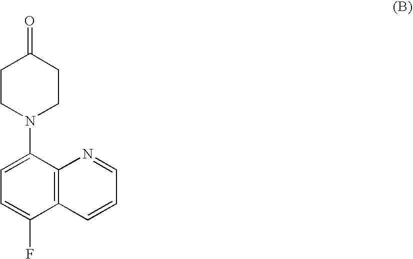
to provide a compound of formula (C); and 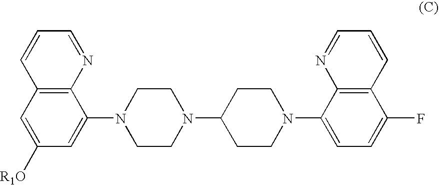
(ii) removing the hydroxyl protecting group R₁ of the compound of formula (C) to provide the compound of formula (M21).

20. A compound of formula (M21), 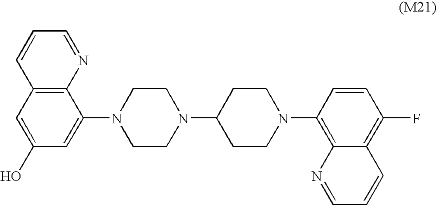
prepared by the method of claim 19.

21. A method for treating a 5-HT_{1A}-related disorder to a patient in need thereof, the method comprising administering to the patient a therapeutically effective amount of at least one metabolite of claim 5.

22. The method of claim 21, wherein the 5-HT_{1A}-related disorder is a cognition-related disorder or an anxiety-related disorder.

23. The method of claim 22, wherein the cognition-related disorder is dementia, Parkinson's disease, Huntington's disease, Alzheimer's disease, cognitive deficits associated with Alzheimer's disease, mild cognitive impairment, or schizophrenia.

24. The method of claim 22, wherein the anxiety-related disorder is attention deficit disorder, obsessive compulsive disorder, substance addiction, withdrawal from substance addiction, premenstrual dysphoric disorder, social anxiety disorder, anorexia nervosa, or bulimia nervosa.

25. The method of claim 21, further comprising administering a second therapeutic agent.

26. The method of claim 25, wherein the second therapeutic agent is an anti-depressant agent, an anti-anxiety agent, anti-psychotic agent, or a cognitive enhancer.

27. The method of claim 25, wherein the second therapeutic agent is a selective serotonin reuptake inhibitor, an SNRI, or a cholinesterase inhibitor.

28. A method for treating Alzheimer's disease, mild cognitive impairment, or depression to a patient in need thereof, the method comprising administering to the patient a therapeutically effective amount of the metabolite of claim 5.

29. A method for treating sexual dysfunction associated with drug treatment, and/or improving sexual function in a patient in need thereof, the method comprising administering to the patient a therapeutically effective amount of the metabolite of claim 5.

30. A radiolabeled compound of formula (G), or an enantiomer, diastereomer, tautomer, or pharmaceutically acceptable salt or solvate thereof: 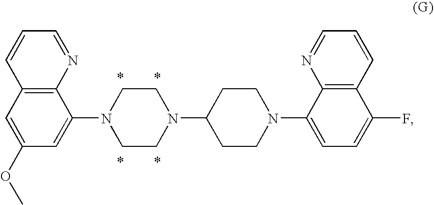
wherein each * represents a carbon-14.

31. A radiolabeled compound of formula (G), or a trisuccinate salt or solvate thereof: 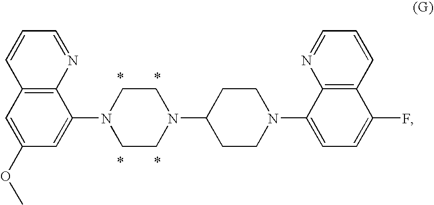
wherein each * represents a carbon-14.

32. A method for preparing a radiolabeled compound of formula (F), wherein each * represents a carbon-14, 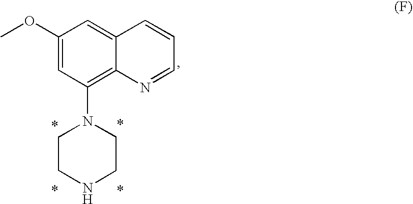
comprising contacting a compound of formula (D), 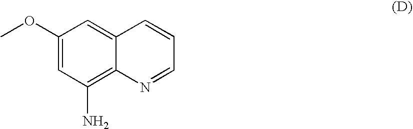
with a radiolabeled compound of formula (E) or a pharmaceutically acceptable salt thereof, wherein each * represents a carbon-14,

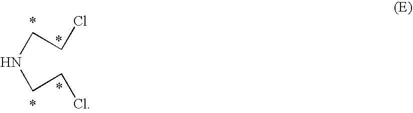

33. The method of claim 32, further comprising contacting a compound of formula (F) with a compound of formula (B), 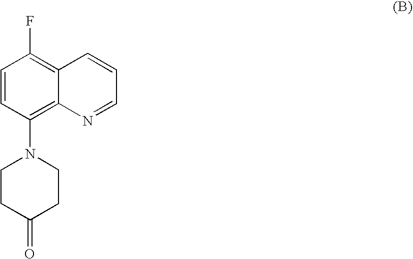
to provide a radiolabeled compound of formula (G), wherein each * represents a carbon-14, [chemical structure]
